SEQUENCE LISTING (249 sequences)

SEQ ID NO: 1 - protein, 117 aa, synthetic construct
QVQLKQSGPGLVQPSQSLSITCTVSGFSLTNYGVHWVRQSPGKGLEWLGVIWSGGSTDYNAAFVSRLSISRDNSKSQVFFKMNSLQADDTAIYYCARSGKWYYFDYWGQGTTLTVSS

SEQ ID NO: 2 - protein, 117 aa, synthetic construct
QVQLKESGPGLVQPSETLSLTCTVSGFSITNYGVHWIRQSPGKGLEWLGVIWSGGSTDYNAAFVSRVTISRDNSKSQVFFKLNSVQADDTAVYYCARSGKWYYFDYWGQGTTVTVSS

SEQ ID NO: 3 - protein, 117 aa, synthetic construct
QVQLQESGPGLVKPSETLSLTCTVSGFSITNYGVHWIRQPPGKGLEWLGVIWSGGSTDYNASLVSRVTISRDTSKSQFFLKLNSVQADDTAVYYCARSGKWYYFDYWGQGTTVTVSS

SEQ ID NO: 4 - protein, 117 aa, synthetic construct
QVQLQESGPGLVKPSETLSLTCTVSGGSITNYGVHWIRQPPGKGLEWLGVIWSGGSTDYNPSLKSRVTISVDTSKNQFSLKLSSVTAADTAVYYCARSGKWYYFDYWGQGTLVTVSS

SEQ ID NO: 5 - protein, 117 aa, synthetic construct
QVQLQQSGPGLVKPSETLSLTCTVSGFSLTNYGVHWVRQPPGKGLEWLGVIWSGGSTDYNPSLKSRLTISRDTSKNQVSFKMSSVTAADTAVYYCARSGKWYYFDYWGQGTLVTVSS

SEQ ID NO: 6 - protein, 117 aa, synthetic construct
QVQLQESGPGLVKPSETLSLTCTVSGGSLTNYGVHWVRQPPGKGLEWIGVIWSGGSTNYNPSLKSRVTISRDTSKNQVSLKLSSVTAADTAVYYCARSGKWYYFDYWGQGTLVTVSS

SEQ ID NO: 7 - protein, 116 aa, synthetic construct
QVQLKQSGPGLVQPSQSLSITCTVSGFSLTTYGVHWVRQSPGKGLEWLGVIWSGGSTDCNGAFISRLSIIKDNSKSQVFFKMNSLQADDTAIYYCARMTHWYFDVWGTGTTVTVSS

SEQ ID NO: 8 - protein, 116 aa, synthetic construct
QVQLKQSGPGLVQPSQSLSITCTVSGFSLTTYGVHWVRQSPGKGLEWLGVIWSGGSTDCNGAFISRLSIIKDNSKSQVFFKMNSLQADDTAIYYCARLTHWYFDVWGTGTTVTVSS

SEQ ID NO: 9 - protein, 116 aa, synthetic construct
QVQLKQSGPGLVQPSQSLSITCTVSGFSLTTYGVHWVRQSPGKGLEWLGVIWSGGSTDCNGAFISRLSIIKDNSKSQVFFKMNSLQADDTAIYYCARHTHWYFDVWGTGTTVTVSS

SEQ ID NO: 10 - protein, 116 aa, synthetic construct
QVQLKQSGPGLVQPSQSLSITCTVSGFSLTTYGVHWVRQSPGKGLEWLGVIWSGGSTDCNGAFISRLSIIKDNSKSQVFFKMNSLQADDTAIYYCARQTHWYFDVWGTGTTVTVSS

SEQ ID NO: 11 - protein, 116 aa, synthetic construct
QVQLKQSGPGLVQPSQSLSITCTVSGFSLTTYGVHWVRQSPGKGLEWLGVIWSGGSTDCNGAFISRLSIIKDNSKSQVFFKMNSLQADDTAIYYCARVTHWYFDVWGTGTTVTVSS

SEQ ID NO: 12 - protein, 116 aa, synthetic construct
QVQLKQSGPGLVQPSQSLSITCTVSGFSLTTYGVHWVRQSPGKGLEWLGVIWSGGSTDCNGAFISRLSIIKDNSKSQVFFKMNSLQADDTAIYYCARTTHWYFDVWGTGTTVTVSS

SEQ ID NO: 13 - protein, 116 aa, synthetic construct
QVQLKQSGPGLVQPSQSLSITCTVSGFSLTTYGVHWVRQSPGKGLEWLGVIWSGGSTDCNGAFISRLSIIKDNSKSQVFFKMNSLQADDTAIYYCARKTHWYFDVWGTGTTVTVSS

SEQ ID NO: 14 - protein, 116 aa, synthetic construct
QVQLKQSGPGLVQPSQSLSITCTVSGFSLTTYGVHWVRQSPGKGLEWLGVIWSGGNTDYNAAFISRLSISKDNSKSQVFFKMNSLQADDTAIYYCARMTHWYFDVWGTGTTVTVSS

SEQ ID NO: 15 - protein, 116 aa, synthetic construct
QVQLKQSGPGLVQPSQSLSITCTVSGFSLSTYGVHWVRQSPGKGLEWLGVIWSGGSTDYNAAFISRLTISKDNSKSQIFFKMNSLQADDTAIYYCARMMSWYFDVWGTGTTVTVSS

SEQ ID NO: 16 - protein, 116 aa, synthetic construct
QVQLKQSGPGLVQPSQSLSITCTVSGFSLATYGVHWVRQSPGKGLEWLGVIWSGGSTDYNAAFISRLSISKDNSKSQVFFKMNSLQADDTAIYYCARNSHWYFDVWGTGTTVTVSS

SEQ ID NO: 17 - protein, 119 aa, synthetic construct
QVQLQQPGAELVKPGASVKLSCKASGYTFTTYWMHWVKQRPGRGLEWIGGIDPYGAHIKYNEMFKTKATLTVDKPSSTAYMQLSSLTSEDSAVYYCARGDYSNWWWTYWGQGTLVTVSA

SEQ ID NO: 18 - protein, 106 aa, synthetic construct
DIQMTQSPASLSVSVGETVTITCRASENIYSNLAWYQQKQGKSPQLLVYAATNLADGVPSRFSGSGSGTQYSLKINSLQSEDFGSYYCQHFWGTPTFGGGTKLEIK

SEQ ID NO: 19 - protein, 106 aa, synthetic construct
DIQMTQSPASLSASVGDTVTITCRASQNIYSNLAWYQQKQGKSPQLLVYAATNLADGVPSRFSGSGSGTQYTLKINSLQSEDFGTYYCQHFWGTPTFGGGTKVEIK

SEQ ID NO: 20 - protein, 106 aa, synthetic construct
DIQMTQSPSSLSASVGDTVTITCRASQNIYSNLAWYQQKQGKAPKLLVYAATNLADGVPSRFSGSGSGTDFTLKINSLQPEDFATYYCQHFWGTPTFGGGTKVEIK

SEQ ID NO: 21 - protein, 106 aa, synthetic construct
DIQMTQSPSSLSASVGDRVTITCRASQSIYSNLAWYQQKPGKAPKLLVYAATNLASGVPSRFSGSGSGTDFTLTISSLQPEDFATYYCQHFWGTPTFGGGTKVEIK

SEQ ID NO: 22 - protein, 106 aa, synthetic construct
DIQMTQSPSSLSASVGDRVTITCRASENIYSNLAWYQQKPGKAPKLLVYAATNLADGVPSRFSGSGSGTDYTLTISSLQPEDFATYYCQHFWGTPTFGGGTKVEIK

SEQ ID NO: 23 - protein, 106 aa, synthetic construct
DIQMTQSPSSLSASVGDRVTITCRASQSIYSNLAWYQQKPGKAPKLLVYAATNLQSGVPSRFSGSGSGTDFTLTISSLQPEDFATYYCQHFWGTPTFGGGTKVEIK

SEQ ID NO: 24 - protein, 107 aa, synthetic construct
DIVMTQSQKFMSTTVGDRVSITCKASQNVNTAVAWYQQKPGQSPKLLIYSASYRYTGVPDRFTGSGSGTDFTLTISNMQSEDLADYFCQQYSNYPLTFGAGTKLELK

SEQ ID NO: 25 - protein, 107 aa, synthetic construct
DIVMTQSQKFMSTTVGDRVSITCKASQNVQTAVAWYQQKPGQSPKLLIYSASYRYTGVPDRFTGSGSGTDFTLTISNMQSEDLADYFCQQYSNYPLTFGAGTKLELK

SEQ ID NO: 26 - protein, 107 aa, synthetic construct
DIVMTQSQKFMSTTVGDRVSITCKASQNVYTAVAWYQQKPGQSPKLLIYSASYRYTGVPDRFTGSGSGTDFTLTISNMQSEDLADYFCQQYSNYPLTFGAGTKLELK

SEQ ID NO: 27 - protein, 107 aa, synthetic construct
DIVMTQSQKFMSTTVGDRVSITCKASQNVVTAVAWYQQKPGQSPKLLIYSASYRYTGVPDRFTGSGSGTDFTLTISNMQSEDLADYFCQQYSNYPLTFGAGTKLELK

SEQ ID NO: 28 - protein, 107 aa, synthetic construct
DIVMTQSQKFMSTTVGDRVSITCKASQNVVTAVAWYQQKPGQSPKLLIYSASNRYTGVPDRFTGSGSGTDFTLTISYMQSEDLADYFCQQYSSYPLTFGAGTKLELK

SEQ ID NO: 29 - protein, 107 aa, synthetic construct
DIVMTQSQKFMSTSVGDRVSVTCKASQNVVTNVAWYQQKPGQSPKALIYSASYRYSGVPDRFTGSGSGTDFTLTISNVQSEDLAEYFCQQYNSYPLTFGAGTKLELK

SEQ ID NO: 30 - protein, 107 aa, synthetic construct
DIVMTQSQKFMSTSVGDRVSVTCKASQNVVTNVAWYQQKPGQSPKALIYSASYRYSGVPDRFTGSGSGTDFTLTISHVQSEDLAEYFCHQYNNYPYTFGGGTKLEIK

SEQ ID NO: 31 - protein, 107 aa, synthetic construct
DIVMTQSQKFMSTTVGDRVSITCKASQNVGTAVAWYQQIPGQSPKLLIYSASNRYTGVPDRFTGSGSGTDFTLTITNMQSEDLADYFCQHYSSYPWTFGGGTKLEIK

SEQ ID NO: 32 - protein, 8 aa, synthetic construct
GFSLTNYG

SEQ ID NO: 33 - protein, 7 aa, synthetic construct
IWSGGST

SEQ ID NO: 34 - protein, 11 aa, synthetic construct
ARSGKWYYFDY

SEQ ID NO: 35 - protein, 8 aa, synthetic construct
GFSITNYG

SEQ ID NO: 36 - protein, 8 aa, synthetic construct
GGSITNYG

SEQ ID NO: 37 - protein, 8 aa, synthetic construct
GGSLTNYG

SEQ ID NO: 38 - protein, 8 aa, synthetic construct
GFSLTTYG

SEQ ID NO: 39 - protein, 10 aa, synthetic construct
ARMTHWYFDV

SEQ ID NO: 40 - protein, 10 aa, synthetic construct
ARLTHWYFDV

SEQ ID NO: 41 - protein, 10 aa, synthetic construct
ARHTHWYFDV

SEQ ID NO: 42 - protein, 10 aa, synthetic construct
ARQTHWYFDV

SEQ ID NO: 43 - protein, 10 aa, synthetic construct
ARVTHWYFDV

SEQ ID NO: 44 - protein, 10 aa, synthetic construct
ARTTHWYFDV

SEQ ID NO: 45 - protein, 10 aa, synthetic construct
ARKTHWYFDV

SEQ ID NO: 46 - protein, 8 aa, synthetic construct
GFSLSTYG

SEQ ID NO: 47 - protein, 10 aa, synthetic construct
ARMMSWYFDV

SEQ ID NO: 48 - protein, 8 aa, synthetic construct
GFSLATYG

SEQ ID NO: 49 - protein, 10 aa, synthetic construct
ARNSHWYFDV

SEQ ID NO: 50 - protein, 8 aa, synthetic construct
GYTFTTYW

SEQ ID NO: 51 - protein, 8 aa, synthetic construct
IDPYGAHI

SEQ ID NO: 52 - protein, 12 aa, synthetic construct
ARGDYSNWWWTY

SEQ ID NO: 53 - protein, 6 aa, synthetic construct
ENIYSN

SEQ ID NO: 54 - skipped

SEQ ID NO: 55 - protein, 8 aa, synthetic construct
QHFWGTPT

SEQ ID NO: 56 - protein, 6 aa, synthetic construct
QNIYSN

SEQ ID NO: 57 - protein, 6 aa, synthetic construct
QSIYSN

SEQ ID NO: 58 - protein, 6 aa, synthetic construct
QNVNTA

SEQ ID NO: 59 - skipped

SEQ ID NO: 60 - protein, 9 aa, synthetic construct
QQYSNYPLT

SEQ ID NO: 61 - protein, 6 aa, synthetic construct
QNVQTA

SEQ ID NO: 62 - protein, 6 aa, synthetic construct
QNVYTA

SEQ ID NO: 63 - protein, 6 aa, synthetic construct
QNVVTA

SEQ ID NO: 64 - protein, 9 aa, synthetic construct
QQYSSYPLT

SEQ ID NO: 65 - protein, 6 aa, synthetic construct
QNVVTN

SEQ ID NO: 66 - protein, 9 aa, synthetic construct
QQYNSYPLT

SEQ ID NO: 67 - protein, 9 aa, synthetic construct
HQYNNYPYT

SEQ ID NO: 68 - protein, 6 aa, synthetic construct
QNVGTA

SEQ ID NO: 69 - protein, 9 aa, synthetic construct
QHYSSYPWT

SEQ ID NO: 70 - protein, 338 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSLCTPSRGS

SEQ ID NO: 71 - protein, 336 aa, synthetic construct
ASTKGPSVFPLAPSSKSTSGGTAALGCLVKDYFPEPVTVSWNSGALTSGVHTFPAVLLCTPSRQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 336 aa, synthetic construct
ASTKGPSVFPLAPSSKSTSGGTAALGCLVKDYFPEPVTVSWNSGALTSGVHTFPAVLQSSLCTPSRGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 336 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSLCTPSRNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 336 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPLCTPSRELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 336 aa, synthetic construct
ASTKGPSVFPLAPSSKSTSGGTAALGCLVKDYFPEPVTVSWNSGALTSGVHTFPAVLLCTPSRQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 336 aa, synthetic construct
ASTKGPSVFPLAPSSKSTSGGTAALGCLVKDYFPEPVTVSWNSGALTSGVHTFPAVLQSSLCTPSRGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 336 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSLCTPSRNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 336 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPLCTPSRELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 336 aa, synthetic construct
ASTKGPSVFPLAPSSKSTSGGTAALGCLVKDYFPEPVTVSWNSGALTSGVHTFPAVLLCTPSRQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 336 aa, synthetic construct
ASTKGPSVFPLAPSSKSTSGGTAALGCLVKDYFPEPVTVSWNSGALTSGVHTFPAVLQSSLCTPSRGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 336 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSLCTPSRNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 336 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPLCTPSRELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 336 aa, synthetic construct
ASTKGPSVFPLAPSSKSTSGGTAALGCLVKDYFPEPVTVSWNSGALTSGVHTFPAVLLCTPSRQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 336 aa, synthetic construct
ASTKGPSVFPLAPSSKSTSGGTAALGCLVKDYFPEPVTVSWNSGALTSGVHTFPAVLQSSLCTPSRGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 336 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSLCTPSRNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 336 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPLCTPSRELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 107 aa, Homo sapiens [sequence omitted]

SEQ ID NO: 88 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 330 aa, Homo sapiens [sequence omitted]

SEQ ID NO: 90 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 91 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 92 - protein, 326 aa, Homo sapiens
STKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 93 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPDRDLCGCYSVSSVLSGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 94 - skipped

SEQ ID NO: 95 - protein, 107 aa, Homo sapiens
RTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 96 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 97 - protein, 107 aa, Mus musculus
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 98 - protein, 107 aa, Rattus norvegicus
RADAAPTVSIFPPSMEQLTSGGATVVCFVNNFYPRDISVKWKIDGSEQRDGVLDSVTDQDSKDSTYSMSSTLSLTKVEYERHNLYTCEVVHKTSSSPVVKSFNRNEC

SEQ ID NO: 99 - protein, 510 aa, Homo sapiens
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 100 - protein, 10 aa, synthetic construct
  VARIANT 1..10: note = Wherein up to 8 copies of G can be deleted
GGGGGGGGGG

SEQ ID NO: 101-102 - skipped (2 entries)

SEQ ID NO: 103 - protein, 6 aa, synthetic construct
  SITE 2: note = Wherein X can be any naturally occurring amino acid
LXTPSR

SEQ ID NO: 104 - protein, 6 aa, synthetic construct
LCTPSR

SEQ ID NO: 105 - protein, 6 aa, synthetic construct
LSTPSR

SEQ ID NO: 106 - protein, 6 aa, synthetic construct
VCTPSR

SEQ ID NO: 107 - protein, 6 aa, synthetic construct
LCSPSR

SEQ ID NO: 108 - protein, 6 aa, synthetic construct
LCAPSR

SEQ ID NO: 109 - protein, 6 aa, synthetic construct
LCVPSR

SEQ ID NO: 110 - protein, 6 aa, synthetic construct
LCGPSR

SEQ ID NO: 111 - protein, 6 aa, synthetic construct
ICTPAR

SEQ ID NO: 112 - protein, 6 aa, synthetic construct
LCTPSK

SEQ ID NO: 113 - protein, 6 aa, synthetic construct
MCTPSK

SEQ ID NO: 114 - protein, 6 aa, synthetic construct
VCTPSK

SEQ ID NO: 115 - protein, 6 aa, synthetic construct
LCSPSK

SEQ ID NO: 116 - protein, 6 aa, synthetic construct
LCAPSK

SEQ ID NO: 117 - protein, 6 aa, synthetic construct
LCVPSK

SEQ ID NO: 118 - protein, 6 aa, synthetic construct
LCGPSK

SEQ ID NO: 119 - protein, 6 aa, synthetic construct
LCTPSA

SEQ ID NO: 120 - protein, 6 aa, synthetic construct
ICTPAA

SEQ ID NO: 121 - protein, 6 aa, synthetic construct
MCTPSA

SEQ ID NO: 122 - protein, 6 aa, synthetic construct
VCTPSA

SEQ ID NO: 123 - protein, 6 aa, synthetic construct
LCSPSA

SEQ ID NO: 124 - protein, 6 aa, synthetic construct
LCAPSA

SEQ ID NO: 125 - protein, 6 aa, synthetic construct
LCVPSA

SEQ ID NO: 126 - protein, 6 aa, synthetic construct
LCGPSA

SEQ ID NO: 127-128 - skipped (2 entries)

SEQ ID NO: 129 - protein, 10 aa, synthetic construct
QKSLSLSPGK

SEQ ID NO: 130 - protein, 12 aa, synthetic construct
  MOD_RES 6: note = FORMYLATION
SPGSLGTPSRGS

SEQ ID NO: 131 - protein, 4 aa, synthetic construct
SPGK

SEQ ID NO: 132 - protein, 6 aa, synthetic construct
KVDNAL

SEQ ID NO: 133 - protein, 6 aa, synthetic construct
QSGNSQ

SEQ ID NO: 134 - protein, 17 aa, synthetic construct
  MOD_RES 7: note = FORMYLATION
KVDNALGTPSRQSGNSQ

SEQ ID NO: 135 - protein, 6 aa, synthetic construct
SWNSGA

SEQ ID NO: 136 - protein, 6 aa, synthetic construct
GVHTFP

SEQ ID NO: 137 - protein, 18 aa, synthetic construct
  MOD_RES 8: note = FORMYLATION
SWNSGALGTPSRGVHTFP

SEQ ID NO: 138 - protein, 6 aa, synthetic construct
ASTKGP

SEQ ID NO: 139 - protein, 7 aa, synthetic construct
KSTSGGT

SEQ ID NO: 140 - protein, 4 aa, synthetic construct
PEPV

SEQ ID NO: 141 - protein, 8 aa, synthetic construct
NSGALTSG

SEQ ID NO: 142 - protein, 21 aa, synthetic construct
NSGALTSGVHTFPAVLQSSGL

SEQ ID NO: 143 - protein, 5 aa, synthetic construct
QSSGL

SEQ ID NO: 144 - protein, 4 aa, synthetic construct
TQTY

SEQ ID NO: 145 - protein, 5 aa, synthetic construct
HKPSN

SEQ ID NO: 146 - protein, 22 aa, synthetic construct
EPKSCDKTHTCPPCPAPELLGG

SEQ ID NO: 147 - protein, 5 aa, synthetic construct
FPPKP

SEQ ID NO: 148 - protein, 5 aa, synthetic construct
ISRTP

SEQ ID NO: 149 - protein, 9 aa, synthetic construct
DVSHEDPEV

SEQ ID NO: 150 - protein, 7 aa, synthetic construct
SHEDPEV

SEQ ID NO: 151 - protein, 9 aa, synthetic construct
DGVEVHNAK

SEQ ID NO: 152 - protein, 5 aa, synthetic construct
QYNST

SEQ ID NO: 153 - protein, 5 aa, synthetic construct
VLTVL

SEQ ID NO: 154 - protein, 7 aa, synthetic construct
NKALPAP

SEQ ID NO: 155 - protein, 9 aa, synthetic construct
SKAKGQPRE

SEQ ID NO: 156 - protein, 7 aa, synthetic construct
KAKGQPR

SEQ ID NO: 157 - protein, 10 aa, synthetic construct
PPSRKELTKN

SEQ ID NO: 158 - protein, 5 aa, synthetic construct
YPSDI

SEQ ID NO: 159 - protein, 7 aa, synthetic construct
NGQPENN

SEQ ID NO: 160 - protein, 10 aa, synthetic construct
TPPVLDSDGS

SEQ ID NO: 161 - protein, 19 aa, synthetic construct
HEALHNHYTQKSLSLSPGK

SEQ ID NO: 162 - protein, 6 aa, synthetic construct
SLSPGK

SEQ ID NO: 163 - skipped

SEQ ID NO: 164 - protein, 12 aa, synthetic construct
PCSRSTSESTAA

SEQ ID NO: 165 - protein, 5 aa, synthetic construct
FPEPV

SEQ ID NO: 166 - protein, 12 aa, synthetic construct
SGALTSGVHTFP

SEQ ID NO: 167 - protein, 6 aa, synthetic construct
QSSGLY

SEQ ID NO: 168 - protein, 5 aa, synthetic construct
VAGPS

SEQ ID NO: 169-171 - skipped (3 entries)

SEQ ID NO: 172 - protein, 5 aa, synthetic construct
VLTVV

SEQ ID NO: 173 - protein, 7 aa, synthetic construct
NKGLPAP

SEQ ID NO: 174 - protein, 9 aa, synthetic construct
SKTKGQPRE

SEQ ID NO: 175 - protein, 5 aa, synthetic construct
MTKNQ

SEQ ID NO: 176-177 - skipped (2 entries)

SEQ ID NO: 178 - protein, 10 aa, synthetic construct
TPPMLDSDGS

SEQ ID NO: 179 - protein, 4 aa, synthetic construct
GNVF

SEQ ID NO: 180-181 - skipped (2 entries)

SEQ ID NO: 182 - protein, 10 aa, synthetic construct
PCSRSTSGGT

SEQ ID NO: 183 - skipped

SEQ ID NO: 184 - protein, 19 aa, synthetic construct
SGALTSGVHTFPAVLQSSG

SEQ ID NO: 185 - skipped

SEQ ID NO: 186 - protein, 10 aa, synthetic construct
RVELKTPLGD

SEQ ID NO: 187 - protein, 7 aa, synthetic construct
CPRCPKP

SEQ ID NO: 188 - protein, 21 aa, synthetic construct
PKSCDTPPPCPRCPAPELLGG

SEQ ID NO: 189-193 - skipped (5 entries)

SEQ ID NO: 194 - protein, 10 aa, synthetic construct
PPSREEMTKN

SEQ ID NO: 195 - skipped

SEQ ID NO: 196 - protein, 8 aa, synthetic construct
SSGQPENN

SEQ ID NO: 197 - skipped

SEQ ID NO: 198 - protein, 7 aa, synthetic construct
HEALHNR

SEQ ID NO: 199 - skipped

SEQ ID NO: 200 - protein, 5 aa, synthetic construct
STKGP

SEQ ID NO: 201-204 - skipped (4 entries)

SEQ ID NO: 205 - protein, 14 aa, synthetic construct
CPAPEFLGGPSREF

SEQ ID NO: 206 - skipped

SEQ ID NO: 207 - protein, 5 aa, synthetic construct
SKIPA

SEQ ID NO: 208 - skipped

SEQ ID NO: 209 - protein, 7 aa, synthetic construct
NKGLPSS

SEQ ID NO: 210 - protein, 10 aa, synthetic construct
SKAKGQPREP

SEQ ID NO: 211 - protein, 10 aa, synthetic construct
PPSQEEMTKN

SEQ ID NO: 212-214 - skipped (3 entries)

SEQ ID NO: 215 - protein, 19 aa, synthetic construct
HEALHNHYTQKSLSLSLGK

SEQ ID NO: 216 - protein, 13 aa, synthetic construct
ASPTSPKVFPLSL

SEQ ID NO: 217 - protein, 5 aa, synthetic construct
QPDGN

SEQ ID NO: 218 - protein, 10 aa, synthetic construct
VQGFFPQEPL

SEQ ID NO: 219 - protein, 11 aa, synthetic construct
SGQGVTARNFP

SEQ ID NO: 220 - protein, 7 aa, synthetic construct
SGDLYTT

SEQ ID NO: 221 - protein, 4 aa, synthetic construct
PATQ

SEQ ID NO: 222 - protein, 4 aa, synthetic construct
HRPA

SEQ ID NO: 223 - protein, 5 aa, synthetic construct
LLGSE

SEQ ID NO: 224 - protein, 8 aa, synthetic construct
GLRDASGV

SEQ ID NO: 225 - protein, 10 aa, synthetic construct
SSGKSAVQGP

SEQ ID NO: 226 - protein, 4 aa, synthetic construct
GCYS

SEQ ID NO: 227 - protein, 4 aa, synthetic construct
CAEP

SEQ ID NO: 228 - protein, 24 aa, synthetic construct
SGNTFRPEVHLLPPPSEELALNEL

SEQ ID NO: 229 - protein, 5 aa, synthetic construct
ARGFS

SEQ ID NO: 230 - protein, 11 aa, synthetic construct
QGSQELPREKY

SEQ ID NO: 231 - protein, 4 aa, synthetic construct
AAED

SEQ ID NO: 232 - protein, 4 aa, synthetic construct
HEAL

SEQ ID NO: 233 - protein, 25 aa, synthetic construct
IDRLAGKPTHVNVSVVMAEVDGTCY

SEQ ID NO: 234 - protein, 6 aa, synthetic construct
RTVAAP

SEQ ID NO: 235 - protein, 5 aa, synthetic construct
YPREA

SEQ ID NO: 236 - protein, 4 aa, synthetic construct
PREA

SEQ ID NO: 237 - protein, 8 aa, synthetic construct
DNALQSGN

SEQ ID NO: 238 - protein, 9 aa, synthetic construct
TEQDSKDST

SEQ ID NO: 239 - protein, 6 aa, synthetic construct
HQGLSS

SEQ ID NO: 240 - protein, 4 aa, synthetic construct
RGEC

SEQ ID NO: 241 - protein, 6 aa, synthetic construct
QPKAAP

SEQ ID NO: 242 - protein, 7 aa, synthetic construct
DFYPGAV

SEQ ID NO: 243 - protein, 8 aa, synthetic construct
DSSPVKAG

SEQ ID NO: 244 - protein, 6 aa, synthetic construct
APTECS

SEQ ID NO: 245 - protein, 6 aa, synthetic construct
  MOD_RES 2: note = FORMYLATION
LGTPSR

SEQ ID NO: 246 - protein, 6 aa, synthetic construct
  MOD_RES 2: note = Formylated and coupled to moiety of interest
LGTPSR

SEQ ID NO: 247 - protein, 7 aa, synthetic construct
SLSLSPG

SEQ ID NO: 248 - protein, 16 aa, synthetic construct
  MOD_RES 10: note = FORMYLATION
SLSLSPGSLGTPSRGS

SEQ ID NO: 249 - protein, 8 aa, synthetic construct
SLSLSPGK

(19) United States
(12) Patent Application Publication
(10) Pub. No.: US 2025/0101098 A1
(43) Pub. Date: Mar. 27, 2025

(54) ANTIBODIES AND ANTIBODY CONJUGATES SPECIFIC FOR NECTIN-4 AND METHODS OF USE THEREOF

(71) Applicant: R.P. Scherer Technologies, LLC, Carson City, NV (US)

(72) Inventors: Dominick YEO, Carson City, NV (US); Maxine BAUZON, Hercules, CA (US); Fangjiu ZHANG, Lafayette, CA (US); Stepan CHUPRAKOV, Walnut Creek, CA (US); Yun C. KIM, Walnut Creek, CA (US); Robyn M. BARFIELD, Berkeley, CA (US); Penelope M. DRAKE, Castro Valley, CA (US)

(21) Appl. No.: 18/291,897

(22) PCT Filed: Jul. 28, 2022
(86) PCT No.: PCT/US2022/038720

§ 371 Date: Jan. 24, 2024

Related U.S. Application Data
(60) Provisional application No. 63/322,914, filed on Mar. 23, 2022.
(60) Provisional application No. 63/227,666, filed on Jul. 30, 2021.
(60) Provisional application No. 63/344,932, filed on May 23, 2022.

(51) Int. Cl. C07K 16/28; A61K 47/68; A61P 35/00

(52) U.S. Cl. CPC C07K 16/2803; A61K 47/68031; A61K 47/6851; A61P 35/00; C07K 2317/24; C07K 2317/732; C07K 2317/92

(57) ABSTRACT

The present disclosure provides antibodies specific for Nectin-4 and antibody conjugates e.g., antibody-drug conjugates (ADCs), comprising such antibodies. The disclosure also encompasses methods of production of such antibodies and antibody conjugates, as well as methods of using the same. Also provided are compositions that include the antibodies and antibody conjugates of the present disclosure, including in some instances, pharmaceutical compositions. In certain aspects, provided are methods of using the ADC that include administering to an individual having a cell proliferative disorder a therapeutically effective amount of the antibodies or antibody conjugates of the present disclosure.

### CROSS-REFERENCE TO RELATED APPLICATIONS

This application claims the benefit of priority to U.S. Provisional Application No. 63/227,666, filed Jul. 30, 2021, U.S. Provisional Application No. 63/322,914, filed Mar. 23, 2022, and U.S. Provisional Application No. 63/344,932, filed May 23, 2022, the disclosures of each of which are incorporated herein by reference.

### INTRODUCTION

The field of protein-small molecule therapeutic conjugates has advanced greatly, providing a number of clinically beneficial drugs with the promise of providing more in the years to come. Protein-conjugate therapeutics can provide several advantages, due to, for example, specificity, multiplicity of functions and relatively low off-target activity, resulting in fewer side effects. Chemical modification of proteins may extend these advantages by rendering them more potent, stable, or multimodal.

A number of standard chemical transformations are commonly used to create and manipulate post-translational modifications on proteins. A number of methods are available to modify the side chains of certain amino acids selectively. For example, carboxylic acid side chains (aspartate and glutamate) may be targeted by initial activation with a water-soluble carbodiimide reagent and subsequent reaction with an amine. Similarly, lysine can be targeted through the use of activated esters or isothiocyanates, and cysteine thiols can be targeted with maleimides and α-halo-carbonyls.

One significant obstacle to the creation of a chemically altered protein therapeutic or reagent is the production of the protein in a biologically active, homogenous form. Conjugation of a drug or detectable label to a polypeptide can be difficult to control, resulting in a heterogeneous mixture of conjugates that differ in the number of drug molecules attached and in the position of chemical conjugation. In some instances, it may be desirable to control the site of conjugation and/or the drug or detectable label conjugated to the polypeptide using the tools of synthetic organic chemistry to direct the precise and selective formation of chemical bonds on a polypeptide.

Nectin-4 (also known as Nectin Cell Adhesion Molecule 4) is a member of the Nectin family. Nectin-4 is a type I transmembrane protein and is a member of the nectin family of adhesion proteins. Nectin family of adhesion proteins are structurally related and exhibit three conserved immunoglobulin-like domains (V, C, and C) in their extracellular regions. Nectin-4 has a molecular weight of about 55 kDa with the molecular weight of the extra-cellular domain of about 36 kDa.

Nectin-4 can form homodimers or heterodimers with Nectin-1. Nectin-4 regulates several cellular activities such as movement, proliferation, differentiation, polarization, and entry of viruses. While other nectin family members are widely expressed in adult tissues, Nectin-4 is primarily confined to the embryo and placenta. In addition, Nectin-4 is overexpressed in a variety of solid tumors, such as ovarian, ductal breast carcinoma, lung adenocarcinoma, and pancreatic cancer. Nectin-4 is emerging as a metastasis-associated protein and may be associated with disease progression and poor prognosis.

### SUMMARY

The present disclosure provides antibodies specific for Nectin-4 and antibody conjugates (e.g., antibody-drug conjugates (ADCs)) comprising such antibodies. The disclosure also encompasses methods of production of such antibodies and antibody conjugates, as well as methods of using the same. Embodiments of each are described in more detail in the sections below. Also provided are compositions that include the antibodies and ADC of the present disclosure, including in some instances, pharmaceutical compositions. In certain aspects, provided are methods of using the ADC that include administering to an individual having a cell proliferative disorder a therapeutically effective amount of the ADC of the present disclosure.

### BRIEF DESCRIPTION OF THE SEQUENCES

SEQ ID NOs: 1 to 17: Heavy chains of the antibodies disclosed herein.

SEQ ID NOs: 18 to 31: Light chains of the antibodies disclosed herein.

SEQ ID NOs: 32 to 69: CDRs of heavy and light chains of the antibodies disclosed herein.

SEQ ID NOs: 70 to 86: Heavy chain constant regions of the antibodies disclosed herein.

SEQ ID NOs: 87 and 88: IgG1 heavy and light chains.

SEQ ID NOs: 89 to 93: Heavy chain constant regions of different Ig isotypes.

SEQ ID NOs: 94 to 98: Light chain constant regions of different types and organisms.

SEQ ID NO: 99: Sequence of human Nectin-4 protein.

SEQ ID NOs: 100 to 101: Flexible linkers including glycine polymers.

SEQ ID NOs: 102 to 126: Examples of sulfatase motifs, before conversion with Formylglycine Generating Enzyme (FGE).

SEQ ID NOs: 127 to 128 and 245 to 246: Examples of sulfatase motifs, after conversion with FGE.

SEQ ID NO: 129: Amino acid sequence in heavy chain constant region where sulfatase motif is inserted.

SEQ ID NOs: 130 to 244: Sequences within the constant regions of different immunoglobulins.

### BRIEF DESCRIPTION OF THE FIGURES

FIG. 1 shows chimeric anti-Nectin-4 antibody binding to recombinant human Nectin-4 protein.

FIG. 2 shows chimeric anti-Nectin-4 antibody binding to recombinant human Nectin-4 protein.

FIG. 3 shows anti-Nectin-4 antibody binding to human Nectin and Nec family members.

FIG. 4 shows 12E11 antibody clone variant binding to human Nectin and Nec family members.

FIG. 5 shows 12E11 antibody clone variant binding to human Nectin and NecI family members.

FIG. 6 shows humanized 5D9 variant binding to human Nectin-4.

FIG. 7 shows humanized 5D9 variant binding to human Nectin-4.

FIG. 8 shows humanized 5D9 variant binding to human Nectin-4.

FIG. 9 shows humanized 5D9 variant binding to human Nectin-4.

FIG. 10 shows in vitro potency of chimeric anti-Nectin-4 ADCs or controls against HEK cells overexpressing human Nectin-4.

FIG. 11 shows in vitro potency of chimeric anti-Nectin-4 ADCs or control against HEK cells overexpressing human Nectin-4.

FIG. 12 shows in vitro potency of chimeric anti-Nectin-4 ADCs or control against HEK cells overexpressing human Nectin-4.

FIG. 13 shows in vitro potency of chimeric anti-Nectin-4 ADCs or controls against HEK cells overexpressing human Nectin-4.

FIG. 14 shows in vitro potency of humanized 5D9 variant anti-Nectin-4 ADCs or controls against HEK cells overexpressing human Nectin-4.

FIG. 15 shows in vitro potency of humanized 5D9 variant anti-Nectin-4 ADCs or controls against SK-BR-3 cells.

FIG. 16 shows in vitro potency of humanized 5D9 variant anti-Nectin-4 ADCs or controls against MDA-MB-468 cells.

FIG. 17 shows in vitro potency of humanized 5D9 variant anti-Nectin-4 ADCs or controls against HEK cells overexpressing human Nectin-4.

FIG. 18 shows in vitro potency of humanized 5D9 variant anti-Nectin-4 ADCs or controls against SK-BR-3 cells.

FIG. 19 in vitro potency of humanized 5D9 variant anti-Nectin-4 ADCs or controls against MDA-MB-468 cells.

FIG. 20 shows in vitro potency of humanized 5D9 variant anti-Nectin-4 ADCs or controls against HEK cells overexpressing human Nectin-4.

FIG. 21 shows in vitro potency of humanized 5D9 variant anti-Nectin-4 ADCs or controls against SK-BR-3 cells.

FIG. 22 shows in vitro potency of humanized 5D9 variant anti-Nectin-4 ADCs or controls against MDA-MB-468 cells.

FIG. 23 shows in vitro potency of humanized 5D9 variant anti-Nectin-4 ADCs or controls against HEK cells overexpressing human Nectin-4.

FIG. 24 shows in vitro potency of humanized 5D9 variant anti-Nectin-4 ADCs or controls against SK-BR-3 cells.

FIG. 25 shows in vitro potency of humanized 5D9 variant anti-Nectin-4 ADCs or controls against MDA-MB-468 cells.

FIG. 26 shows in vitro potency of humanized 5D9 variant anti-Nectin-4 ADCs or controls against HEK cells overexpressing human Nectin-4.

FIG. 27 shows in vitro potency of h5D9 variant anti-Nectin-4 ADCs or controls against SK-BR-3 cells.

FIG. 28 shows in vitro potency of humanized 5D9 variant anti-Nectin-4 ADCs or controls against MDA-MB-468 cells.

FIG. 29 shows in vivo efficacy of a double-tagged aldehyde tagged Enfortumab antibody conjugated to Compound 20 against NCI-H1781 xenograft model.

FIG. 30A shows a site map showing possible modification sites for generation of an aldehyde tagged Ig polypeptide. The upper sequence is the amino acid sequence of the conserved region of an IgG1 light chain polypeptide (SEQ ID NO: 87) and shows possible modification sites in an Ig light chain; the lower sequence is the amino acid sequence of the conserved region of an Ig heavy chain polypeptide (SEQ ID NO: 88) (GenBank Accession No. AAG00909) and shows possible modification sites in an Ig heavy chain. The heavy and light chain numbering is based on the full-length heavy and light chains.

FIG. 30B depicts an alignment of Homo sapiens immunoglobulin heavy chain constant regions for IgG1 (SEQ ID NO: 89; GenBank P01857.1), IgG2 (SEQ ID NO: 90; GenBank P01859.2), IgG3 (SEQ ID NO: 91; GenBank P01860.2), IgG4 (SEQ ID NO: 92; GenBank AAB59394.1), and IgA (SEQ ID NO: 93; GenBank AAAT74070), showing modification sites at which aldehyde tags can be provided in an immunoglobulin heavy chain. The heavy and light chain numbering is based on the full heavy and light chains.

FIG. 30C depicts an alignment of immunoglobulin light chain constant regions, showing modification sites at which aldehyde tags can be provided in an immunoglobulin light chain. Seq1=Homo sapiens kappa light chain constant region; GenBank CAA75031.1; SEQ ID NO: 94. Seq2=Homo sapiens kappa light chain constant region; GenBank BAC0168.1; SEQ ID NO: 95. Seq3=Homo sapiens lambda light chain constant region; GenBank CAA75033; SEQ ID NO: 96. Seq4=Mus musculus light chain constant region; GenBank AAB09710.1; SEQ ID NO: 97. Seq5=Rattus norvegicus light chain constant region; GenBank AAD10133; SEQ ID NO: 98.

FIG. 30D depicts an alignment of immunoglobulin light chain constant regions, showing modification sites at which aldehyde tags can be provided in an immunoglobulin light chain. Seq1=Homo sapiens kappa light chain constant region; GenBank CAA75031.1; SEQ ID NO:52. Seq2=Homo sapiens kappa light chain constant region; GenBank BAC0168.1; SEQ ID NO:53. Seq3=Homo sapiens lambda light chain constant region; GenBank CAA75033; SEQ ID NO:54. Seq4=Mus musculus light chain constant region; GenBank AAB09710.1; SEQ ID NO:55. Seq5=Rattus norvegicus light chain constant region; GenBank AAD10133; SEQ ID NO:56.

FIG. 31 shows a graph of an NCI-H1781 xenograft study with a single 2.5 or 7.5 mg/kg intravenous dose of the listed anti-nectin-4 ADC on Day 0. VH4/VL1 Compound 8 (RED-601) and VH4/VL5 Compound 8 both use the internal 91N tag and deliver half the payload dose as compared to Padcev. The isotype control ADC had minimal activity.

FIG. 32 shows a graph of an NCI-H1781 xenograft study with a single 2.5 or 7.5 mg/kg intravenous dose of the listed anti-nectin-4 or isotype control ADC on Day 0. VH4/VL1 Compound 25 (RED-694) was made in a DAR4 format using the 91N tag and in a DAR8 format using the 91N/116E double tag combination. Padcev (generic) was included as a comparator. The isotype control Compound 25 ADC had minimal activity.

FIG. 33 shows a graph of an NCI-H1781 xenograft study with a single 2.5 or 7.5 mg/kg intravenous dose of the listed anti-nectin-4 or isotype control ADC on Day 0. VH4/VL5 Compound 25 (RED-694) was made in a DAR4 format using the 91N tag and in a DAR8 format using the 91N/116E double tag combination. Padcev (generic) was included as a comparator. The isotype control Compound 25 ADC had minimal activity.

FIG. 34. Double-tagged Nectin-4 VH4/VL1 antibody conjugated to Compound 8 yields a DAR of 3.74 as determined by PLRP.

FIG. 35. Double-tagged Nectin-4 VH4/VL1 antibody conjugated to Compound 8 is 98.5% monomeric as determined by SEC.

FIG. 36. Double-tagged Nectin-4 VH4/VL5 antibody conjugated to Compound 8 yields a DAR of 3.73 as determined by PLRP.

FIG. 37. Double-tagged Nectin-4 VH4/VL5 antibody conjugated to Compound 8 is 98.0% monomeric as determined by SEC.

FIG. 38. Double-tagged Nectin-4 VH4/VL1 antibody conjugated to Compound 25 yields a DAR of 6.89 as determined by PLRP.

FIG. 39. Double-tagged Nectin-4 VH4/VL1 antibody conjugated to Compound 25 is 98.7% monomeric as determined by SEC.

FIG. 40. Double-tagged Nectin-4 VH4/VL5 antibody conjugated to Compound 25 yields a DAR of 6.86 as determined by PLRP.

FIG. 41. Double-tagged Nectin-4 VH4/VL5 antibody conjugated to Compound 25 is 96.6% monomeric as determined by SEC.

FIG. 42. Single-tagged Nectin-4 VH4/VL1 antibody conjugated to Compound 25 yields a DAR of 3.16 as determined by PLRP.

FIG. 43. Single-tagged Nectin-4 VH4/VL1 antibody conjugated to Compound 25 is 97.2% monomeric as determined by SEC.

FIG. 44. Single-tagged Nectin-4 VH4/VL5 antibody conjugated to Compound 25 yields a DAR of 3.25 as determined by PLRP.

FIG. 45. Single-tagged Nectin-4 VH4/VL5 antibody conjugated to Compound 25 yields a DAR of 3.25 as determined by PLRP.

FIG. 46. Clinical observations in rats repeatedly dosed with rat cross-reactive nectin-4 ADCs. Arrows indicate dosing days. There were no observations in animals dosed with the Compound 25 conjugate, whereas the clinical observations in the vedotin dosing group averaged 2.5 on Day 17 and culminated in the death of an animal.

FIG. 47. Red blood cell counts in rats repeatedly dosed with vehicle or ADCs.

FIG. 48. Neutrophil counts in rats repeatedly dosed with vehicle or ADCs.

FIG. 49. Reticulocyte counts in rats repeatedly dosed with vehicle or ADCs.

FIG. 50. Lymphocyte counts in rats repeatedly dosed with vehicle or ADCs.

FIG. 51. Platelet counts in rats repeatedly dosed with vehicle or ADCs.

FIG. 52. Alanine amino transferase counts in rats repeatedly dosed with vehicle or ADCs.

FIG. 53. Aspartate amino transferase counts in rats repeatedly dosed with vehicle or ADCs.

FIG. 54 shows toxicokinetic analysis of rat plasma samples from the Multi-dose non-GLP rat toxicology study #2. The analysis confirms dosing levels and shows improved in vivo stability of the enfortumab Compound 8 conjugate relative to the enfortumab vedotin conjugate.

### DEFINITIONS

“Alkyl” refers to monovalent saturated aliphatic hydrocarbyl groups having from 1 to 10 carbon atoms and such as 1 to 6 carbon atoms, or 1 to 5, or 1 to 4, or 1 to 3 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain (except the C₁ carbon atom) have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

“Alkylene” refers to divalent aliphatic hydrocarbyl groups preferably having from 1 to 6 and more preferably 1 to 3 carbon atoms that are either straight-chained or branched, and which are optionally interrupted with one or more groups selected from —O—, —NR¹⁰—, —NR¹⁰C(O)—, —C(O)NR¹⁰— and the like. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Substituted alkylene” refers to an alkylene group having from 1 to 3 hydrogens replaced with substituents as described for carbons in the definition of “substituted” below.

The term “alkane” refers to alkyl group and alkylene group, as defined herein.

The term “alkylaminoalkyl,” “alkylaminoalkenyl,” and “alkylaminoalkynyl” refers to the groups R′NHR″— where R′ is alkyl group as defined herein and R″ is alkylene, alkenylene or alkynylene group as defined herein.

The term “alkaryl” or “aralkyl” refers to the groups -alkylene-aryl and -substituted alkylene-aryl where alkylene, substituted alkylene and aryl are defined herein.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “alkoxyamino” refers to the group —NH-alkoxy, wherein alkoxy is defined herein.

The term “haloalkoxy” refers to the groups alkyl-O— wherein one or more hydrogen atoms on the alkyl group have been substituted with a halo group and include, by way of examples, groups such as trifluoromethoxy, and the like.

The term “haloalkyl” refers to a substituted alkyl group as described above, wherein one or more hydrogen atoms on the alkyl group have been substituted with a halo group. Examples of such groups include, without limitation, fluoroalkyl groups, such as trifluoromethyl, difluoromethyl, trifluoroethyl and the like.

The term “alkylalkoxy” refers to the groups -alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

The term “alkylthioalkoxy” refers to the group -alkylene-S-alkyl, alkylene-S-substituted alkyl, substituted alkylene-S-alkyl and substituted alkylene-S-substituted alkyl wherein alkyl, substituted alkyl, alkylene and substituted alkylene are as defined herein.

“Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, —SO-aryl, —SO-heteroaryl, —SO₂-alkyl, —SO₂-substituted alkyl, —SO₂-aryl and —SO₂-heteroaryl.

“Alkynyl” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH).

The term “substituted alkynyl” refers to an alkynyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, —SO-aryl, —SO-heteroaryl, —SO₂-alkyl, —SO₂-substituted alkyl, —SO₂-aryl, and —SO₂-heteroaryl.

“Alkynyloxy” refers to the group —O-alkynyl, wherein alkynyl is as defined herein. Alkynyloxy includes, by way of example, ethynyloxy, propynyloxy, and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

“Acylamino” refers to the groups —NR²⁰C(O)alkyl, —NR²⁰C(O)substituted alkyl, NR²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” or the term “aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR²¹C(O)NR²²R²³ where R²¹, R²², and R²³ are independently selected from hydrogen, alkyl, aryl or cycloalkyl, or where two R groups are joined to form a heterocyclyl group.

The term “alkoxycarbonylamino” refers to the group —NRC(O)OR where each R is independently hydrogen, alkyl, substituted alkyl, aryl, heteroaryl, or heterocyclyl wherein alkyl, substituted alkyl, aryl, heteroaryl, and heterocyclyl are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.

“Aminosulfonyl” refers to the group —SO₂NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and where R²¹ and R²² are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Sulfonylamino” refers to the group —NR²¹SO₂R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Aryloxy” refers to the group —O-aryl, wherein aryl is as defined herein, including, by way of example, phenoxy, naphthoxy, and the like, including optionally substituted aryl groups as also defined herein.

“Amino” refers to the group —NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “azido” refers to the group —N₃.

“Carboxyl,” “carboxy” or “carboxylate” refers to —CO₂H or salts thereof.

“Carboxyl ester” or “carboxy ester” or the terms “carboxyalkyl” or “carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” or “carbonate” refers to the groups —O—C(O)O— alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O— substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkenyl” refers to non-aromatic cyclic alkyl groups of from 3 to 10 carbon atoms having single or multiple rings and having at least one double bond and preferably from 1 to 2 double bonds.

The term “substituted cycloalkenyl” refers to cycloalkenyl groups having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkynyl” refers to non-aromatic cycloalkyl groups of from 5 to 10 carbon atoms having single or multiple rings and having at least one triple bond.

“Cycloalkoxy” refers to —O-cycloalkyl.

“Cycloalkenyloxy” refers to —O-cycloalkenyl.

“Halo” or “halogen” refers to fluoro, chloro, bromo, and iodo.

“Hydroxy” or “hydroxyl” refers to the group —OH.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic. To satisfy valence requirements, any heteroatoms in such heteroaryl rings may or may not be bonded to H or a substituent group, e.g., an alkyl group or other substituent as described herein. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

The term “heteroaralkyl” refers to the groups -alkylene-heteroaryl where alkylene and heteroaryl are defined herein. This term includes, by way of example, pyridylmethyl, pyridylethyl, indolylmethyl, and the like.

“Heteroaryloxy” refers to —O-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from nitrogen, sulfur, or oxygen, where, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties. To satisfy valence requirements, any heteroatoms in such heterocyclic rings may or may not be bonded to one or more H or one or more substituent group(s), e.g., an alkyl group or other substituent as described herein.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Heterocyclyloxy” refers to the group —O-heterocyclyl.

The term “heterocyclylthio” refers to the group heterocyclic-S—.

The term “heterocyclene” refers to the diradical group formed from a heterocycle, as defined herein.

The term “hydroxyamino” refers to the group —NHOH.

“Nitro” refers to the group —NO₂.

“Oxo” refers to the atom (═O).

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cylcoalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

“Sulfonyloxy” refers to the group —OSO₂-alkyl, OSO₂-substituted alkyl, OSO₂-alkenyl, OSO₂-substituted alkenyl, OSO₂-cycloalkyl, OSO₂-substituted cycloalkyl, OSO₂-cycloalkenyl, OSO₂-substituted cylcoalkenyl, OSO₂-aryl, OSO₂-substituted aryl, OSO₂-heteroaryl, OSO₂-substituted heteroaryl, OSO₂-heterocyclic, and OSO₂ substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

“Thiol” refers to the group —SH.

“Thioxo” or the term “thioketo” refers to the atom (═S).

“Alkylthio” or the term “thioalkoxy” refers to the group —S-alkyl, wherein alkyl is as defined herein. In certain embodiments, sulfur may be oxidized to —S(O)—. The sulfoxide may exist as one or more stereoisomers.

The term “substituted thioalkoxy” refers to the group —S-substituted alkyl.

The term “thioaryloxy” refers to the group aryl-S— wherein the aryl group is as defined herein including optionally substituted aryl groups also defined herein.

The term “thioheteroaryloxy” refers to the group heteroaryl-S— wherein the heteroaryl group is as defined herein including optionally substituted aryl groups as also defined herein.

The term “thioheterocyclooxy” refers to the group heterocyclyl-S— wherein the heterocyclyl group is as defined herein including optionally substituted heterocyclyl groups as also defined herein.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O-M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸OR⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃ M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸OR⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

It will be appreciated that the term “or a salt or solvate or stereoisomer thereof” is intended to include all permutations of salts, solvates and stereoisomers, such as a solvate of a pharmaceutically acceptable salt of a stereoisomer of subject compound.

The terms “antibodies” and “immunoglobulin” include antibodies or immunoglobulins of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the term are Fab′, Fv, F(ab′)₂, and or other antibody fragments that retain specific binding to antigen, and monoclonal antibodies. An antibody may be monovalent or bivalent.

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH₁) of the heavy chain. Fab fragments differ from Fab′ fragments by the addition of a few residues at the carboxyl terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the VH and VL domains of antibody, wherein these domains are present in a single polypeptide chain. In some aspects, the Fv polypeptide further comprises a polypeptide linker between the VH and VL domains, which enables the sFv to form the desired structure for antigen binding.

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites.

As used herein, the term “affinity” refers to the equilibrium constant for the reversible binding of two agents and is expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. A subject anti-Nectin-4 antibody binds specifically to an epitope within a Nectin-4 polypeptide, e.g., a human Nectin-4 polypeptide, for example, a glycosylated Nectin-4 or a fragment thereof. Non-specific binding would refer to binding with an affinity of less than about 10⁻⁷ M, e.g., binding with an affinity of 10⁻⁶ M, 10⁻⁵ M, 10⁻⁴ M, etc.

The term “specifically binds” in the context of an antibody and an antigen means that the antibody binds to or associates with the antigen with an affinity or Kₐ (that is, an equilibrium association constant of a particular binding interaction with units of 1/M) of, for example, greater than or equal to about 10⁵ M⁻¹.

“High affinity” binding refers to binding with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (K_{D}) of a particular binding interaction with units of M (e.g., 10⁻⁵ M to 10⁻¹³ M, or less). In some embodiments, specific binding means the antibody binds to the antigen with a K_{D} of less than or equal to about 10⁻⁵ M, less than or equal to about 10−⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the antibody for an antigen can be readily determined using conventional techniques, e.g., by competitive ELISA (enzyme-linked immunosorbent assay), equilibrium dialysis, by using surface plasmon resonance (SPR) technology (e.g., the BIAcore 2000 instrument, using general procedures outlined by the manufacturer); by radioimmunoassay; or the like.

As used herein, the term “CDR” or “complementarity determining region” is intended to mean the non-contiguous antigen combining sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison.

TABLE 1
CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

Throughout the present disclosure, the numbering of the residues in an immunoglobulin heavy chain and in an immunoglobulin light chain is that as in MacCallum et al., Sequences of Proteins of Immunological Interest, 5th Ed. Public Health Service, National Institutes of Health, Bethesda, Md. (1991), expressly incorporated herein by reference.

As used herein, the term “framework” when used in reference to an antibody variable region is intended to mean all amino acid residues outside the CDR regions within the variable region of an antibody. A variable region framework is generally a discontinuous amino acid sequence between about 100-120 amino acids in length but is intended to reference only those amino acids outside of the CDRs. As used herein, the term “framework region” is intended to mean each domain of the framework that is separated by the CDRs.

A “native Ig polypeptide” is a polypeptide comprising an amino acid sequence which lacks an aldehyde-tagged constant region as described herein. A native polypeptide may comprise a natural sequence constant region, or may comprise a constant region with pre-existing amino acid sequence modifications (such as additions, deletions and/or substitutions).

In the context of an Ig polypeptide, the term “constant region” is well understood in the art, and refers to a C-terminal region of an Ig heavy chain, or an Ig light chain. An Ig heavy chain constant region includes CH1, CH2, and CH3 domains (and CH4 domains, where the heavy chain is a μ or an ε heavy chain). In a native Ig heavy chain, the CH1, CH2, CH3 (and, if present, CH4) domains begin immediately after (C-terminal to) the heavy chain variable (VH) region, and are each from about 100 amino acids to about 130 amino acids in length. In a native Ig light chain, the constant region begins immediately after (C-terminal to) the light chain variable (VL) region, and is about 100 amino acids to 120 amino acids in length.

An “epitope” is a site on an antigen (e.g., a site on Nectin-4) to which an antibody binds. Epitopes can be formed both from contiguous amino acids or noncontiguous amino acids juxtaposed by folding (e.g., tertiary folding) of a protein. Epitopes formed from contiguous amino acids are typically retained on exposure to denaturing solvents whereas epitopes formed by folding are typically lost on treatment with denaturing solvents. An epitope typically includes at least 3, and more usually, at least 5 or 8-10 amino acids in a linear or spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996). Several commercial laboratories offer epitope mapping services. Epitopes bound by an antibody immunoreactive with a membrane associated antigen can reside on the surface of the cell (e.g., in the extracellular region of a transmembrane protein), so that such epitopes are considered cell-surface accessible, solvent accessible, and/or cell-surface exposed.

By “genetically-encodable” as used in reference to an amino acid sequence of polypeptide, peptide or protein means that the amino acid sequence is composed of amino acid residues that are capable of production by transcription and translation of a nucleic acid encoding the amino acid sequence, where transcription and/or translation may occur in a cell or in a cell-free in vitro transcription/translation system.

The term “control sequences” refers to DNA sequences that facilitate expression of an operably linked coding sequence in a particular expression system, e.g., mammalian cell, bacterial cell, cell-free synthesis, etc. The control sequences that are suitable for prokaryote systems, for example, include a promoter, optionally an operator sequence, and a ribosome binding site. Eukaryotic cell systems may utilize promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate the initiation of translation. Generally, “operably linked” means that the DNA sequences being linked are contiguous, and, in the case of a secretory leader, contiguous and in reading frame. Linking is accomplished by ligation or through amplification reactions. Synthetic oligonucleotide adaptors or linkers may be used for linking sequences in accordance with conventional practice.

The term “expression cassette” as used herein refers to a segment of nucleic acid, usually DNA, that can be inserted into a nucleic acid (e.g., by use of restriction sites compatible with ligation into a construct of interest or by homologous recombination into a construct of interest or into a host cell genome). In general, the nucleic acid segment comprises a polynucleotide that encodes a polypeptide of interest, and the cassette and restriction sites are designed to facilitate insertion of the cassette in the proper reading frame for transcription and translation. Expression cassettes can also comprise elements that facilitate expression of a polynucleotide encoding a polypeptide of interest in a host cell, e.g., a mammalian host cell. These elements may include, but are not limited to: a promoter, a minimal promoter, an enhancer, a response element, a terminator sequence, a polyadenylation sequence, and the like.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In some embodiments, the antibody will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated antibody includes the antibody in situ within recombinant cells since at least one component of the antibody's natural environment will not be present. In some instances, isolated antibody will be prepared by at least one purification step.

The term “natural antibody” refers to an antibody in which the heavy and light chains of the antibody have been made and paired by the immune system of a multi-cellular organism. Spleen, lymph nodes, bone marrow and serum are examples of tissues that produce natural antibodies. For example, the antibodies produced by the antibody producing cells isolated from a first animal immunized with an antigen are natural antibodies.

The term “humanized antibody” or “humanized immunoglobulin” refers to a non-human (e.g., mouse or rabbit) antibody containing one or more amino acids (in a framework region, a constant region or a CDR, for example) that have been substituted with a correspondingly positioned amino acid from a human antibody. In general, humanized antibodies produce a reduced immune response in a human host, as compared to a non-humanized version of the same antibody. Antibodies can be humanized using a variety of techniques known in the art including, for example, CDR-grafting, veneering or resurfacing, chain shuffling, and the like. In certain embodiments, framework substitutions are identified by modeling of the interactions of the CDR and framework residues to identify framework residues important for antigen binding and sequence comparison to identify unusual framework residues at particular positions. Accordingly, the antibodies described above may be humanized using methods that are well known in the art.

In certain embodiments, the antibody molecules disclosed herein include a heavy chain comprising a variable heavy chain region as provided herein and a human IgG1 constant region having the amino acid sequence set forth in UniProt: P01857-1, version 1. In certain embodiments, the antibody molecules disclosed herein include a light chain comprising a variable light chain region as provided herein and a human light chain constant region. In certain embodiments, the human light chain constant region is a human kappa light chain constant region having the amino acid set forth in UniProtKB/Swiss-Prot: P01834.2. In certain embodiments, the human IgG1 heavy chain constant region present in the subject antibodies may include mutations, e.g., substitutions to modulate Fc function. For example, the LALAPG effector function mutations (L234A, L235A, and P329G) or the N297A mutation may be introduced to reduce antibody dependent cellular cytotoxicity (ADCC). In some cases, only L234A and L235A mutations are used without the P329G mutation. The numbering of the substitutions is based on the EU numbering system. The “EU numbering system” or “EU index” is generally used when referring to a residue in an immunoglobulin heavy chain constant region (e.g., the EU index reported in Kabat et al., Sequences of Proteins of Immunological Interest, 5th Ed. Public Health Service, National Institutes of Health, Bethesda, MD. (1991)). The “EU index as in Kabat” refers to the residue numbering of the human IgG 1 EU antibody.

The term “chimeric antibodies” refer to antibodies whose light and heavy chain genes have been constructed, typically by genetic engineering, from antibody variable and constant region genes belonging to different species. For example, the variable segments of the genes from a mouse monoclonal antibody may be joined to human constant segments, such as gamma 1 and gamma 3. An example of a therapeutic chimeric antibody is a hybrid protein composed of the variable or antigen-binding domain from a mouse antibody and the constant or effector domain from a human antibody, although domains from other mammalian species may be used.

The terms “polypeptide,” “peptide,” and “protein” are used interchangeably herein to refer to a polymeric form of amino acids of any length. Unless specifically indicated otherwise, “polypeptide,” “peptide,” and “protein” can include genetically coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, proteins which contain at least one N-terminal methionine residue (e.g., to facilitate production in a recombinant host cell); immunologically tagged proteins; and the like. In the context of an antibody, it is clear that a chain or a domain comprises a polypeptide.

“Native amino acid sequence” or “parent amino acid sequence” are used interchangeably herein to refer to the amino acid sequence of a polypeptide prior to modification to include a modified amino acid residue.

The terms “amino acid analog,” “unnatural amino acid,” and the like may be used interchangeably, and include amino acid-like compounds that are similar in structure and/or overall shape to one or more amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y). Amino acid analogs also include natural amino acids with modified side chains or backbones. Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as Cl or Br, etc.), deletion of a group, substitution of a covalent bond (single bond for double bond, etc.), or combinations thereof. For example, amino acid analogs may include α-hydroxy acids, and α-amino acids, and the like.

The terms “amino acid side chain” or “side chain of an amino acid” and the like may be used to refer to the substituent attached to the α-carbon of an amino acid residue, including natural amino acids, unnatural amino acids, and amino acid analogs. An amino acid side chain can also include an amino acid side chain as described in the context of the modified amino acids and/or conjugates described herein.

The term “conjugated” generally refers to a chemical linkage, either covalent or non-covalent, usually covalent, that proximally associates one molecule of interest with a second molecule of interest. In some embodiments, the agent is selected from a half-life extending moiety, a labeling agent, and a therapeutic agent. For half-life extension, for example, the antibodies of the present disclosure can optionally be modified to provide for improved pharmacokinetic profile (e.g., by PEGylation, hyperglycosylation, and the like). Modifications that can enhance serum half-life are of interest.

The term “carbohydrate” and the like may be used to refer to monomers and/or polymers of monosaccharides, disaccharides, oligosaccharides, and polysaccharides. The term sugar may be used to refer to the smaller carbohydrates, such as monosaccharides, disaccharides. The term “carbohydrate derivative” includes compounds where one or more functional groups of a carbohydrate of interest are substituted (replaced by any convenient substituent), modified (converted to another group using any convenient chemistry) or absent (e.g., eliminated or replaced by H). A variety of carbohydrates and carbohydrate derivatives are available and may be adapted for use in the subject compounds and conjugates.

As used herein the term “isolated” is meant to describe a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “substantially purified” refers to a compound that is removed from its natural environment and is at least 60% free, at least 75% free, at least 80% free, at least 85% free, at least 90% free, at least 95% free, at least 98% free, or more than 98% free, from other components with which it is naturally associated.

The term “physiological conditions” is meant to encompass those conditions compatible with living cells, e.g., predominantly aqueous conditions of a temperature, pH, salinity, etc. that are compatible with living cells.

By “reactive partner” is meant a molecule or molecular moiety that specifically reacts with another reactive partner to produce a reaction product. Exemplary reactive partners include a cysteine or serine of a sulfatase motif and Formylglycine Generating Enzyme (FGE), which react to form a reaction product of a converted aldehyde tag containing a formylglycine (fGly) in lieu of cysteine or serine in the motif. Other exemplary reactive partners include an aldehyde of an fGly residue of a converted aldehyde tag (e.g., a reactive aldehyde group) and an “aldehyde-reactive reactive partner,” which comprises an aldehyde-reactive group and a moiety of interest, and which reacts to form a reaction product of a polypeptide having the moiety of interest conjugated to the polypeptide through the fGly residue.

“N-terminus” refers to the terminal amino acid residue of a polypeptide having a free amine group, which amine group in non-N-terminus amino acid residues normally forms part of the covalent backbone of the polypeptide.

“C-terminus” refers to the terminal amino acid residue of a polypeptide having a free carboxyl group, which carboxyl group in non-C-terminus amino acid residues normally forms part of the covalent backbone of the polypeptide.

By “internal site” as used in referenced to a polypeptide or an amino acid sequence of a polypeptide means a region of the polypeptide that is not at the N-terminus or at the C-terminus.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, e.g., arresting its development; and (c) relieving the disease, e.g., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc.

A “therapeutically effective amount” or “efficacious amount” refers to the amount of a subject anti-Nectin-4 Ab that, when administered to a mammal or other subject for treating a disease, is sufficient to effect such treatment for the disease. The “therapeutically effective amount” will vary depending on the anti-Nectin-4 Ab, the disease and its severity and the age, weight, etc., of the subject to be treated.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an antibody” includes a plurality of such antibodies and reference to “the CDR” includes reference to one or more CDRs and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides antibodies specific for Nectin-4. The disclosure also provides antibody conjugates, e.g., ADCs, comprising such antibodies specific for Nectin-4. The disclosure provides methods of production of such antibodies and conjugates, as well as methods of using them. Embodiments of each are described in more detail in the sections below. Also provided are compositions that include the antibodies and/or ADCs of the present disclosure, including in some instances, pharmaceutical compositions. In certain aspects, provided are methods of using the ADC that include administering to an individual having a cell proliferative disorder a therapeutically effective amount of the ADC of the present disclosure.

### Nectin-4 Antibodies and Antibody-Drug Conjugates Thereof

As summarized above, the present disclosure provides antibodies specific for Nectin-4 and conjugates (e.g., antibody-drug-conjugates (ADCs)) of such antibodies. In addition, the present disclosure provides anti-Nectin-4 antibodies comprising a fGly residue.

### Antibody-Drug Conjugates

The present disclosure provides a conjugate, e.g., ADC of antibodies specific for Nectin-4. By “conjugate” is meant a polypeptide (e.g., an antibody) is covalently attached to a moiety of interest (e.g., a drug or active agent). For example, an antibody-drug conjugate according to the present disclosure includes one or more drugs or active agents covalently attached to an antibody. In certain embodiments, the polypeptide (e.g., antibody) and the one or more drugs or active agents are bound to each other through one or more functional groups and covalent bonds. For example, the one or more functional groups and covalent bonds can include a linker, such as a cleavable linker, as described herein.

In certain embodiments, the conjugate is a polypeptide conjugate, which includes a polypeptide (e.g., an antibody) conjugated to one or more other moieties. In certain embodiments, the one or more moieties conjugated to the polypeptide can each independently be any of a variety of moieties of interest such as, but not limited to, a drug, an active agent, a detectable label, a water-soluble polymer, or a moiety for immobilization of the polypeptide to a membrane or a surface. In certain embodiments, the conjugate is a drug conjugate, where a polypeptide is an antibody, thus providing an antibody-drug conjugate. For instance, the conjugate can be a drug conjugate, where a polypeptide is conjugated to one or more drugs or active agents. Various types of drugs and active agents may be used in the conjugates and are described in more detail below.

The one or more drugs or active agents can be conjugated to the polypeptide (e.g., antibody) at any desired site of the polypeptide. Thus, the present disclosure provides, for example, a polypeptide having one or more drugs or active agents conjugated at a site at or near the C-terminus of the polypeptide. Other examples include a polypeptide having one or more drugs or active agents conjugated at a position at or near the N-terminus of the polypeptide. Examples also include a polypeptide having one or more drugs or active agents conjugated at a position between the C-terminus and the N-terminus of the polypeptide (e.g., at an internal site of the polypeptide). Combinations of the above are also possible where the polypeptide is conjugated to more than one drugs or active agents.

In certain embodiments, a conjugate of the present disclosure includes one or more drugs or active agents conjugated to an amino acid residue of a polypeptide at the α-carbon of an amino acid residue. Stated another way, a conjugate includes a polypeptide where the side chain of one or more amino acid residues in the polypeptide has been modified and attached to one or more drugs or active agents (e.g., attached to one or more drugs or active agents through a linker as described herein). For example, a conjugate includes a polypeptide where the α-carbon of one or more amino acid residues in the polypeptide has been modified and attached to one or more drugs or active agents (e.g., attached to one or more drugs or active agents through a linker as described herein).

Embodiments of the present disclosure include conjugates where a polypeptide is conjugated to one or more moieties, such as 2 moieties, 3 moieties, 4 moieties, 5 moieties, 6 moieties, 7 moieties, 8 moieties, 9 moieties, or 10 or more moieties. The moieties may be conjugated to the polypeptide at one or more sites in the polypeptide. For example, one or more moieties may be conjugated to a single amino acid residue of the polypeptide. In some cases, one moiety is conjugated to an amino acid residue of the polypeptide. In other embodiments, two moieties may be conjugated to the same amino acid residue of the polypeptide. In other embodiments, a first moiety is conjugated to a first amino acid residue of the polypeptide and a second moiety is conjugated to a second amino acid residue of the polypeptide. In other embodiments, two moieties may be conjugated to a first amino acid residue of the polypeptide and two moieties may be conjugated to a second amino acid residue of the polypeptide. Combinations of the above are also possible, for example where a polypeptide is conjugated to a first moiety at a first amino acid residue and conjugated to two other moieties at a second amino acid residue. Other combinations are also possible, such as, but not limited to, a polypeptide conjugated to first and second moieties at a first amino acid residue and conjugated to third and fourth moieties at a second amino acid residue, etc.

The one or more amino acid residues of the polypeptide that are conjugated to the one or more moieties of interest may be naturally occurring amino acids, unnatural amino acids, or combinations thereof. For instance, the conjugate may include one or more drugs or active agents conjugated to a naturally occurring amino acid residue of the polypeptide. In other instances, the conjugate may include one or more drugs or active agents conjugated to an unnatural amino acid residue of the polypeptide. One or more drugs or active agents may be conjugated to the polypeptide at a single natural or unnatural amino acid residue as described herein. One or more natural or unnatural amino acid residues in the polypeptide may be conjugated to the moiety or moieties as described herein. For example, two (or more) amino acid residues (e.g., natural or unnatural amino acid residues) in the polypeptide may each be conjugated to one or more moieties, such that multiple sites in the polypeptide are conjugated to the one or more moieties of interest.

In certain embodiments, the polypeptide (e.g., antibody) and the moieties of interest (e.g., drugs or active agents) are conjugated through a conjugation moiety. For example, the polypeptide and the moieties of interest may each be bound (e.g., covalently bonded) to the conjugation moiety, thus indirectly binding the polypeptide and the moieties of interest together through the conjugation moiety. In some cases, the conjugation moiety includes a hydrazinyl-indolyl or a hydrazinyl-pyrrolo-pyridinyl compound, or a derivative of a hydrazinyl-indolyl or a hydrazinyl-pyrrolo-pyridinyl compound. For instance, a general scheme for coupling moieties of interest to a polypeptide through a hydrazinyl-indolyl or a hydrazinyl-pyrrolo-pyridinyl conjugation moiety is shown in the general reaction scheme below. Hydrazinyl-indolyl and hydrazinyl-pyrrolo-pyridinyl conjugation moieties are also referred to herein as a hydrazino-iso-Pictet-Spengler (HIPS) conjugation moiety and an aza-hydrazino-iso-Pictet-Spengler (azaHIPS) conjugation moiety, respectively.

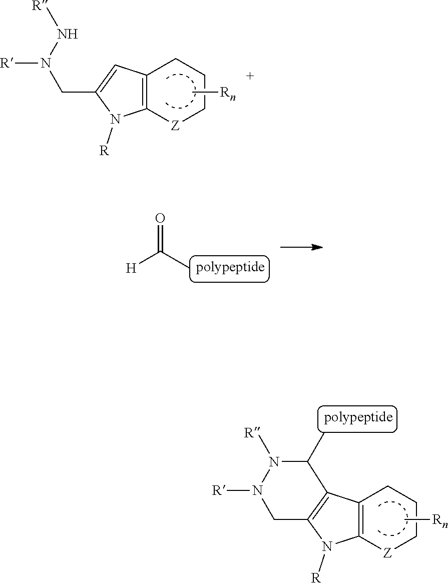

In the reaction scheme above, each R independently includes a moiety of interest (e.g., drug or active agent) that is conjugated to the polypeptide (e.g., conjugated to the polypeptide through a linker as described herein), where n is 0 or an integer from 1 to 4. As shown in the reaction scheme above, a conjugation moiety (e.g., a hydrazinyl-indolyl or a hydrazinyl-pyrrolo-pyridinyl conjugation moiety) is attached to one or more drugs or active agents, R. A polypeptide (e.g., antibody) that includes a 2-formylglycine residue (fGly) is reacted with the conjugation moiety to produce a polypeptide (e.g., antibody) conjugate, thus attaching the one or more drugs or active agents to the polypeptide through the conjugation moiety.

As described herein, the moieties of interest (also referred to herein as a “payload”) can be any of a variety of moieties such as, but not limited to, chemical entities, such as detectable labels, or a drugs or active agents. R′ and R″ may each independently be any desired substituent, such as, but not limited to, hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. Z may be CR²¹, NR²², N, O or S, where R²¹ and R²² are each independently selected from any of the substituents described for R′ and R″ above.

Other hydrazinyl-indolyl or hydrazinyl-pyrrolo-pyridinyl conjugation moieties are also possible, as shown in the conjugates and compounds described herein. For example, the hydrazinyl-indolyl or hydrazinyl-pyrrolo-pyridinyl conjugation moieties may be attached (e.g., covalently attached) to one or more linkers. As such, embodiments of the present disclosure include a hydrazinyl-indolyl or hydrazinyl-pyrrolo-pyridinyl conjugation moiety attached to one or more drugs or active agents through a corresponding linker. Thus, conjugates of the present disclosure may include one or more linkers, where each linker attaches one or more corresponding drugs or active agents to the hydrazinyl-indolyl or hydrazinyl-pyrrolo-pyridinyl conjugation moiety. In some cases, the hydrazinyl-indolyl or hydrazinyl-pyrrolo-pyridinyl conjugation moiety and one or more linkers may be viewed overall as a “branched linker”, where the hydrazinyl-indolyl or hydrazinyl-pyrrolo-pyridinyl conjugation moiety is attached to two or more “branches”, where each branch includes a linker attached to a drug or active agent.

For example, in some instances of the reaction scheme above, n is 0, and thus one R group (e.g., drug or active agent) is attached to the hydrazinyl-indolyl or hydrazinyl-pyrrolo-pyridinyl conjugation moiety through a linker. In other instances, n is 1, and thus two R groups (e.g., drugs or active agents) are attached to the hydrazinyl-indolyl or hydrazinyl-pyrrolo-pyridinyl conjugation moiety each via their own corresponding linker. In these instances, the hydrazinyl-indolyl or hydrazinyl-pyrrolo-pyridinyl conjugation moiety and two linkers may be viewed overall as a branched linker.

Combinations of the same or different payloads may be conjugated to the polypeptide through a branched linker. In certain embodiments, two payloads (e.g., drugs, active agents or detectable labels) attached to a branched linker are the same payload (e.g., drug, active agent or detectable label). For example, a first branch of a branched linker may be attached to a payload (e.g., drug, active agent or detectable label) and a second branch of the branched linker may be attached to the same payload (e.g., drug, active agent or detectable label) as the first branch.

In other embodiments, two payloads (e.g., drugs, active agents or detectable labels) attached to a branched linker are different payloads (e.g., drugs, active agents or detectable labels). For example, a first branch of a branched linker may be attached to a first payload (e.g., a first drug, active agent or detectable label) and a second branch of the branched linker may be attached to a second payload (e.g., a second drug, active agent or detectable label) different from the first payload (e.g., the first drug, active agent or detectable label) attached to the first branch.

Various embodiments of the linkers that may couple the hydrazinyl-indolyl or hydrazinyl-pyrrolo-pyridinyl conjugation moiety to the drugs or active agents are described in detail herein. For example, in some instances, the linker is a cleavable linker, such as a cleavable linker as described herein.

In certain embodiments, the polypeptide may be conjugated to one or more moieties of interest, where one or more amino acids of the polypeptide are modified before conjugation to the one or more moieties of interest. Modification of one or more amino acids of the polypeptide may produce a polypeptide that contains one or more reactive groups suitable for conjugation to the one or more moieties of interest. In some cases, the polypeptide may include one or more modified amino acid residues to provide one or more reactive groups suitable for conjugation to the one or more moieties of interest. For example, an amino acid of the polypeptide may be modified to include a reactive aldehyde group (e.g., a reactive aldehyde). A reactive aldehyde may be included in an “aldehyde tag” or “ald-tag”, which as used herein refers to an amino acid sequence derived from a sulfatase motif (e.g., L(C/S)TPSR) that has been converted by action of a formylglycine generating enzyme (FGE) to contain a 2-formylglycine residue (referred to herein as “fGly”). The fGly residue generated by an FGE may also be referred to as a “formylglycine”. Stated differently, the term “aldehyde tag” is used herein to refer to an amino acid sequence that includes a “converted” sulfatase motif (i.e., a sulfatase motif in which a cysteine or serine residue has been converted to fGly by action of an FGE, e.g., L(fGly)TPSR (SEQ ID NO: 245)). A converted sulfatase motif may be produced from an amino acid sequence that includes an “unconverted” sulfatase motif (i.e., a sulfatase motif in which the cysteine or serine residue has not been converted to fGly by an FGE, but is capable of being converted, e.g., an unconverted sulfatase motif with the sequence: L(C/S)TPSR). By “conversion” as used in the context of action of a formylglycine generating enzyme (FGE) on a sulfatase motif refers to biochemical modification of a cysteine or serine residue in a sulfatase motif to a formylglycine (fGly) residue (e.g., Cys to fGly, or Ser to fGly). Additional aspects of aldehyde tags and uses thereof in site-specific protein modification are described in U.S. Pat. Nos. 7,985,783 and 8,729,232, the disclosures of each of which are incorporated herein by reference.

In some cases, to produce the conjugate, the polypeptide containing the fGly residue may be conjugated to the one or more moieties of interest by reaction of the fGly with a compound (e.g., a compound containing a hydrazinyl-indolyl or a hydrazinyl-pyrrolo-pyridinyl conjugation moiety, as described above). For example, an fGly-containing polypeptide may be contacted with a reactive partner under conditions suitable to provide for conjugation of one or more drugs or active agents to the polypeptide. In some instances, the reactive partner may include a hydrazinyl-indolyl or a hydrazinyl-pyrrolo-pyridinyl conjugation moiety as described above. For example, one or more drugs or active agents may be attached to a hydrazinyl-indolyl or a hydrazinyl-pyrrolo-pyridinyl conjugation moiety. In some cases, the one or more drugs or active agents are attached to a hydrazinyl-indolyl or a hydrazinyl-pyrrolo-pyridinyl conjugation moiety, such as covalently attached to a hydrazinyl-indolyl or a hydrazinyl-pyrrolo-pyridinyl, where each drug or active agent is attached through a corresponding linker to the hydrazinyl-indolyl or a hydrazinyl-pyrrolo-pyridinyl conjugation moiety.

In certain embodiments, a conjugate of the present disclosure includes a polypeptide (e.g., an antibody) having at least one amino acid residue that has been attached to one or more moieties of interest (e.g., one or more drugs or active agents). In order to make the conjugate, an amino acid residue of the polypeptide may be modified and then coupled to one or more drugs or active agents attached to a hydrazinyl-indolyl or a hydrazinyl-pyrrolo-pyridinyl conjugation moiety as described above. In certain embodiments, an amino acid residue of the polypeptide (e.g., antibody) is a cysteine or serine residue that is converted to an fGly residue, as described above. In certain embodiments, the converted amino acid residue (e.g., fGly residue) is conjugated to one or more drugs or active agents containing a hydrazinyl-indolyl or a hydrazinyl-pyrrolo-pyridinyl conjugation moiety as described above to provide a conjugate of the present disclosure where the one or more drugs or active agents are conjugated to the polypeptide through the hydrazinyl-indolyl or hydrazinyl-pyrrolo-pyridinyl conjugation moiety. As used herein, the term fGly′ refers to the amino acid residue of the polypeptide (e.g., antibody) that is coupled to the one or more moieties of interest (e.g., one or more drugs or active agents).

In certain embodiments, the conjugate includes a polypeptide (e.g., an antibody) having at least one amino acid residue attached to a hydrazinyl-indolyl or a hydrazinyl-pyrrolo-pyridinyl conjugation moiety as described herein, which in turn is attached to one or more drugs or active agents through one or more corresponding linkers. For instance, the conjugate may include a polypeptide (e.g., an antibody) having at least one amino acid residue (fGly′) that is conjugated to the one or more moieties of interest (e.g., one or more drugs or active agents) as described above.

Aspects of the present disclosure include a conjugate of formula (I):

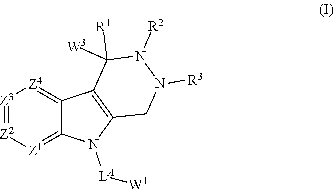

wherein:
Z¹, Z², Z³ and Z⁴ are each independently selected from CR⁴, N and C-L^{B}-W²;
R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L^{A} is a first linker;
L^{B} is a second linker;
W¹ is a first drug;
W² is a second drug; and
W³ is a polypeptide.

The substituents related to conjugates of formula (I) are described in more detail below.

In certain embodiments, Z¹, Z², Z³ and Z⁴ are each independently selected from CR⁴, N and C-L^{B}-W². In certain embodiments, Z¹ is CR⁴. In certain embodiments, Z¹ is N. In certain embodiments, Z¹ is C-L^{B}-W². In certain embodiments, Z² is CR⁴. In certain embodiments, Z² is N. In certain embodiments, Z² is C-L^{B}-W². In certain embodiments, Z³ is CR⁴. In certain embodiments, Z³ is N. In certain embodiments, Z³ is C-L^{B}-W². In certain embodiments, Z⁴ is CR⁴. In certain embodiments, Z⁴ is N. In certain embodiments, Z⁴ is C-L^{B}-W².

Combinations of various Z¹, Z², Z³ and Z⁴ are possible. For example, in some instances, Z¹ is CR⁴, Z² is CR⁴, Z³ is CR⁴, and Z⁴ is CR⁴. In some instances, Z¹ is N, Z² is CR⁴, Z³ is CR⁴, and Z⁴ is CR⁴. In some instances, Z¹ is C-L^{B}-W², Z² is CR⁴, Z³ is CR⁴, and Z⁴ is CR⁴. In some instances, Z¹ is CR⁴, Z² is C-L^{B}-W², Z³ is CR⁴, and Z⁴ is CR⁴. In some instances, Z¹ is CR⁴, Z² is CR⁴, Z³ is C-L^{B}-W², and Z⁴ is CR⁴. In some instances, Z¹ is CR⁴, Z² is CR⁴, Z³ is CR⁴, and Z⁴ is C-L^{B}-W².

In certain embodiments, R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, substituted heterocyclyl. In certain embodiments, R¹ is hydrogen. In certain embodiments, R¹ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹ is alkynyl or substituted alkynyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl.

In certain embodiments, R² is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R² is hydrogen. In certain embodiments, R² is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R² is methyl. In certain embodiments, R² is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R² is alkynyl or substituted alkynyl. In certain embodiments, R² is alkoxy or substituted alkoxy. In certain embodiments, R² is amino or substituted amino. In certain embodiments, R² is carboxyl or carboxyl ester. In certain embodiments, R² is acyl or acyloxy. In certain embodiments, R² is acyl amino or amino acyl. In certain embodiments, R² is alkylamide or substituted alkylamide. In certain embodiments, R² is sulfonyl. In certain embodiments, R² is thioalkoxy or substituted thioalkoxy. In certain embodiments, R² is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R² is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R² is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R² is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R³ is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R³ is hydrogen. In certain embodiments, R³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³ is methyl. In certain embodiments, R³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³ is alkynyl or substituted alkynyl. In certain embodiments, R³ is alkoxy or substituted alkoxy. In certain embodiments, R³ is amino or substituted amino. In certain embodiments, R³ is carboxyl or carboxyl ester. In certain embodiments, R³ is acyl or acyloxy. In certain embodiments, R³ is acyl amino or amino acyl. In certain embodiments, R³ is alkylamide or substituted alkylamide. In certain embodiments, R³ is sulfonyl. In certain embodiments, R³ is thioalkoxy or substituted thioalkoxy. In certain embodiments, R³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiment, both R² and R³ are methyl.

In certain embodiments, R² and R³ are optionally cyclically linked to form a 5 or 6-membered heterocyclyl. In certain embodiments, R² and R³ are cyclically linked to form a 5 or 6-membered heterocyclyl. In certain embodiments, R² and R³ are cyclically linked to form a 5-membered heterocyclyl. In certain embodiments, R² and R³ are cyclically linked to form a 6-membered heterocyclyl.

In certain embodiments, each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

The various possibilities for each R⁴ are described in more detail as follows. In certain embodiments, R⁴ is hydrogen. In certain embodiments, each R⁴ is hydrogen. In certain embodiments, R⁴ is halogen, such as F, Cl, Br or I. In certain embodiments, R⁴ is F. In certain embodiments, R⁴ is Cl. In certain embodiments, R⁴ is Br. In certain embodiments, R⁴ is I. In certain embodiments, R⁴ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R⁴ is methyl. In certain embodiments, R⁴ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R⁴ is alkynyl or substituted alkynyl. In certain embodiments, R⁴ is alkoxy or substituted alkoxy. In certain embodiments, R⁴ is amino or substituted amino. In certain embodiments, R⁴ is carboxyl or carboxyl ester. In certain embodiments, R⁴ is acyl or acyloxy. In certain embodiments, R⁴ is acyl amino or amino acyl. In certain embodiments, R⁴ is alkylamide or substituted alkylamide. In certain embodiments, R⁴ is sulfonyl. In certain embodiments, R⁴ is thioalkoxy or substituted thioalkoxy. In certain embodiments, R⁴ is aryl or substituted aryl, such as C₅₋₈ aryl or C₅₋₈ substituted aryl, such as a C₅ aryl or C₅ substituted aryl, or a C₆ aryl or C₆ substituted aryl (e.g., phenyl or substituted phenyl). In certain embodiments, R⁴ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R⁴ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R⁴ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, L^{A} is a first linker. Examples of linkers that can be used in the conjugates of the present disclosure are described in more detail below.

In certain embodiments, L^{B} is a second linker. Examples of linkers that can be used in the conjugates of the present disclosure are described in more detail below.

In certain embodiments, W¹ is a first drug (or a first active agent). Examples of drugs and active agents that can be used in the conjugates of the present disclosure are described in more detail herein.

In certain embodiments, W² is a second drug (or a second active agent).

Examples of drugs and active agents that can be used in the conjugates of the present disclosure are described in more detail herein.

In certain embodiments, W³ is a polypeptide (e.g., an antibody). In certain embodiments, W³ comprises one or more fGly′ residues as described herein. In certain embodiments, the polypeptide is attached to the rest of the conjugate through an fGly′ residue as described herein. Examples of polypeptides and antibodies that can be used in the conjugates of the present disclosure are described in more detail herein.

In certain embodiments, the conjugate of formula (I) includes a first linker, L^{A}. The first linker, L^{A}, may be utilized to bind a first moiety of interest (e.g., a first drug or active agent) to a polypeptide (e.g., an antibody) through a conjugation moiety. The first linker, L^{A}, may be bound (e.g., covalently bonded) to the conjugation moiety (e.g., as described herein). For example, the first linker, L^{A}, may attach a hydrazinyl-indolyl or a hydrazinyl-pyrrolo-pyridinyl conjugation moiety to a first drug or active agent. The hydrazinyl-indolyl or hydrazinyl-pyrrolo-pyridinyl conjugation moiety may be used to conjugate the first linker, L^{A}, (and thus the first drug or active agent) to a polypeptide, such as an antibody.

For example, as shown in formula (I) above, L^{A} is attached to W³ through a conjugation moiety, and thus W³ is indirectly bonded to the linker L^{A} through the hydrazinyl-indolyl or a hydrazinyl-pyrrolo-pyridinyl conjugation moiety. As described above, W³ is a polypeptide (e.g., an antibody), and thus L^{A} is attached through the hydrazinyl-indolyl or a hydrazinyl-pyrrolo-pyridinyl conjugation moiety to the polypeptide (antibody), e.g., the linker L^{A} is indirectly bonded to the polypeptide (antibody) through the hydrazinyl-indolyl or a hydrazinyl-pyrrolo-pyridinyl conjugation moiety.

Any convenient linker may be utilized for the first linker L^{A} in the subject conjugates and compounds. In certain embodiments, the first linker L^{A} may include a group selected from alkyl, substituted alkyl, alkenyl, substituted alkenyl, alkynyl, substituted alkynyl, alkoxy, substituted alkoxy, amino, substituted amino, carboxyl, carboxyl ester, acyl amino, alkylamide, substituted alkylamide, aryl, substituted aryl, heteroaryl, substituted heteroaryl, cycloalkyl, substituted cycloalkyl, heterocyclyl, and substituted heterocyclyl. In certain embodiments, the first linker L^{A} may include an alkyl or substituted alkyl group. In certain embodiments, the first linker L^{A} may include an alkenyl or substituted alkenyl group. In certain embodiments, the first linker L^{A} may include an alkynyl or substituted alkynyl group. In certain embodiments, the first linker L^{A} may include an alkoxy or substituted alkoxy group. In certain embodiments, the first linker L^{A} may include an amino or substituted amino group. In certain embodiments, the first linker L^{A} may include a carboxyl or carboxyl ester group. In certain embodiments, the first linker L^{A} may include an acyl amino group. In certain embodiments, the first linker L^{A} may include an alkylamide or substituted alkylamide group. In certain embodiments, the first linker L^{A} may include an aryl or substituted aryl group. In certain embodiments, the first linker L^{A} may include a heteroaryl or substituted heteroaryl group. In certain embodiments, the first linker L^{A} may include a cycloalkyl or substituted cycloalkyl group. In certain embodiments, the first linker L^{A} may include a heterocyclyl or substituted heterocyclyl group.

In certain embodiments, the first linker L^{A} may include a polymer. For example, the polymer may include a polyalkylene glycol and derivatives thereof, including polyethylene glycol, methoxypolyethylene glycol, polyethylene glycol homopolymers, polypropylene glycol homopolymers, copolymers of ethylene glycol with propylene glycol (e.g., where the homopolymers and copolymers are unsubstituted or substituted at one end with an alkyl group), polyvinyl alcohol, polyvinyl ethyl ethers, polyvinylpyrrolidone, combinations thereof, and the like. In certain embodiments, the polymer is a polyalkylene glycol. In certain embodiments, the polymer is a polyethylene glycol. Other linkers are also possible, as shown in the conjugates and compounds described in more detail below.

In some embodiments, L^{A} is a first linker described by the formula:

-(L¹)ₐ-(L²)_{b}-(L³)_{c}-(L⁴)_{d}-(L⁵)ₑ-(L⁶)_{f}-,
wherein L¹, L², L³, L⁴, L⁵ and L⁶ are each independently a linker subunit, and a, b, c, d, e and f are each independently 0 or 1.

In certain embodiments, the sum of a, b, c, d, e and f is 0 to 6. In certain embodiments, the sum of a, b, c, d, e and f is 0. In certain embodiments, the sum of a, b, c, d, e and f is 1. In certain embodiments, the sum of a, b, c, d, e and f is 2. In certain embodiments, the sum of a, b, c, d, e and f is 3. In certain embodiments, the sum of a, b, c, d, e and f is 4. In certain embodiments, the sum of a, b, c, d, e and f is 5. In certain embodiments, the sum of a, b, c, d, e and f is 6. In certain embodiments, a, b, c, d, e and f are each 1. In certain embodiments, a, b, c, d and e are each 1 and f is 0. In certain embodiments, a, b, c and d are each 1 and e and f are each 0. In certain embodiments, a, b, and c are each 1 and d, e and f are each 0. In certain embodiments, a and b are each 1 and c, d, e and f are each 0. In certain embodiments, a is 1 and b, c, d, e and f are each 0.

In certain embodiments, the linker subunit L¹ is attached to the hydrazinyl-indolyl or a hydrazinyl-pyrrolo-pyridinyl conjugation moiety (e.g., as shown in formula (I) above). In certain embodiments, the linker subunit L², if present, is attached to the first drug or active agent W¹. In certain embodiments, the linker subunit L³, if present, is attached to the first drug or active agent W¹. In certain embodiments, the linker subunit L⁴, if present, is attached to the first drug or active agent W¹. In certain embodiments, the linker subunit L⁵, if present, is attached to the first drug or active agent W¹. In certain embodiments, the linker subunit L⁶, if present, is attached to the first drug or active agent W¹.

Any convenient linker subunits may be utilized in the first linker L^{A}. Linker subunits of interest include, but are not limited to, units of polymers such as polyethylene glycols, polyethylenes and polyacrylates, amino acid residue(s), carbohydrate-based polymers or carbohydrate residues and derivatives thereof, polynucleotides, alkyl groups, aryl groups, heterocyclic groups, combinations thereof, and substituted versions thereof. In some embodiments, each of L¹, L², L³, L⁴, L⁵ and L⁶ (if present) comprise one or more groups independently selected from a polyethylene glycol, a modified polyethylene glycol, an amino acid residue, an alkyl group, a substituted alkyl, an aryl group, a substituted aryl group, and a diamine (e.g., a linking group that includes an alkylene diamine).

In some embodiments, L¹ (if present) comprises a polyethylene glycol, a modified polyethylene glycol, an amino acid residue, an alkyl group, a substituted alkyl, an aryl group, a substituted aryl group, or a diamine. In some embodiments, L¹ comprises a polyethylene glycol. In some embodiments, L¹ comprises a modified polyethylene glycol. In some embodiments, L¹ comprises an amino acid residue. In some embodiments, L¹ comprises an alkyl group or a substituted alkyl. In some embodiments, L¹ comprises an aryl group or a substituted aryl group. In some embodiments, L¹ comprises a diamine (e.g., a linking group comprising an alkylene diamine).

In some embodiments, L² (if present) comprises a polyethylene glycol, a modified polyethylene glycol, an amino acid residue, an alkyl group, a substituted alkyl, an aryl group, a substituted aryl group, or a diamine. In some embodiments, L² comprises a polyethylene glycol. In some embodiments, L² comprises a modified polyethylene glycol. In some embodiments, L² comprises an amino acid residue. In some embodiments, L² comprises an alkyl group or a substituted alkyl. In some embodiments, L² comprises an aryl group or a substituted aryl group. In some embodiments, L² comprises a diamine (e.g., a linking group comprising an alkylene diamine).

In some embodiments, L³ (if present) comprises a polyethylene glycol, a modified polyethylene glycol, an amino acid residue, an alkyl group, a substituted alkyl, an aryl group, a substituted aryl group, or a diamine. In some embodiments, L³ comprises a polyethylene glycol. In some embodiments, L³ comprises a modified polyethylene glycol. In some embodiments, L³ comprises an amino acid residue. In some embodiments, L³ comprises an alkyl group or a substituted alkyl. In some embodiments, L³ comprises an aryl group or a substituted aryl group. In some embodiments, L³ comprises a diamine (e.g., a linking group comprising an alkylene diamine).

In some embodiments, L⁴ (if present) comprises a polyethylene glycol, a modified polyethylene glycol, an amino acid residue, an alkyl group, a substituted alkyl, an aryl group, a substituted aryl group, or a diamine. In some embodiments, L⁴ comprises a polyethylene glycol. In some embodiments, L⁴ comprises a modified polyethylene glycol. In some embodiments, L⁴ comprises an amino acid residue. In some embodiments, L⁴ comprises an alkyl group or a substituted alkyl. In some embodiments, L⁴ comprises an aryl group or a substituted aryl group. In some embodiments, L⁴ comprises a diamine (e.g., a linking group comprising an alkylene diamine).

In some embodiments, L⁵ (if present) comprises a polyethylene glycol, a modified polyethylene glycol, an amino acid residue, an alkyl group, a substituted alkyl, an aryl group, a substituted aryl group, or a diamine. In some embodiments, L⁵ comprises a polyethylene glycol. In some embodiments, L⁵ comprises a modified polyethylene glycol. In some embodiments, L⁵ comprises an amino acid residue. In some embodiments, L⁵ comprises an alkyl group or a substituted alkyl. In some embodiments, L⁵ comprises an aryl group or a substituted aryl group. In some embodiments, L⁵ comprises a diamine (e.g., a linking group comprising an alkylene diamine).

In some embodiments, L⁶ (if present) comprises a polyethylene glycol, a modified polyethylene glycol, an amino acid residue, an alkyl group, a substituted alkyl, an aryl group, a substituted aryl group, or a diamine. In some embodiments, L⁶ comprises a polyethylene glycol. In some embodiments, L⁶ comprises a modified polyethylene glycol. In some embodiments, L⁶ comprises an amino acid residue. In some embodiments, L⁶ comprises an alkyl group or a substituted alkyl. In some embodiments, L⁶ comprises an aryl group or a substituted aryl group. In some embodiments, L⁶ comprises a diamine (e.g., a linking group comprising an alkylene diamine).

In some embodiments, L^{A} is a first linker comprising -(L¹)ₐ-(L²)_{b}-(L³)_{c}-(L⁴)_{d}-(L⁵)ₑ-(L⁶)_{f}-, where:
-(L¹)ₐ- is -(T¹-V¹)ₐ-;
-(L²)_{b}- is -(T²-V²)_{b}-;
-(L³)_{c}- is -(T³-V³)_{c}-;
-(L⁴)_{d}- is -(T⁴-V⁴)_{d}-;
-(L⁵)ₑ- is -(T⁵-V⁵)ₑ-; and
-(L⁶)_{f}- is -(T⁶-V⁶)_{f}-,
wherein T¹, T², T³, T⁴, T⁵ and T⁶, if present, are tether groups;
V¹, V², V³, V⁴, V⁵ and V⁶, if present, are covalent bonds or linking functional groups; and
a, b, c, d, e and f are each independently 0 or 1.

In certain embodiments, the sum of a, b, c, d, e and f is 0 to 6. In certain embodiments, the sum of a, b, c, d, e and f is 0. In certain embodiments, the sum of a, b, c, d, e and f is 1. In certain embodiments, the sum of a, b, c, d, e and f is 2. In certain embodiments, the sum of a, b, c, d, e and f is 3. In certain embodiments, the sum of a, b, c, d, e and f is 4. In certain embodiments, the sum of a, b, c, d, e and f is 5. In certain embodiments, the sum of a, b, c, d, e and f is 6. In certain embodiments, a, b, c, d, e and f are each 1. In certain embodiments, a, b, c, d and e are each 1 and f is 0. In certain embodiments, a, b, c and d are each 1 and e and f are each 0. In certain embodiments, a, b, and c are each 1 and d, e and f are each 0. In certain embodiments, a and b are each 1 and c, d, e and f are each 0. In certain embodiments, a is 1 and b, c, d, e and f are each 0.

As described above, in certain embodiments, L¹ is attached to the hydrazinyl-indolyl or a hydrazinyl-pyrrolo-pyridinyl conjugation moiety (e.g., as shown in formula (I) above). As such, in certain embodiments, T¹ is attached to the hydrazinyl-indolyl or a hydrazinyl-pyrrolo-pyridinyl conjugation moiety (e.g., as shown in formula (I) above). In certain embodiments, V¹ is attached to the first drug or active agent. In certain embodiments, L², if present, is attached to the first drug or active agent. As such, in certain embodiments, T², if present, is attached to the first drug or active agent, or V², if present, is attached to the first drug or active agent. In certain embodiments, L³, if present, is attached to the first drug or active agent. As such, in certain embodiments, T³, if present, is attached to the first drug or active agent, or V³, if present, is attached to the first drug or active agent. In certain embodiments, L⁴, if present, is attached to the first drug or active agent. As such, in certain embodiments, T⁴, if present, is attached to the first drug or active agent, or V⁴, if present, is attached to the first drug or active agent. In certain embodiments, L⁵, if present, is attached to the first drug or active agent. As such, in certain embodiments, T⁵, if present, is attached to the first drug or active agent, or V⁵, if present, is attached to the first drug or active agent. In certain embodiments, L⁶, if present, is attached to the first drug or active agent. As such, in certain embodiments, T⁶, if present, is attached to the first drug or active agent, or V⁶, if present, is attached to the first drug or active agent.

In certain embodiments, the conjugate of formula (I) includes a second linker, L^{B}. The second linker, L^{B}, may be utilized to bind a second moiety of interest (e.g., a second drug or active agent) to a polypeptide (e.g., an antibody) through a conjugation moiety. The second linker, L^{B}, may be bound (e.g., covalently bonded) to the conjugation moiety (e.g., as described herein). For example, the second linker, L^{B}, may attach a hydrazinyl-indolyl or a hydrazinyl-pyrrolo-pyridinyl conjugation moiety to a second drug or active agent. The hydrazinyl-indolyl or hydrazinyl-pyrrolo-pyridinyl conjugation moiety may be used to conjugate the second linker, L^{B}, (and thus the second drug or active agent) to a polypeptide, such as an antibody.

For example, as shown in formula (I) above, L^{B} may be attached to W³ through a conjugation moiety, and thus W³ may be indirectly bonded to the second linker L^{B} through the hydrazinyl-indolyl or a hydrazinyl-pyrrolo-pyridinyl conjugation moiety. As described above, W³ is a polypeptide (e.g., an antibody), and thus L^{B} may be attached through the hydrazinyl-indolyl or a hydrazinyl-pyrrolo-pyridinyl conjugation moiety to the polypeptide (antibody), e.g., the linker L^{B} may be indirectly bonded to the polypeptide (antibody) through the hydrazinyl-indolyl or a hydrazinyl-pyrrolo-pyridinyl conjugation moiety.

Any convenient linker may be utilized for the second linker L^{B} in the subject conjugates and compounds. In certain embodiments, the second linker L^{B} may include a group selected from alkyl, substituted alkyl, alkenyl, substituted alkenyl, alkynyl, substituted alkynyl, alkoxy, substituted alkoxy, amino, substituted amino, carboxyl, carboxyl ester, acyl amino, alkylamide, substituted alkylamide, aryl, substituted aryl, heteroaryl, substituted heteroaryl, cycloalkyl, substituted cycloalkyl, heterocyclyl, and substituted heterocyclyl. In certain embodiments, the second linker L^{B} may include an alkyl or substituted alkyl group. In certain embodiments, the second linker L^{B} may include an alkenyl or substituted alkenyl group. In certain embodiments, the second linker L^{B} may include an alkynyl or substituted alkynyl group. In certain embodiments, the second linker L^{B} may include an alkoxy or substituted alkoxy group. In certain embodiments, the second linker L^{B} may include an amino or substituted amino group. In certain embodiments, the second linker L^{B} may include a carboxyl or carboxyl ester group. In certain embodiments, the second linker L^{B} may include an acyl amino group. In certain embodiments, the second linker L^{B} may include an alkylamide or substituted alkylamide group. In certain embodiments, the second linker L^{B} may include an aryl or substituted aryl group. In certain embodiments, the second linker L^{B} may include a heteroaryl or substituted heteroaryl group. In certain embodiments, the second linker L^{B} may include a cycloalkyl or substituted cycloalkyl group. In certain embodiments, the second linker L^{B} may include a heterocyclyl or substituted heterocyclyl group.

In certain embodiments, the second linker L^{B} may include a polymer. For example, the polymer may include a polyalkylene glycol and derivatives thereof, including polyethylene glycol, methoxypolyethylene glycol, polyethylene glycol homopolymers, polypropylene glycol homopolymers, copolymers of ethylene glycol with propylene glycol (e.g., where the homopolymers and copolymers are unsubstituted or substituted at one end with an alkyl group), polyvinyl alcohol, polyvinyl ethyl ethers, polyvinylpyrrolidone, combinations thereof, and the like. In certain embodiments, the polymer is a polyalkylene glycol. In certain embodiments, the polymer is a polyethylene glycol. Other linkers are also possible, as shown in the conjugates and compounds described in more detail below.

In some embodiments, L^{B} is a second linker described by the formula:

-(L⁷)_{g}-(L⁸)ₕ-(L⁹)ᵢ-(L¹⁰)ⱼ-(L¹¹)ₖ-(L¹²)ₗ-(L¹³)ₘ,
wherein L⁷, L⁸, L⁹, L¹⁰, L¹¹, L¹² and L¹³ are each independently a linker subunit, and g, h, i, j, k, l and m are each independently 0 or 1.

In certain embodiments, the sum of g, h, i, j, k, l and m is 0 to 7. In certain embodiments, the sum of g, h, i, j, k, l and m is 0. In certain embodiments, the sum of g, h, i, j, k, l and m is 1. In certain embodiments, the sum of g, h, i, j, k, l and m is 2. In certain embodiments, the sum of g, h, i, j, k, l and m is 3. In certain embodiments, the sum of g, h, i, j, k, l and m is 4. In certain embodiments, the sum of g, h, i, j, k, l and m is 5. In certain embodiments, the sum of g, h, i, j, k, l and m is 6. In certain embodiments, the sum of g, h, i, j, k, l and m is 7. In certain embodiments, g, h, i, j, k, l and m are each 1. In certain embodiments, g, h, i, j, k and l are each 1 and m is 0. In certain embodiments, g, h, i, j and k are each 1 and l and m are each 0. In certain embodiments, g, h, i and j are each 1 and k, l and m are each 0. In certain embodiments, g, h, and i are each 1 and j, k, l and m are each 0. In certain embodiments, g and h are each 1 and i, j, k, l and m are each 0. In certain embodiments, g is 1 and h, i, j, k, l and m are each 0. In certain embodiments, g, h, i, j, k, l and m are each 0.

In certain embodiments, the linker subunit L⁷ is attached to the hydrazinyl-indolyl or a hydrazinyl-pyrrolo-pyridinyl conjugation moiety (e.g., as shown in formula (I) above). In certain embodiments, the linker subunit L⁸, if present, is attached to the second drug or active agent W². In certain embodiments, the linker subunit L⁹, if present, is attached to the second drug or active agent W². In certain embodiments, the linker subunit L¹⁰, if present, is attached to the second drug or active agent W². In certain embodiments, the linker subunit L¹¹, if present, is attached to the second drug or active agent W². In certain embodiments, the linker subunit L¹², if present, is attached to the second drug or active agent W². In certain embodiments, the linker subunit L¹³, if present, is attached to the second drug or active agent W².

Any convenient linker subunits may be utilized in the second linker L^{B}. Linker subunits of interest include, but are not limited to, units of polymers such as polyethylene glycols, polyethylenes and polyacrylates, amino acid residue(s), carbohydrate-based polymers or carbohydrate residues and derivatives thereof, polynucleotides, alkyl groups, aryl groups, heterocyclic groups, combinations thereof, and substituted versions thereof. In some embodiments, each of L⁷, L⁸, L⁹, L¹⁰, L¹¹, L¹² and L¹³ (if present) comprise one or more groups independently selected from a polyethylene glycol, a modified polyethylene glycol, an amino acid residue, an alkyl group, a substituted alkyl, an aryl group, a substituted aryl group, and a diamine (e.g., a linking group that includes an alkylene diamine).

In some embodiments, L⁷ (if present) comprises a polyethylene glycol, a modified polyethylene glycol, an amino acid residue, an alkyl group, a substituted alkyl, an aryl group, a substituted aryl group, or a diamine. In some embodiments, L⁷ comprises a polyethylene glycol. In some embodiments, L⁷ comprises a modified polyethylene glycol. In some embodiments, L⁷ comprises an amino acid residue. In some embodiments, L⁷ comprises an alkyl group or a substituted alkyl. In some embodiments, L⁷ comprises an aryl group or a substituted aryl group. In some embodiments, L⁷ comprises a diamine (e.g., a linking group comprising an alkylene diamine).

In some embodiments, L⁸ (if present) comprises a polyethylene glycol, a modified polyethylene glycol, an amino acid residue, an alkyl group, a substituted alkyl, an aryl group, a substituted aryl group, or a diamine. In some embodiments, L⁸ comprises a polyethylene glycol. In some embodiments, L⁸ comprises a modified polyethylene glycol. In some embodiments, L⁸ comprises an amino acid residue. In some embodiments, L⁸ comprises an alkyl group or a substituted alkyl. In some embodiments, L⁸ comprises an aryl group or a substituted aryl group. In some embodiments, L⁸ comprises a diamine (e.g., a linking group comprising an alkylene diamine).

In some embodiments, L⁹ (if present) comprises a polyethylene glycol, a modified polyethylene glycol, an amino acid residue, an alkyl group, a substituted alkyl, an aryl group, a substituted aryl group, or a diamine. In some embodiments, L⁹ comprises a polyethylene glycol. In some embodiments, L⁹ comprises a modified polyethylene glycol. In some embodiments, L⁹ comprises an amino acid residue. In some embodiments, L⁹ comprises an alkyl group or a substituted alkyl. In some embodiments, L⁹ comprises an aryl group or a substituted aryl group. In some embodiments, L⁹ comprises a diamine (e.g., a linking group comprising an alkylene diamine).

In some embodiments, L¹⁰ (if present) comprises a polyethylene glycol, a modified polyethylene glycol, an amino acid residue, an alkyl group, a substituted alkyl, an aryl group, a substituted aryl group, or a diamine. In some embodiments, L¹⁰ comprises a polyethylene glycol. In some embodiments, L¹⁰ comprises a modified polyethylene glycol. In some embodiments, L¹⁰ comprises an amino acid residue. In some embodiments, L¹⁰ comprises an alkyl group or a substituted alkyl. In some embodiments, L¹⁰ comprises an aryl group or a substituted aryl group. In some embodiments, L¹⁰ comprises a diamine (e.g., a linking group comprising an alkylene diamine).

In some embodiments, L¹¹ (if present) comprises a polyethylene glycol, a modified polyethylene glycol, an amino acid residue, an alkyl group, a substituted alkyl, an aryl group, a substituted aryl group, or a diamine. In some embodiments, L¹¹ comprises a polyethylene glycol. In some embodiments, L¹¹ comprises a modified polyethylene glycol. In some embodiments, L¹¹ comprises an amino acid residue. In some embodiments, L¹¹ comprises an alkyl group or a substituted alkyl. In some embodiments, L¹¹ comprises an aryl group or a substituted aryl group. In some embodiments, L¹¹ comprises a diamine (e.g., a linking group comprising an alkylene diamine).

In some embodiments, L¹² (if present) comprises a polyethylene glycol, a modified polyethylene glycol, an amino acid residue, an alkyl group, a substituted alkyl, an aryl group, a substituted aryl group, or a diamine. In some embodiments, L¹² comprises a polyethylene glycol. In some embodiments, L¹² comprises a modified polyethylene glycol. In some embodiments, L¹² comprises an amino acid residue. In some embodiments, L¹² comprises an alkyl group or a substituted alkyl. In some embodiments, L¹² comprises an aryl group or a substituted aryl group. In some embodiments, L¹² comprises a diamine (e.g., a linking group comprising an alkylene diamine).

In some embodiments, L¹³ (if present) comprises a polyethylene glycol, a modified polyethylene glycol, an amino acid residue, an alkyl group, a substituted alkyl, an aryl group, a substituted aryl group, or a diamine. In some embodiments, L¹³ comprises a polyethylene glycol. In some embodiments, L¹³ comprises a modified polyethylene glycol. In some embodiments, L¹³ comprises an amino acid residue. In some embodiments, L¹³ comprises an alkyl group or a substituted alkyl. In some embodiments, L¹³ comprises an aryl group or a substituted aryl group. In some embodiments, L¹³ comprises a diamine (e.g., a linking group comprising an alkylene diamine).

In some embodiments, L^{B} is a second linker comprising -(L⁷)_{g}-(L⁸)ₕ-(L⁹)ᵢ-(L¹⁰)ⱼ-(L¹¹)ₖ-(L¹²)ₗ-(L¹³)ₘ-, where:
-(L⁷)_{g}- is -(T⁷-V⁷)_{g}-;
-(L⁸)ₕ- is -(T⁸-V⁸)ₕ-;
-(L⁹)ᵢ- is -(T⁹-V⁹)ᵢ-;
-(L¹⁰)ⱼ- is -(T¹⁰-V¹⁰)ⱼ-;
-(L¹¹)ₖ- is -(T¹¹-V¹¹)ₖ-;
-(L¹²)ₗ- is -(T¹²-V¹²)ₗ-; and
-(L¹³)ₘ- is -(T¹³-V¹³)ₘ-,
wherein T⁷, T⁸, T⁹, T¹⁰, T¹¹, T¹² and T¹³, if present, are tether groups;
V⁷, V⁸, V⁹, V¹⁰, V¹¹, V¹² and V¹³, if present, are covalent bonds or linking functional groups; and
g, h, i, j, k, l and m are each independently 0 or 1.

In certain embodiments, the sum of g, h, i, j, k, l and m is 0 to 7. In certain embodiments, the sum of g, h, i, j, k, l and m is 0. In certain embodiments, the sum of g, h, i, j, k, l and m is 1. In certain embodiments, the sum of g, h, i, j, k, l and m is 2. In certain embodiments, the sum of g, h, i, j, k, l and m is 3. In certain embodiments, the sum of g, h, i, j, k, l and m is 4. In certain embodiments, the sum of g, h, i, j, k, l and m is 5. In certain embodiments, the sum of g, h, i, j, k, l and m is 6. In certain embodiments, the sum of g, h, i, j, k, l and m is 7. In certain embodiments, g, h, i, j, k, l and m are each 1. In certain embodiments, g, h, i, j, k and l are each 1 and m is 0. In certain embodiments, g, h, i, j and k are each 1 and l and m are each 0. In certain embodiments, g, h, i and j are each 1 and k, l and m are each 0. In certain embodiments, g, h, and i are each 1 and j, k, l and m are each 0. In certain embodiments, g and h are each 1 and i, j, k, l and m are each 0. In certain embodiments, g is 1 and h, i, j, k, l and m are each 0. In certain embodiments, g, h, i, j, k, l and m are each 0.

As described above, in certain embodiments, L⁷ is attached to the hydrazinyl-indolyl or a hydrazinyl-pyrrolo-pyridinyl conjugation moiety (e.g., as shown in formula (I) above). As such, in certain embodiments, T⁷ is attached to the hydrazinyl-indolyl or a hydrazinyl-pyrrolo-pyridinyl conjugation moiety (e.g., as shown in formula (I) above). In certain embodiments, V⁷ is attached to the second drug or active agent. In certain embodiments, L⁸, if present, is attached to the second drug or active agent. As such, in certain embodiments, T⁸, if present, is attached to the second drug or active agent, or V⁸, if present, is attached to the second drug or active agent. In certain embodiments, L⁹, if present, is attached to the second drug or active agent. As such, in certain embodiments, T⁹, if present, is attached to the second drug or active agent, or V⁹, if present, is attached to the second drug or active agent. In certain embodiments, L¹⁰, if present, is attached to the second drug or active agent. As such, in certain embodiments, T¹⁰, if present, is attached to the second drug or active agent, or V10⁴, if present, is attached to the second drug or active agent. In certain embodiments, L¹¹, if present, is attached to the second drug or active agent. As such, in certain embodiments, T¹¹, if present, is attached to the second drug or active agent, or V¹¹, if present, is attached to the second drug or active agent. In certain embodiments, L¹², if present, is attached to the second drug or active agent. As such, in certain embodiments, T¹², if present, is attached to the second drug or active agent, or V¹², if present, is attached to the second drug or active agent. In certain embodiments, L¹³, if present, is attached to the second drug or active agent. As such, in certain embodiments, T¹³, if present, is attached to the second drug or active agent, or V¹³, if present, is attached to the second drug or active agent.

Regarding the tether groups, T¹, T², T³, T⁴, T⁵, T⁶, T⁷, T⁸, T⁹, T¹⁰, T¹, T¹² and T¹³, any convenient tether groups may be utilized in the subject linkers. In some embodiments, T¹, T², T³, T⁴, T⁵, T⁶, T⁷, T⁸, T⁹, T¹⁰, T¹, T¹² and T¹³ each comprise one or more groups independently selected from a covalent bond, a (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, where each w is an integer from 1 to 20, each n is an integer from 1 to 30, each p is an integer from 1 to 20, and each x is an integer from 1 to 12.

In certain embodiments, the tether group (e.g., T¹, T², T³, T⁴, T⁵, T⁶, T⁷, T⁸, T⁹, T¹⁰, T¹¹, T¹² and/or T¹³) includes a (C₁-C₁₂)alkyl or a substituted (C₁-C₁₂)alkyl. In certain embodiments, (C₁-C₁₂)alkyl is a straight chain or branched alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, (C₁-C₁₂)alkyl may be an alkyl or substituted alkyl, such as C₁-C₁₂ alkyl, or C₁-C₁₀ alkyl, or C₁-C₆ alkyl, or C₁-C₃ alkyl. In some instances, (C₁-C₁₂)alkyl is a C₂-alkyl. For example, (C₁-C₁₂)alkyl may be an alkylene or substituted alkylene, such as C₁-C₁₂ alkylene, or C₁-C₁₀ alkylene, or C₁-C₆ alkylene, or C₁-C₃ alkylene. In some instances, (C₁-C₁₂)alkyl is a C₁-alkylene (e.g., CH₂). In some instances, (C₁-C₁₂)alkyl is a C₂-alkylene (e.g., CH₂CH₂). In some instances, (C₁-C₁₂)alkyl is a C₃-alkylene (e.g., CH₂CH₂CH₂).

In certain embodiments, substituted (C₁-C₁₂)alkyl is a straight chain or branched substituted alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, substituted (C₁-C₁₂)alkyl may be a substituted alkyl, such as substituted C₁-C₁₂ alkyl, or substituted C₁-C₁₀ alkyl, or substituted C₁-C₆ alkyl, or substituted C₁-C₃ alkyl. In some instances, substituted (C₁-C₁₂)alkyl is a substituted C₂-alkyl. For example, substituted (C₁-C₁₂)alkyl may be a substituted alkylene, such as substituted C₁-C₁₂ alkylene, or substituted C₁-C₁₀ alkylene, or substituted C₁-C₆ alkylene, or substituted C₁-C₃ alkylene. In some instances, substituted (C₁-C₁₂)alkyl is a substituted C₁-alkylene (e.g., C₁-alkylene substituted with —SO₃H). In some instances, substituted (C₁-C₁₂)alkyl is a substituted C₂-alkylene. In some instances, substituted (C₁-C₁₂)alkyl is a substituted C₃-alkylene. For example, substituted (C₁-C₁₂)alkyl may include C₁-C₁₂ alkylene (e.g., C₃-alkylene or C₅-alkylene) substituted with a (PEG)ₖ group as described herein (e.g., —CONH(PEG)ₖ, such as —CONH(PEG)₃ or —CONH(PEG)₅; or —NHCO(PEG)ₖ, such as —NHCO(PEG)₇), or may include C₁-C₁₂ alkylene (e.g., C₃-alkylene) substituted with a —CONHCH₂CH₂SO₃H group, or may include C₁-C₁₂ alkylene (e.g., C₅-alkylene) substituted with a —NHCOCH₂SO₃H group.

In certain embodiments, the tether group (e.g., T¹, T², T³, T⁴, T⁵, T⁶, T⁷, T⁸, T⁹, T¹⁰, T¹, T¹² and/or T¹³) includes an aryl, substituted aryl, heteroaryl, substituted heteroaryl, cycloalkyl, substituted cycloalkyl, heterocyclyl, or substituted heterocyclyl. In some instances, the tether group (e.g., T¹, T², T³, T⁴, T⁵, T⁶, T⁷, T⁸, T⁹, T¹⁰, T¹, T¹² and/or T¹³) includes an aryl or substituted aryl. For example, the aryl can be phenyl. In some cases, the substituted aryl is a substituted phenyl. The substituted phenyl can be substituted with one or more substituents selected from (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl. In some instances, the substituted aryl is a substituted phenyl, where the substituent includes a cleavable moiety as described herein (e.g., an enzymatically cleavable moiety, such as a glycoside or glycoside derivative).

In some instances, the tether group (e.g., T¹, T², T³, T⁴, T⁵, T⁶, T⁷, T⁸, T⁹, T₁₀, T₁₁, T¹² and/or T¹³) includes a heteroaryl or substituted heteroaryl, such triazolyl (e.g., 1,2,3-triazolyl). In some instances, the tether group (e.g., T¹, T², T³, T⁴, T⁵, T⁶, T⁷, T⁸, T⁹, T¹⁰, T¹¹, T¹² and/or T¹³) includes a cycloalkyl or substituted cycloalkyl. In some instances, the tether group (e.g., T¹, T², T³, T⁴, T⁵, T⁶, T⁷, T⁸, T⁹, T¹⁰, T¹¹, T¹² and/or T¹³) includes a heterocyclyl or substituted heterocyclyl. In some instances, the substituent on the substituted heteroaryl, substituted cycloalkyl or substituted heterocyclyl includes a cleavable moiety as described herein (e.g., an enzymatically cleavable moiety, such as a glycoside or glycoside derivative).

In certain embodiments, the tether group (e.g., T¹, T², T³, T⁴, T⁵, T⁶, T⁷, T⁸, T⁹, T¹⁰, T¹¹, T¹² and/or T¹³) includes an ethylene diamine (EDA) moiety, e.g., an EDA containing tether group. In certain embodiments, (EDA) w includes one or more EDA moieties, such as where w is an integer from 1 to 50, such as from 1 to 40, from 1 to 30, from 1 to 20, from 1 to 12 or from 1 to 6, such as 1, 2, 3, 4, 5 or 6). The linked ethylene diamine (EDA) moieties may optionally be substituted at one or more convenient positions with any convenient substituents, e.g., with an alkyl, a substituted alkyl, an acyl, a substituted acyl, an aryl or a substituted aryl. In certain embodiments, the EDA moiety is described by the structure:

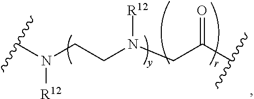

where y is an integer from 1 to 6, or is 0 or 1, and each R¹² is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, y is 1, 2, 3, 4, 5 or 6. In certain embodiments, y is 1 and r is 0. In certain embodiments, y is 1 and r is 1. In certain embodiments, y is 2 and r is 0. In certain embodiments, y is 2 and r is 1. In certain embodiments, each R¹² is independently selected from hydrogen, an alkyl, a substituted alkyl, an aryl and a substituted aryl. In certain embodiments, any two adjacent R¹² groups of the EDA may be cyclically linked, e.g., to form a piperazinyl ring. In certain embodiments, y is 1 and the two adjacent R¹² groups are an alkyl group, cyclically linked to form a piperazinyl ring. In certain embodiments, y is 1 and the adjacent R¹² groups are selected from hydrogen, an alkyl (e.g., methyl) and a substituted alkyl (e.g., lower alkyl-OH, such as ethyl-OH or propyl-OH).

In certain embodiments, the tether group (e.g., T¹, T², T³, T⁴, T⁵, T⁶, T⁷, T⁸, T⁹, T¹⁰, T¹¹, T¹² and/or T¹³) includes a 4-amino-piperidine (4AP) moiety (also referred to herein as piperidin-4-amino, P4A). The 4AP moiety may optionally be substituted at one or more convenient positions with any convenient substituents, e.g., with an alkyl, a substituted alkyl, a polyethylene glycol moiety, an acyl, a substituted acyl, an aryl or a substituted aryl. In certain embodiments, the 4AP moiety is described by the structure:

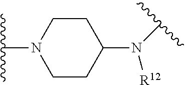

where R¹² is selected from hydrogen, alkyl, substituted alkyl, a polyethylene glycol moiety (e.g., a polyethylene glycol or a modified polyethylene glycol), alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R¹² is a polyethylene glycol moiety. In certain embodiments, R¹² is a carboxy modified polyethylene glycol.

In certain embodiments, R¹² includes a polyethylene glycol moiety described by the formula: (PEG)ₖ, which may be represented by the structure:

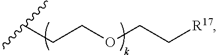

where k is an integer from 1 to 20, such as from 1 to 18, or from 1 to 16, or from 1 to 14, or from 1 to 12, or from 1 to 10, or from 1 to 8, or from 1 to 6, or from 1 to 4, or 1 or 2, such as 1, 2, 3, 4, 5, 6, 7, 8, 9, 10, 11, 12, 13, 14, 15, 16, 17, 18, 19 or 20. In some instances, k is 2. In certain embodiments, R¹⁷ is selected from OH, COOH, OR, or COOR, where R is selected from alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl. In certain embodiments, R¹⁷ is COOH. In certain embodiments, R¹⁷ is OH. In certain embodiments, R¹⁷ is OCH₃.

In certain embodiments, a tether group (e.g., T¹, T², T³, T⁴, T⁵, T⁶, T⁷, T⁸, T⁹, T¹⁰, T¹¹, T¹² and/or T¹³) includes (PEG)ₙ, where (PEG), is a polyethylene glycol or a modified polyethylene glycol linking unit. In certain embodiments, (PEG)ₙ is described by the structure:

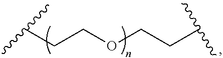

where n is an integer from 1 to 50, such as from 1 to 40, from 1 to 30, from 1 to 20, from 1 to 12 or from 1 to 6, such as 1, 2, 3, 4, 5, 6, 7, 8, 9, 10, 11, 12, 13, 14, 15, 16, 17, 18, 19 or 20. In some instances, n is 2. In some instances, n is 3. In some instances, n is 6. In some instances, n is 12.

In certain embodiments, a tether group (e.g., T¹, T², T³, T⁴, T⁵, T⁶, T⁷, T⁸, T⁹, T¹⁰, T¹¹, T¹² and/or T¹³) includes (AA)ₚ, where AA is an amino acid residue. Any convenient amino acids may be utilized. Amino acids of interest include but are not limited to, L- and D-amino acids, naturally occurring amino acids such as any of the 20 primary alpha-amino acids and beta-alanine, non-naturally occurring amino acids (e.g., amino acid analogs), such as a non-naturally occurring alpha-amino acid or a non-naturally occurring beta-amino acid, etc. In certain embodiments, p is an integer from 1 to 50, such as from 1 to 40, from 1 to 30, from 1 to 20, from 1 to 12 or from 1 to 6, such as 1, 2, 3, 4, 5, 6, 7, 8, 9, 10, 11, 12, 13, 14, 15, 16, 17, 18, 19 or 20. In certain embodiments, p is 1. In certain embodiments, p is 2.

In certain embodiments, a tether group (e.g., T¹, T², T³, T⁴, T⁵, T⁶, T⁷, T⁸, T⁹, T¹⁰, T¹¹, T¹² and/or T¹³) includes an amino acid analog. Amino acid analogs include compounds that are similar in structure and/or overall shape to one or more amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y). Amino acid analogs also include natural amino acids with modified side chains or backbones. Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as Cl or Br, etc.), deletion of a group, substitution of a covalent bond (single bond for double bond, etc.), or combinations thereof. For example, amino acid analogs may include α-hydroxy acids, and α-amino acids, and the like. Examples of amino acid analogs include, but are not limited to, sulfoalanine, and the like.

In certain embodiments, a tether group (e.g., T¹, T², T³, T⁴, T⁵, T⁶, T⁷, T⁸, T⁹, T¹⁰, T¹¹, T¹² and/or T¹³) includes a moiety described by the formula —(CR¹³OH)ₓ—, where x is 0 or x is an integer from 1 to 50, such as from 1 to 40, from 1 to 30, from 1 to 20, from 1 to 12 or from 1 to 6, such as 1, 2, 3, 4, 5, 6, 7, 8, 9, 10, 11 or 12. In certain embodiments, x is 1. In certain embodiments, x is 2. In certain embodiments, R¹³ is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R¹³ is hydrogen. In certain embodiments, R¹³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹³ is alkynyl or substituted alkynyl. In certain embodiments, R¹³ is alkoxy or substituted alkoxy. In certain embodiments, R¹³ is amino or substituted amino. In certain embodiments, R¹³ is carboxyl or carboxyl ester. In certain embodiments, R¹³ is acyl or acyloxy. In certain embodiments, R¹³ is acyl amino or amino acyl. In certain embodiments, R¹³ is alkylamide or substituted alkylamide. In certain embodiments, R¹³ is sulfonyl. In certain embodiments, R¹³ is thioalkoxy or substituted thioalkoxy. In certain embodiments, R¹³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹³ is cycloalkyl or substituted cycloalkyl, such as C₃₋₈ cycloalkyl or C₃₋₈ substituted cycloalkyl, such as a C₃₋₆ Cycloalkyl or C₃₋₆ substituted cycloalkyl, or a C₃₋₅ cycloalkyl or C₃₋₅ substituted cycloalkyl. In certain embodiments, R¹³ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R¹³ is selected from hydrogen, alkyl, substituted alkyl, aryl, and substituted aryl. In these embodiments, alkyl, substituted alkyl, aryl, and substituted aryl are as described above for R¹³.

In certain embodiments, the tether group (e.g., T¹, T², T³, T⁴, T⁵, T⁶, T⁷, T⁸, T⁹, T¹⁰, T¹¹, T¹² and/or T¹³) includes an acetal group, a disulfide, a hydrazine, or an ester. In some embodiments, the tether group includes an acetal group. In some embodiments, the tether group includes a hydrazine. In some embodiments, the tether group includes a disulfide. In some embodiments, the tether group includes an ester.

In certain embodiments, a tether group (e.g., T¹, T², T³, T⁴, T⁵, T⁶, T⁷, T⁸, T⁹, T¹⁰, T¹¹, T¹² and/or T¹³) includes a meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), or para-hydroxy-phenyl (PHP).

In some embodiments, a tether group includes a MABO group described by the following structure:

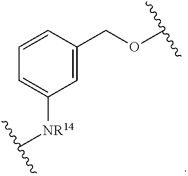

In some embodiments, a tether group includes a MABC group described by the following structure:

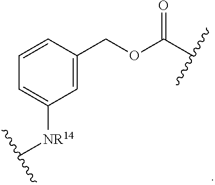

In some embodiments, a tether group includes a PABO group described by the following structure:

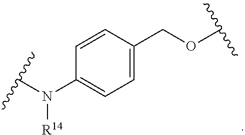

In some embodiments, a tether group includes a PABC group described by the following structure:

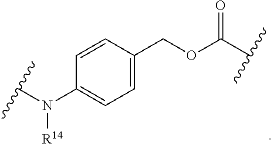

In some embodiments, a tether group includes a PAB group described by the following structure:

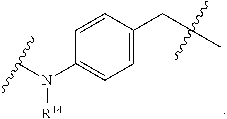

In some embodiments, a tether group includes a PABA group described by the following structure:

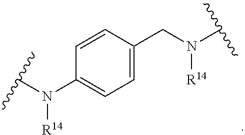

In some embodiments, a tether group includes a PAP group described by the following structure:

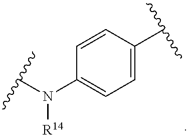

In some embodiments, a tether group includes a PHP group described by the following structure:

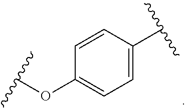 [SMILES: *Oc1ccc(*)cc1]

In certain embodiments, each R¹⁴ is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In certain embodiments, R¹⁴ is hydrogen. In certain embodiments, each R¹⁴ is hydrogen. In certain embodiments, R¹⁴ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹⁴ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹⁴ is alkynyl or substituted alkynyl. In certain embodiments, R¹⁴ is alkoxy or substituted alkoxy. In certain embodiments, R¹⁴ is amino or substituted amino. In certain embodiments, R¹⁴ is carboxyl or carboxyl ester. In certain embodiments, R¹⁴ is acyl or acyloxy. In certain embodiments, R¹⁴ is acyl amino or amino acyl. In certain embodiments, R¹⁴ is alkylamide or substituted alkylamide. In certain embodiments, R¹⁴ is sulfonyl. In certain embodiments, R¹⁴ is thioalkoxy or substituted thioalkoxy. In certain embodiments, R¹⁴ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹⁴ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹⁴ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹⁴ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In some embodiments of the MABO, MABC, PABO, PABC, PAB, PABA, PAP, and PHP tether structures shown above, the phenyl ring may be substituted with one or more additional groups selected from halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In certain embodiments, one or more of the tether groups T¹, T², T³, T⁴, T⁵, T⁶, T⁷, T⁸, T⁹, T¹⁰, T¹, T¹² and/or T¹³ is each optionally substituted with a glycoside or glycoside derivative. For example, in some instances, T¹, T², T³, T⁴, T⁵ and T⁶ are each optionally substituted with a glycoside. In some instances, T⁷, T⁸, T⁹, T¹⁰, T¹¹, T¹² and T¹³ are each optionally substituted with a glycoside. In certain embodiments, the glycoside or glycoside derivative is selected from a glucuronide, a galactoside, a glucoside, a mannoside, a fucoside, O-GlcNAc, and O-GalNAc.

In certain embodiments, the MABO, MABC, PABO, PABC, PAB, PABA, PAP, and PHP tether structures shown above may be substituted with an one or more additional groups selected from a glycoside and a glycoside derivative. For example, in some embodiments of the MABO, MABC, PABO, PABC, PAB, PABA, PAP, and PHP tether structures shown above, the phenyl ring may be substituted with one or more additional groups selected from a glycoside and a glycoside derivative. In certain embodiments, the glycoside or glycoside derivative is selected from a glucuronide, a galactoside, a glucoside, a mannoside, a fucoside, O-GlcNAc, and O-GalNAc.

For example, in some embodiments, the glycoside or glycoside derivative can be selected from the following structures:

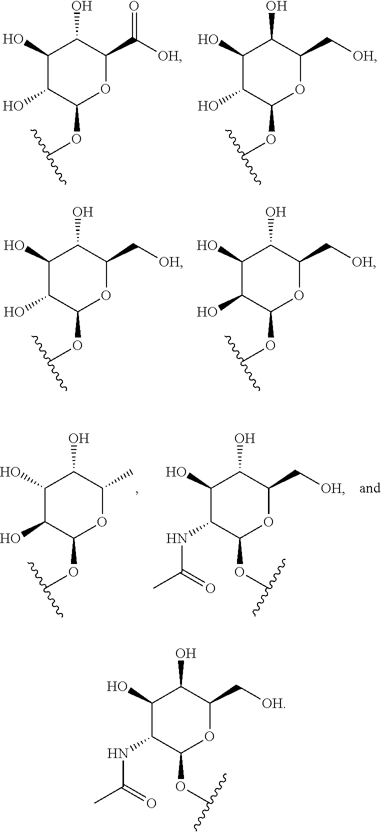

Regarding the linking functional groups, V¹, V², V³, V⁴, V⁵, V⁶, V⁷, V⁸, V°, V¹⁰, V¹¹, V¹² and V¹³ any convenient linking functional groups may be utilized in the subject linkers. Linking functional groups of interest include, but are not limited to, amino, carbonyl, amido, oxycarbonyl, carboxy, sulfonyl, sulfoxide, sulfonylamino, aminosulfonyl, thio, oxy, phospho, phosphoramidate, thiophosphoraidate, and the like. In some embodiments, V¹, V², V³, V⁴, V⁵, V⁶, V⁷, V⁸, V⁹, V¹⁰, V¹¹, V¹² and V¹³ are each independently selected from a covalent bond, —CO—, —NR¹⁵—, —NR¹⁵ (CH₂)_{q}—, —NR¹⁵ (C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, where q is an integer from 1 to 6. In certain embodiments, q is an integer from 1 to 6 (e.g., 1, 2, 3, 4, 5 or 6). In certain embodiments, q is 1. In certain embodiments, q is 2. In certain embodiments, q is 3. In certain embodiments, q is 4. In certain embodiments, q is 5. In certain embodiments, q is 6.

In some embodiments, each R¹⁵ is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In certain embodiments, R¹⁵ is hydrogen. In certain embodiments, each R¹⁵ is hydrogen. In certain embodiments, R¹⁵ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹⁵ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹⁵ is alkynyl or substituted alkynyl. In certain embodiments, R¹⁵ is alkoxy or substituted alkoxy. In certain embodiments, R¹⁵ is amino or substituted amino. In certain embodiments, R¹⁵ is carboxyl or carboxyl ester. In certain embodiments, R¹⁵ is acyl or acyloxy. In certain embodiments, R¹⁵ is acyl amino or amino acyl. In certain embodiments, R¹⁵ is alkylamide or substituted alkylamide. In certain embodiments, R¹⁵ is sulfonyl. In certain embodiments, R¹⁵ is thioalkoxy or substituted thioalkoxy. In certain embodiments, R¹⁵ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹⁵ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹⁵ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹⁵ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl. In these embodiments, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl are as described above for R¹⁵.

As described above, in some embodiments, L^{A} is a first linker comprising -(T¹-V¹)ₐ-(T²-V²)_{b}-(T³-V³)_{c}-(T⁴-V⁴)_{d}-(T⁵-V⁵)ₑ-(T⁶-V⁶)_{f}-, where a, b, c, d, e and f are each independently 0 or 1.

In some embodiments, in the first linker L^{A}:
T¹ is selected from a (C₁-C₁₂)alkyl and a substituted (C₁-C₁₂)alkyl;
T², T³, T⁴, T⁵ and T⁶ are each independently selected from (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), MABO, MABC, PABO, PABC, PAB, PABA, PAP, PHP, an acetal group, a disulfide, a hydrazine, and an ester; and
V¹, V², V³, V⁴, V⁵ and V⁶ are each independently selected from a covalent bond, —CO—, —NR¹⁵, —NR¹⁵ (CH₂)_{q}—, —NR¹⁵ (C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein q is an integer from 1 to 6;
wherein:
(PEG)ₙ is

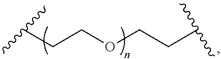

where n is an integer from 1 to 30;
EDA is an ethylene diamine moiety having the following structure:

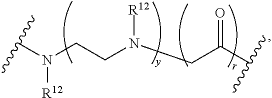

where y is an integer from 1 to 6 and r is 0 or 1;
4-amino-piperidine (4AP) is

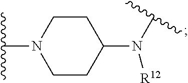

AA is an amino acid residue, where p is an integer from 1 to 20; and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring;
each R¹³ is independently selected from hydrogen, alkyl, substituted alkyl, aryl, and substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

In certain embodiments, T¹, T², T³, T⁴, T⁵ and T⁶ and V¹, V², V³, V⁴, V⁵ and V⁶ are selected from the following:

wherein:
T¹ is (C₁-C₁₂)alkyl and V¹ is —CO—;
T² is an amino acid analog and V² is —NH—;
T³ is (PEG), and V³ is —CO—;
T⁴ is AA and V⁴ is absent;
T⁵ is PABC and V⁵ is absent; and
f is 0; or
   wherein:
T¹ is (C₁-C₁₂)alkyl and V¹ is —CONH—;
T² is (PEG), and V² is —CO—;
T³ is AA and V³ is absent;
T⁴ is PABC and V⁴ is absent; and
e and f are each 0; or
   wherein:
T¹ is (C₁-C₁₂)alkyl and V¹ is —CONH—;
T² is substituted (C₁-C₁₂)alkyl and V² is —CO—;
T³ is AA and V³ is absent;
T⁴ is PABC and V⁴ is absent; and
e and f are each 0.

In certain embodiments, the left-hand side of the above linker structure for the first linker L^{A} is attached to the hydrazinyl-indolyl or a hydrazinyl-pyrrolo-pyridinyl conjugation moiety, and the right-hand side of the above linker structure for the first linker L^{A} is attached to the first drug or active agent.

As described above, in some embodiments, L^{B} is a second linker comprising -(T⁷-V⁷)_{g}-(T⁸-V⁸)ₕ-(T⁹-V⁹)ᵢ-(T¹⁰-V¹⁰)ⱼ-(T¹¹-V¹¹)ₖ-(T¹²-V¹²)ₗ-(T¹³-V¹³)ₘ-, where g, h, i, j, k, l and m are each independently 0 or 1.

In some embodiments, in the second linker L^{B}:
T⁷ is selected from a (C₁-C₁₂)alkyl and a substituted (C₁-C₁₂)alkyl;
T⁸, T⁹, T¹⁰, T¹¹, T¹² and T¹³ are each independently selected from (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), MABO, MABC, PABO, PABC, PAB, PABA, PAP, PHP, an acetal group, a disulfide, a hydrazine, and an ester; and
V⁷, V⁸, V⁹, V¹⁰, V¹¹, V¹² and V¹³ are each independently selected from a covalent bond, —CO—, —NR¹⁵—, —NR¹⁵ (CH₂)_{q}—, —NR¹⁵ (C₆H₄)—, —CONR¹⁵—, —NR¹⁵CO—, —C(O)O—, —OC(O)—, —O—, —S—, —S(O)—, —SO₂—, —SO₂NR¹⁵, —NR¹⁵SO₂— and —P(O)OH—, wherein q is an integer from 1 to 6;

wherein:
(PEG)ₙ is

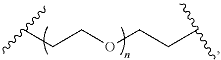

where n is an integer from 1 to 30;
EDA is an ethylene diamine moiety having the following structure:

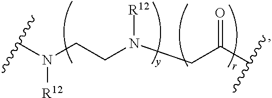

where y is an integer from 1 to 6 and r is 0 or 1;
4-amino-piperidine (4AP) is

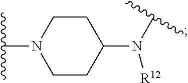

AA is an amino acid residue, where p is an integer from 1 to 20; and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring;
each R¹³ is independently selected from hydrogen, alkyl, substituted alkyl, aryl, and substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

Any convenient tether groups may be utilized for T⁷, T⁸, T⁹, T¹⁰, T¹¹, T¹² and T¹³. For example, any of the tether groups described above in relation to T¹, T², T³, T⁴, T⁵ and T⁶ may be used for the tether groups T⁷, T⁸, T⁹, T¹⁰, T¹¹, T¹² and T¹³.

Any convenient linking functional groups may be utilized for V⁷, V⁸, V⁹, V¹⁰, V¹¹, V¹² and V¹³. For example, any of the linking functional groups described above in relation to V¹, V², V³, V⁴, V⁵ and V⁶ may be used for the linking functional groups V⁷, V⁸, V⁹, V¹⁰, V¹¹, V¹² and V¹³.

In certain embodiments, each R¹³ is independently selected from hydrogen, alkyl, substituted alkyl, aryl, and substituted aryl. In these embodiments, alkyl, substituted alkyl, aryl, and substituted aryl are as described above for R¹³.

In certain embodiments, each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl. In these embodiments, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl are as described above for R¹⁵. In these embodiments, various possible substituents are as described above for R¹⁵.

In certain embodiments of the second linker L^{B}, one or more of the tether groups T⁷, T⁸, T⁹, T¹⁰, T¹¹, T¹² and T¹³ is each optionally substituted with a glycoside or glycoside derivative. In certain embodiments, the glycoside or glycoside derivative is selected from a glucuronide, a galactoside, a glucoside, a mannoside, a fucoside, O-GlcNAc, and O-GalNAc.

In certain embodiments of the second linker L^{B}, the MABO, MABC, PABO, PABC, PAB, PABA, PAP, and PHP tether structures shown above may be substituted with an one or more additional groups selected from a glycoside and a glycoside derivative. For example, in some embodiments of the MABO, MABC, PABO, PABC, PAB, PABA, PAP, and PHP tether structures shown above, the phenyl ring may be substituted with one or more additional groups selected from a glycoside and a glycoside derivative. In certain embodiments, the glycoside or glycoside derivative is selected from a glucuronide, a galactoside, a glucoside, a mannoside, a fucoside, O-GlcNAc, and O-GalNAc.

In certain embodiments, T⁷, T⁸, T⁹, T¹⁰, T¹¹, T¹² and T¹³ and V⁷, V⁸, V⁹, V¹⁰, V¹¹, V¹² and V¹³ are selected from the following:

wherein:
T⁷ is absent and V⁷ is —NHCO—;
T⁸ is (C₁-C₁₂)alkyl and V⁸ is —CONH—;
T⁹ is (PEG), and V⁹ is —CO—;
T¹⁰ is AA and V¹⁰ is absent; and
T¹¹ is PABC and V¹¹ is absent; and
l and m are each 0; or
   wherein:
T⁷ is absent and V⁷ is —NHCO—;
T⁸ is (C₁-C₁₂)alkyl and V⁸ is —CONH—;
T⁹ is substituted (C₁-C₁₂)alkyl and V⁹ is —CO—;
T¹⁰ is AA and V¹⁰ is absent;
T¹¹ is PABC and V¹¹ is absent; and
l and m are each 0.

In certain embodiments, the left-hand side of the above linker structure for the second linker L^{B} is attached to the hydrazinyl-indolyl or a hydrazinyl-pyrrolo-pyridinyl conjugation moiety, and the right-hand side of the above linker structure for the second linker L^{B} is attached to the second drug or active agent.

In certain embodiments, the conjugate is an antibody-drug conjugate where the antibody and the one or more drugs or active agents are linked together by linkers as described above. In some instances, the linker (e.g., L^{A} and/or L^{B}) is a cleavable linker. A cleavable linker is a linker that includes one or more cleavable moieties, where the cleavable moiety includes one or more bonds that can dissociate under certain conditions, thus separating the cleavable linker into two or more separable portions. For example, the cleavable moiety may include one or more covalent bonds, which under certain conditions, can dissociate or break apart to separate the cleavable linker into two or more portions. As such the linkers that are included in an antibody-drug conjugate can be cleavable linkers, such that under appropriate conditions, the cleavable linker is cleaved to separate or release the drug from the antibody at a desired target site of action for the drug.

In some instances, a cleavable linker includes two cleavable moieties, such as a first cleavable moiety and a second cleavable moiety. The cleavable moieties can be configured such that cleavage of both cleavable moieties is needed in order to separate or release the drug from the antibody at a desired target site of action for the drug. For example, cleavage of a cleavable linker can be achieved by initially cleaving one of the two cleavable moieties and then cleaving the other of the two cleavable moieties. In certain embodiments, a cleavable linker includes a first cleavable moiety and a second cleavable moiety that hinders cleavage of the first cleavable moiety. By “hinders cleavage” is meant that the presence of an uncleaved second cleavable moiety reduces the likelihood or substantially inhibits the cleavage of the first cleavable moiety, thus substantially reducing the amount or preventing the cleavage of the cleavable linker. For instance, the presence of uncleaved second cleavable moiety can hinder cleavage of the first cleavable moiety. The hinderance of cleavage of the first cleavable moiety by the presence of the second cleavable moiety, in turn, substantially reduces the amount or prevents the release of the drug from the antibody. For example, the premature release of the drug from the antibody can be substantially reduced or prevented until the antibody-drug conjugate is at or near the desired target site of action for the drug.

In some cases, since the second cleavable moiety hinders cleavage of the first cleavable moiety, cleavage of the cleavable linker can be achieved by initially cleaving the second cleavable moiety and then cleaving the first cleavable moiety. Cleavage of the second cleavable moiety can reduce or eliminate the hinderance on the cleavage of the first cleavable moiety, thus allowing the first cleavable moiety to be cleaved. Cleavage of the first cleavable moiety can result in the cleavable linker dissociating or separating into two or more portions as described above to release the drug from the antibody-drug conjugate. In some instances, cleavage of the first cleavable moiety does not substantially occur in the presence of an uncleaved second cleavable moiety. By substantially is meant that about 10% or less cleavage of the first cleavable moiety occurs in the presence of an uncleaved second cleavable moiety, such as about 9% or less, or about 8% or less, or about 7% or less, or about 6% or less, or about 5% or less, or about 4% or less, or about 3% or less, or about 2% or less, or about 1% or less, or about 0.5% or less, or about 0.1% or less cleavage of the first cleavable moiety occurs in the presence of an uncleaved second cleavable moiety.

Stated another way, the second cleavable moiety can protect the first cleavable moiety from cleavage. For instance, the presence of uncleaved second cleavable moiety can protect the first cleavable moiety from cleavage, and thus substantially reduce or prevent premature release of the drug from the antibody until the antibody-drug conjugate is at or near the desired target site of action for the drug. As such, cleavage of the second cleavable moiety exposes the first cleavable moiety (e.g., deprotects the first cleavable moiety), thus allowing the first cleavable moiety to be cleaved, which results in cleavage of the cleavable linker, which, in turn, separates or releases the drug from the antibody at a desired target site of action for the drug as described above. In certain instances, cleavage of the second cleavable moiety exposes the first cleavable moiety to subsequent cleavage, but cleavage of the second cleavable moiety does not in and of itself result in cleavage of the cleavable linker (i.e., cleavage of the first cleavable moiety is still needed in order to cleave the cleavable linker).

The cleavable moieties included in the cleavable linker may each be an enzymatically cleavable moiety. For example, the first cleavable moiety can be a first enzymatically cleavable moiety and the second cleavable moiety can be a second enzymatically cleavable moiety. An enzymatically cleavable moiety is a cleavable moiety that can be separated into two or more portions as described above through the enzymatic action of an enzyme. The enzymatically cleavable moiety can be any cleavable moiety that can be cleaved through the enzymatic action of an enzyme, such as, but not limited to, an ester, a peptide, a glycoside, and the like. In some instances, the enzyme that cleaves the enzymatically cleavable moiety is present at a desired target site of action, such as the desired target site of action of the drug that is to be released from the antibody-drug conjugate. In some cases, the enzyme that cleaves the enzymatically cleavable moiety is not present in a significant amount in other areas, such as in whole blood, plasma or serum. As such, the cleavage of an enzymatically cleavable moiety can be controlled such that substantial cleavage occurs at the desired site of action, whereas cleavage does not significantly occur in other areas or before the antibody-drug conjugate reaches the desired site of action.

For example, as described herein, antibody-drug conjugates of the present disclosure can be used for the treatment of cancer, such as for the delivery of a cancer therapeutic drug to a desired site of action where the cancer cells are present. In some cases, enzymes, such as an esterase that cleaves ester bonds or a glycosidase that cleaves glycosidic bonds, can be a biomarker for cancer that is overexpressed in cancer cells. The overexpression, and thus localization, of certain enzymes in cancer can be used in the context of the enzymatically cleavable moieties included in the cleavable linkers of the antibody-drug conjugates of the present disclosure to specifically release the drug at the desired site of action (i.e., the site of the cancer (and overexpressed enzyme)). Thus, in some embodiments, the enzymatically cleavable moiety is a cleavable moiety (e.g., an ester or a glycoside) that can be cleaved by an enzyme that is overexpressed in cancer cells. For instance, the enzyme can be an esterase. As such, in some instances, the enzymatically cleavable moiety is a cleavable moiety (e.g., an ester) that can be cleaved by an esterase enzyme. In some instances, the enzyme can be a glycosidase. As such, in some instances, the enzymatically cleavable moiety is a cleavable moiety (e.g., a glycoside or glycoside derivative) that can be cleaved by a glycosidase enzyme.

In certain embodiments, the enzymatically cleavable moiety is an ester bond. For example, the first cleavable moiety described above (i.e., the cleavable moiety protected from premature cleavage by the second cleavable moiety) can include an ester. The presence of uncleaved second cleavable moiety can protect the first cleavable moiety (ester) from cleavage by an esterase enzyme, and thus substantially reduce or prevent premature release of the drug from the antibody until the antibody-drug conjugate is at or near the desired target site of action for the drug. In some instances, a portion of the linker adjacent to the first cleavable moiety is linked to or includes a substituent, where the substituent comprises the second cleavable moiety. In some instances, the second cleavable moiety includes a glycoside or glycoside derivative.

In some embodiments, the enzymatically cleavable moiety is sugar moiety, such as a glycoside (or glyosyl) or glycoside derivative. In some cases, the glycoside or glycoside derivative can facilitate an increase in the hydrophilicity of the cleavable linker as compared to a cleavable linker that does not include the glycoside or glycoside derivative. The glycoside or glycoside derivative can be any glycoside or glycoside derivative suitable for use in the cleavable linker and that can be cleaved through the enzymatic action of an enzyme. For example, the second cleavable moiety (i.e., the cleavable moiety that protects the first cleavable moiety from premature cleavage) can be a glycoside or glycoside derivative. For instance, in some embodiments, the first cleavable moiety includes an ester and the second cleavable moiety includes a glycoside or glycoside derivative. In certain embodiments, the second cleavable moiety is a glycoside or glycoside derivative selected from a glucuronide, a galactoside, a glucoside, a mannoside, a fucoside, O-GlcNAc, and O-GalNAc. In some instances, the second cleavable moiety is a glucuronide. In some instances, the second cleavable moiety is a galactoside. In some instances, the second cleavable moiety is a glucoside. In some instances, the second cleavable moiety is a mannoside. In some instances, the second cleavable moiety is a fucoside. In some instances, the second cleavable moiety is O-GlcNAc. In some instances, the second cleavable moiety is O-GalNAc.

The glycoside or glycoside derivative can be attached (covalently bonded) to the cleavable linker through a glycosidic bond. The glycosidic bond can link the glycoside or glycoside derivative to the cleavable linker through various types of bonds, such as, but not limited to, an O-glycosidic bond (an O-glycoside), an N-glycosidic bond (a glycosylamine), an S-glycosidic bond (a thioglycoside), or C-glycosidic bond (a C-glycoside or C-glycosyl). In some instances, the glycosidic bond is an O-glycosidic bond (an O-glycoside). In some cases, the glycoside or glycoside derivative can be cleaved from the cleavable linker it is attached to by an enzyme (e.g., through enzymatically-mediated hydrolysis of the glycosidic bond). A glycoside or glycoside derivative can be removed or cleaved from the cleavable linker by any convenient enzyme that is able to carry out the cleavage (hydrolysis) of the glycosidic bond that attaches the glycoside or glycoside derivative to the cleavable linker. An example of an enzyme that can be used to mediate the cleavage (hydrolysis) of the glycosidic bond that attaches the glycoside or glycoside derivative to the cleavable linker is a glycosidase, such as a glucuronidase, a galactosidase, a glucosidase, a mannosidase, a fucosidase, and the like. Other suitable enzymes may also be used to mediate the cleavage (hydrolysis) of the glycosidic bond that attaches the glycoside or glycoside derivative to the cleavable linker. In some cases, the enzyme used to mediate the cleavage (hydrolysis) of the glycosidic bond that attaches the glycoside or glycoside derivative to the cleavable linker is found at or near the desired site of action for the drug of the antibody-drug conjugate. For instance, the enzyme can be a lysosomal enzyme, such as a lysosomal glycosidase, found in cells at or near the desired site of action for the drug of the antibody-drug conjugate. In some cases, the enzyme is an enzyme found at or near the target site where the enzyme that mediates cleavage of the first cleavable moiety is found.

Examples of conjugates according to the present disclosure include, but are not limited to, the following structures:

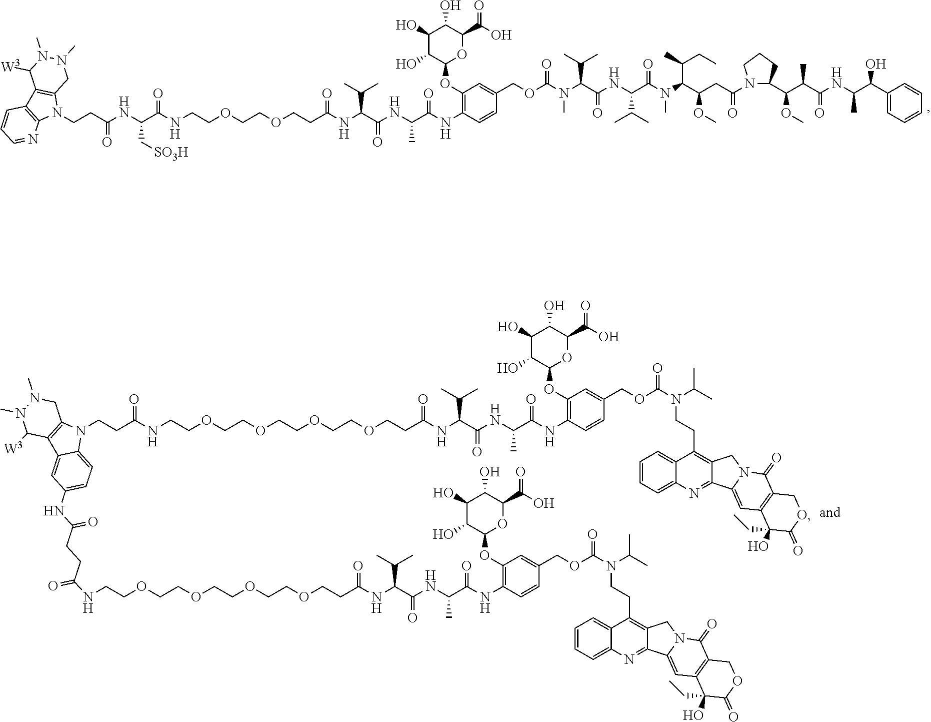

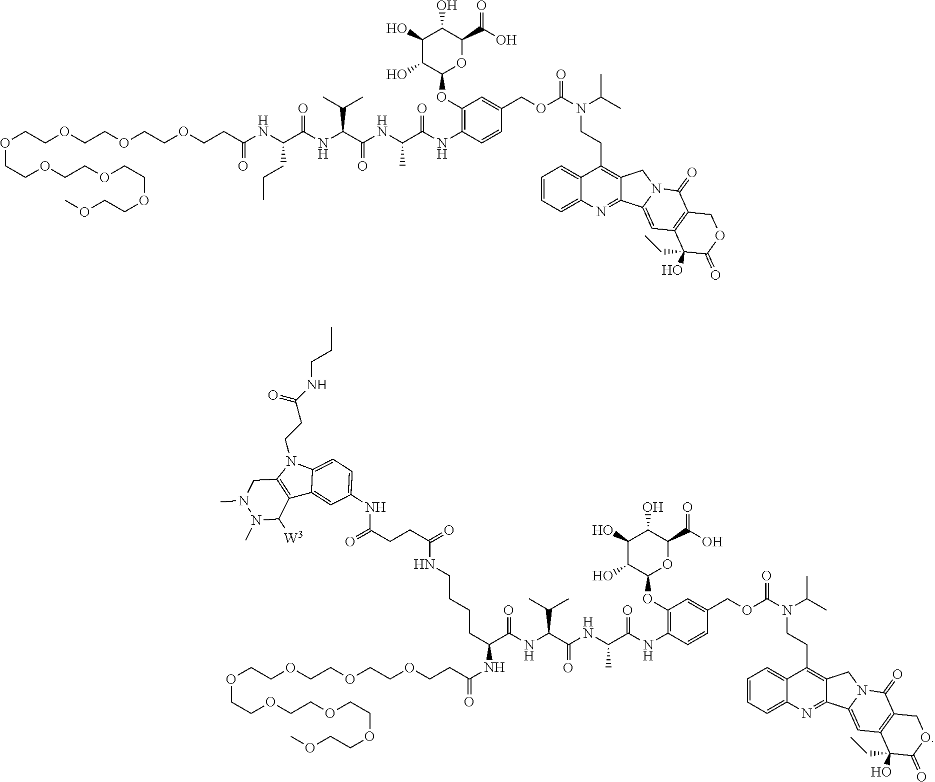

Any of the chemical entities, linkers and conjugation moieties set forth in the structures above may be adapted for use in the subject compounds and conjugates.

Additional disclosure related to hydrazinyl-indolyl and hydrazinyl-pyrrolo-pyridinyl compounds and methods for producing a conjugate is found in U.S. Pat. Nos. 9,310,374 and 9,493,413, the disclosures of each of which are incorporated herein by reference. Additional disclosure related to cleavable linkers is found in WO 2020/154437, filed Jan. 22, 2020, PCT/US2021/060193, filed Nov. 19, 2021, and PCT/US2022/012347, filed Jan. 13, 2022, the disclosures of which are incorporated herein by reference. Additional disclosure related to branched linkers is found in WO 2020/154437, filed Jan. 22, 2020, PCT/US2021/060193, filed Nov. 19, 2021, and PCT/US2022/018534, filed Mar. 2, 2022, the disclosures of which are incorporated herein by reference.

### Compounds Useful for Producing Conjugates

The present disclosure provides compounds useful for producing the conjugates described herein. In certain embodiments, the compound can be attached to one or more drugs or active agents and may also include a hydrazinyl-indolyl or hydrazinyl-pyrrolo-pyridinyl conjugation moiety useful for conjugation of the one or more drugs or active agents to a polypeptide (e.g., an antibody). For example, the conjugation moiety in the compound may be conjugated to a polypeptide (e.g., antibody), thus indirectly binding the one or more drugs or active agents and the polypeptide (antibody) together.

In certain embodiments, the compound is a compound of formula (II):

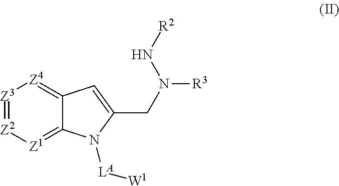

wherein:
Z¹, Z², Z³ and Z⁴ are each independently selected from CR⁴, N and C-L^{B}-W²;
R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L^{A} is a first linker;
L^{B} is a second linker;
W¹ is a first drug; and
W² is a second drug.

Regarding compounds of formula (II), the substituents Z¹, Z², Z³, Z⁴, R², R³, R⁴, L^{A}, L^{B}, W¹, and W² are as described above in relation to the conjugates of formula (I). Similarly, regarding the first linker L^{A} and the second linker L^{B} of formula (II), the T¹, T², T³, T⁴, T⁵, T⁶, V¹, V², V³, V⁴, V⁵ and V⁶, and T⁷, T⁸, T⁹, T¹⁰, T¹¹, T¹², T¹³, V⁷, V⁸, V⁹, V¹⁰, V¹¹, V¹² and V¹³ substituents are as described above in relation to the conjugates of formula (I).

For example, in some instances, T¹, T², T³, T⁴, T⁵ and T⁶ and V¹, V², V³, V⁴, V⁵ and V⁶ are selected from the following:

wherein:
T¹ is (C₁-C₁₂)alkyl and V¹ is —CO—;
T² is an amino acid analog and V² is —NH—;
T³ is (PEG)ₙ and V³ is —CO—;
T⁴ is AA and V⁴ is absent;
T⁵ is PABC and V⁵ is absent; and
f is 0; or
   wherein:
T¹ is (C₁-C₁₂)alkyl and V¹ is —CONH—;
T² is (PEG), and V² is —CO—;
T³ is AA and V³ is absent;
T⁴ is PABC and V⁴ is absent; and
e and f are each 0; or
   wherein:
T¹ is (C₁-C₁₂)alkyl and V¹ is —CONH—;
T² is substituted (C₁-C₁₂)alkyl and V² is —CO—;
T³ is AA and V³ is absent;
T⁴ is PABC and V⁴ is absent; and
e and f are each 0.

For example, in some instances, T⁷, T⁸, T⁹, T¹⁰, T¹¹, T¹² and T¹³ and V⁷, V⁸, V⁹, V¹⁰, V¹¹, V¹² and V¹³ are selected from the following:

wherein:
T⁷ is absent and V⁷ is —NHCO—;
T⁸ is (C₁-C₁₂)alkyl and V⁸ is —CONH—;
T⁹ is (PEG), and V⁹ is —CO—;
T¹⁰ is AA and V¹⁰ is absent; and
T¹¹ is PABC and V¹¹ is absent; and
l and m are each 0; or
   wherein:
T⁷ is absent and V⁷ is —NHCO—;
T⁸ is (C₁-C₁₂)alkyl and V⁸ is —CONH—;
T⁹ is substituted (C₁-C₁₂)alkyl and V⁹ is —CO—;
T¹⁰ is AA and V¹⁰ is absent;
T¹¹ is PABC and V¹¹ is absent; and
l and m are each 0.

Compounds of formula (II) can be used in conjugation reactions described herein, where one or more drugs or active agents attached to a hydrazinyl-indolyl or a hydrazinyl-pyrrolo-pyridinyl conjugation moiety are conjugated to a polypeptide (e.g., antibody) to form an antibody-drug conjugate.

Examples of compounds according to the present disclosure include, but are not limited to, the following structures:

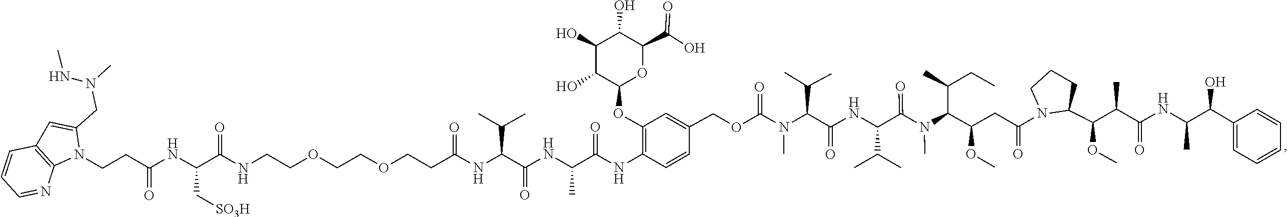 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCOCCNC(=O)[C@H](CS(=O)(=O)O)NC(=O)CCn2c(CN(C)NC)cc3cccnc32)C(C)C)c(O[C@@H]2O[C@H](C(=O)O)[C@@H](O)[C@H](O)[C@H]2O)c1)C(C)C]

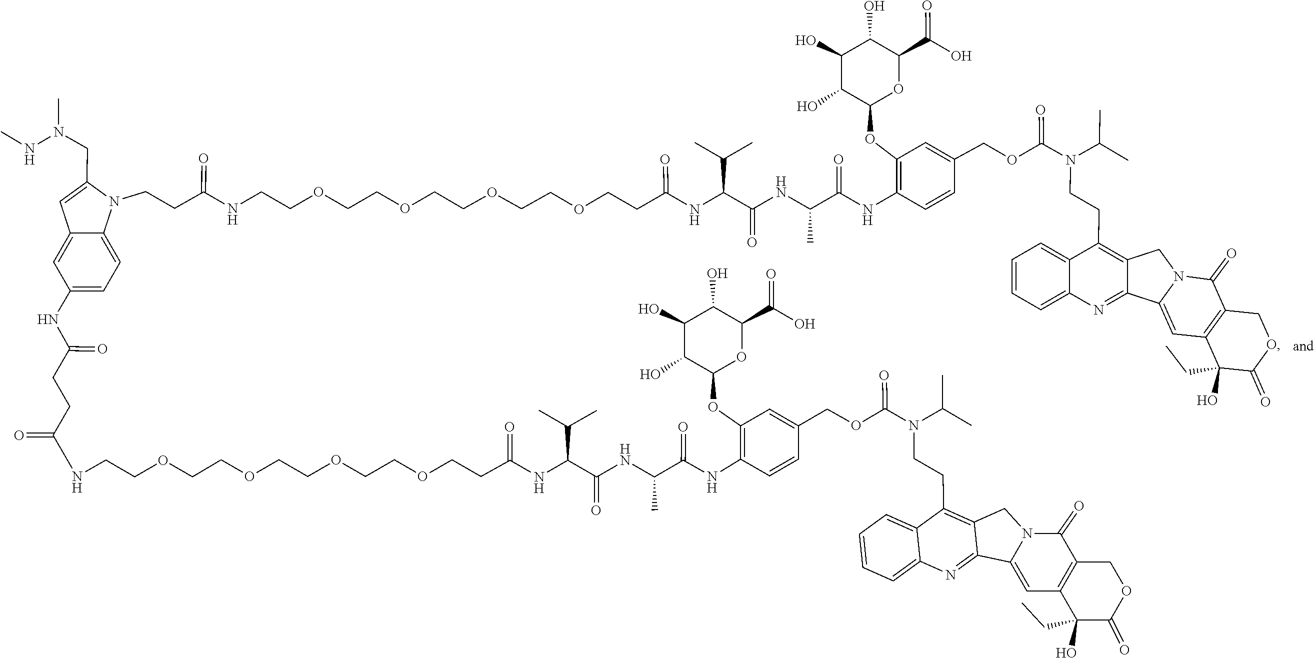 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)CCOCCOCCOCCOCCNC(=O)CCC(=O)Nc2ccc3c(c2)cc(CN(C)NC)n3CCC(=O)NCCOCCOCCOCCOCCC(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)C(C)C)c(O[C@@H]2O[C@H](C(=O)O)[C@@H](O)[C@H](O)[C@H]2O)c1)C(C)C ; "and"]

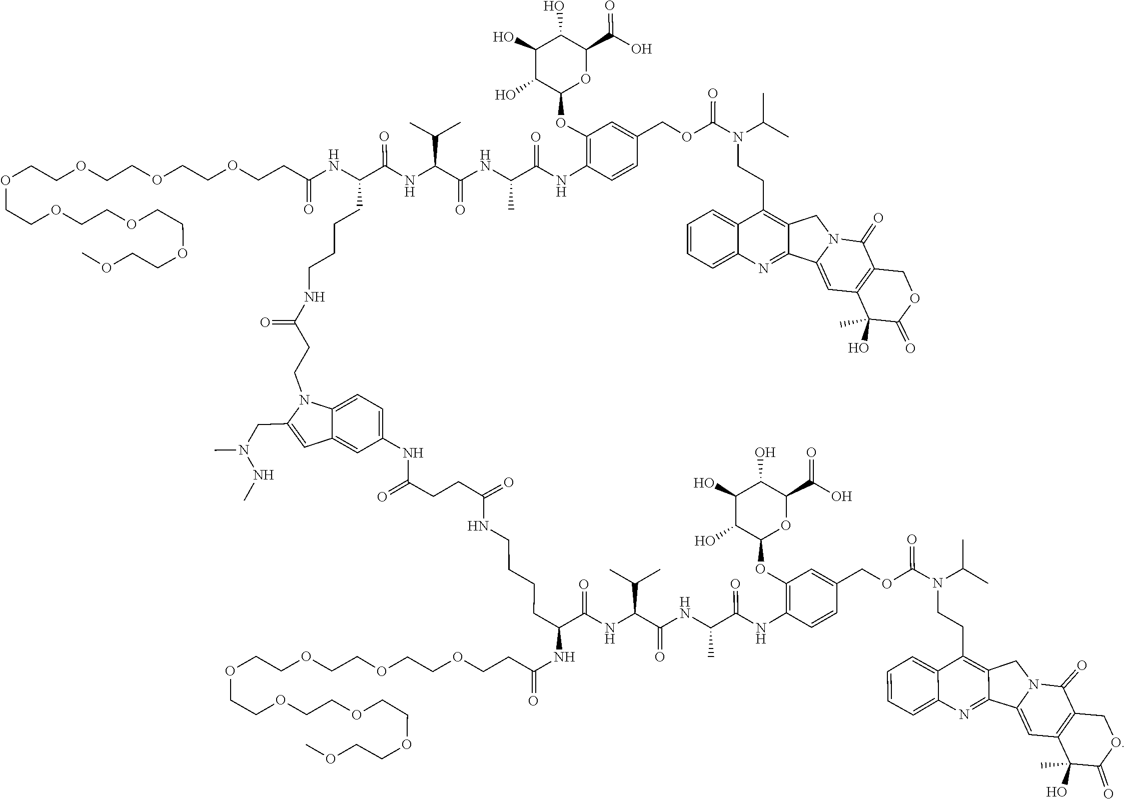 [SMILES: CNN(C)Cc1cc2cc(NC(=O)CCC(=O)NCCCC[C@H](NC(=O)CCOCCOCCOCCOCCOCCOCCOCCOC)C(=O)N[C@H](C(=O)N[C@@H](C)C(=O)Nc3ccc(COC(=O)N(CCc4c5c(nc6ccccc46)-c4cc6c(c(=O)n4C5)COC(=O)[C@@]6(C)O)C(C)C)cc3O[C@@H]3O[C@H](C(=O)O)[C@@H](O)[C@H](O)[C@H]3O)C(C)C)ccc2n1CCC(=O)NCCCC[C@H](NC(=O)CCOCCOCCOCCOCCOCCOCCOCCOC)C(=O)N[C@H](C(=O)N[C@@H](C)C(=O)Nc1ccc(COC(=O)N(CCc2c3c(nc4ccccc24)-c2cc4c(c(=O)n2C3)COC(=O)[C@@]4(C)O)C(C)C)cc1O[C@@H]1O[C@H](C(=O)O)[C@@H](O)[C@H](O)[C@H]1O)C(C)C]

Any of the chemical entities, linkers and conjugation moieties set forth in the structures above may be adapted for use in the subject compounds and conjugates.

### Anti-Nectin-4 Antibodies

As noted above, a subject conjugate can comprise, as substituent W² an anti-Nectin-4 antibody, where the amino acid sequence of the anti-Nectin-4 antibody has been modified to include a 2-formylglycine (fGly) residue. As used herein, amino acids may be referred to by their standard name, their standard three letter abbreviation and/or their standard one letter abbreviation, such as: Alanine or Ala or A; Cysteine or Cys or C; Aspartic acid or Asp or D; Glutamic acid or Glu or E; Phenylalanine or Phe or F; Glycine or Gly or G; Histidine or His or H; Isoleucine or Ile or I; Lysine or Lys or K; Leucine or Leu or L; Methionine or Met or M; Asparagine or Asn or N; Proline or Pro or P; Glutamine or Gln or Q; Arginine or Arg or R; Serine or Ser or S: Threonine or Thr or T: Valine or Val or V; Tryptophan or Trp or W; and Tyrosine or Tyr or Y.

Various protein sequences identified in this disclosure are provided in the Table 2 below and are referenced throughout based on their sequence identifiers (SEQ ID NO.)

TABLE 2
Variable heavy chain (VH) and variable light chain (VL) sequences
and sequence identifiers of various antibodies disclosed herein.
First, second, and third CDR sequences in each VH and VL
sequences are bolded and underlined. First framework (FW1)
region is before the first CDR, the second
framework (FW2) region is after the first CDR, the
third framework (FW3) region is after the
second CDR, and the fourth framework (FW4) region
is after the third CDR.

| Description | | SEQ ID |
|---|---|---|
| of the protein | Sequence | NO: |
| 5D9 Parental | QVQLKQSGPGLVQPSQSLSITCTVSGFSLTNYGVHWVRQSPGKGL | 1 |
| VH | EWLGVIWSGGSTDYNAAFVSRLSISRDNSKSQVFFKMNSLQADD | |
| | TAIYYCARSGKWYYFDYWGQGTTLTVSS | |
| 5D9 VH | QVQLKESGPGLVQPSETLSLTCTVSGFSITNYGVHWIRQSPGKGL | 2 |
| variant 1 | EWLGVIWSGGSTDYNAAFVSRVTISRDNSKSQVFFKLNSVQADDT | |
| | AVYYCARSGKWYYFDYWGQGTTVTVSS | |
| 5D9 VH | QVQLQESGPGLVKPSETLSLTCTVSGFSITNYGVHWIRQPPGKGL | 3 |
| variant 2 | EWLGVIWSGGSTDYNASLVSRVTISRDTSKSQFFLKLNSVQADDT | |
| | AVYYCARSGKWYYFDYWGQGTTVTVSS | |
| 5D9 VH | QVQLQESGPGLVKPSETLSLTCTVSGGSITNYGVHWIRQPPGKGL | 4 |
| variant 3 | EWLGVIWSGGSTDYNPSLKSRVTISVDTSKNQFSLKLSSVTAADT | |
| | AVYYCARSGKWYYFDYWGQGTLVTVSS | |
| 5D9 VH | QVQLQQSGPGLVKPSETLSLTCTVSGESLTNYGVHWVRQPPGKG | 5 |
| variant 4 | LEWLGVIWSGGSTDYNPSLKSRLTISRDTSKNQVSFKMSSVTAA | |
| | DTAVYYCARSGKWYYFDYWGQGTLVTVSS | |
| 5D9 VH | QVQLQESGPGLVKPSETLSLTCTVSGGSLTNYGVHWVRQPPGKG | 6 |
| variant 5 | LEWIGVIWSGGSTNYNPSLKSRVTISRDTSKNQVSLKLSSVTAAD | |
| | TAVYYCARSGKWYYFDYWGQGTLVTVSS | |
| 12E11 | QVQLKQSGPGLVQPSQSLSITCTVSGFSLTTYGVHWVRQSPGKGL | 7 |
| Parental VH | EWLGVIWSGGSTDCNGAFISRLSIIKDNSKSQVFFKMNSLQADDT | |
| | AIYYCARMTHWYFDVWGTGTTVTVSS | |
| 12E11 | QVQLKQSGPGLVQPSQSLSITCTVSGFSLTTYGVHWVRQSPGKGL | 8 |
| variant | EWLGVIWSGGSTDCNGAFISRLSIIKDNSKSQVFFKMNSLQADDT | |
| HC 31 VH | AIYYCARLTHWYFDVWGTGTTVTVSS | |
| 12 E11 | QVQLKQSGPGLVQPSQSLSITCTVSGFSLTTYGVHWVRQSPGKGL | 9 |
| variant | EWLGVIWSGGSTDCNGAFISRLSIIKDNSKSQVFFKMNSLQADDT | |
| HC 11 VH | AIYYCARHTHWYFDVWGTGTTVTVSS | |
| 12E11 | QVQLKQSGPGLVQPSQSLSITCTVSGFSLTTYGVHWVRQSPGKGL | 10 |
| variant | EWLGVIWSGGSTDCNGAFISRLSIIKDNSKSQVFFKMNSLQADDT | |
| HC 42 VH | AIYYCARQTHWYFDVWGTGTTVTVSS | |
| 12E11 | QVQLKQSGPGLVQPSQSLSITCTVSGFSLTTYGVHWVRQSPGKGL | 11 |
| variant | EWLGVIWSGGSTDCNGAFISRLSIIKDNSKSQVFFKMNSLQADDT | |
| HC 61 VH | AIYYCARVTHWYFDVWGTGTTVTVSS | |
| 12E11 | QVQLKQSGPGLVQPSQSLSITCTVSGFSLTTYGVHWVRQSPGKGL | 12 |
| variant | EWLGVIWSGGSTDCNGAFISRLSIIKDNSKSQVFFKMNSLQADDT | |
| HC 53 VH | AIYYCARTTHWYFDVWGTGTTVTVSS | |
| 12E11 | QVQLKQSGPGLVQPSQSLSITCTVSGFSLTTYGVHWVRQSPGKGL | 13 |
| variant | EWLGVIWSGGSTDCNGAFISRLSIIKDNSKSQVFFKMNSLQADDT | |
| HC 22 VH | AIYYCARKTHWYFDVWGTGTTVTVSS | |
| 3C12 VH | QVQLKQSGPGLVQPSQSLSITCTVSGFSLTTYGVHWVRQSPGKGL | 14 |
| | EWLGVIWSGGNTDYNAAFISRLSISKDNSKSQVFFKMNSLQADDT | |
| | AIYYCARMTHWYFDVWGTGTTVTVSS | |
| 6C8 VH | QVQLKQSGPGLVQPSQSLSITCTVSGFSLSTYGVHWVRQSPGKGL | 15 |
| | EWLGVIWSGGSTDYNAAFISRLTISKDNSKSQIFFKMNSLQADDT | |
| | AIYYCARMMSWYFDVWGTGTTVTVSS | |
| 7E8 VH | QVQLKQSGPGLVQPSQSLSITCTVSGFSLATYGVHWVRQSPGKGL | 16 |
| | EWLGVIWSGGSTDYNAAFISRLSISKDNSKSQVFFKMNSLQADDT | |
| | AIYYCARNSHWYFDVWGTGTTVTVSS | |
| 7H10 VH | QVQLQQPGAELVKPGASVKLSCKASGYTFTTYWMHWVKQRPG | 17 |
| | RGLEWIGGIDPYGAHIKYNEMFKTKATLTVDKPSSTAYMQLSSLT | |
| | SEDSAVYYCARGDYSNWWWTYWGQGTLVTVSA | |
| 5D9 Parental | DIQMTQSPASLSVSVGETVTITCRASENIYSNLAWYQQKQGKSPQ | 18 |
| VL | LLVYAATNLADGVPSRFSGSGSGTQYSLKINSLQSEDFGSYYCQH | |
| | FWGTPTFGGGTKLEIK | |
| 5D9 VL | DIQMTQSPASLSASVGDTVTITCRASQNIYSNLAWYQQKQGKSPQ | 19 |
| variant 1 | LLVYAATNLADGVPSRFSGSGSGTQYTLKINSLQSEDFGTYYCQH | |
| | FWGTPTFGGGTKVEIK | |
| 5D9 VL | DIQMTQSPSSLSASVGDTVTITCRASQNIYSNLAWYQQKQGKAPK | 20 |
| variant 2 | LLVYAATNLADGVPSRFSGSGSGTDFTLKINSLQPEDFATYYCQH | |
| | FWGTPTFGGGTKVEIK | |
| 5D9 VL | DIQMTQSPSSLSASVGDRVTITCRASQSIYSNLAWYQQKPGKAPK | 21 |
| variant 3 | LLVYAATNLASGVPSRFSGSGSGTDFTLTISSLQPEDFATYYCQH | |
| | EWGTPTFGGGTKVEIK | |
| 5D9 VL | DIQMTQSPSSLSASVGDRVTITCRASENIYSNLAWYQQKPGKAPK | 22 |
| variant 4 | LLVYAATNLADGVPSRFSGSGSGTDYTLTISSLQPEDFATYYCH | |
| | FWGTPTFGGGTKVEIK | |
| 5D9 VL | DIQMTQSPSSLSASVGDRVTITCRASQSIYSNLAWYQQKPGKAPK | 23 |
| variant 5 | LLVYAATNLQSGVPSRFSGSGSGTDFTLTISSLQPEDFATYYCQH | |
| | FWGTPTFGGGTKVEIK | |
| 12E11 | DIVMTQSQKFMSTTVGDRVSITCKASQNVNTAVAWYQQKPGQSP | 24 |
| Parental VL | KLLIYSASYRYTGVPDRFTGSGSGTDFTLTISNMQSEDLADYFC | |
| | QQYSNYPLTFGAGTKLELK | |
| 12E11 | DIVMTQSQKFMSTTVGDRVSITCKASQNVQTAVAWYQQKPGQSPK | 25 |
| variant | LLIYSASYRYTGVPDRFTGSGSGTDFTLTISNMQSEDLADYFCQQ | |
| LC 72 | YSNYPLTFGAGTKLELK | |
| 12E11 | DIVMTQSQKFMSTTVGDRVSITCKASQNVYTAVAWYQQKPGQSPK | 26 |
| variant | LLIYSASYRYTGVPDRFTGSGSGTDFTLTISNMQSEDLADYFCQQ | |
| LC 91 | YSNYPLTFGAGTKLELK | |
| 12E11 | DIVMTQSQKFMSTTVGDRVSITCKASQNVVTAVAWYQQKPGQSP | 27 |
| variant | KLLIYSASYRYTGVPDRFTGSGSGTDFTLTISNMQSEDLADYFC | |
| LC 83 | QQYSNYPLTFGAGTKLELK | |
| 3C12 VL | DIVMTQSQKFMSTTVGDRVSITCKASQNVVTAVAWYQQKPGQSP | 28 |
| | KLLIYSASNRYTGVPDRFTGSGSGTDFTLTISYMQSEDLADYFC | |
| | QQYSSYPLTFGAGTKLELK | |
| 6C8 VL | DIVMTQSQKFMSTSVGDRVSVTCKASQNVVTNVAWYQQKPGQSP | 29 |
| | KALIYSASYRYSGVPDRFTGSGSGTDFTLTISNVQSEDLAEYFC | |
| | QQYNSYPLTFGAGTKLELK | |
| 7E8 VL | DIVMTQSQKFMSTSVGDRVSVTCKASQNVVTNVAWYQQKPGQSP | 30 |
| | KALIYSASYRYSGVPDRFTGSGSGTDFTLTISHVQSEDLAEYFC | |
| | HQYNNYPYTFGGGTKLEIK | |
| 7H10 VL | DIVMTQSQKFMSTTVGDRVSITCKASQNVGTAVAWYQQIPGQSPK | 31 |
| | LLIYSASNRYTGVPDRFTGSGSGTDFTLTITNMQSEDLADYFCQH | |
| | YSSYPWTFGGGTKLEIK | |

The CDR sequences and the corresponding sequence identifiers are provided in the Table 3 below:

TABLE 3
CDR sequences of various VH and VL chains disclosed herein.

| | | SEQ | | SEQ | | SEQ |
|---|---|---|---|---|---|---|
| Description | CDR1 | ID | CDR2 | ID | | ID |
| of the protein | Sequence | NO: | Sequence | NO: | CDR3 Sequence | NO: |
| 5D9 Parental | GFSLTNYG | 32 | IWSGGST | 33 | ARSGKWYYFDY | 34 |
| VH | | | | | | |
| 5D9 VH | GFSITNYG | 35 | IWSGGST | 33 | ARSGKWYYFDY | 34 |
| variant 1 | | | | | | |
| 5D9 VH | GFSITNYG | 35 | IWSGGST | 33 | ARSGKWYYFDY | 34 |
| variant 2 | | | | | | |
| 5D9 VH | GGSITNYG | 36 | IWSGGST | 33 | ARSGKWYYFDY | 34 |
| variant 3 | | | | | | |
| 5D9 VH | GFSLTNYG | 32 | IWSGGST | 33 | ARSGKWYYFDY | 34 |
| variant 4 | | | | | | |
| 5D9 VH | GGSLTNYG | 37 | IWSGGST | 33 | ARSGKWYYFDY | 34 |
| variant 5 | | | | | | |
| 12E11 | GFSLTTYG | 38 | IWSGGST | 33 | ARMTHWYFDV | 39 |
| Parental VH | | | | | | |
| 12E11 | GFSLTTYG | 38 | IWSGGST | 33 | ARLTHWYFDV | 40 |
| variant | | | | | | |
| HC 31 VH | | | | | | |
| 12 E11 | GFSLTTYG | 38 | IWSGGST | 33 | ARHTHWYFDV | 41 |
| variant | | | | | | |
| HC 11 VH | | | | | | |
| 12E11 | GFSLTTYG | 38 | IWSGGST | 33 | ARQTHWYFDV | 42 |
| variant | | | | | | |
| HC 42 VH | | | | | | |
| 12E11 | GFSLTTYG | 38 | IWSGGST | 33 | ARVTHWYFDV | 43 |
| variant | | | | | | |
| HC 61 VH | | | | | | |
| 12E11 | GFSLTTYG | 38 | IWSGGST | 33 | ARTTHWYFDV | 44 |
| variant | | | | | | |
| HC 53 VH | | | | | | |
| 12E11 | GFSLTTYG | 38 | IWSGGST | 33 | ARKTHWYFDV | 45 |
| variant | | | | | | |
| HC 22 VH | | | | | | |
| 3C12 VH | GFSLTTYG | 38 | IWSGGNT | 33 | ARMTHWYFDV | 39 |
| 6C8 VH | GFSLSTYG | 46 | IWSGGST | 33 | ARMMSWYFDV | 47 |
| 7E8 VH | GFSLATYG | 48 | IWSGGST | 33 | ARNSHWYFDV | 49 |
| 7H10 VH | GYTFTTYW | 50 | IDPYGAHI | 51 | ARGDYSNWWW | 52 |
| | | | | | TY | |
| 5D9 Parental | ENIYSN | 53 | AAT | 54 | QHFWGTPT | 55 |
| VL | | | | | | |
| 5D9 VL | QNIYSN | 56 | AAT | 54 | QHFWGTPT | 55 |
| variant 1 | | | | | | |
| 5D9 VL | QNIYSN | 56 | AAT | 54 | QHFWGTPT | 55 |
| variant 2 | | | | | | |
| 5D9 VL | QSIYSN | 57 | AAT | 54 | QHFWGTPT | 55 |
| variant 3 | | | | | | |
| 5D9 VL | ENIYSN | 53 | AAT | 54 | QHFWGTPT | 55 |
| variant 4 | | | | | | |
| 5D9 VL | QSIYSN | 57 | AAT | 54 | QHFWGTPT | 55 |
| variant 5 | | | | | | |
| 12E11 | QNVNTA | 58 | SAS | 59 | QQYSNYPLT | 60 |
| Parental VL | | | | | | |
| 12E11 | QNVQTA | 61 | SAS | 59 | QQYSNYPLT | 60 |
| variant | | | | | | |
| LC 72 VL | | | | | | |
| 12E11 | QNVYTA | 62 | SAS | 59 | QQYSNYPLT | 60 |
| variant | | | | | | |
| LC 91 VL | | | | | | |
| 12E11 | QNVVTA | 63 | SAS | 59 | QQYSNYPLT | 60 |
| variant | | | | | | |
| LC 83 VL | | | | | | |
| 3C12 VL | QNVVTA | 63 | SAS | 59 | QQYSSYPLT | 64 |
| 6C8 VL | QNVVTN | 65 | SAS | 59 | QQYNSYPLT | 66 |
| 7E8 VL | QNVVTN | 65 | SAS | 59 | HQYNNYPYT | 67 |
| 7H10 VL | QNVGTA | 68 | SAS | 59 | QHYSSYPWT | 69 |

According to some embodiments, an antibody of the present disclosure specifically binds to Nectin-4 and comprises:
a variable heavy chain (VH) chain comprising heavy chain CDRs 1-3 (HCDRs 1-3) of a VH chain having a sequence selected from SEQ ID NOs: 1 to 17; and
a variable light chain (VL) chain comprising light chain CDRs 1-3 (LCDRs 1-3) of a VL chain having a sequence selected from SEQ ID NOs: 18 to 31.

According to some embodiments, an antibody of the present disclosure specifically binds to Nectin-4 and comprises:
a VH chain comprising a sequence selected from SEQ ID NOs: 1 to 17; and
a VL chain comprising a sequence selected from SEQ ID NOs: 18 to 31.

In some cases, an antibody of the present disclosure specifically binds to Nectin-4 and comprises:
a VH chain comprising HCDRs 1-3 of a VH chain having a sequence selected from SEQ ID NOs: 1 to 6; and
a VL chain comprising LCDRs 1-3 of a VL chain having a sequence selected from SEQ ID NOs: 18 to 23.

Thus, in certain embodiments, an antibody of the present disclosure specifically binds to Nectin-4 and comprises:
a VH chain comprising a sequence selected from SEQ ID NOs: 1 to 6; and
a VL chain comprising a sequence selected from SEQ ID NOs: 18 to 23.

An antibody comprising the any combination of heavy and light chains indicated in the Table 4 below are envisioned.

TABLE 4
Combinations of HV and HL chains in certain
embodiments of the invention.

| | | | | | |
|---|---|---|---|---|---|
| 5D9 Parental | 5D9 | 5D9 | 5D9 | 5D9 | 5D9 |
| VH/ | Parental | Parental | Parental | Parental | Parental |
| 5D9 Parental | VH/ | VH/ | VH/ | VH/ | VH/ |
| VL | 5D9 VL | 5D9 VL | 5D9 VL | 5D9 VL | 5D9 VL |
| | variant 1 | variant 2 | variant 3 | variant 4 | variant 5 |
| 5D9 VH | 5D9 VH | 5D9 VH | 5D9 VH | 5D9 VH | 5D9 VH |
| variant 1/ | variant 1/ | variant 1/ | variant 1/ | variant 1/ | variant 1/ |
| 5D9 Parental | 5D9 VL | 5D9 VL | 5D9 VL | 5D9 VL | 5D9 VL |
| VL | variant 1 | variant 2 | variant 3 | variant 4 | variant 5 |
| 5D9 VH | 5D9 VH | 5D9 VH | 5D9 VH | 5D9 VH | 5D9 VH |
| variant 2/ | variant 2/ | variant 2/ | variant 2/ | variant 2/ | variant 2/ |
| 5D9 Parental | 5D9 VL | 5D9 VL | 5D9 VL | 5D9 VL | 5D9 VL |
| VL | variant 1 | variant 2 | variant 3 | variant 4 | variant 5 |
| 5D9 VH | 5D9 VH | 5D9 VH | 5D9 VH | 5D9 VH | 5D9 VH |
| variant 3/ | variant 3/ | variant 3/ | variant 3/ | variant 3/ | variant 3/ |
| 5D9 Parental | 5D9 VL | 5D9 VL | 5D9 VL | 5D9 VL | 5D9 VL |
| VL | variant 1 | variant 2 | variant 3 | variant 4 | variant 5 |
| 5D9 VH | 5D9 VH | 5D9 VH | 5D9 VH | 5D9 VH | 5D9 VH |
| variant V4/ | variant 4/ | variant 4/ | variant 4/ | variant 4/ | variant 4/ |
| 5D9 Parental | 5D9 VL | 5D9 VL | 5D9 VL | 5D9 VL | 5D9 VL |
| VL | variant 1 | variant 2 | variant 3 | variant 4 | variant 5 |
| 5D9 VH | 5D9 VH | 5D9 VH | 5D9 VH | 5D9 VH | 5D9 VH |
| variant V5/ | variant 5/ | variant 5/ | variant 5/ | variant 5/ | variant 5/ |
| 5D9 Parental | 5D9 VL | 5D9 VL | 5D9 VL | 5D9 VL | 5D9 VL |
| VL | variant 1 | variant 2 | variant 3 | variant 4 | variant 5 |

According to some embodiments, an antibody of the present disclosure specifically binds to Nectin-4 and comprises:
a VH chain comprising HCDRs 1-3 of a VH chain having a sequence selected from SEQ ID NOs: 7 to 13; and
a VL chain comprising LCDRs 1-3 of a VL chain having a sequence selected from SEQ ID NOs: 24 to 27.

In some cases, an antibody of the present disclosure specifically binds to Nectin-4 and comprises:
a VH chain comprising a sequence selected from SEQ ID NOs: 7 to 13; and
a VL chain comprising a sequence selected from SEQ ID NOs: 24 to 27.

In certain embodiments, an antibody comprising the any combination of heavy and light chains indicated in the Table 5 below are envisioned.

TABLE 5
Combinations of HV and HL chains in certain embodiments
of the invention.

| | | | |
|---|---|---|---|
| 12E11 Parental VH/ | 12E11 | 12E11 | 12E11 |
| 12E11 Parental VL | Parental VH/ | Parental VH/ | Parental VH/ |
| | 12E11 variant | 12E11 variant | 12E11 variant |
| | LC 72 VL | LC 91 VL | LC 83 VL |
| 12E11 variant | 12E11 variant | 12E11 variant | 12E11 variant |
| HC 31 VH/ | HC 31 VH/ | HC 31 VH/ | HC 31 VH/ |
| 12E11 Parental VL | 12E11 variant | 12E11 variant | 12E11 variant |
| | LC 72 VL | LC 91 VL | LC 83 VL |
| 12 E11 variant | 12 E11 variant | 12 E11 variant | 12 E11 variant |
| HC 11 VH/ | HC 11 VH/ | HC 11 VH/ | HC 11 VH/ |
| 12E11 Parental VL | 12E11 variant | 12E11 variant | 12E11 variant |
| | LC 72 VL | LC 91 VL | LC 83 VL |
| 12E11 variant | 12E11 variant | 12E11 variant | 12E11 variant |
| HC 42 VH/ | HC 42 VH/ | HC 42 VH/ | HC 42 VH/ |
| 12E11 Parental VL | 12E11 variant | 12E11 variant | 12E11 variant |
| | LC 72 VL | LC 91 VL | LC 83 VL |
| 12E11 variant | 12E11 variant | 12E11 variant | 12E11 variant |
| HC 61 VH/ | HC 61 VH/ | HC 61 VH/ | HC 61 VH/ |
| 12E11 Parental VL | 12E11 variant | 12E11 variant | 12E11 variant |
| | LC 72 VL | LC 91 VL | LC 83 VL |
| 12E11 variant | 12E11 variant | 12E11 variant | 12E11 variant |
| HC 53 VH/ | HC 53 VH/ | HC 53 VH/ | HC 53 VH/ |
| 12E11 Parental VL | 12E11 variant | 12E11 variant | 12E11 variant |
| | LC 72 VL | LC 91 VL | LC 83 VL |
| 12E11 variant | 12E11 variant | 12E11 variant | 12E11 variant |
| HC 22 VH/ | HC 22 VH/ | HC 22 VH/ | HC 22 VH/ |
| 12E11 Parental VL | 12E11 variant | 12E11 variant | 12E11 variant |
| | LC 72 VL | LC 91 VL | LC 83 VL |

According to some embodiments, an antibody of the present disclosure specifically binds to Nectin-4 and comprises:
a VH chain comprising HCDRs 1-3 of a VH chain having the sequence of SEQ ID NO: 14; and
a VL chain comprising LCDRs 1-3 of a VL chain having the sequence of SEQ ID NO: 28.

According to some embodiments, an antibody of the present disclosure specifically binds to Nectin-4 and comprises:
a VH chain comprising the sequence of SEQ ID NO: 14; and
a VL chain comprising the sequence of SEQ ID NOs: 28.

According to some embodiments, an antibody of the present disclosure specifically binds to Nectin-4 and comprises:
a VH chain comprising HCDRs 1-3 of a VH chain having the sequence of SEQ ID NO: 15; and
a VL chain comprising LCDRs 1-3 of a VL chain having the sequence of SEQ ID NO: 29.

According to some embodiments, an antibody of the present disclosure specifically binds to Nectin-4 and comprises:
a VH chain comprising the sequence of SEQ ID NO: 15; and
a VL chain comprising the sequence of SEQ ID NOs: 29.

According to some embodiments, an antibody of the present disclosure specifically binds to Nectin-4 and comprises:
a VH chain comprising HCDRs 1-3 of a VH chain having the sequence of SEQ ID NO: 16; and
a VL chain comprising LCDRs 1-3 of a VL chain having the sequence of SEQ ID NO: 30.

According to some embodiments, an antibody of the present disclosure specifically binds to Nectin-4 and comprises:
a VH chain comprising the sequence of SEQ ID NO: 16; and
a VL chain comprising the sequence of SEQ ID NO: 30.

According to some embodiments, an antibody of the present disclosure specifically binds to Nectin-4 and comprises:
a VH chain comprising HCDRs 1-3 of a VH chain having the sequence of SEQ ID NO: 17; and
a VL chain comprising LCDRs 1-3 of a VL chain having the sequence of SEQ ID NO: 31.

According to some embodiments, an antibody of the present disclosure specifically binds to Nectin-4 and comprises:
a VH chain comprising the sequence of SEQ ID NO: 17; and
a VL chain comprising the sequence of SEQ ID NOs: 31.

In certain embodiments, the VH chain of an anti-Nectin-4 antibody comprises the HCDRs 1-3 of a VH chain having a sequence selected from SEQ ID NOs: 1 to 17 and comprises an amino acid sequence having 80% or greater, 85% or greater, 90% or greater, 95% or greater, 99% or greater, or 100% sequence identity to the amino acid sequence set forth in a sequence selected from SEQ ID NOs: 1 to 17. In certain embodiments, any amino acid differences between the VH chain of an anti-Nectin-4 antibody of the present disclosure and a sequence selected from SEQ ID NOs: 1 to 17 may be limited to regions outside of the CDRs, e.g., in one or more of the framework regions (FR), e.g., FR1, FR2, FR3, and/or FR4.

In certain embodiments, the VL chain of an anti-Nectin-4 antibody comprises the LCDRs 1-3 of a VL chain having a sequence selected from SEQ ID NOs: 18 to 31 and comprises an amino acid sequence having 80% or greater, 85% or greater, 90% or greater, 95% or greater, 99% or greater, or 100% sequence identity to the amino acid sequence set forth in a sequence selected from SEQ ID NOs: 18 to 31.

In certain embodiments, any amino acid differences between the VL chain of an anti-Nectin-4 antibody of the present disclosure and a sequence selected from SEQ ID NOs: 18 to 31 may be limited to regions outside of the CDRs, e.g., in one or more of FR1, FR2, FR3, and/or FR4.

In certain embodiments, an anti-Nectin-4 antibody of the present disclosure can comprise: a) a heavy chain comprising a VH region having the amino acid sequence set forth in a sequence selected from SEQ ID NOs: 1 to 17 and a heavy chain constant region having the amino acid sequence set forth in any one of SEQ ID NOs: 70 to 86, wherein the C present in the sequence LCTPSR in the constant region is replaced by fGly; and b) a light chain comprising the VL region having the amino acid sequence set forth in a sequence selected from SEQ ID NOS: 18 to 31.

In certain embodiments, an anti-Nectin-4 antibody of the present disclosure can comprise: a) a heavy chain comprising a VH region comprising an amino acid sequence at least 85% identical (e.g., at least 90%, at least 95%, at least 96%, at least 97%, at least 98%, or at least 99% identical) to the amino acid sequence set forth in a sequence selected from SEQ ID NOs: 1 to 17 and a heavy chain constant region comprising an amino acid sequence at least 85% identical (e.g., at least 90%, at least 95%, at least 96%, at least 97%, at least 98%, or at least 99% identical to the amino acid sequence set forth in any one of SEQ ID NOs: 70 to 86, wherein the C present in the sequence LCTPSR in the constant region is replaced by fGly′, wherein fGly′ refers to the amino acid residue conjugated to a moiety of interest; and b) a light chain comprising a VL region comprising an amino acid sequence at least 85% identical (e.g., at least 90%, at least 95%, at least 96%, at least 97%, at least 98%, or at least 99% identical) to the amino acid sequence set forth in a sequence selected from SEQ ID NOs: 18 to 31.

The anti-Nectin-4 antibodies of the present disclosure may bind to Nectin-1 protein, for example, a recombinant Nectin-4 protein, with an EC50 of about 0.1-1 nM, e.g., 0.2-0.9 nM, 0.3-0.7 nM, or 0.4-0.6 nM as measured by ELISA. The concentration of an antibody that provides half maximal response (e.g., half of the maximum fluorescence intensity) is measured as the EC50.

The anti-Nectin-4 antibodies of the present disclosure may bind to cancerous tissue and may show no or insignificant binding (e.g., insignificant binding as measured by immunohistochemistry or binding undetectable by immunohistochemistry) to normal tissue. For example, the anti-Nectin-4 antibodies described herein may bind to human solid tumors, such as ovarian, ductal breast carcinoma, lung adenocarcinoma, and pancreatic cancer that have cancerous cells while showing no detectable binding to human normal tissues, such as ovarian, breast, lung, and pancreas that do not have cancerous cells.

The antibodies find use in a variety of research, diagnostic, and therapeutic applications, including for performing any of the methods described in U.S. Patent Application Nos. 20210130459, 20200231670, 20180243434, 20110301056, 20100285597, 20080268476, the disclosure of each of which is incorporated herein by reference in its entirety.

A subject antibody specifically binds a Nectin-4 polypeptide, where the epitope comprises amino acid residues within a human Nectin-4 antigen comprising the amino acid sequence set forth in SEQ ID NO: 99:

(SEQ ID NO: 99)
MPLSLGAEMWGPEAWLLLLLLLASFTGRCPAGELETSDVVTVVLG
QDAKLPCFYRGDSGEQVGQVAWARVDAGEGAQELALLHSKYGLHV
SPAYEGRVEQPPPPRNPLDGSVLLRNAVQADEGEYECRVSTFPAG
SFQARLRLRVLVPPLPSLNPGPALEEGQGLTLAASCTAEGSPAPS
VTWDTEVKGTTSSRSFKHSRSAAVTSEFHLVPSRSMNGQPLTCVV
SHPGLLQDQRITHILHVSFLAEASVRGLEDQNLWHIGREGAMLKC
LSEGQPPPSYNWTRLDGPLPSGVRVDGDTLGFPPLTTEHSGIYVC
HVSNEFSSRDSQVTVDVLDPQEDSGKQVDLVSASVVVVGVIAALL
FCLLVVVVVLMSRYHRRKAQQMTQKYEEELTLTRENSIRRLHSHH
TDPRSQPEESVGLRAEGHPDSLKDNSSCSVMSEEPEGRSYSTLTT
VREIETQTELLSPGSGRAEEEEDQDEGIKQAMNHFVQENGTLRAK
PTGNGIYINGRGHLV.

In certain embodiments, the Nectin-4 epitope bound by the anti-Nectin-4 antibodies disclosed herein is present on Nectin-4 expressed by HEK cells overexpressing human Nectin-4 or SK-BR-3 breast cancer cells.

A subject antibody exhibits high affinity binding to Nectin-4. For example, a subject antibody binds to Nectin-4 with an affinity of at least about 10⁻⁷ M, at least about 10⁻⁸ M, at least about 10⁻⁹ M, at least about 10⁻¹⁰ M, at least about 10⁻¹¹ M, or at least about 10⁻¹² M, or greater than 10⁻¹² M. A subject antibody binds to an epitope present on Nectin-4 with an affinity of from about 10⁻⁷ M to about 10⁻⁸ M, from about 10⁻⁸ M to about 10⁻⁹ M, from about 10⁻⁹ M to about 10⁻¹⁰ M, from about 10⁻¹⁰ M to about 10⁻¹¹ M, or from about 10⁻¹¹ M to about 10⁻¹² M, or greater than 10⁻¹² M.

An anti-Nectin-4 antibody of the present disclosure can in some cases induce apoptosis in a cell that expresses Nectin-4 on its cell surface.

A “Nectin-4 antigen” or “Nectin-4 polypeptide” can comprises an amino acid sequence having at least about 75%, at least about 80%, at least about 90%, at least about 95%, at least about 98%, at least about 99%, or 100%, amino acid sequence identity to SEQ ID NO: 99.

As used herein the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes; and numerous immunoglobulin variable region genes. Full-length immunoglobulin light chains (about 25 kD or 214 amino acids) are encoded by a variable region gene at the N-terminus (about 110 amino acids) and a kappa or lambda constant region at the C-terminus. Full-length immunoglobulin heavy chains (about 50 kD or 446 amino acids) are encoded by a variable region gene at the N-terminus (about 116 amino acids) and one of the other aforementioned constant region genes at the C-terminus, e.g., gamma (encoding about 330 amino acids). In some embodiments, a subject antibody comprises full-length immunoglobulin heavy chain and a full-length immunoglobulin light chain.

In some embodiments, a subject antibody does not comprise a full-length immunoglobulin heavy chain and a full-length immunoglobulin light chain, and instead comprises antigen-binding fragments of a full-length immunoglobulin heavy chain and a full-length immunoglobulin light chain. In some embodiments, the antigen-binding fragments are contained on separate polypeptide chains; in other embodiments, the antigen-binding fragments are contained within a single polypeptide chain. The term “antigen-binding fragment” refers to one or more fragments of a full-length antibody that are capable of specifically binding to Nectin-4, as described above. Examples of binding fragments include (i) a Fab fragment (a monovalent fragment consisting of the VL, VH, CL and CH1 domains; (ii) a F(ab′)₂ fragment (a bivalent fragment comprising two Fab fragments linked by a disulfide bridge at the hinge region; (iii) a Fd fragment (consisting of the VH and CH1 domains); (iv) a Fv fragment (consisting of the VH and VL domains of a single arm of an antibody); (v) a dAb fragment (consisting of the VH domain); (vi) an isolated CDR; (vii) a single chain Fv (scFv) (consisting of the VH and VL domains of a single arm of an antibody joined by a synthetic linker using recombinant means such that the VH and VL domains pair to form a monovalent molecule); (viii) diabodies (consisting of two scFvs in which the VH and VL domains are joined such that they do not pair to form a monovalent molecule; the VH of each one of the scFv pairs with the VL domain of the other scFv to form a bivalent molecule); (ix) bi-specific antibodies (consisting of at least two antigen binding regions, each region binding a different epitope). In some embodiments, a subject antibody fragment is a Fab fragment. In some embodiments, a subject antibody fragment is a single-chain antibody (scFv).

In some embodiments, a subject antibody is a recombinant or modified antibody, e.g., a chimeric, humanized, deimmunized or an in vitro generated antibody. The term “recombinant” or “modified” antibody as used herein is intended to include all antibodies that are prepared, expressed, created, or isolated by recombinant means, such as (i) antibodies expressed using a recombinant expression vector transfected into a host cell; (ii) antibodies isolated from a recombinant, combinatorial antibody library; (iii) antibodies isolated from an animal (e.g. a mouse) that is transgenic for human immunoglobulin genes; or (iv) antibodies prepared, expressed, created, or isolated by any other means that involves splicing of human immunoglobulin gene sequences to other DNA sequences. Such recombinant antibodies include humanized, CDR grafted, chimeric, deimmunized, and in vitro generated antibodies; and can optionally include constant regions derived from human germline immunoglobulin sequences.

Full length bispecific antibodies may be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy chain disulfide bonds in the hinge regions of the parent monospecific antibodies are reduced. The resulting free cysteines of one of the parent monospecific antibodies form an inter heavy-chain disulfide bond with cysteine residues of a second parent monospecific antibody molecule and simultaneously CH3 domains of the parent antibodies release and reform by dissociation-association. The CH3 domains of the Fab arms may be engineered to favor heterodimerization over homodimerization. The resulting product is a bispecific antibody having two Fab arms or half molecules which each bind a distinct epitope.

The “knob-in-hole” strategy (see, e.g., PCT Intl. Publ. No. WO 2006/028936) may be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob.” Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T3945/Y407A, T366W/T394S, F405W/T394S and T366W/T366S/L368A/Y407V.

Other strategies such as promoting heavy chain heterodimerization using electrostatic interactions by substituting positively charged residues at one CH3 surface and negatively charged residues at a second CH3 surface may be used, as described in US Pat. Publ. No. US2010/0015133; US Pat. Publ. No. US2009/0182127; US Pat. Publ. No. U82010/028637 or US Pat. Publ. No. US2011/0123532. In other strategies, heterodimerization may be promoted by following substitutions (expressed as modified position in the first CH3 domain of the first heavy chain/modified position in the second CH3 domain of the second heavy chain): L351 Y/F405A/Y407V/T394W, T366I/K392M/T394W/F405A/Y407V, T366L/K392M/T394W/F405A/Y407V, L351Y/Y407A/T366A/K409F, L351Y/Y407A/T366V/K409F, Y407A/T366A/K409F, or T350V/L351Y/F405A/Y407V, T350V/T366L/K392L/T394W as described in U.S. Pat. Pub. No. US2012/0149876 or U.S. Pat. Pub. No. US2013/0195849.

Also provided are single chain bispecific antibodies. In some embodiments, a single chain bispecific antibody of the present disclosure is a bispecific scFv. A subject antibody can be humanized. The constant region(s), if present, can also be substantially or entirely from a human immunoglobulin.

Methods of making humanized antibodies are known in the art. The substitution of mouse CDRs into a human variable domain framework can result in retention of their correct spatial orientation where, e.g., the human variable domain framework adopts the same or similar conformation to the mouse variable framework from which the CDRs originated. This can be achieved by obtaining the human variable domains from human antibodies whose framework sequences exhibit a high degree of sequence identity with the murine variable framework domains from which the CDRs were derived. The heavy and light chain variable framework regions can be derived from the same or different human antibody sequences. The human antibody sequences can be the sequences of naturally occurring human antibodies or can be consensus sequences of several human antibodies.

Having identified the complementarity determining regions of the murine donor immunoglobulin and appropriate human acceptor immunoglobulins, the next step is to determine which, if any, residues from these components should be substituted to optimize the properties of the resulting humanized antibody. In general, substitution of human amino acid residues with murine should be minimized, because introduction of murine residues increases the risk of the antibody eliciting a human-anti-mouse-antibody (HAMA) response in humans. Art-recognized methods of determining immune response can be performed to monitor a HAMA response in a particular patient or during clinical trials. Patients administered humanized antibodies can be given an immunogenicity assessment at the beginning and throughout the administration of said therapy. The HAMA response is measured, for example, by detecting antibodies to the humanized therapeutic reagent, in serum samples from the patient using a method known to one in the art, including surface plasmon resonance technology (BIACORE) and/or solid-phase ELISA analysis. In many embodiments, a subject humanized antibody does not substantially elicit a HAMA response in a human subject.

Certain amino acids from the human variable region framework residues are selected for substitution based on their possible influence on CDR conformation and/or binding to antigen. The unnatural juxtaposition of murine CDR regions with human variable framework region can result in unnatural conformational restraints, which, unless corrected by substitution of certain amino acid residues, lead to loss of binding affinity. The selection of amino acid residues for substitution can be determined, in part, by computer modeling. Computer hardware and software for producing three-dimensional images of immunoglobulin molecules are known in the art. In general, molecular models are produced starting from solved structures for immunoglobulin chains or domains thereof. The chains to be modeled are compared for amino acid sequence similarity with chains or domains of solved three-dimensional structures, and the chains or domains showing the greatest sequence similarity is/are selected as starting points for construction of the molecular model. Chains or domains sharing at least 50% sequence identity are selected for modeling, and preferably those sharing at least 60%, 70%, 80%, 90% sequence identity or more are selected for modeling. The solved starting structures are modified to allow for differences between the actual amino acids in the immunoglobulin chains or domains being modeled, and those in the starting structure. The modified structures are then assembled into a composite immunoglobulin. Finally, the model is refined by energy minimization and by verifying that all atoms are within appropriate distances from one another and that bond lengths and angles are within chemically acceptable limits.

When framework residues, as defined by Kabat, supra, constitute structural loop residues as defined by Chothia, supra, the amino acids present in the mouse antibody may be selected for substitution into the humanized antibody. Residues which are “adjacent to a CDR region” include amino acid residues in positions immediately adjacent to one or more of the CDRs in the primary sequence of the humanized immunoglobulin chain, for example, in positions immediately adjacent to a CDR as defined by Kabat, or a CDR as defined by Chothia (See e.g., Chothia and Lesk JMB 196:901 (1987)). These amino acids are particularly likely to interact with the amino acids in the CDRs and, if chosen from the acceptor, to distort the donor CDRs and reduce affinity. Moreover, the adjacent amino acids may interact directly with the antigen (Amit et al., Science, 233:747 (1986)) and selecting these amino acids from the donor may be desirable to keep all the antigen contacts that provide affinity in the original antibody.

In some embodiments, a subject antibody comprises scFv multimers. For example, in some embodiments, a subject antibody is an scFv dimer (e.g., comprises two tandem scFv (scFv₂)), an scFv trimer (e.g., comprises three tandem scFv (scFv₃)), an scFv tetramer (e.g., comprises four tandem scFv (scFv₄)), or is a multimer of more than four scFv (e.g., in tandem). The scFv monomers can be linked in tandem via linkers of from about 2 amino acids to about 10 amino acids in length, e.g., 2 aa, 3 aa, 4 aa, 5 aa, 6 aa, 7 aa, 8 aa, 9 aa, or 10 aa in length. Suitable linkers include, e.g., (Gly)ₓ, where x is an integer from 2 to 10, glycine-serine polymers, and the like.

In some embodiments, a subject antibody comprises a constant region of an immunoglobulin (e.g., an Fc region). The Fc region, if present, can be a human Fc region. If constant regions are present, the antibody can contain both light chain and heavy chain constant regions. The antibodies described herein include antibodies having all types of constant regions, including IgM, IgG, IgD, IgA and IgE, and any isotype, including IgG1, IgG2, IgG3 and IgG4. An example of a suitable heavy chain Fc region is a human isotype IgG1 Fc. Light chain constant regions can be lambda or kappa. A subject antibody (e.g., a subject humanized antibody) can comprise sequences from more than one class or isotype. Antibodies can be expressed as tetramers containing two light and two heavy chains, as separate heavy chains, light chains, as Fab, Fab′ F(ab′)2, and Fv, or as single chain antibodies in which heavy and light chain variable domains are linked through a spacer.

In some embodiments, an anti-Nectin-4 antibody of the present disclosure may include one or more amino acid substitutions introduced in the Fc region. In some embodiments, the one or more of the amino acid substitutions may be at the positions 239, 298, 326, 330 and 332 in the Fc region. In some embodiments, an anti-Nectin-4 antibody of the present disclosure may include one or more of the following amino acid substitutions introduced in the Fc region: I332E; S239D/A330L/I332E; S239D/S298A/I332E; S239D/K326T/I332E; S239D/S298A/K326T/I332E; or S239D/A330L/I332E/D356E/L³⁵⁸M.

In some embodiments, a subject antibody comprises a free thiol (—SH) group at the carboxyl terminus, where the free thiol group can be used to attach the antibody to a second polypeptide (e.g., another antibody, including a subject antibody), a scaffold, a carrier, etc.

In some embodiments, a subject antibody comprises one or more non-naturally occurring amino acids. In some embodiments, the non-naturally encoded amino acid comprises a carbonyl group, an acetyl group, an aminooxy group, a hydrazine group, a hydrazide group, a semicarbazide group, an azide group, or an alkyne group. Inclusion of a non-naturally occurring amino acid can provide for linkage to a polymer, a second polypeptide, a scaffold, etc. Examples of such non-naturally-occurring amino acids include, but are not limited to, N-acetylglucosaminyl-L-serine, N-acetylglucosaminyl-L-threonine, and O-phosphotyrosine.

The present disclosure also provides anti-Nectin-4 antibodies having an attached moiety of interest, e.g., a detectable label, drug, half-life-extending moiety, and the like. Modification of antibodies can be accomplished by a variety of synthetic and/or recombinant methods. The moiety or moieties attached to an antibody can provide for one or more of a wide variety of functions or features. Exemplary moieties include detectable labels (e.g., dye labels (e.g., chromophores, fluorophores), biophysical probes (spin labels, nuclear magnetic resonance (NMR) probes), fluorescence Resonance Energy Transfer (FRET)-type labels (e.g., at least one member of a FRET pair, including at least one member of a fluorophore/quencher pair), Bioluminescence Resonance Energy Transfer (BRET)-type labels (e.g., at least one member of a BRET pair), immunodetectable tags (e.g., FLAG, His (6), and the like); water soluble polymers (e.g., PEGylation); purification tags (e.g., to facilitate isolation by affinity chromatography (e.g., attachment of a FLAG epitope; membrane localization domains (e.g., lipids or glycophosphatidylinositol (GPI)-type anchors); immobilization tags (e.g., to facilitate attachment of the polypeptide to a surface, including selective attachment); drugs (e.g., to facilitate drug targeting, e.g., through attachment of the drug to an antibody); and the like.

In some embodiments, a subject antibody is linked (e.g., covalently linked) to a polymer (e.g., a polymer other than a polypeptide). Suitable polymers include, e.g., biocompatible polymers, and water-soluble biocompatible polymers. Suitable polymers include synthetic polymers and naturally-occurring polymers. Suitable polymers include, e.g., substituted or unsubstituted straight or branched chain polyalkylene, polyalkenylene or polyoxyalkylene polymers or branched or unbranched polysaccharides, e.g., a homo- or hetero-polysaccharide.

Suitable polymers include, e.g., ethylene vinyl alcohol copolymer (commonly known by the generic name EVOH or by the trade name EVAL); polybutylmethacrylate; poly(hydroxyvalerate); poly(L-lactic acid); polycaprolactone; poly(lactide-co-glycolide); poly(hydroxybutyrate); poly(hydroxybutyrate-co-valerate); polydioxanone; polyorthoester; polyanhydride; poly(glycolic acid); poly(D,L-lactic acid); poly(glycolic acid-co-trimethylene carbonate); polyphosphoester; polyphosphoester urethane; poly(amino acids); cyanoacrylates; poly(trimethylene carbonate); poly(iminocarbonate); copoly(ether-esters) (e.g., poly(ethylene oxide)-poly(lactic acid) (PEO/PLA) co-polymers); polyalkylene oxalates; polyphosphazenes; biomolecules, such as fibrin, fibrinogen, cellulose, starch, collagen and hyaluronic acid; polyurethanes; silicones; polyesters; polyolefins; polyisobutylene and ethylene-alphaolefin copolymers; acrylic polymers and copolymers; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins; polyurethanes; rayon; rayon-triacetate; cellulose; cellulose acetate; cellulose butyrate; cellulose acetate butyrate; cellophane; cellulose nitrate; cellulose propionate; cellulose ethers; amorphous Teflon; poly(ethylene glycol); and carboxymethyl cellulose.

Suitable synthetic polymers include unsubstituted and substituted straight or branched chain poly(ethyleneglycol), poly(propyleneglycol) poly(vinylalcohol), and derivatives thereof, e.g., substituted poly(ethyleneglycol) such as methoxypoly(ethyleneglycol), and derivatives thereof. Suitable naturally-occurring polymers include, e.g., albumin, amylose, dextran, glycogen, and derivatives thereof.

Suitable polymers can have an average molecular weight in a range of from 500 Da to 50000 Da, e.g., from 5000 Da to 40000 Da, or from 25000 to 40000 Da. For example, in some embodiments, where a subject antibody comprises a poly(ethylene glycol) (PEG) or methoxypoly(ethyleneglycol) polymer, the PEG or methoxypoly(ethyleneglycol) polymer can have a molecular weight in a range of from about 0.5 kiloDaltons (kDa) to 1 kDa, from about 1 kDa to 5 kDa, from 5 kDa to 10 kDa, from 10 kDa to 25 kDa, from 25 kDa to 40 kDa, or from 40 kDa to 60 kDa.

In some embodiments, a subject antibody is covalently linked to a PEG polymer. In some embodiments, a subject scFv multimer is covalently linked to a PEG polymer. PEG suitable for conjugation to a protein is generally soluble in water at room temperature, and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons. The PEG conjugated to the subject antibody can be linear or branched. Branched PEG derivatives include star-PEG's and multi-armed PEG's.

A subject antibody can be glycosylated, e.g., a subject antibody can comprise a covalently linked carbohydrate or polysaccharide moiety. Glycosylation of antibodies is typically either N-linked or O-linked. Addition of glycosylation sites to an antibody is conveniently accomplished by altering the amino acid sequence such that it contains N- or O-linked glycosylation sites. Similarly, removal of glycosylation sites can be accomplished by amino acid alteration within the native glycosylation sites of an antibody.

A subject antibody can be covalently linked to a second moiety (e.g., a lipid, a polypeptide other than a subject antibody, a synthetic polymer, a carbohydrate, and the like) using for example, glutaraldehyde, a homobifunctional cross-linker, or a heterobifunctional cross-linker. Glutaraldehyde cross-links polypeptides via their amino moieties. Homobifunctional cross-linkers (e.g., a homobifunctional imidoester, a homobifunctional N-hydroxysuccinimidyl (NHS) ester, or a homobifunctional sulfhydryl reactive cross-linker) contain two or more identical reactive moieties and can be used in a one-step reaction procedure in which the cross-linker is added to a solution containing a mixture of the polypeptides to be linked. Homobifunctional NHS ester and imido esters cross-link amine containing polypeptides. In a mild alkaline pH, imido esters react only with primary amines to form imidoamides, and overall charge of the cross-linked polypeptides is not affected. Homobifunctional sulfhydryl reactive cross-linkers includes bismalcimidhexane (BMH), 1,5-difluoro-2,4-dinitrobenzene (DFDNB), and 1,4-di-(3′,2′-pyridyldithio) propinoamido butane (DPDPB).

Heterobifunctional cross-linkers have two or more different reactive moieties (e.g., amine reactive moiety and a sulfhydryl-reactive moiety) and are cross-linked with one of the polypeptides via the amine or sulfhydryl reactive moiety, then reacted with the other polypeptide via the non-reacted moiety. Multiple heterobifunctional haloacetyl cross-linkers are available, as are pyridyl disulfide cross-linkers. Carbodiimides are a classic example of heterobifunctional cross-linking reagents for coupling carboxyls to amines, which results in an amide bond.

A subject antibody will in some embodiments comprise a “radiopaque” label, e.g., a label that can be easily visualized using for example x-rays. Radiopaque materials are well known to those of skill in the art. The most common radiopaque materials include iodide, bromide or barium salts. Other radiopaque materials are also known and include, but are not limited to organic bismuth derivatives, radiopaque multiurethanes, organobismuth composites, radiopaque barium multimer complexes, and the like.

In some embodiments, a subject antibody comprises a polyamine modification. A subject antibody can be modified with polyamines that are either naturally occurring or synthetic. Useful naturally occurring polyamines include putrescine, spermidine, spermine, 1,3-deaminopropane, norspermidine, syn-homospermidine, thermine, thermospermine, caldopentamine, homocaldopentamine, and canavalmine. Putrescine, spermidine and spermine are particularly useful. Synthetic polyamines are composed of the empirical formula C_{X}H_{Y}N_{Z}, can be cyclic or acyclic, branched or unbranched, hydrocarbon chains of 3-12 carbon atoms that further include 1-6 NR or N(R)₂ moieties, wherein R is H, (C₁-C₄)alkyl, phenyl, or benzyl. Polyamines can be linked to an antibody using any standard crosslinking method.

### Methods for Modification of Antibodies

An anti-Nectin-4 antibody conjugate of the present disclosure can include: 1) Ig heavy chain constant region conjugated to a moiety of interest; and an Ig light chain constant region conjugated to a moiety of interest; 2) an Ig heavy chain constant region conjugated to a moiety of interest; and an Ig light chain constant region that is not conjugated to a moiety of interest; or 3) an Ig heavy chain constant region that is not conjugated to a moiety of interest; and an Ig light chain constant region conjugated to a moiety of interest. A subject anti-Nectin-4 antibody conjugate can also include VH and/or VL domains conjugated to a moiety of interest.

In one example, the antibody can be modified to include a 2-formylglycine residue, which can serve as a chemical handle for attachment of a heterologous moiety. For example, the heavy and/or light chain constant region of an anti-Nectin-4 of the present disclosure can be modified to include an amino acid sequence of a sulfatase motif which is capable of being converted by action of a 2-formylglycine generating enzyme (FGE) to contain a 2-formylglycine (fGly). Such sulfatase motifs may also be referred to herein as an FGE-modification site. Action of FGE is directed in a sequence-specific manner in that the FGE acts at a sulfatase motif positioned within the immunoglobulin polypeptide. The moiety of interest is provided as a component of a reactive partner for reaction with an aldehyde of the fGly residue of a converted aldehyde tag of the tagged Ig polypeptide. A wide range of commercially available reagents can be used to accomplish attachment of a moiety of interest to an fGly residue of an aldehyde tagged Ig polypeptide. For example, aminooxy, hydrazide, or thiosemicarbazide derivatives of a number of moieties of interest are suitable reactive partners, and are readily available or can be generated using standard chemical methods.

As noted above, the amino acid sequence of an anti-Nectin-4 antibody can be modified to include a sulfatase motif that contains a serine or cysteine residue that is capable of being converted (oxidized) to a 2-formylglycine (fGly) residue by action of a formylglycine generating enzyme (FGE) either in vivo (e.g., at the time of translation of an aldehyde tag-containing protein in a cell) or in vitro (e.g., by contacting an aldehyde tag-containing protein with an FGE in a cell-free system). Such sulfatase motifs may also be referred to herein as an FGE-modification site.

### Sulfatase Motifs

A minimal sulfatase motif of an aldehyde tag is usually 5 or 6 amino acid residues in length, usually no more than 6 amino acid residues in length. Sulfatase motifs provided in an Ig polypeptide are at least 5 or 6 amino acid residues, and can be, for example, from 5 to 16, 6-16, 5-15, 6-15, 5-14, 6-14, 5-13, 6-13, 5-12, 6-12, 5-11, 6-11, 5-10, 6-10, 5-9, 6-9, 5-8, or 6-8 amino acid residues in length, so as to define a sulfatase motif of less than 16, 15, 14, 13, 12, 11, 10, 9, 8 or 7 amino acid residues in length.

In certain embodiments, polypeptides of interest include those where one or more amino acid residues, such as 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more amino acid residues have been inserted, deleted, substituted (replaced) relative to the native amino acid sequence to provide for a sequence of a sulfatase motif in the polypeptide. In certain embodiments, the polypeptide includes a modification (insertion, addition, deletion, and/or substitution/replacement) of less than 20, 19, 18, 17, 16, 15, 14, 13, 12, 11, 10, 9, 8, 7, 6, 5, 4, 3 or 2 amino acid residues of the amino acid sequence relative to the native amino acid sequence of the polypeptide. Where an amino acid sequence native to the polypeptide (e.g., anti-Nectin-4 antibody) contains one or more residues of the desired sulfatase motif, the total number of modifications of residues can be reduced, e.g., by site-specification modification (insertion, addition, deletion, substitution/replacement) of amino acid residues flanking the native amino acid residues to provide a sequence of the desired sulfatase motif. In certain embodiments, the extent of modification of the native amino acid sequence of the target anti-Nectin-4 polypeptide is minimized, so as to minimize the number of amino acid residues that are inserted, deleted, substituted (replaced), or added (e.g., to the N- or C-terminus). Minimizing the extent of amino acid sequence modification of the target anti-Nectin-4 polypeptide may minimize the impact such modifications may have upon anti-Nectin-4 function and/or structure.

It should be noted that while aldehyde tags of particular interest are those comprising at least a minimal sulfatase motif (also referred to a “consensus sulfatase motif”), it will be readily appreciated that longer aldehyde tags are both contemplated and encompassed by the present disclosure and can find use in the compositions and methods of the present disclosure. Aldehyde tags can thus comprise a minimal sulfatase motif of 5 or 6 residues, or can be longer and comprise a minimal sulfatase motif which can be flanked at the N- and/or C-terminal sides of the motif by additional amino acid residues. Aldehyde tags of, for example, 5 or 6 amino acid residues are contemplated, as well as longer amino acid sequences of more than 5, 6, 7, 8, 9, 10, 11, 12, 13, 14, 15, 16, 17, 18, 19, 20 or more amino acid residues.

An aldehyde tag can be present at or near the C-terminus of an Ig heavy chain; e.g., an aldehyde tag can be present within 1, 2, 3, 4, 5, 6, 7, 8, 9, or 10 amino acids of the C-terminus of a native, wild-type Ig heavy chain. An aldehyde tag can be present within a CH1 domain of an Ig heavy chain. An aldehyde tag can be present within a CH2 domain of an Ig heavy chain. An aldehyde tag can be present within a CH3 domain of an Ig heavy chain. An aldehyde tag can be present in an Ig light chain constant region, e.g., in a kappa light chain constant region or a lambda light chain constant region.

In certain embodiments, the sulfatase motif used may be described by the formula:

X¹Z¹X²Z²X³Z³(SEQ ID NO: 102) (I′), where
Z¹ is cysteine or serine (which can also be represented by (C/S));
Z² is either a proline or alanine residue (which can also be represented by (P/A));
Z³ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), usually A, G, L, V, or I;
X¹ is present or absent and, when present, can be any amino acid, though usually an aliphatic amino acid, a sulfur-containing amino acid, or a polar, uncharged amino acid, (e.g., other than an aromatic amino acid or a charged amino acid), usually L, M, V, S or T, more usually L, M, S or V, with the proviso that when the sulfatase motif is at the N-terminus of the target polypeptide, X¹ is present; and
X² and X³ independently can be any amino acid, though usually an aliphatic amino acid, a polar, uncharged amino acid, or a sulfur containing amino acid (e.g., other than an aromatic amino acid or a charged amino acid), e.g., S, T, A, V, G or C; e.g., S, T, A, V or G. In one example, the aldehyde tag is of the formula L (C/S)TPSR (SEQ ID NO: 103), e.g., LCTPSR (SEQ ID NO: 104) or LSTPSR (SEQ ID NO: 105). Thus, the present disclosure provides antibodies that include an aldehyde-tagged Ig heavy chain and/or an aldehyde-tagged Ig light chain, where the aldehyde-tagged Ig antibody comprises an Ig constant region amino acid sequence of the heavy and/or light chain contains such a sulfatase motif.

For example, in some embodiments, the amino acid sequence of an anti-Nectin-4 heavy and/or light chain can be modified to provide a sequence of at least 5 amino acids of the formula X¹Z¹X²Z²X³Z³, where
Z¹ is cysteine or serine;
Z² is a proline or alanine residue;
Z³ is an aliphatic amino acid or a basic amino acid;
X¹ is present or absent and, when present, is any amino acid, with the proviso that when the heterologous sulfatase motif is at an N-terminus of the polypeptide, X¹ is present;
X² and X³ are each independently any amino acid,
where the sequence is within or adjacent a solvent-accessible loop region of the Ig constant region, and wherein the sequence is not at the C-terminus of the Ig heavy chain.

The sulfatase motif is generally selected so as to be capable of conversion by a selected FGE, e.g., an FGE present in a host cell in which the aldehyde tagged polypeptide is expressed or an FGE which is to be contacted with the aldehyde tagged polypeptide in a cell-free in vitro method.

For example, where the FGE is a eukaryotic FGE (e.g., a mammalian FGE, including a human FGE), the sulfatase motif can be of the formula:

X¹CX²PX³Z³ (I″)
where
X¹ may be present or absent and, when present, can be any amino acid, e.g., an aliphatic amino acid, a sulfur-containing amino acid, or a polar, uncharged amino acid, (e.g., other than an aromatic amino acid or a charged amino acid), e.g., L, M, S or V, with the proviso that when the sulfatase motif is at the N-terminus of the target polypeptide, X¹ is present;
X² and X³ independently can be any amino acid, e.g., an aliphatic amino acid, a sulfur-containing amino acid, or a polar, uncharged amino acid, (e.g., other than an aromatic amino acid or a charged amino acid), e.g., S, T, A, V, G, or C, e.g., S, T, A, V or G; and
Z³ is a basic amino acid (e.g., arginine (R), and may be lysine (K) or histidine (H), e.g., lysine), or an aliphatic amino acid (alanine (A), glycine (G), leucine (L), valine (V), isoleucine (I), or proline (P), e.g., A, G, L, V, or I.

Specific examples of sulfatase motifs include LCTPSR (SEQ ID NO: 104), MCTPSR (SEQ ID NO: 105), VCTPSR (SEQ ID NO: 106), LCSPSR (SEQ ID NO: 107), LCAPSR (SEQ ID NO: 108), LCVPSR (SEQ ID NO: 109), LCGPSR (SEQ ID NO: 110), ICTPAR (SEQ ID NO: 111), LCTPSK (SEQ ID NO: 112), MCTPSK (SEQ ID NO: 113), VCTPSK (SEQ ID NO: 114), LCSPSK (SEQ ID NO: 115), LCAPSK (SEQ ID NO: 116), LCVPSK (SEQ ID NO: 117), LCGPSK (SEQ ID NO: 118), LCTPSA (SEQ ID NO: 119), ICTPAA (SEQ ID NO: 120), MCTPSA (SEQ ID NO: 121), VCTPSA (SEQ ID NO: 122), LCSPSA (SEQ ID NO: 123), LCAPSA (SEQ ID NO: 124), LCVPSA (SEQ ID NO: 125), and LCGPSA (SEQ ID NO: 126).

fGly-Containing Sequences

In general, the FGE used to facilitate conversion of cysteine or serine to fGly in a sulfatase motif of an aldehyde tag of a target polypeptide is selected according to the sulfatase motif present in the aldehyde tag. The FGE can be native to the host cell in which the aldehyde tagged polypeptide is expressed, or the host cell can be genetically modified to express an appropriate FGE. In some embodiments it may be desired to use a sulfatase motif compatible with a human FGE, and express the aldehyde tagged protein in a human cell that expresses the FGE or in a host cell, usually a mammalian cell, genetically modified to express a human FGE. In general, an FGE suitable for use in generating an fGly-modified antibody can be obtained from naturally occurring sources or synthetically produced. For example, an appropriate FGE can be derived from biological sources which naturally produce an FGE or which are genetically modified to express a recombinant gene encoding an FGE. Nucleic acids encoding a number of FGEs are known in the art and readily.

Following action of an FGE on the sulfatase motif, Z¹ is oxidized to generate a 2-formylglycine (fGly) residue. Furthermore, following both FGE-mediated conversion and reaction with a reactive partner comprising a moiety of interest, the fGly position at Z¹ in the formula above is covalently bound to the moiety of interest (e.g., detectable label, water soluble polymer, polypeptide, drug, active agent, etc.). Thus, the present disclosure provides an anti-Nectin-4 antibody having an amino acid sequence modified to comprise an fGly moiety.

Upon action of FGE on the anti-Nectin-4 heavy and/or light chain, the serine or the cysteine in the sulfatase motif is modified to fGly. Thus, the fGly-containing sulfatase motif can be of the formula:

X¹(fGly)X²Z²X³Z³(SEQ ID NO: 127) (I′″)

where
fGly is the formylglycine residue;
Z² is either a proline or alanine residue (which can also be represented by (P/A));
Z³ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (e.g., other than an aromatic amino acid or a charged amino acid), e.g., L, M, V, S or T, e.g., L, M or V, with the proviso that when the sulfatase motif is at the N-terminus of the target polypeptide, X¹ is present; and
X² and X³ independently can be any amino acid, e.g., an aliphatic amino acid, a sulfur-containing amino acid, or a polar, uncharged amino acid, (e.g., other than an aromatic amino acid or a charged amino acid), e.g., S, T, A, V, G or C, e.g., S, T, A, V or G.

As described above, to produce the conjugate, the polypeptide containing the fGly residue may be conjugated to a drug or active agent by reaction of the fGly with a reactive moiety (e.g., hydrazinyl-indolyl or a hydrazinyl-pyrrolo-pyridinyl coupling moiety, as described above) of a linker attached to the drug or active agent to produce an fGly′-containing sulfatase motif. As used herein, the term fGly′ refers to the amino acid residue of the sulfatase motif that is coupled to the drug or active agent through a linker as described herein. Thus, the present disclosure provides an anti-Nectin-4 antibody conjugate (also referred to herein as an “anti-Nectin-4 conjugate”).

In certain embodiments, the anti-Nectin-4 conjugate comprises an fGly′-containing sulfatase motif of the formula:

X¹(fGly′)X²Z²X³Z³(SEQ ID NO: 128) (II)
where
fGly′ is the amino acid residue coupled to the drug or active agent through a linker as described herein;
Z² is either a proline or alanine residue (which can also be represented by (P/A));
Z³ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (e.g., other than an aromatic amino acid or a charged amino acid), e.g., L, M, V, S or T, e.g., L, M or V, with the proviso that when the sulfatase motif is at the N-terminus of the target polypeptide, X¹ is present; and
X² and X³ independently can be any amino acid, e.g., an aliphatic amino acid, a sulfur-containing amino acid, or a polar, uncharged amino acid, (e.g., other than an aromatic amino acid or a charged amino acid), e.g., S, T, A, V, G or C, e.g., S, T, A, V or G.

In certain embodiments, the sequence of formula (II) is positioned at a C-terminus of a heavy chain constant region of the anti-Nectin-4 antibody. In some instances, the heavy chain constant region comprises a sequence of the formula (II):

X¹(fGly′)X²Z²X³Z³ (II)
where
fGly′ is the amino acid residue coupled to the drug or active agent through a linker as described herein;
Z² is either a proline or alanine residue (which can also be represented by (P/A));
Z³ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (e.g., other than an aromatic amino acid or a charged amino acid), e.g., L, M, V, S or T, e.g., L, M or V, with the proviso that when the sulfatase motif is at the N-terminus of the target polypeptide, X¹ is present;
X² and X³ independently can be any amino acid, e.g., an aliphatic amino acid, a sulfur-containing amino acid, or a polar, uncharged amino acid, (e.g., other than an aromatic amino acid or a charged amino acid), e.g., S, T, A, V, G or C, e.g., S, T, A, V or G; and wherein the sequence is C-terminal to the amino acid sequence QKSLSLSPGK (SEQ ID NO: 129), and where the sequence may include 1, 2, 3, 4, 5, or from 5 to 10, amino acids that are not present in a native, wild-type heavy Ig chain constant region.

In certain embodiments, the heavy chain constant region comprises the sequence SLSLSPGSL(fGly′)TPSRGS (SEQ ID NO: 248) at the C-terminus of the Ig heavy chain, e.g., in place of a native SLSLSPGK (SEQ ID NO: 249) sequence.

In certain embodiments, the heavy chain constant region comprises the sequence SPGSL(fGly′)TPSRGS (SEQ ID NO: 130) at the C-terminus of the Ig heavy chain, e.g., in place of a native SPGK (SEQ ID NO: 131) sequence.

In certain embodiments, the amino acid residue coupled to the drug or active agent (fGly′) is positioned in a light chain constant region of the anti-Nectin-4 antibody. In certain embodiments, the light chain constant region comprises a sequence of the formula (II):

X¹(fGly′)X²Z²X³Z³ (II)
where
fGly′ is the amino acid residue coupled to the drug or active agent through a linker as described herein;
Z² is either a proline or alanine residue (which can also be represented by (P/A));
Z³ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (e.g., other than an aromatic amino acid or a charged amino acid), e.g., L, M, V, S or T, e.g., L, M or V, with the proviso that when the sulfatase motif is at the N-terminus of the target polypeptide, X¹ is present;
X² and X³ independently can be any amino acid, e.g., an aliphatic amino acid, a sulfur-containing amino acid, or a polar, uncharged amino acid, (e.g., other than an aromatic amino acid or a charged amino acid), e.g., S, T, A, V, G or C, e.g., S, T, A, V or G; and
wherein the sequence is C-terminal to the amino acid sequence KVDNAL (SEQ ID NO: 132) and/or is N-terminal to the amino acid sequence QSGNSQ (SEQ ID NO: 133).

In certain embodiments, the light chain constant region comprises the sequence KVDNAL(fGly′)TPSRQSGNSQ (SEQ ID NO: 134).

In certain embodiments, the amino acid residue coupled to the drug or active agent (fGly′) is positioned in a heavy chain CH1 region of the anti-Nectin-4 antibody. In certain embodiments, the heavy chain CH1 region comprises a sequence of the formula (II):

X¹(fGly′)X²Z²X³Z³ (II)
where
fGly′ is the amino acid residue coupled to the drug or active agent through a linker as described herein;
Z² is either a proline or alanine residue (which can also be represented by (P/A));
Z³ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (e.g., other than an aromatic amino acid or a charged amino acid), e.g., L, M, V, S or T, e.g., L, M or V, with the proviso that when the sulfatase motif is at the N-terminus of the target polypeptide, X¹ is present;
X² and X³ independently can be any amino acid, e.g., an aliphatic amino acid, a sulfur-containing amino acid, or a polar, uncharged amino acid, (e.g., other than an aromatic amino acid or a charged amino acid), e.g., S, T, A, V, G or C, e.g., S, T, A, V or G; and wherein the sequence is C-terminal to the amino acid sequence SWNSGA (SEQ ID NO: 135) and/or is N-terminal to the amino acid sequence GVHTFP (SEQ ID NO: 136).

In certain embodiments, the heavy chain CH1 region comprises the sequence SWNSGAL(fGly′)TPSRGVHTFP (SEQ ID NO: 137).

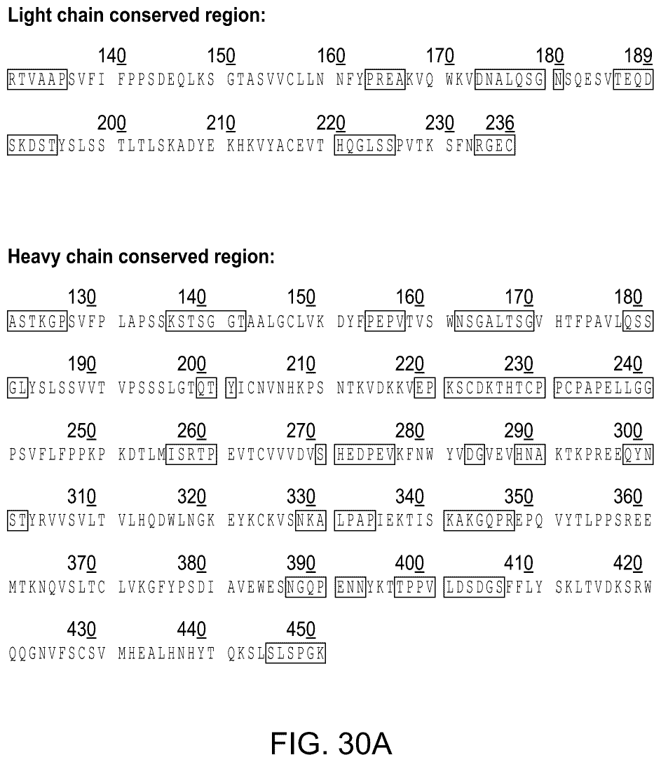

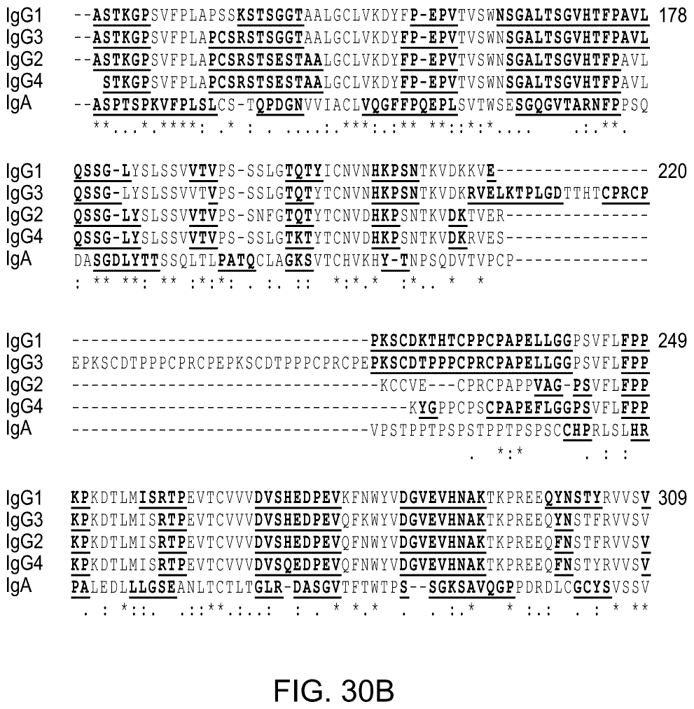

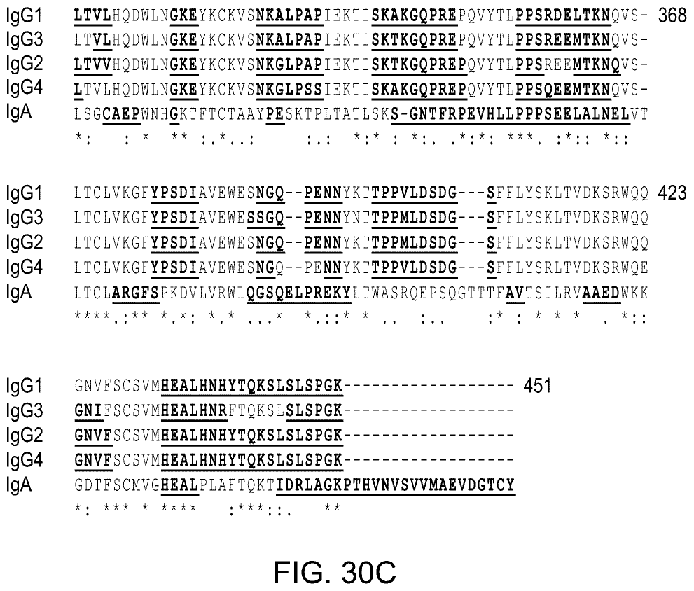

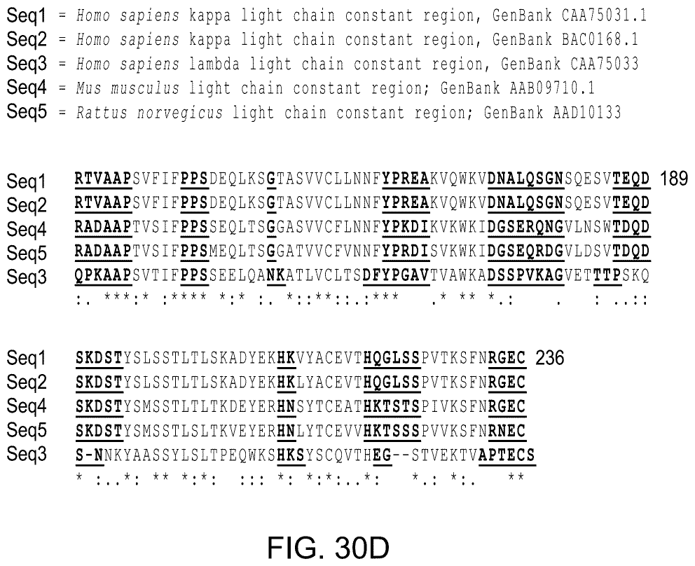

FIG. 30A depicts a site map showing possible modification sites for generation of an aldehyde tagged Ig polypeptide. The upper sequence is the amino acid sequence of the conserved region of an IgG1 light chain polypeptide (SEQ ID NO: 87) and shows possible modification sites in an Ig light chain; the lower sequence is the amino acid sequence of the conserved region of an Ig heavy chain polypeptide (SEQ ID NO: 88) (GenBank Accession No. AAG00909) and shows possible modification sites in an Ig heavy chain. The heavy and light chain numbering is based on the full-length heavy and light chains.

FIG. 30B depicts an alignment of Homo sapiens immunoglobulin heavy chain constant regions for IgG1 (SEQ ID NO: 89; GenBank P01857.1), IgG2 (SEQ ID NO: 90; GenBank P01859.2), IgG3 (SEQ ID NO: 91; GenBank P01860.2), IgG4 (SEQ ID NO: 92; GenBank AAB59394.1), and IgA (SEQ ID NO: 93; GenBank AAAT74070), showing modification sites at which aldehyde tags can be provided in an immunoglobulin heavy chain. The heavy and light chain numbering is based on the full heavy and light chains.

FIG. 30C depicts an alignment of immunoglobulin light chain constant regions, showing modification sites at which aldehyde tags can be provided in an immunoglobulin light chain. Seq1=Homo sapiens kappa light chain constant region; GenBank CAA75031.1; SEQ ID NO: 94. Seq2=Homo sapiens kappa light chain constant region; GenBank BAC0168.1; SEQ ID NO: 95. Seq3=Homo sapiens lambda light chain constant region; GenBank CAA75033; SEQ ID NO: 96. Seq4=Mus musculus light chain constant region; GenBank AAB09710.1; SEQ ID NO: 97. Seq5=Rattus norvegicus light chain constant region; GenBank AAD10133; SEQ ID NO: 98.

In some embodiments the sulfatase motif is at a position other than, or in addition to, the C-terminus of the Ig polypeptide heavy chain. An isolated aldehyde-tagged anti-Nectin-4 polypeptide can comprise a heavy chain constant region amino acid sequence modified to include a sulfatase motif as described herein, where the sulfatase motif is in or adjacent a surface-accessible loop region of the anti-Nectin-4 polypeptide heavy chain constant region.

Exemplary surface-accessible loop regions of an IgG1 heavy chain include: 1) ASTKGP (SEQ ID NO: 138); 2) KSTSGGT (SEQ ID NO: 139); 3) PEPV (SEQ ID NO: 140); 4) NSGALTSG (SEQ ID NO: 141); 5) NSGALTSGVHTFPAVLQSSGL (SEQ ID NO: 142); 6) QSSGL (SEQ ID NO: 143); 7) VTV; 8) QTY; 9) TQTY (SEQ ID NO: 144); 10) HKPSN (SEQ ID NO: 145); 11) EPKSCDKTHTCPPCPAPELLGG (SEQ ID NO: 146); 12) FPPKP (SEQ ID NO: 147); 13) ISRTP (SEQ ID NO: 148); 14) DVSHEDPEV (SEQ ID NO: 149); 15) SHEDPEV (SEQ ID NO: 150); 16) DG; 17) DGVEVHNAK (SEQ ID NO: 151); 18) HNA; 19) QYNST (SEQ ID NO: 152); 20) VLTVL (SEQ ID NO: 153); 21) GKE; 22) NKALPAP (SEQ ID NO: 154); 23) SKAKGQPRE (SEQ ID NO: 155); 24) KAKGQPR (SEQ ID NO: 156); 25) PPSRKELTKN (SEQ ID NO: 157); 26) YPSDI (SEQ ID NO: 158); 27) NGQPENN (SEQ ID NO: 159); 28) TPPVLDSDGS (SEQ ID NO: 160); 29) HEALHNHYTQKSLSLSPGK (SEQ ID NO: 161); and 30) SLSPGK (SEQ ID NO: 162).

Exemplary surface-accessible loop regions of an IgG2 heavy chain include 1) ASTKGP (SEQ ID NO: 163); 2) PCSRSTSESTAA (SEQ ID NO: 164); 3) FPEPV (SEQ ID NO: 165); 4) SGALTSGVHTFP (SEQ ID NO: 166); 5) QSSGLY (SEQ ID NO: 167); 6) VTV; 7) TQT; 8) HKP; 9) DK; 10) VAGPS (SEQ ID NO: 168); 11) FPPKP (SEQ ID NO: 169); 12) RTP; 13) DVSHEDPEV (SEQ ID NO: 170); 14) DGVEVHNAK (SEQ ID NO: 171); 15) FN; 16) VLTVV (SEQ ID NO: 172); 17) GKE; 18) NKGLPAP (SEQ ID NO: 173); 19) SKTKGQPRE (SEQ ID NO: 174); 20) PPS; 21) MTKNQ (SEQ ID NO: 175); 22) YPSDI (SEQ ID NO: 176); 23) NGQPENN (SEQ ID NO: 177); 24) TPPMLDSDGS (SEQ ID NO: 178); 25) GNVF (SEQ ID NO: 179); and 26) HEALHNHYTQKSLSLSPGK (SEQ ID NO: 180).

Exemplary surface-accessible loop regions of an IgG3 heavy chain include 1) ASTKGP (SEQ ID NO: 181); 2) PCSRSTSGGT (SEQ ID NO: 182); 3) FPEPV (SEQ ID NO: 183); 4) SGALTSGVHTFPAVLQSSG (SEQ ID NO: 184); 5) V; 6) TQT; 7) HKPSN (SEQ ID NO: 185); 8) RVELKTPLGD (SEQ ID NO: 186); 9) CPRCPKP (SEQ ID NO: 187); 10) PKSCDTPPPCPRCPAPELLGG (SEQ ID NO: 188); 11) FPPKP (SEQ ID NO: 189); 12) RTP; 13) DVSHEDPEV (SEQ ID NO: 190); 14) DGVEVHNAK (SEQ ID NO: 191); 15) YN; 16) VL; 17) GKE; 18) NKALPAP (SEQ ID NO: 192); 19) SKTKGQPRE (SEQ ID NO: 193); 20) PPSREEMTKN (SEQ ID NO: 194); 21) YPSDI (SEQ ID NO: 195); 22) SSGQPENN (SEQ ID NO: 196); 23) TPPMLDSDGS (SEQ ID NO: 197); 24) GNI; 25) HEALHNR (SEQ ID NO: 198); and 26) SLSPGK (SEQ ID NO: 199).

Exemplary surface-accessible loop regions of an IgG4 heavy chain include 1) STKGP (SEQ ID NO: 200); 2) PCSRSTSESTAA (SEQ ID NO: 201); 3) FPEPV (SEQ ID NO: 202); 4) SGALTSGVHTFP (SEQ ID NO: 203); 5) QSSGLY (SEQ ID NO: 204); 6) VTV; 7) TKT; 8) HKP; 9) DK; 10) YG; 11) CPAPEFLGGPS (SEQ ID NO: 205); 12) FPPKP (SEQ ID NO: 206); 13) RTP; 14) DVSQEDPEV (SEQ ID NO: 207); 15) DGVEVHNAK (SEQ ID NO: 208); 16) FN; 17) VL; 18) GKE; 19) NKGLPSS (SEQ ID NO: 209); 20) SKAKGQPREP (SEQ ID NO: 210); 21) PPSQEEMTKN (SEQ ID NO: 211); 22) YPSDI (SEQ ID NO: 212); 23) NG; 24) NN; 25) TPPVLDSDGS (SEQ ID NO: 213); 26) GNVF (SEQ ID NO: 214); and 27) HEALHNHYTQKSLSLSLGK (SEQ ID NO: 215).

Exemplary surface-accessible loop regions of an IgA heavy chain include 1) ASPTSPKVFPLSL (SEQ ID NO: 216); 2) QPDGN (SEQ ID NO: 217); 3) VQGFFPQEPL (SEQ ID NO: 218); 4) SGQGVTARNFP (SEQ ID NO: 219); 5) SGDLYTT (SEQ ID NO: 220); 6) PATQ (SEQ ID NO: 221); 7) GKS; 8) YT; 9) CHP; 10) HRPA (SEQ ID NO: 222); 11) LLGSE (SEQ ID NO: 223); 12) GLRDASGV (SEQ ID NO: 224); 13) SSGKSAVQGP (SEQ ID NO: 225); 14) GCYS (SEQ ID NO: 226); 15) CAEP (SEQ ID NO: 227); 16) PE; 17) SGNTFRPEVHLLPPPSEELALNEL (SEQ ID NO: 228); 18) ARGFS (SEQ ID NO: 229); 19) QGSQELPREKY (SEQ ID NO: 230); 20) AV; 21) AAED (SEQ ID NO: 231); 22) HEAL (SEQ ID NO: 232); and 23) IDRLAGKPTHVNVSVVMAEVDGTCY (SEQ ID NO: 233).

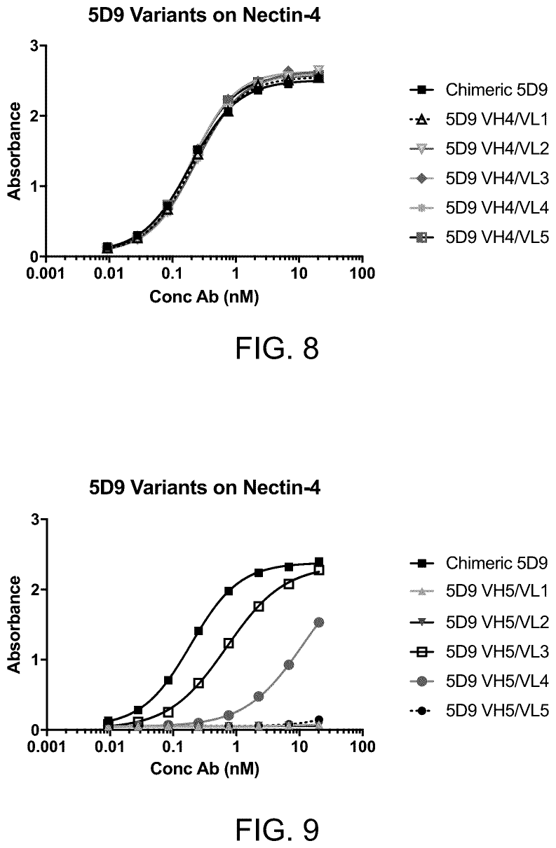

Exemplary surface-accessible loop regions of an Ig light chain (e.g., a human kappa light chain) include: 1) RTVAAP (SEQ ID NO: 234); 2) PPS; 3) Gly (see, e.g., Gly at position 150 of the human kappa light chain sequence depicted in FIG. 8C); 4) YPREA (SEQ ID NO: 235); 5) PREA (SEQ ID NO: 236); 6) DNALQSGN (SEQ ID NO: 237); 7) TEQDSKDST (SEQ ID NO: 238); 8) HK; 9) HQGLSS (SEQ ID NO: 239); and 10) RGEC (SEQ ID NO: 240).

Exemplary surface-accessible loop regions of an Ig lambda light chain include QPKAAP (SEQ ID NO: 241), PPS, NK, DFYPGAV (SEQ ID NO: 242), DSSPVKAG (SEQ ID NO: 243), TTP, SN, HKS, EG, and APTECS (SEQ ID NO: 244).

The constant region of the HC of an anti-Nectin-4 antibody as disclosed herein may be selected from one of the following sequences:

CT-Tagged (Aldehyde Tag-in bold)
(SEQ ID NO: 70)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSREEMTKNQVSLTCLVKGFYPSDIAVEWESNGQP
ENNYKTTPPVLDSDGSFFLYSKLTVDKSRWQQGNVFSCSVMHEAL
HNHYTQKSLSLSPGSLCTPSRGS

In SEQ ID NO: 70, the italicized residues at the C-terminus of the heavy chain constant region replace a lysine residue at the C-terminus of a standard IgG1 heavy chain. The bolded residues (LCTPSR (SEQ ID NO: 104)) among the italicized residues constitute the aldehyde tag, where the C is converted to an fGly residue by FGE upon expression of the heavy chain to produce L(fGly)TPSR (SEQ ID NO: 245). The fGly can be modified to fGly′ to produce L(fGly′)TPSR (SEQ ID NO: 246). fGly′ refers to the amino acid residue of the anti-Nectin-4 antibody that is coupled to the moiety of interest (e.g., a drug). The non-bolded residues among the italicized residues are additional residues that are different from a standard IgG1 heavy chain sequence.

58Q-1 (Aldehyde Tag-In Bold and Substitution of “EEM” with “DEL”)

(SEQ ID NO: 71)
ASTKGPSVFPLAPSSKSTSGGTAALGCLVKDYFPEPVTVSWNSGA
LTSGVHTFPAVLLCTPSRQSSGLYSLSSVVTVPSSSLGTQTYICN
VNHKPSNTKVDKKVEPKSCDKTHTCPPCPAPELLGGPSVFLFPPK
PKDTLMISRTPEVTCVVVDVSHEDPEVKFNWYVDGVEVHNAKTKP
REEQYNSTYRVVSVLTVLHQDWLNGKEYKCKVSNKALPAPIEKTI
SKAKGQPREPQVYTLPPSRDELTKNQVSLTCLVKGFYPSDIAVEW
ESNGQPENNYKTTPPVLDSDGSFFLYSKLTVDKSRWQQGNVFSCS
VMHEALHNHYTQKSLSLSPGK.

61G-1 (Aldehyde Tag-In Bold and Substitution of “EEM” with “DEL”)

(SEQ ID NO: 72)
ASTKGPSVFPLAPSSKSTSGGTAALGCLVKDYFPEPVTVSWNSGA
LTSGVHTFPAVLQSSLCTPSRGLYSLSSVVTVPSSSLGTQTYICN
VNHKPSNTKVDKKVEPKSCDKTHTCPPCPAPELLGGPSVFLFPPK
PKDTLMISRTPEVTCVVVDVSHEDPEVKFNWYVDGVEVHNAKTKP
REEQYNSTYRVVSVLTVLHQDWLNGKEYKCKVSNKALPAPIEKTI
SKAKGQPREPQVYTLPPSRDELTKNQVSLTCLVKGFYPSDIAVEW
ESNGQPENNYKTTPPVLDSDGSFFLYSKLTVDKSRWQQGNVFSCS
VMHEALHNHYTQKSLSLSPGK.

91N-1 (Aldehyde Tag-In Bold and Substitution of “EEM” with “DEL”)

(SEQ ID NO: 73)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
LCTPSRNTKVDKKVEPKSCDKTHTCPPCPAPELLGGPSVFLFPPK
PKDTLMISRTPEVTCVVVDVSHEDPEVKFNWYVDGVEVHNAKTKP
REEQYNSTYRVVSVLTVLHQDWLNGKEYKCKVSNKALPAPIEKTI
SKAKGQPREPQVYTLPPSRDELTKNQVSLTCLVKGFYPSDIAVEW
ESNGQPENNYKTTPPVLDSDGSFFLYSKLTVDKSRWQQGNVFSCS
VMHEALHNHYTQKSLSLSPGK.

116E-1 (Aldehyde Tag-In Bold and Substitution of “EEM” with “DEL”)

(SEQ ID NO: 74)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPLCTPSRELLGGPSVFLFPPK
PKDTLMISRTPEVTCVVVDVSHEDPEVKFNWYVDGVEVHNAKTKP
REEQYNSTYRVVSVLTVLHQDWLNGKEYKCKVSNKALPAPIEKTI
SKAKGQPREPQVYTLPPSRDELTKNQVSLTCLVKGFYPSDIAVEW
ESNGQPENNYKTTPPVLDSDGSFFLYSKLTVDKSRWQQGNVFSCS
VMHEALHNHYTQKSLSLSPGK.

### 58Q-2 (Aldehyde Tag-In Bold)

(SEQ ID NO: 75)
ASTKGPSVFPLAPSSKSTSGGTAALGCLVKDYFPEPVTVSWNSGA
LTSGVHTFPAVLLCTPSRQSSGLYSLSSVVTVPSSSLGTQTYICN
VNHKPSNTKVDKKVEPKSCDKTHTCPPCPAPELLGGPSVFLFPPK
PKDTLMISRTPEVTCVVVDVSHEDPEVKFNWYVDGVEVHNAKTKP
REEQYNSTYRVVSVLTVLHQDWLNGKEYKCKVSNKALPAPIEKTI
SKAKGQPREPQVYTLPPSREEMTKNQVSLTCLVKGFYPSDIAVEW
ESNGQPENNYKTTPPVLDSDGSFFLYSKLTVDKSRWQQGNVFSCS
VMHEALHNHYTQKSLSLSPGK.

### 61G-2 (Aldehyde Tag-In Bold)

(SEQ ID NO: 76)
ASTKGPSVFPLAPSSKSTSGGTAALGCLVKDYFPEPVTVSWNSGALTSG
VHTFPAVLQSSLCTPSRGLYSLSSVVTVPSSSLGTQTYICNVNHKPSNT
KVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFF
LYSKLTVDKSRWQQGNVFSCSVMHEALHNHYTQKSLSLSPGK.

### 91N-2 (Aldehyde Tag-In Bold)

(SEQ ID NO: 77)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSLCTPSRNT
KVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFF
LYSKLTVDKSRWQQGNVFSCSVMHEALHNHYTQKSLSLSPGK.

### 116E-2 (Aldehyde Tag-In Bold)

(SEQ ID NO: 78)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPLCTPSRELLGGPSVFLFPPKPKDTLMISRTPE
VTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFF
LYSKLTVDKSRWQQGNVFSCSVMHEALHNHYTQKSLSLSPGK.

58Q-3 (Aldehyde Tag-In Bold and Substitution of “KKV” with “KRV” and Substitution of “EEM” with “DEL”)

(SEQ ID NO: 79)
ASTKGPSVFPLAPSSKSTSGGTAALGCLVKDYFPEPVTVSWNSGALTSG
VHTFPAVLLCTPSRQSSGLYSLSSVVTVPSSSLGTQTYICNVNHKPSNT
KVDKRVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFF
LYSKLTVDKSRWQQGNVFSCSVMHEALHNHYTQKSLSLSPGK.

61G-3 (Aldehyde Tag-In Bold and Substitution of “KKV” with “KRV” and Substitution of “EEM” with “DEL”)

(SEQ ID NO: 80)
ASTKGPSVFPLAPSSKSTSGGTAALGCLVKDYFPEPVTVSWNSGALTSG
VHTFPAVLQSSLCTPSRGLYSLSSVVTVPSSSLGTQTYICNVNHKPSNT
KVDKRVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFF
LYSKLTVDKSRWQQGNVFSCSVMHEALHNHYTQKSLSLSPGK.

91N-3 (Aldehyde Tag-In Bold and Substitution of “KKV” with “KRV” and Substitution of “EEM” with “DEL”)

(SEQ ID NO: 81)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSLCTPSRNT
KVDKRVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFF
LYSKLTVDKSRWQQGNVFSCSVMHEALHNHYTQKSLSLSPGK.

116E-3 (Aldehyde Tag-In Bold and Substitution of “KKV” with “KRV” and Substitution of “EEM” with “DEL”)

(SEQ ID NO: 82)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSCDKTHTCPPCPAPLCTPSRELLGGPSVFLFPPKPKDTLMISRTPE
VTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFF
LYSKLTVDKSRWQQGNVFSCSVMHEALHNHYTQKSLSLSPGK.

58Q-4 (Aldehyde Tag-In Bold and Substitution of “KKV” with “KRV”)

(SEQ ID NO: 83)
ASTKGPSVFPLAPSSKSTSGGTAALGCLVKDYFPEPVTVSWNSGALTSG
VHTFPAVLLCTPSRQSSGLYSLSSVVTVPSSSLGTQTYICNVNHKPSNT
KVDKRVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFF
LYSKLTVDKSRWQQGNVFSCSVMHEALHNHYTQKSLSLSPGK.

61G-4 (Aldehyde Tag-In Bold and Substitution of “KKV” with “KRV”)

(SEQ ID NO: 84)
ASTKGPSVFPLAPSSKSTSGGTAALGCLVKDYFPEPVTVSWNSGALTSG
VHTFPAVLQSSLCTPSRGLYSLSSVVTVPSSSLGTQTYICNVNHKPSNT
KVDKRVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFF
LYSKLTVDKSRWQQGNVFSCSVMHEALHNHYTQKSLSLSPGK.

91N-4 (Aldehyde Tag-In Bold and Substitution of “KKV” with “KRV”)

(SEQ ID NO: 85)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSLCTPSRNT
KVDKRVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFF
LYSKLTVDKSRWQQGNVFSCSVMHEALHNHYTQKSLSLSPGK.

116E-4 (Aldehyde Tag-In Bold and Substitution of “KKV” with “KRV”)

(SEQ ID NO: 86)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSCDKTHTCPPCPAPLCTPSRELLGGPSVFLFPPKPKDTLMISRTPE
VTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFF
LYSKLTVDKSRWQQGNVFSCSVMHEALHNHYTQKSLSLSPGK.

The bolded residues (LCTPSR) constitute the aldehyde tag, where the C is converted to an fGly residue by FGE upon expression of the heavy chain. The fGly can be converted to fGly′. fGly′ refers to the amino acid residue of the anti-Nectin-4 antibody that is coupled to the moiety of interest (e.g., a drug).

### Drugs and Active Agents

In some cases, an anti-Nectin-4 antibody of the present disclosure has a drug or active agent (e.g., W¹ in conjugates of formula (I) described herein) covalently linked to the heavy and/or light chain of the antibody. For example, an antibody conjugate of the present disclosure can include as substituent W¹ a drug or active agent and, if present, can include as substituent W² a second drug or active agent. Any of a number of drugs are suitable for use, or can be modified to be rendered suitable for use, as a reactive partner to conjugate to an antibody. “Drugs” include small molecule drugs, peptidic drugs, toxins (e.g., cytotoxins), and the like.

“Small molecule drug” as used herein refers to a compound, e.g., an organic compound, which exhibits a pharmaceutical activity of interest and which is generally of a molecular weight of no greater than about 800 Da, or no greater than 2000 Da, but can encompass molecules of up to 5 kDa and can be as large as about 10 kDa. A small inorganic molecule refers to a molecule containing no carbon atoms, while a small organic molecule refers to a compound containing at least one carbon atom.

In certain embodiments, the drug or active agent can be a maytansine. “Maytansine”, “maytansine moiety”, “maytansine active agent moiety” and “maytansinoid” refer to a maytansine and analogs and derivatives thereof, and pharmaceutically active maytansine moieties and/or portions thereof. A maytansine conjugated to the polypeptide can be any of a variety of maytansinoid moieties such as, but not limited to, maytansine and analogs and derivatives thereof as described herein (e.g., deacylmaytansine).

In certain embodiments, the drug or active agent can be an auristatin, or an analog or derivative thereof, or a pharmaceutically active auristatin moiety and/or a portion thereof. An auristatin conjugated to the polypeptide can be any of a variety of auristatin moieties such as, but not limited to, an auristatin and analogs and derivatives thereof as described herein. Examples of drugs that find use in the conjugates and compounds described herein include, but are not limited to an auristatin or an auristatin derivative, such as monomethyl auristatin D (MMAD), monomethyl auristatin E (MMAE), monomethyl auristatin F (MMAF), derivatives thereof, and the like.

In certain embodiments, the drug or active agent can be a duocarmycin, or an analog or derivative thereof, or a pharmaceutically active duocarmycin moiety and/or a portion thereof. A duocarmycin conjugated to the polypeptide can be any of a variety of duocarmycin moieties such as, but not limited to, a duocarmycin and analogs and derivatives thereof as described herein. Examples of drugs that find use in the conjugates and compounds described herein include, but are not limited to a duocarmycin or a duocarmycin derivative, such as duocarmycin A, duocarmycin B1, duocarmycin B2, duocarmycin C₁, duocarmycin C₂, duocarmycin D, duocarmycin SA, and CC-1065, derivatives thereof, and the like. In some embodiments, the duocarmycin is a duocarmycin analog, such as, but not limited to, adozelesin, bizelesin, or carzelesin.

In certain embodiments, the drug or active agent can be a topoisomerase inhibitor, such as a camptothecine, or an analog or derivative thereof, or a pharmaceutically active camptothecine moiety and/or a portion thereof. A camptothecine conjugated to the subject antibody can be any of a variety of camptothecine moieties such as, but not limited to, a camptothecine and analogs and derivatives thereof as described herein. Examples of drugs that find use in the conjugates described herein include, but are not limited to a camptothecine or a camptothecine derivative, such as SN-38, Belotecan, Exatecan, 9-aminocamptothecin (9-AC), derivatives thereof, and the like.

In certain embodiments, the drug or active agent (e.g., W¹ and/or W²) in formulae (I) and (II) described herein is a camptothecine, or analog or derivative thereof. For example, in some instances, the camptothecine, or analog or derivative thereof, is a compound of formula (III):

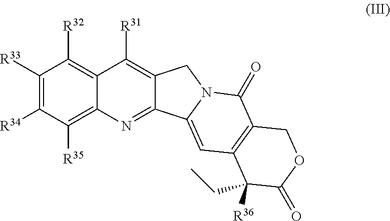

wherein:
R³¹ and R³² are each independently selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl, or R³¹ and R³² are optionally cyclically linked to form a 5 or 6-membered cycloalkyl or heterocyclyl ring;
R³³ and R³⁴ are each independently selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl, or R³³ and R³⁴ are optionally cyclically linked to form a 5 or 6-membered cycloalkyl or heterocyclyl ring;
R³⁵ is selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl;
R³⁶ is selected from OH and OC(O) R³⁷; and
R³⁷ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl.

In certain embodiments of formula (III), the linker L^{A} in formula (I) or formula (II) is attached to a compound of formula (III) at R³¹, R³², R³³, R³⁴, R³⁵ or R³⁶. In certain embodiments of formula (III), the linker L^{B} in formula (I) or formula (II) is attached to a compound of formula (III) at R³¹, R³², R³³, R³⁴, R³⁵ or R³⁶.

In certain embodiments, R³¹ and R³² are each independently selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl, or R³¹ and R³² are optionally cyclically linked to form a 5 or 6-membered cycloalkyl or heterocyclyl ring.

In certain embodiments, R³¹ is selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl. In certain embodiments, R³¹ is hydrogen. In certain embodiments, R³¹ is halogen (e.g., F, Cl, Br, I). In certain embodiments, R³¹ is hydroxy. In certain embodiments, R³¹ is amino or substituted amino. In certain embodiments, R³¹ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³¹ is methyl. In certain embodiments, R³¹ is C₂ substituted alkyl, such as —CH₂CH₂NH(CH(CH₃)₂). In certain embodiments, R³¹ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³¹ is alkynyl or substituted alkynyl. In certain embodiments, R³¹ is alkoxy or substituted alkoxy. In certain embodiments, R³¹ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³¹ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³¹ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³¹ is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R³² is selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl. In certain embodiments, R³² is hydrogen. In certain embodiments, R³² is halogen (e.g., F, Cl, Br, I). In certain embodiments, R³² is hydroxy. In certain embodiments, R³² is amino or substituted amino. In certain embodiments, R³² is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³² is methyl. In certain embodiments, R³² is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³² is alkynyl or substituted alkynyl. In certain embodiments, R³² is alkoxy or substituted alkoxy. In certain embodiments, R³² is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³² is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³² is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³² is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R³¹ and R³² are optionally cyclically linked to form a 5 or 6-membered cycloalkyl or heterocyclyl ring. In certain embodiments, R³¹ and R³² are cyclically linked to form a 5 or 6-membered cycloalkyl. In certain embodiments, R³¹ and R³² are cyclically linked to form a 5 or 6-membered heterocyclyl. In certain embodiments, R³¹ and R³² are cyclically linked to form a 5-membered cycloalkyl. In certain embodiments, R³¹ and R³² are cyclically linked to form a 6-membered cycloalkyl. In certain embodiments, R³¹ and R³² are cyclically linked to form a 5-membered heterocyclyl. In certain embodiments, R³¹ and R³² are cyclically linked to form a 6-membered heterocyclyl.

In certain embodiments, R³³ and R³⁴ are each independently selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl, or R³³ and R³⁴ are optionally cyclically linked to form a 5 or 6-membered cycloalkyl or heterocyclyl ring.

In certain embodiments, R³³ is selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl. In certain embodiments, R³³ is hydrogen. In certain embodiments, R³³ is halogen (e.g., F, Cl, Br, I). In certain embodiments, R³³ is hydroxy. In certain embodiments, R³³ is amino or substituted amino. In certain embodiments, R³³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³³ is methyl. In certain embodiments, R³³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³³ is alkynyl or substituted alkynyl. In certain embodiments, R³³ is alkoxy or substituted alkoxy. In certain embodiments, R³³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³³ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³³ is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R³⁴ is selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl. In certain embodiments, R³⁴ is hydrogen. In certain embodiments, R³⁴ is halogen (e.g., F, Cl, Br, I). In certain embodiments, R³⁴ is hydroxy. In certain embodiments, R³⁴ is amino or substituted amino. In certain embodiments, R³⁴ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³⁴ is methyl. In certain embodiments, R³⁴ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³⁴ is alkynyl or substituted alkynyl. In certain embodiments, R³⁴ is alkoxy or substituted alkoxy. In certain embodiments, R³⁴ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³⁴ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³⁴ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³⁴ is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R³³ and R³⁴ are optionally cyclically linked to form a 5 or 6-membered cycloalkyl or heterocyclyl ring. In certain embodiments, R³³ and R³⁴ are cyclically linked to form a 5 or 6-membered cycloalkyl. In certain embodiments, R³³ and R³⁴ are cyclically linked to form a 5 or 6-membered heterocyclyl. In certain embodiments, R³³ and R³⁴ are cyclically linked to form a 5-membered cycloalkyl. In certain embodiments, R³³ and R³⁴ are cyclically linked to form a 6-membered cycloalkyl. In certain embodiments, R³³ and R³⁴ are cyclically linked to form a 5-membered heterocyclyl. In certain embodiments, R³³ and R³⁴ are cyclically linked to form a 6-membered heterocyclyl.

In certain embodiments, R³⁵ is selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl. In certain embodiments, R³⁵ is hydrogen. In certain embodiments, R³⁵ is halogen (e.g., F, Cl, Br, I). In certain embodiments, R³⁵ is hydroxy. In certain embodiments, R³⁵ is amino or substituted amino. In certain embodiments, R³⁵ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³⁵ is methyl. In certain embodiments, R³⁵ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³⁵ is alkynyl or substituted alkynyl. In certain embodiments, R³⁵ is alkoxy or substituted alkoxy. In certain embodiments, R³⁵ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³⁵ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³⁵ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³⁵ is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R³⁶ is selected from OH and OC(O) R³⁷. In certain embodiments, R³⁶ is OH. In certain embodiments, R³⁶ is OC(O) R³⁷.

In certain embodiments, R³⁷ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl. In certain embodiments, R³⁷ is hydrogen. In certain embodiments, R³⁷ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³⁷ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³⁷ is alkynyl or substituted alkynyl. In certain embodiments, R³⁷ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³⁷ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³⁷ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³⁷ is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, the compound of formula (III) has the structure of formula (IIIa):

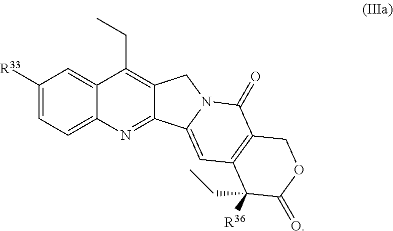

In certain embodiments of the compound of formula (IIIa), R³³ is as described above.

In certain embodiments of the compound of formula (IIIa), R³⁶ is as described above.

In certain embodiments of the compound of formula (IIIa), R³³ is OH and the linker L^{A} or L^{B} is attached at R³⁶. In certain embodiments of the compound of formula (IIIa), the linker L^{A} or L^{B} is attached at R³³ and R³⁶ is OH.

In certain embodiments, the compound of formula (III) has the structure of formula (IIIb):

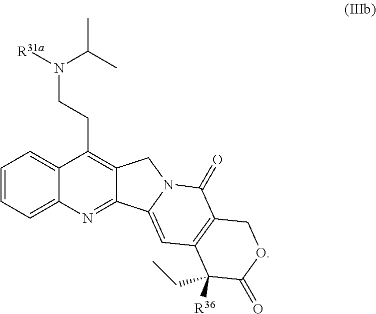

In certain embodiments of the compound of formula (IIIb), R^{31a} is selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl. In certain embodiments, R^{31a} is hydrogen. In certain embodiments, R^{31a} is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R^{31a} is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R^{31a} is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R^{31a} is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R^{31a} is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl. In certain embodiments, R^{31a} is carboxyl. In certain embodiments, R^{31a} is carboxyl ester. In certain embodiments, R^{31a} is acyl. In certain embodiments, R^{31a} is sulfonyl.

In certain embodiments of the compound of formula (IIIb), R³⁶ is as described above.

In certain embodiments of the compound of formula (IIIb), R^{31a} is selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl, and the linker L^{A} or L^{B} is attached at R³⁶. In certain embodiments of the compound of formula (IIIb), the linker L^{A} or L^{B} is attached at R^{31a} and R³⁶ is OH.

In certain embodiments, the compound of formula (III) has the structure of formula (IIIc):

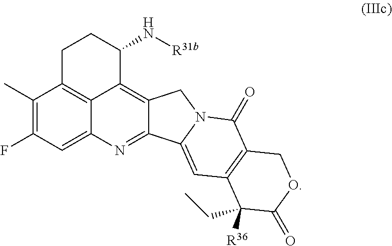

In certain embodiments of the compound of formula (IIIc), R^{31b} is selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl. In certain embodiments, R^{31b} is hydrogen. In certain embodiments, R^{31b} is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R^{31b} is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R^{31b} is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R^{31b} is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R^{31b} is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl. In certain embodiments, R^{31b} is carboxyl. In certain embodiments, R^{31b} is carboxyl ester. In certain embodiments, R^{31b} is acyl. In certain embodiments, R^{31b} is sulfonyl.

In certain embodiments of the compound of formula (IIIc), R³⁶ is as described above.

In certain embodiments of the compound of formula (IIIc), R^{31b} is selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl, and the linker L^{A} or L^{B} is attached at R³⁶. In certain embodiments of the compound of formula (IIIc), the linker L^{A} or L^{B} is attached at R^{31b} and R³⁶ is OH.

In certain embodiments, the compound of formula (III) has the structure of formula (IIId):

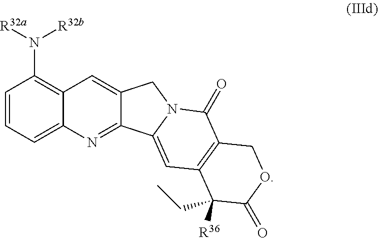

In certain embodiments of the compound of formula (IIId), R^{32a} and R^{32b} are each independently selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl.

In certain embodiments of the compound of formula (IIId), R^{32a} is selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl. In certain embodiments, R^{32a} is hydrogen. In certain embodiments, R^{32a} is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R^{32a} is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R^{32a} is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R^{32a} is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R^{32a} is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl. In certain embodiments, R^{32a} is carboxyl. In certain embodiments, R^{32a} is carboxyl ester. In certain embodiments, R^{32a} is acyl. In certain embodiments, R^{32a} is sulfonyl.

In certain embodiments of the compound of formula (IIId), R^{32b} is selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl. In certain embodiments, R^{32b} is hydrogen. In certain embodiments, R^{32b} is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R^{32b} is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R^{32b} is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R^{32b} is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R^{32b} is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl. In certain embodiments, R^{32b} is carboxyl. In certain embodiments, R^{32b} is carboxyl ester. In certain embodiments, R^{32b} is acyl. In certain embodiments, R^{32b} is sulfonyl.

In certain embodiments of the compound of formula (IIId), R³⁶ is as described above.

In certain embodiments of the compound of formula (IIId), R^{32a} and R^{32b} are each independently selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl, and the linker L^{A} or L^{B} is attached at R³⁶. In certain embodiments of the compound of formula (IIId), the linker L^{A} or L^{B} is attached at R^{32a} or R^{32b} and R³⁶ is OH. In certain embodiments of the compound of formula (IIId), the linker L^{A} or L^{B} is attached at R^{32a} and R³⁶ is OH. In certain embodiments of the compound of formula (IIId), the linker L^{A} or L^{B} is attached at R^{32b} and R³⁶ is OH.

In certain embodiments, the drug is selected from a cytotoxin, a kinase inhibitor, a selective estrogen receptor modulator, an immunostimulatory agent, a toll-like receptor (TLR) agonist, an oligonucleotide, an aptamer, a cytokine, a steroid, and a peptide.

For example, a cytotoxin can include any compound that leads to cell death (e.g., necrosis or apoptosis) or a decrease in cell viability.

Kinase inhibitors can include, but are not limited to, Adavosertib, Afatinib, Axitinib, Bosutinib, Cetuximab, Cobimetinib, Crizotinib, Cabozantinib, Dacomitinib, Dasatinib, Entrectinib, Erdafitinib, Erlotinib, Fostamatinib, Gefitinib, Ibrutinib, Imatinib, Lapatinib, Lenvatinib, Mubritinib, Nilotinib, Pazopanib, Pegaptanib, Ruxolitinib, Sorafenib, Sunitinib, Tucatinib, Vandetanib, Vemurafenib, and the like.

For example, selective estrogen receptor modulators include, but are not limited to, Endoxifen, Tamoxifen, Afimoxifene, Toremifene, and the like.

Immunostimulatory agents can include, but are not limited to, vaccines (e.g., bacterial or viral vaccines), colony stimulating factors, interferons, interleukins, and the like. TLR agonists include, but are not limited to, imiquimod, resiquimod, and the like.

Oligonucleotide dugs include, but are not limited to, fomivirsen, pegaptanib, mipomersen, eteplirsen, defibrotide, nusinersen, golodirsen, viltolarsen, volanesorsen, inotersen, tofersen, tominersen, and the like.

Aptamer drugs include, but are not limited to, pegaptanib, AS1411, REG1, ARC1779, NU172, ARC1905, E10030, NOX-A12, NOX-E36, and the like.

Cytokines include, but are not limited to, Albinterferon Alfa-2B, Aldesleukin, ALT-801, Anakinra, Ancestim, Avotermin, Balugrastim, Bempegaldesleukin, Binetrakin, Cintredekin Besudotox, CTCE-0214, Darbepoetin alfa, Denileukin diftitox, Dulanermin, Edodekin alfa, Emfilermin, Epoetin delta, Erythropoietin, Human interleukin-2, Interferon alfa, Interferon alfa-2c, Interferon alfa-n1, Interferon alfa-n3, Interferon alfacon-1, Interferon beta-1a, Interferon beta-1b, Interferon gamma-1b, Interferon Kappa, Interleukin-1 alpha, Interleukin-10, Interleukin-7, Lenograstim, Leridistim, Lipegfilgrastim, Lorukafusp alfa, Maxy-G34, Methoxy polyethylene glycol-epoetin beta, Molgramostim, Muplestim, Nagrestipen, Oprelvekin, Pegfilgrastim, Pegilodecakin, Peginterferon alfa-2a, Peginterferon alfa-2b, Peginterferon beta-1a, Peginterferon lambda-1a, Recombinant CD40-ligand, Regramostim, Romiplostim, Sargramostim, Thrombopoietin, Tucotuzumab celmoleukin, Viral Macrophage-Inflammatory Protein, and the like.

Steroid drugs include, but are not limited to, prednisolone, betamethasone, dexamethasone, hydrocortisone, methylprednisolone, deflazacort, and the like.

“Peptide drug” as used herein refers to amino-acid containing polymeric compounds, and is meant to encompass naturally-occurring and non-naturally-occurring peptides, oligopeptides, cyclic peptides, polypeptides, and proteins, as well as peptide mimetics. The peptide drugs may be obtained by chemical synthesis or be produced from a genetically encoded source (e.g., recombinant source). Peptide drugs can range in molecular weight, and can be from 200 Da to 10 kDa or greater in molecular weight. Suitable peptides include, but are not limited to, cytotoxic peptides; angiogenic peptides; anti-angiogenic peptides; peptides that activate B cells; peptides that activate T cells; anti-viral peptides; peptides that inhibit viral fusion; peptides that increase production of one or more lymphocyte populations; anti-microbial peptides; growth factors; growth hormone-releasing factors; vasoactive peptides; anti-inflammatory peptides; peptides that regulate glucose metabolism; an anti-thrombotic peptide; an anti-nociceptive peptide; a vasodilator peptide; a platelet aggregation inhibitor; an analgesic; and the like.

Additional examples of drugs that find use in the conjugates and compounds described herein include, but are not limited to Tubulysin M, Calicheamicin, a STAT3 inhibitor, alpha-Amanitin, an aurora kinase inhibitor, belotecan, and an anthracycline.

In some cases, the drug is a toxin, e.g., a cytotoxin. Ribosome inactivating proteins (RIPs), which are a class of proteins ubiquitous in higher plants, are examples of such cytotoxins. Suitable cytotoxins include, but are not limited to, ricin, abrin, diphtheria toxin, a Pseudomonas exotoxin (e.g., PE35, PE37, PE38, PE40, etc.), saporin, gelonin, a pokeweed anti-viral protein (PAP), botulinum toxin, bryodin, momordin, and bouganin.

In some cases, the drug is a cancer chemotherapeutic agent. Cancer chemotherapeutic agents include non-peptidic (e.g., non-proteinaccous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones. Peptidic compounds can also be used.

Suitable cancer chemotherapeutic agents include dolastatin and active analogs and derivatives thereof; and auristatin and active analogs and derivatives thereof. Suitable cancer chemotherapeutic agents also include maytansinoids and active analogs and derivatives thereof; and duocarmycins and active analogs and derivatives thereof.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, copthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation; therefore, compounds that bind to the estrogen receptor are used to block this activity.

Other suitable chemotherapeutic agents include metal complexes, e.g., cisplatin (cis-DDP), carboplatin, etc.; urcas, e.g., hydroxyurca; and hydrazines, e.g., N-methylhydrazine; epidophyllotoxin; a topoisomerase inhibitor; procarbazine; mitoxantrone; leucovorin; tegafur; etc. Other anti-proliferative agents of interest include immunosuppressants, e.g., mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl) propoxy) quinazoline); etc.

Taxanes are suitable for use. “Taxanes” include paclitaxel, as well as any active taxane derivative or pro-drug. “Paclitaxel” (which should be understood herein to include analogues, formulations, and derivatives such as, for example, docetaxel, TAXOL□, TAXOTERE□ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., TAXOTERE□□docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives; piperazino and piperazino derivatives.

Embodiments of the present disclosure include conjugates where an antibody is conjugated to one or more drug moieties, such as 2 drug moieties, 3 drug moieties, 4 drug moieties, 5 drug moieties, 6 drug moieties, 7 drug moieties, 8 drug moieties, 9 drug moieties, 10 drug moieties, 11 drug moieties, 12 drug moieties, 13 drug moieties, 14 drug moieties, 15 drug moieties, 16 drug moieties, 17 drug moieties, 18 drug moieties, 19 drug moieties, or 20 or more drug moieties. The drug moieties may be conjugated to the antibody at one or more sites in the antibody, as described herein. In certain embodiments, the conjugates have an average drug-to-antibody ratio (DAR) (molar ratio) in the range of from 0.1 to 20, or from 0.5 to 20, or from 1 to 20, such as from 1 to 19, or from 1 to 18, or from 1 to 17, or from 1 to 16, or from 1 to 15, or from 1 to 14, or from 1 to 13, or from 1 to 12, or from 1 to 11, or from 1 to 10, or from 1 to 9, or from 1 to 8, or from 1 to 7, or from 1 to 6, or from 1 to 5, or from 1 to 4, or from 1 to 3, or from 1 to 2. In certain embodiments, the conjugates have an average DAR from 1 to 10, such as 1, 2, 3, 4, 5, 6, 7, 8, 9, or 10. In certain embodiments, the conjugates have an average DAR of 1 to 10. In certain embodiments, the conjugates have an average DAR of 1 to 5, such as 4. In certain embodiments, the conjugates have an average DAR of 5 to 10, such as 8. By average is meant the arithmetic mean.

Drugs to be conjugated to a polypeptide may be modified to incorporate a reactive partner for reaction with the polypeptide. Where the drug is a peptide drug, the reactive moiety (e.g., aminooxy or hydrazide can be positioned at an N-terminal region, the N-terminus, a C-terminal region, the C-terminus, or at a position internal to the peptide. For example, an example of a method involves synthesizing a peptide drug having an aminooxy group. In this example, the peptide is synthesized from a Boc-protected precursor. An amino group of a peptide can react with a compound comprising a carboxylic acid group and oxy-N-Boc group. As an example, the amino group of the peptide reacts with 3-(2,5-dioxopyrrolidin-1-yloxy) propanoic acid. Other variations on the compound comprising a carboxylic acid group and oxy-N-protecting group can include different number of carbons in the alkylene linker and substituents on the alkylene linker. The reaction between the amino group of the peptide and the compound comprising a carboxylic acid group and oxy-N-protecting group occurs through standard peptide coupling chemistry. Examples of peptide coupling reagents that can be used include, but not limited to, DCC (dicyclohexylcarbodiimide), DIC (diisopropylcarbodiimide), di-p-toluoylcarbodiimide, BDP (1-benzotriazole diethylphosphate-1-cyclohexyl-3-(2-morpholinylethyl) carbodiimide), EDC (1-(3-dimethylaminopropyl-3-ethyl-carbodiimide hydrochloride), cyanuric fluoride, cyanuric chloride, TFFH (tetramethyl fluoroformamidinium hexafluorophosphosphate), DPPA (diphenylphosphorazidate), BOP (benzotriazol-1-yloxytris(dimethylamino)phosphonium hexafluorophosphate), HBTU (O-benzotriazol-1-yl-N,N,N′,N′-tetramethyluronium hexafluorophosphate), TBTU (O-benzotriazol-1-yl-N,N,N′,N′-tetramethyluronium tetrafluoroborate), TSTU (O—(N-succinimidyl)-N,N,N′,N′-tetramethyluronium tetrafluoroborate), HATU (N-[(dimethylamino)-1-H-1,2,3-triazolo[4,5,6]-pyridin-1-ylmethylene]-N-methylmethanaminium hexafluorophosphate N-oxide), BOP-Cl (bis(2-oxo-3-oxazolidinyl)phosphinic chloride), PyBOP ((1-H-1,2,3-benzotriazol-1-yloxy)-tris(pyrrolidino)phosphonium tetrafluorophopsphate), BrOP (bromotris(dimethylamino)phosphonium hexafluorophosphate), DEPBT (3-(diethoxyphosphoryloxy)-1,2,3-benzotriazin-4 (3H)-one) PyBrOP (bromotris(pyrrolidino)phosphonium hexafluorophosphate). As a non-limiting example, HOBt and DIC can be used as peptide coupling reagents.

Deprotection to expose the amino-oxy functionality is performed on the peptide comprising an N-protecting group. Deprotection of the N-oxysuccinimide group, for example, occurs according to standard deprotection conditions for a cyclic amide group. Deprotecting conditions can be found in Greene and Wuts, Protective Groups in Organic Chemistry, 3rd Ed., 1999, John Wiley & Sons, NY and Harrison et al. Certain deprotection conditions include a hydrazine reagent, amino reagent, or sodium borohydride. Deprotection of a Boc protecting group can occur with TFA. Other reagents for deprotection include, but are not limited to, hydrazine, methylhydrazine, phenylhydrazine, sodium borohydride, and methylamine. The product and intermediates can be purified by conventional means, such as HPLC purification.

The ordinarily skilled artisan will appreciate that factors such as pH and steric hindrance (e.g., the accessibility of the amino acid residue to reaction with a reactive partner of interest) are of importance. Modifying reaction conditions to provide for optimal conjugation conditions is well within the skill of the ordinary artisan, and is routine in the art. Where conjugation is conducted with a polypeptide present in or on a living cell, the conditions are selected so as to be physiologically compatible. For example, the pH can be dropped temporarily for a time sufficient to allow for the reaction to occur but within a period tolerated by the cell (e.g., from about 30 min to 1 hour). Physiological conditions for conducting modification of polypeptides on a cell surface can be similar to those used in a ketone-azide reaction in modification of cells bearing cell-surface azides (see, e.g., U.S. Pat. No. 6,570,040).

Small molecule compounds containing, or modified to contain, an cx-nucleophilic group that serves as a reactive partner with a compound or conjugate disclosed herein are also contemplated for use as drugs in the polypeptide-drug conjugates of the present disclosure. General methods are known in the art for chemical synthetic schemes and conditions useful for synthesizing a compound of interest (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

### Methods of Producing Antibody

A subject antibody can be produced by any known method, e.g., conventional synthetic methods for protein synthesis; recombinant DNA methods, etc.

Where a subject antibody is a single chain polypeptide, it can be synthesized using standard chemical peptide synthesis techniques. Where a polypeptide is chemically synthesized, the synthesis may proceed via liquid-phase or solid-phase. Solid phase polypeptide synthesis (SPPS), in which the C-terminal amino acid of the sequence is attached to an insoluble support followed by sequential addition of the remaining amino acids in the sequence, is an example of a suitable method for the chemical synthesis of a subject antibody. Various forms of SPPS, such as Fmoc and Boc, are available for synthesizing a subject antibody.

Standard recombinant methods can be used for production of a subject antibody. For example, nucleic acids encoding light and heavy chain variable regions, optionally linked to constant regions, are inserted into expression vectors. The light and heavy chains can be cloned in the same or different expression vectors. The DNA segments encoding immunoglobulin chains are operably linked to control sequences in the expression vector(s) that ensure the expression of immunoglobulin polypeptides. Expression control sequences include, but are not limited to, promoters (e.g., naturally-associated or heterologous promoters), signal sequences, enhancer elements, and transcription termination sequences. The expression control sequences can be eukaryotic promoter systems in vectors capable of transforming or transfecting eukaryotic host cells (e.g., COS or CHO cells). Once the vector has been incorporated into the appropriate host, the host is maintained under conditions suitable for high level expression of the nucleotide sequences, and the collection and purification of the antibodies.

Because of the degeneracy of the code, a variety of nucleic acid sequences can encode each immunoglobulin amino acid sequence. The desired nucleic acid sequences can be produced by de novo solid-phase DNA synthesis or by polymerase chain reaction (PCR) mutagenesis of an earlier prepared variant of the desired polynucleotide.

Suitable expression vectors are typically replicable in the host organisms either as episomes or as an integral part of the host chromosomal DNA. Commonly, expression vectors contain selection markers (e.g., ampicillin-resistance, hygromycin-resistance, tetracycline resistance, kanamycin resistance or neomycin resistance) to permit detection of those cells transformed with the desired DNA sequences.

Escherichia coli is an example of a prokaryotic host cell that can be used for cloning a subject antibody-encoding polynucleotide. Other microbial hosts suitable for use include bacilli, such as Bacillus subtilis, and other enterobacteriaceae, such as Salmonella, Serratia, and various Pseudomonas species. Other microbes, such as yeast, are also useful for expression. Saccharomyces (e.g., S. cerevisiae) and Pichia are examples of suitable yeast host cells.

In addition to microorganisms, mammalian cells (e.g., mammalian cells grown in in vitro cell culture) can also be used to express and produce the polypeptides of the present invention (e.g., polynucleotides encoding immunoglobulins or fragments thereof). Suitable mammalian host cells include CHO cell lines, various Cos cell lines, HeLa cells, myeloma cell lines, and transformed B-cells or hybridomas. Expression vectors for these cells can include expression control sequences, such as an origin of replication, a promoter, and an enhancer, and necessary processing information sites, such as ribosome binding sites, RNA splice sites, polyadenylation sites, and transcriptional terminator sequences. Examples of suitable expression control sequences are promoters derived from immunoglobulin genes, SV40, adenovirus, bovine papilloma virus, cytomegalovirus and the like.

Once synthesized (either chemically or recombinantly), the whole antibodies, their dimers, individual light and heavy chains, or other forms of a subject antibody (e.g., scFv, etc.) can be purified according to standard procedures of the art, including ammonium sulfate precipitation, affinity columns, column chromatography, high performance liquid chromatography (HPLC) purification, gel electrophoresis, and the like (see generally Scopes, Protein Purification (Springer-Verlag, N.Y., (1982)). A subject antibody can be substantially pure, e.g., at least about 80% to 85% pure, at least about 85% to 90% pure, at least about 90% to 95% pure, or 98% to 99%, or more, pure, e.g., free from contaminants such as cell debris, macromolecules other than a subject antibody, etc.

### Compositions

The antibodies and/or the antibody-conjugates, e.g., ADCs, of the present disclosure can be formulated in a variety of different ways. In general, where the conjugate is a polypeptide-drug conjugate, the conjugate is formulated in a manner compatible with the drug conjugated to the polypeptide, the condition to be treated, and the route of administration to be used.

In some embodiments, provided is a pharmaceutical composition that includes any of the antibodies or the conjugates, e.g., ADCs, of the present disclosure and a pharmaceutically-acceptable excipient.

The antibody or the antibody-conjugate, e.g., ADC, can be provided in any suitable form, e.g., in the form of a pharmaceutically acceptable salt, and can be formulated for any suitable route of administration, e.g., oral, topical or parenteral administration. Where the conjugate is provided as a liquid injectable (such as in those embodiments where they are administered intravenously or directly into a tissue), the conjugate can be provided as a ready-to-use dosage form, or as a reconstitutable storage-stable powder or liquid composed of pharmaceutically acceptable carriers and excipients.

Methods for formulating the antibodies and/or the conjugates can be adapted from those readily available. For example, the antibodies and/or the conjugates can be provided in a pharmaceutical composition comprising a therapeutically effective amount of an antibody and/or a conjugate and a pharmaceutically acceptable carrier (e.g., saline). The pharmaceutical composition may optionally include other additives (e.g., buffers, stabilizers, preservatives, and the like). In some embodiments, the formulations are suitable for administration to a mammal, such as those that are suitable for administration to a human.

For example, the present disclosure provides a composition comprising a subject antibody or antibody-conjugate. A subject antibody or antibody-conjugate composition can comprise, in addition to a subject antibody or antibody-conjugate, one or more of: a salt, e.g., NaCl, MgCl₂, KCl, MgSO₄, etc.; a buffering agent, e.g., a Tris buffer, N-(2-Hydroxyethyl) piperazine-N′-(2-ethanesulfonic acid) (HEPES), 2-(N-Morpholino) ethanesulfonic acid (MES), 2-(N-Morpholino) ethanesulfonic acid sodium salt (MES), 3-(N-Morpholino) propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; glycerol; and the like.

In certain embodiments, the present disclosure provides compositions, including pharmaceutical compositions, comprising a subject antibody and/or antibody-conjugate. In general, a formulation comprises an effective amount of a subject antibody and/or antibody-conjugate. An “effective amount” means a dosage sufficient to produce a desired result, e.g., reduction in the number of cancerous cells. In some cases, the desired result is at least a reduction in a symptom of a malignancy, as compared to a control.

### Formulations

In the subject methods, a subject antibody and/or antibody-conjugate can be administered to the host using any convenient means capable of resulting in the desired therapeutic effect or diagnostic effect. Thus, the antibody and/or antibody-conjugate can be incorporated into a variety of formulations for therapeutic administration. More particularly, a subject antibody and/or antibody-conjugate can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, a subject antibody and/or antibody-conjugate can be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, a subject antibody and/or antibody-conjugate can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

A subject antibody and/or antibody-conjugate can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions comprising a subject antibody and/or antibody-conjugate are prepared by mixing the antibody and/or antibody-conjugate having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however, solutions comprising antibacterial agents may be used for the production of pharmaceutical compositions for parenteral administration.

Exemplary antibody and/or antibody-conjugate concentrations in a subject pharmaceutical composition may range from about 1 mg/mL to about 200 mg/ml or from about 50 mg/mL to about 200 mg/mL, or from about 150 mg/mL to about 200 mg/mL.

An aqueous formulation of the antibody and/or antibody-conjugate may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A lyoprotectant may also be added in order to protect the labile active ingredient (e.g., a protein) against destabilizing conditions during the lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 nM to 500 nM.

In some embodiments, a subject formulation includes a subject antibody and/or antibody-conjugate, and one or more agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

For example, a subject formulation can be a liquid or lyophilized formulation suitable for parenteral administration, and can comprise: about 1 mg/mL to about 200 mg/ml of a subject antibody conjugate; about 0.001% to about 1% of at least one surfactant; about 1 mM to about 100 mM of a buffer; optionally about 10 mM to about 500 mM of a stabilizer; and about 5 mM to about 305 mM of a tonicity agent; and has a pH of about 4.0 to about 7.0.

As another example, a subject parenteral formulation is a liquid or lyophilized formulation comprising about 1 mg/mL to about 200 mg/mL of a subject antibody conjugate; 0.04% Tween 20 w/v; 20 mM L-histidine; and 250 mM Sucrose; and has a pH of 5.5.

The term “unit dosage form,” as used herein, refers to physically discrete units suitable as unitary dosages for human and animal subjects, each unit containing a predetermined quantity of an antibody conjugate of the present disclosure calculated in an amount sufficient to produce the desired effect in association with a pharmaceutically acceptable diluent, carrier or vehicle. The specifications for a subject antibody conjugate may depend on the particular antibody conjugate employed and the effect to be achieved, and the pharmacodynamics associated with each antibody conjugate in the host.

A subject antibody and/or antibody-conjugate can be administered as an injectable formulation. Typically, injectable compositions are prepared as liquid solutions or suspensions; solid forms suitable for solution in, or suspension in, liquid vehicles prior to injection may also be prepared. The preparation may also be emulsified or the antibody conjugate encapsulated in liposome vehicles.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, a subject antibody and/or antibody-conjugate is formulated in a controlled release formulation. Sustained-release preparations may be prepared using methods well known in the art. Suitable examples of sustained-release preparations include semipermeable matrices of solid hydrophobic polymers containing the antibody conjugate in which the matrices are in the form of shaped articles, e.g., films or microcapsules. Examples of sustained-release matrices include polyesters, copolymers of L-glutamic acid and ethyl-L-glutamate, non-degradable ethylene-vinyl acetate, hydrogels, polylactides, degradable lactic acid-glycolic acid copolymers and poly-D-(−)-3-hydroxybutyric acid. Possible loss of biological activity and possible changes in immunogenicity of antibodies comprised in sustained-release preparations may be prevented by using appropriate additives, by controlling moisture content and by developing specific polymer matrix compositions.

Physical systems include, but are not limited to, reservoir systems with rate-controlling membranes, such as microencapsulation, macroencapsulation, and membrane systems; reservoir systems without rate-controlling membranes, such as hollow fibers, ultra microporous cellulose triacetate, and porous polymeric substrates and foams; monolithic systems, including those systems physically dissolved in non-porous, polymeric, or elastomeric matrices (e.g., nonerodible, crodible, environmental agent ingression, and degradable), and materials physically dispersed in non-porous, polymeric, or elastomeric matrices (e.g., nonerodible, erodible, environmental agent ingression, and degradable); laminated structures, including reservoir layers chemically similar or dissimilar to outer control layers; and other physical methods, such as osmotic pumps, or adsorption onto ion-exchange resins.

Chemical systems include, but are not limited to, chemical erosion of polymer matrices (e.g., heterogencous, or homogeneous erosion), or biological erosion of a polymer matrix (e.g., heterogeneous, or homogeneous).

### Dosages

A suitable dosage can be determined by an attending physician or other qualified medical personnel, based on various clinical factors. As is well known in the medical arts, dosages for any one patient depend upon many factors, including the patient's size, body surface area, age, the particular compound to be administered, sex of the patient, time, and route of administration, general health, and other drugs being administered concurrently. A subject antibody and/or antibody-conjugate may be administered in amounts between 1 ng/kg body weight and 20 mg/kg body weight per dose, e.g., between 0.1 mg/kg body weight to 10 mg/kg body weight, e.g., between 0.5 mg/kg body weight to 5 mg/kg body weight; however, doses below or above this exemplary range are envisioned, especially considering the aforementioned factors. If the regimen is a continuous infusion, it can also be in the range of 1 μg to 10 mg per kilogram of body weight per minute.

Those of skill will readily appreciate that dose levels can vary as a function of the specific antibody and/or antibody-conjugate, the severity of the symptoms and the susceptibility of the subject to side effects. Preferred dosages for a given compound are readily determinable by those of skill in the art by a variety of means.

### Routes of Administration

A subject antibody and/or antibody-conjugate is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, subcutaneous, intradermal, topical application, intravenous, intraarterial, rectal, nasal, oral, and other enteral and parenteral routes of administration. Routes of administration may be combined, if desired, or adjusted depending upon the antibody conjugate and/or the desired effect. A subject antibody and/or antibody-conjugate composition can be administered in a single dose or in multiple doses. In some embodiments, a subject antibody and/or antibody-conjugate composition is administered orally. In some embodiments, a subject antibody and/or antibody-conjugate composition is administered via an inhalational route. In some embodiments, a subject antibody and/or antibody-conjugate composition is administered intranasally. In some embodiments, a subject antibody and/or antibody-conjugate composition is administered locally. In some embodiments, a subject antibody and/or antibody-conjugate composition is administered intracranially. In some embodiments, a subject antibody and/or antibody-conjugate composition is administered intravenously.

The antibody and/or antibody-conjugate can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, intrahepatic, and intravenous routes, e.g., any route of administration other than through the alimentary canal. Parenteral administration can be carried to effect systemic or local delivery of a subject antibody. Where systemic delivery is desired, administration typically involves invasive or systemically absorbed topical or mucosal administration of pharmaceutical preparations.

A subject antibody and/or antibody-conjugate can also be delivered to the subject by enteral administration. Enteral routes of administration include, but are not necessarily limited to, oral and rectal (e.g., using a suppository) delivery.

By treatment is meant at least an amelioration of the symptoms associated with the pathological condition afflicting the host, where amelioration is used in a broad sense to refer to at least a reduction in the magnitude of a parameter, e.g., symptom, associated with the pathological condition being treated, such as a breast cancer, pancreatic cancer, or lung cancer. As such, treatment also includes situations where the pathological condition, or at least symptoms associated therewith, are completely inhibited, e.g., prevented from happening, or stopped, e.g., terminated, such that the host no longer suffers from the pathological condition, or at least the symptoms that characterize the pathological condition.

In some embodiments, a subject antibody and/or antibody-conjugate is administered by injection, e.g., for systemic delivery (e.g., intravenous infusion) or to a local site.

A variety of hosts (wherein the term “host” is used interchangeably herein with the terms “subject,” “individual,” and “patient”) are treatable according to the subject methods. Generally, such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the hosts will be humans.

### Treatment Methods

The present disclosure provides methods of treating a disease or disorder associated with or caused by a Nectin-4-positive cell, e.g., a cancerous Nectin-4-positive cell or an autoreactive Nectin-4-positive cell.

### Treating Malignancies

The present disclosure provides methods of treating a malignancy, including a solid tumor or a hematologic malignancy, the methods generally involving administering to an individual in need thereof (e.g., an individual having a malignancy) an effective amount of a subject antibody and/or antibody-conjugate, alone (e.g., in monotherapy) or in combination (e.g., in combination therapy) with one or more additional therapeutic agents.

Malignancies include, e.g., HCC, non-Hodgkin's lymphoma, Burkitt's lymphoma, multiple myeloma, chronic lymphocytic leukemia, hairy cell leukemia, prolymphocytic leukemia, anal cancer, appendix cancer, bile duct cancer (e.g., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, vulvar cancer, and the like.

In some embodiments, an effective amount of a subject antibody and/or antibody-conjugate is an amount that, when administered alone (e.g., in monotherapy) or in combination (e.g., in combination therapy) with one or more additional therapeutic agents, in one or more doses, is effective to reduce the number of cancerous cells in an individual by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the number of cancerous cells in the individual in the absence of treatment with the antibody conjugate.

In some instances, the cancer is a solid tumor, such as ovarian, ductal breast carcinoma, lung adenocarcinoma, and pancreatic cancer.

Aspects of the present disclosure include a method of delivering a drug to a target site in a subject. The method includes administering to the subject a pharmaceutical composition comprising a conjugate according to the present disclosure, where the administering is effective to release a therapeutically effective amount of the drug from the conjugate at the target site in the subject.

In some embodiments, multiple doses of an ADC are administered. The frequency of administration of an ADC can vary depending on any of a variety of factors, e.g., severity of the symptoms, condition of the subject, etc. For example, in some embodiments, an ADC is administered once per month, twice per month, three times per month, every other week, once per week (qwk), twice per week, three times per week, four times per week, five times per week, six times per week, every other day, daily (qd/od), twice a day (bds/bid), or three times a day (tds/tid), etc.

### Combination Therapy

In some embodiments, a subject method of treating a malignancy involves administering a subject antibody and/or antibody-conjugate and one or more additional therapeutic agents. Suitable additional therapeutic agents include, but are not limited to, a cancer chemotherapeutic agent (as described above).

In some embodiments, the treatment method may include administering to the subject a therapeutically effective amount of an immunomodulatory therapeutic agent. The immunomodulatory therapeutic agent may be an immune checkpoint inhibitor or interleukin. The immune checkpoint inhibitor may inhibit A2AR, B7-H3, B7-H4, BTLA, CTLA-4, CD277, IDO, KIR, PD-1, LAG-3, TIM-3, TIGIT or VISTA. The immune checkpoint inhibitor that inhibits PD-1 signaling may be an anti-PD-1 antibody. The anti-PD-1 antibody may be nivolumab, pembrolizumab, atezolizumab, durvalumab, or avelumab. The immune checkpoint inhibitor that inhibits CTLA-4 may be an anti-CTLA-4 antibody. The anti-CTLA-4 antibody may be ipilimumab.

### Subjects Suitable for Treatment

A variety of subjects are suitable for treatment with a subject method. Suitable subjects include any individual, e.g., a human, who has a malignancy; who has been diagnosed with a malignancy; who has had a malignancy and is at risk for recurrence of the malignancy; who has been treated for a malignancy with an agent other than a subject anti-Nectin-4 antibody conjugate (e.g., who has been treated with a cancer chemotherapeutic agent) and who has not responded to the agent; or who has been treated for a malignancy with an agent other than a subject anti-Nectin-4 antibody conjugate (e.g., who has been treated with a cancer chemotherapeutic agent) and who initially responded to the agent but subsequently ceased to respond (e.g., relapsed). A subject can have a solid tumor, such as ovarian, ductal breast carcinoma, lung adenocarcinoma, and pancreatic cancer.

### EMBODIMENTS

Certain embodiments of the present disclosure are described in the clauses listed below. These embodiments are illustrative only and not intended to be limiting in scope.

1. A conjugate of formula (I):

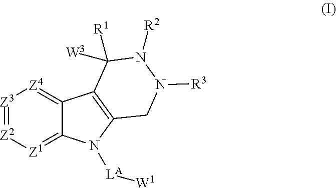

wherein:
Z¹, Z², Z³ and Z⁴ are each independently selected from CR⁴, N and C-L^{B}-W²;
R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L^{A} is a first linker;
L^{B} is a second linker;
W¹ is a first drug;
W² is a second drug; and
W³ is an anti-Nectin-4 antibody.

2. The conjugate of clause 1, wherein Z¹ is CR⁴.

3. The conjugate of clause 1, wherein Z¹ is N.

4. The conjugate of clause 1, wherein Z³ is CR⁴.

5. The conjugate of clause 1, wherein Z³ is C-L^{B}-W².

6. The conjugate of any one of clauses 1-5, wherein L^{A} comprises:
-(T¹-V¹)ₐ-(T²-V²)_{b}-(T³-V³)_{c}-(T⁴-V⁴)_{d}-(T⁵-V⁵)ₑ-(T⁶-V⁶)_{f}-,
   wherein
a, b, c, d, e and f are each independently 0 or 1;
T¹, T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
V¹, V², V³, V⁴, V⁵ and V⁶ are each independently selected from the group consisting of a covalent bond, —CO—, —NR¹⁵—, —NR¹⁵ (CH₂)_{q}—, —NR¹⁵ (C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, an alkyl, a substituted alkyl, an aryl, and a substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

7. The conjugate of clause 6, wherein T¹, T², T³, T⁴, T⁵ and T⁶ are each optionally substituted with a glycoside.

8. The conjugate of clause 6, wherein MABO, MABC, PABO, PABC, PAB, PABA, PAP and PHP are each optionally substituted with a glycoside.

9. The conjugate of any one of clauses 7-8, wherein the glycoside is selected from a glucuronide, a galactoside, a glucoside, a mannoside, a fucoside, O-GlcNAc, and O-GalNAc.

10. The conjugate of any one of clauses 6-9,

wherein:
T¹ is (C₁-C₁₂)alkyl and V¹ is —CO—;
T² is an amino acid analog and V² is —NH—;
T³ is (PEG), and V³ is —CO—;
T⁴ is AA and V⁴ is absent;
T⁵ is PABC and V⁵ is absent; and
f is 0; or
   wherein:
T¹ is (C₁-C₁₂)alkyl and V¹ is —CONH—;
T² is (PEG), and V² is —CO—;
T³ is AA and V³ is absent;
T⁴ is PABC and V⁴ is absent; and
e and f are each 0; or
   wherein:
T¹ is (C₁-C₁₂)alkyl and V¹ is —CONH—;
T² is substituted (C₁-C₁₂)alkyl and V² is —CO—;
T³ is AA and V³ is absent;
T⁴ is PABC and V⁴ is absent; and
e and f are each 0.

11. The conjugate of any one of clauses 1-10, wherein L^{B} comprises:
-(T⁷-V⁷)_{g}-(T⁸-V⁸)ₕ-(T⁹-V⁹)ᵢ-(T¹⁰-V¹⁰)ⱼ-(T¹¹-V¹¹)ₖ-(T¹²-V¹²)ₗ-(T¹³-V¹³)ₘ-,
   wherein
g, h, i, j, k, l and m are each independently 0 or 1;
T⁷, T⁸, T⁹, T¹⁰, T¹¹, T¹² and T¹³ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
V⁷, V⁸, V⁹, V¹⁰, V¹¹, V¹² and V¹³ are each independently selected from the group consisting of a covalent bond, —CO—, —NR¹⁵, —NR¹⁵ (CH₂)_{q}—, —NR¹⁵ (C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, an alkyl, a substituted alkyl, an aryl, and a substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

12. The conjugate of clause 11, wherein T⁷, T⁸, T⁹, T¹⁰, T¹¹, T¹² and T¹³ are each optionally substituted with a glycoside.

13. The conjugate of clause 11, wherein MABO, MABC, PABO, PABC, PAB, PABA, PAP and PHP are each optionally substituted with a glycoside.

14. The conjugate of any one of clauses 12-13, wherein the glycoside is selected from a glucuronide, a galactoside, a glucoside, a mannoside, a fucoside, O-GlcNAc, and O-GalNAc.

15. The conjugate of any one of clauses 11-14, wherein:
T⁷ is absent and V⁷ is —NHCO—;
T⁸ is (C₁-C₁₂)alkyl and V⁸ is —CONH—;
T⁹ is (PEG), and V⁹ is —CO—;
T¹⁰ is AA and V¹⁰ is absent; and
T¹¹ is PABC and V¹¹ is absent; and
l and m are each 0; or
   wherein:
T⁷ is absent and V⁷ is —NHCO—;
T⁸ is (C₁-C₁₂)alkyl and V⁸ is —CONH—;
T⁹ is substituted (C₁-C₁₂)alkyl and V⁹ is —CO—;
T¹⁰ is AA and V¹⁰ is absent;
T¹¹ is PABC and V¹ is absent; and
l and m are each 0.

16. The conjugate of any one of clauses 1-15, wherein the conjugate is selected from:

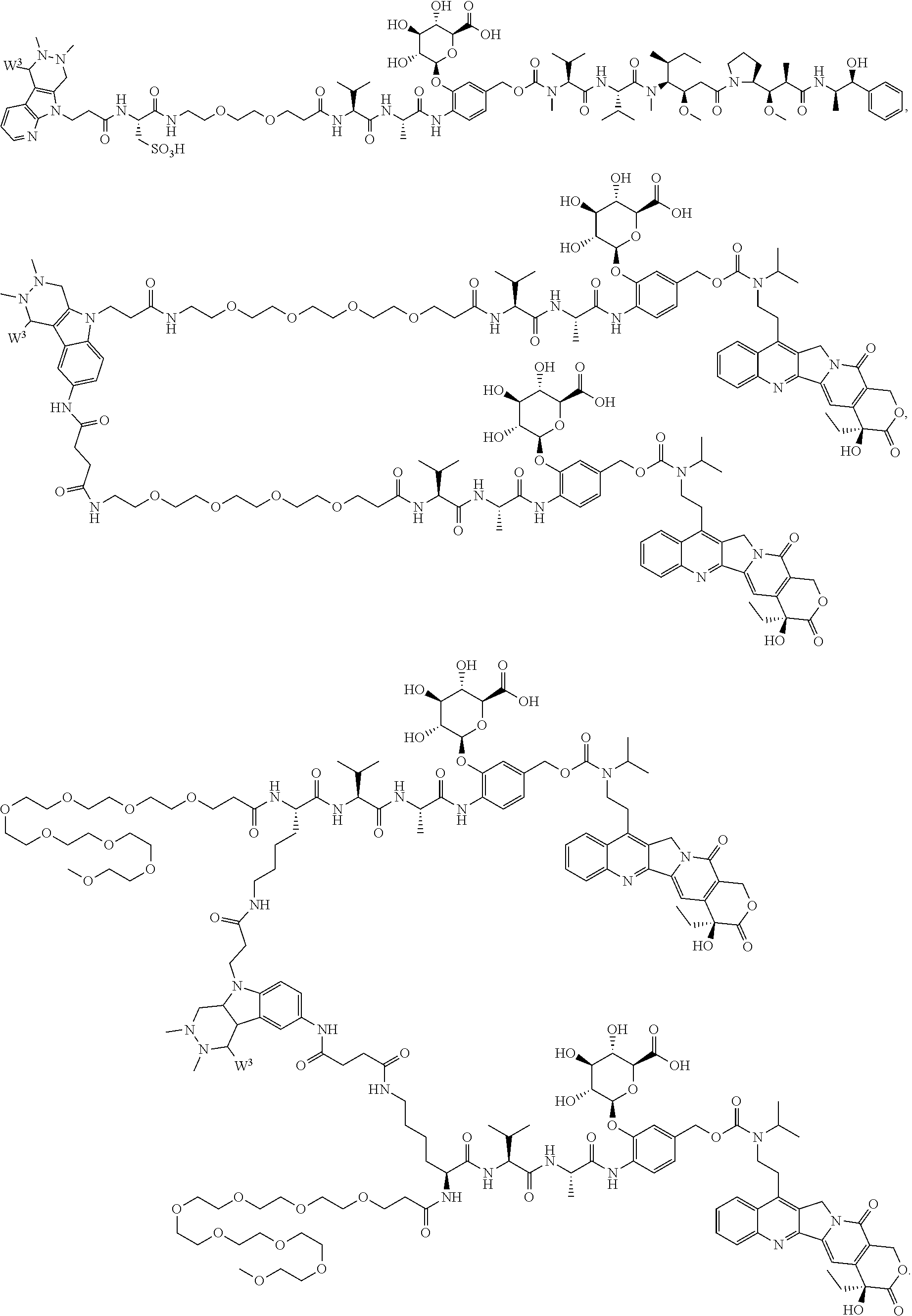

17. The conjugate of any one of clauses 1 to 16, wherein the anti-Nectin-4 antibody is an IgG1 antibody.

18. The conjugate of clause 17, wherein the anti-Nectin-4 antibody is an IgG1 kappa antibody.

19. The conjugate of any one of clauses 1 to 18, wherein the anti-Nectin-4 antibody comprises a sequence of the formula (II):

X¹(fGly′)X²Z²X³Z³(SEQ ID NO: 128) (II),

wherein
X¹ is present or absent and, when present, can be any amino acid, with the proviso that when the sequence is at the N-terminus of the conjugate, X¹ is present;
fGly′ is an amino acid residue coupled to the first drug or the second drug through the first linker or the second linker, respectively;
X² and X³ are each independently any amino acid;
Z² is either a proline or alanine residue; and
Z³ is a basic amino acid or an aliphatic amino acid.

20. The conjugate of clause 19, wherein the sequence is L(fGly′)TPSR (SEQ ID NO: 246).

21. The conjugate of clause 19, wherein
Z³ is selected from R, K, H, A, G, L, V, I, and P;
X¹ is selected from L, M, S, and V; and
X² and X³ are each independently selected from S, T, A, V, G, and C.

22. The conjugate of any one of clauses 19 to 21, wherein the sequence is positioned at a C-terminus of a heavy chain constant region of the anti-Nectin-4 antibody.

23. The conjugate of clause 22, wherein the heavy chain constant region comprises a sequence of the formula (II):

X¹(fGly′)X²Z²X³Z³(SEQ ID NO:128) (II),

wherein
X¹ is present or absent and, when present, can be any amino acid, with the proviso that when the sequence is at the N-terminus of the conjugate, X¹ is present;
fGly′ is an amino acid residue coupled to the first drug or the second drug through the first linker or the second linker, respectively
X² and X³ are each independently any amino acid;
Z² is either a proline or alanine residue;
Z³ is a basic amino acid or an aliphatic amino acid, and
wherein the sequence is C-terminal to the amino acid sequence SLSLSPG (SEQ ID NO: 247).

24 The conjugate of clause 23, wherein the heavy chain constant region comprises the sequence SPGSL(fGly′)TPSRGS (SEQ ID NO: 130).

25. The conjugate of clause 23, wherein
Z³ is selected from R, K, H, A, G, L, V, I, and P;
X¹ is selected from L, M, S, and V; and
X² and X³ are each independently selected from S, T, A, V, G, and C.

26. The conjugate of any one of clauses 22 to 25, wherein the heavy chain constant region of the anti-Nectin-4 antibody comprises an amino acid sequence at least 85%, at least 90%, at least 95%, at least 96%, at least 97%, at least 98%, or at least 99% identical to the amino acid sequence set forth in a sequence selected from SEQ ID NOs: 70 and comprises the fGly′ residue instead of C in the sequence LCTPSR (SEQ ID NO: 104).

27. The conjugate of any one of clauses 19 to 21, wherein the heavy chain constant region of the anti-Nectin-4 antibody comprises an amino acid sequence at least 85%, at least 90%, at least 95%, at least 96%, at least 97%, at least 98%, or at least 99% identical to the amino acid sequence set forth in any one of SEQ ID NOs: 71, 75, 79, and 83 and comprises the fGly′ residue instead of C in the sequence LCTPSR (SEQ ID NO: 104).

28 The conjugate of any one of clauses 19 to 21, wherein the heavy chain constant region of the anti-Nectin-4 antibody comprises an amino acid sequence at least 85%, at least 90%, at least 95%, at least 96%, at least 97%, at least 98%, or at least 99% identical to the amino acid sequence set forth in any one of SEQ ID NOs: 72, 76, 80, and 84 and comprises the fGly′ residue instead of C in the sequence LCTPSR (SEQ ID NO: 104).

29 The conjugate of any one of clauses 19 to 21, wherein the heavy chain constant region of the anti-Nectin-4 antibody comprises an amino acid sequence at least 85%, at least 90%, at least 95%, at least 96%, at least 97%, at least 98%, or at least 99% identical to the amino acid sequence set forth in any one of SEQ ID NOs: 73, 77, 81, and 85 and comprises the fly′ residue instead of C in the sequence LCTPSR (SEQ ID NO: 104).

30. The conjugate of any one of clauses 19 to 21, wherein the heavy chain constant region of the anti-Nectin-4 antibody comprises an amino acid sequence at least 85%, at least 90%, at least 95%, at least 96%, at least 97%, at least 98%, or at least 99% identical to the amino acid sequence set forth in any one of SEQ ID NOs: 74, 78, 82, and 86 and comprises the fGly′ residue instead of C in the sequence LCTPSR (SEQ ID NO: 104).

31. The conjugate of any one of clauses 19 to 21, wherein the fGly′ residue is positioned in a light chain constant region of the anti-Nectin-4 antibody.

32. The conjugate of clause 31, wherein the light chain constant region comprises a sequence of the formula (II):

X¹(fGly′)X²Z²X³Z³(SEQ ID NO:128) (II),

wherein
X¹ is present or absent and, when present, can be any amino acid, with the proviso that when the sequence is at the N-terminus of the conjugate, X¹ is present;
fGly′ is the amino acid residue coupled to the drug through the linker;
X² and X³ are each independently any amino acid;
Z² is either a proline or alanine residue;
Z³ is a basic amino acid or an aliphatic amino acid, and
wherein the sequence is C-terminal to the amino acid sequence KVDNAL (SEQ ID NO: 132), and/or is N-terminal to the sequence QSGNSQ (SE ID NO: 133).

33. The conjugate of clause 32, wherein the light chain constant region comprises the sequence KVDNAL(fGly′)TPSRQSGNSQ (SEQ ID NO: 134).

34. The conjugate of clause 33, wherein
Z³ is selected from R, K, H, A, G, L, V, I, and P;
X¹ is selected from L, M, S, and V; and
X² and X³ are each independently selected from S, T, A, V, G, and C.

35. The conjugate of any one of clauses 19 to 21, wherein the fGly′ residue is positioned in a heavy chain CH1 region of the anti-Nectin-4 antibody.

36. The conjugate of clause 35, wherein the light chain constant region comprises a sequence of the formula (II):

X¹(fGly′)X²Z²X³Z³(SEQ ID NO:128) (II),

wherein
X¹ is present or absent and, when present, can be any amino acid, with the proviso that when the sequence is at the N-terminus of the conjugate, X¹ is present;
fGly′ is the amino acid residue coupled to the drug through the linker;
X² and X³ are each independently any amino acid;
Z² is either a proline or alanine residue;
Z³ is a basic amino acid or an aliphatic amino acid, and
wherein the sequence is C-terminal to the amino acid sequence SWNSGA (SEQ ID NO: 135) and/or is N-terminal to the amino acid sequence GVHTFP (SEQ ID NO: 136).

37. The conjugate of clause 36, wherein the heavy chain CH1 region comprises the sequence SWNSGAL(fGly′)TPSRGVHTFP (SEQ ID NO: 137).

38. The conjugate of clause 29, wherein
Z³ is selected from R, K, H, A, G, L, V, I, and P;
X¹ is selected from L, M, S, and V; and
X² and X³ are each independently selected from S, T, A, V, G, and C.

39 The conjugate of any one of clauses 19 to 21, wherein the fGly′ residue is positioned in a heavy chain CH₂ region of the anti-Nectin-4 antibody.

40. The conjugate of any one of clauses 19 to 21, wherein the fGly′ residue is positioned in a heavy chain CH₃ region of the anti-Nectin-4 antibody.

41. The conjugate of any one of clauses 1 to 40, wherein the anti-Nectin-4 antibody competes for binding to Nectin-4 with an anti-Nectin-4 antibody comprising:
a variable heavy chain (VH) chain comprising heavy chain CDRs 1-3 (HCDRs 1-3) of a VH chain having a sequence selected from SEQ ID NOs: 1 to 17; and
a variable light chain (VL) chain comprising light chain CDRs 1-3 (LCDRs 1-3) of a VL chain having a sequence selected from SEQ ID NOs: 18 to 31.

42. The conjugate of any one of clauses 1 to 40, wherein the anti-Nectin-4 antibody comprises:
a VH chain comprising heavy chain CDRs 1-3 (HCDRs 1-3) of a VH chain having a sequence selected from SEQ ID NOs: 1 to 17; and
a VL chain comprising light chain CDRs 1-3 (LCDRs 1-3) of a VL chain having a sequence selected from SEQ ID NOs: 18 to 31.

43. The conjugate of clause 42, wherein the antibody that specifically binds to Nectin-4 comprises:
a VH chain comprising a sequence selected from SEQ ID NOs: 1 to 17; and
a VL chain comprising a sequence selected from SEQ ID NOs: 18 to 31.

44. The conjugate of clause 42, wherein the antibody that specifically binds to Nectin-4 comprises:
a VH chain comprising HCDRs 1-3 of a VH chain having a sequence selected from SEQ ID NOs: 1 to 6; and
a VL chain comprising LCDRs 1-3 of a VL chain having a sequence selected from SEQ ID NOs: 18 to 23.

45. The conjugate of clause 42, wherein the antibody that specifically binds to Nectin-4 comprises:
a VH chain comprising a sequence selected from SEQ ID NOs: 1 to 6; and
a VL chain comprising a sequence selected from SEQ ID NOs: 18 to 23.

46. The conjugate of clause 42, wherein the antibody that specifically binds to Nectin-4 comprises:
a VH chain comprising HCDRs 1-3 of a VH chain having a sequence selected from SEQ ID NOs: 7 to 13; and
a VL chain comprising LCDRs 1-3 of a VL chain having a sequence selected from SEQ ID NOs: 24 to 27.

47. The conjugate of clause 42, wherein the antibody that specifically binds to Nectin-4 comprises:
a VH chain comprising a sequence selected from SEQ ID NOs: 7 to 13; and
a VL chain comprising a sequence selected from SEQ ID NOs: 24 to 27.

48. The conjugate of clause 42, wherein the antibody that specifically binds to Nectin-4 comprises:
a VH chain comprising HCDRs 1-3 of a VH chain having a sequence selected from SEQ ID NOs: 14 to 17; and
a VL chain comprising LCDRs 1-3 of a VL chain having a sequence selected from SEQ ID NOs: 28 to 31.

49. The conjugate of clause 42, wherein the antibody that specifically binds to Nectin-4 comprises:
a VH chain comprising a sequence selected from SEQ ID NOs: 14 to 17; and
a VL chain comprising a sequence selected from SEQ ID NOs: 28 to 31.

50. The conjugate of clause 42, wherein the antibody that specifically binds to Nectin-4 comprises:
the VH chain of an anti-Nectin-4 antibody comprising the HCDRs 1-3 of a VH chain having a sequence selected from SEQ ID NOs: 1 to 17 and an amino acid sequence having 80% or greater, 85% or greater, 90% or greater, 95% or greater, 99% or greater, or 100% sequence identity to the amino acid sequence set forth in a sequence selected from SEQ ID NOs: 1 to 17, wherein any amino acid differences between the VH chain of an anti-Nectin-4 antibody and a sequence selected from SEQ ID NOs: 1 to 17 is in the regions outside of the CDRs; and
the VL chain of an anti-Nectin-4 antibody comprises the LCDRs 1-3 of a VL chain having a sequence selected from SEQ ID NOs: 18 to 31 and comprises an amino acid sequence having 80% or greater, 85% or greater, 90% or greater, 95% or greater, 99% or greater, or 100% sequence identity to the amino acid sequence set forth in a sequence selected from SEQ ID NOs: 18 to 31, wherein any amino acid differences between the VL chain of an anti-Nectin-4 antibody and a sequence selected from SEQ ID NOs: 18 to 31 is within the regions outside of the CDRs.

51. The conjugate of any one of clauses 42-50, wherein the anti-Nectin-4 antibody comprises: a heavy chain constant region having the amino acid sequence set forth in any one of SEQ ID NOs: 70 to 86, wherein the C present in the sequence LCTPSR in the constant region is replaced by fGly.

52. A pharmaceutical composition comprising:
a conjugate of any one of clauses 1 to 51; and
a pharmaceutically-acceptable excipient.

53. A method comprising:
administering to a subject an effective amount of the conjugate of any one of clauses 1 to 51 or the pharmaceutical composition of clause 52.

54. A method of treating cancer in a subject, the method comprising:
administering to the subject a therapeutically effective amount of the conjugate of any one of clauses 1 to 51 or the pharmaceutical composition of clause 52, wherein the administering is effective to treat cancer in the subject.

55. The method according to clause 54, wherein the cancer is ovarian cancer, ductal breast carcinoma, lung adenocarcinoma, and pancreatic cancer.

56. The method according to clause 55, wherein the cancer is characterized by cancer cells expressing Nectin-4.

57. The method according to clause 56, wherein the conjugate binds to Nectin-4.

58. The method of any one of clauses 53 to 57, further comprising administering to the subject a therapeutically effective amount of an immunomodulatory therapeutic agent.

59. The method of clause 58, wherein the immunomodulatory therapeutic agent is an immune checkpoint inhibitor or interleukin.

60. The method of clause 59, wherein the immune checkpoint inhibitor inhibits A2AR, B7-H3, B7-H4, BTLA, CTLA-4, CD277, IDO, KIR, PD-1, LAG-3, TIM-3, TIGIT and VISTA.

61. The method of clause 60, wherein the immune checkpoint inhibitor that inhibits PD-1 signaling is an anti-PD-1 antibody.

62. The method of clause 61, wherein the anti-PD-1 antibody is nivolumab, pembrolizumab, atezolizumab, durvalumab, or avelumab.

63. The method of clause 60, wherein the immune checkpoint inhibitor that inhibits CTLA-4 is an anti-CTLA-4 antibody.

64 The method of clause 63, wherein the anti-CTLA-4 antibody is ipilimumab.

65. A method of delivering a drug to a target site in a subject, the method comprising:
administering to the subject the conjugate of any one of clauses 1 to 51 or the pharmaceutical composition of clause 52, wherein the administering is effective to release a therapeutically effective amount of the drug from the conjugate at the target site in the subject.

66. An anti-Nectin-4 antibody, comprising:
a variable heavy chain (VH) chain comprising heavy chain CDRs 1-3 (HCDRs 1-3) of a VH chain having a sequence selected from SEQ ID NOs: 1 to 17; and
a variable light chain (VL) chain comprising light chain CDRs 1-3 (LCDRs 1-3) of a VL chain having a sequence selected from SEQ ID NOs: 18 to 31.

67. The anti-Nectin-4 antibody of clause 66, comprising:
a VH chain comprising a sequence selected from SEQ ID NOs: 1 to 17; and
a VL chain comprising a sequence selected from SEQ ID NOs: 18 to 31.

68. The anti-Nectin-4 antibody of clause 66, comprising:
a VH chain comprising HCDRs 1-3 of a VH chain having a sequence selected from SEQ ID NOs: 1 to 6; and
a VL chain comprising LCDRs 1-3 of a VL chain having a sequence selected from SEQ ID NOs: 18 to 23.

69. The anti-Nectin-4 antibody of clause 66, comprising:
a VH chain comprising a sequence selected from SEQ ID NOs: 1 to 6; and
a VL chain comprising a sequence selected from SEQ ID NOs: 18 to 23.

70. The anti-Nectin-4 antibody of clause 66, comprising:
a VH chain comprising HCDRs 1-3 of a VH chain having a sequence selected from SEQ ID NOs: 7 to 13; and
a VL chain comprising LCDRs 1-3 of a VL chain having a sequence selected from SEQ ID NOs: 24 to 27.

71. The anti-Nectin-4 antibody of clause 66, comprising:
a VH chain comprising a sequence selected from SEQ ID NOs: 7 to 13; and
a VL chain comprising a sequence selected from SEQ ID NOs: 24 to 27.

72. The anti-Nectin-4 antibody of clause 66, comprising:
a VH chain comprising HCDRs 1-3 of a VH chain having a sequence selected from SEQ ID NOs: 14 to 17; and
a VL chain comprising LCDRs 1-3 of a VL chain having a sequence selected from SEQ ID NOs: 28 to 31.

73 The anti-Nectin-4 antibody of clause 66, comprising:
a VH chain comprising a sequence selected from SEQ ID NOs: 14 to 17; and
a VL chain comprising a sequence selected from SEQ ID NOs: 28 to 31.

74. The anti-Nectin-4 antibody of clause 66, comprising:
the VH chain of an anti-Nectin-4 antibody comprising the HCDRs 1-3 of a VH chain having a sequence selected from SEQ ID NOs: 1 to 17 and an amino acid sequence having 80% or greater, 85% or greater, 90% or greater, 95% or greater, 99% or greater, or 100% sequence identity to the amino acid sequence set forth in a sequence selected from SEQ ID NOs: 1 to 17, wherein any amino acid differences between the VH chain of an anti-Nectin-4 antibody and a sequence selected from SEQ ID NOs: 1 to 17 is in the regions outside of the CDRs; and
the VL chain of an anti-Nectin-4 antibody comprises the LCDRs 1-3 of a VL chain having a sequence selected from SEQ ID NOs: 18 to 31 and comprises an amino acid sequence having 80% or greater, 85% or greater, 90% or greater, 95% or greater, 99% or greater, or 100% sequence identity to the amino acid sequence set forth in a sequence selected from SEQ ID NOs: 18 to 31, wherein any amino acid differences between the VL chain of an anti-Nectin-4 antibody and a sequence selected from SEQ ID NOs: 18 to 31 is within the regions outside of the CDRs.

75. The anti-Nectin-4 antibody of any one of clauses 66-74, comprising: a heavy chain constant region having the amino acid sequence set forth in any one of SEQ ID NOs: 70 to 86, wherein the C present in the sequence LCTPSR in the constant region is replaced by fGly.

76. A pharmaceutical composition comprising:
an antibody of any one of clauses 66 to 75; and
a pharmaceutically-acceptable excipient.

77. A method comprising:
administering to a subject an effective amount of the antibody of any one of clauses 66 to 75 or the pharmaceutical composition of clause 76.

78. A method of treating cancer in a subject, the method comprising:
administering to the subject a therapeutically effective amount of the antibody of any one of clauses 66 to 75 or the pharmaceutical composition of clause 76, wherein the administering is effective to treat cancer in the subject.

79 The method according to clause 78, wherein the cancer is ovarian cancer, ductal breast carcinoma, lung adenocarcinoma, and pancreatic cancer.

80. The method according to clause 79, wherein the cancer is characterized by cancer cells expressing Nectin-4.

81. The method according to clause 80, wherein the conjugate binds to Nectin-4.

82. The method of any one of clauses 78 to 81, further comprising administering to the subject a therapeutically effective amount of an immunomodulatory therapeutic agent.

83. The method of clause 82, wherein the immunomodulatory therapeutic agent is an immune checkpoint inhibitor or interleukin.

84. The method of clause 83, wherein the immune checkpoint inhibitor inhibits A2AR, B7-H3, B7-H4, BTLA, CTLA-4, CD277, IDO, KIR, PD-1, LAG-3, TIM-3, TIGIT and VISTA.

85. The method of clause 84, wherein the immune checkpoint inhibitor that inhibits PD-1 signaling is an anti-PD-1 antibody.

86. The method of clause 85, wherein the anti-PD-1 antibody is nivolumab, pembrolizumab, atezolizumab, durvalumab, or avelumab.

87. The method of clause 84, wherein the immune checkpoint inhibitor that inhibits CTLA-4 is an anti-CTLA-4 antibody.

88. The method of clause 87, wherein the anti-CTLA-4 antibody is ipilimumab.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular (ly); i.p., intraperitoneal (ly); s.c., subcutaneous (ly); and the like. Commercially available reagents referred to in the Examples were used according to manufacturer's instructions unless otherwise indicated. The source of cells identified in the Examples and throughout the specification by ECACC accession numbers is the European Collection of Cell Cultures (ECACC), Salisbury, England. Unless otherwise defined, all technical and scientific terms used herein have the same meaning as commonly understood by one of ordinary skill in the art to which this invention belongs. Exemplary methods and materials are described below although methods and materials similar or equivalent to those described herein can also be used in the practice or testing of the present invention. The materials, methods, and examples are illustrative only and not intended to be limiting in scope.

### General Synthetic Procedures

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any purification protocol known in the art, including chromatography, such as HPLC, preparative thin layer chromatography, flash column chromatography and ion exchange chromatography. Any suitable stationary phase can be used, including normal and reversed phases as well as ionic resins. In certain embodiments, the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4^{th} edition, Vol. 15/1, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974. The protecting groups may be removed at a convenient subsequent stage using methods known from the art.

The subject compounds can be synthesized via a variety of different synthetic routes using commercially available starting materials and/or starting materials prepared by conventional synthetic methods. A variety of examples of synthetic routes that can be used to synthesize the compounds disclosed herein are described in the schemes below.

### Example 1: Anti-Nectin-4 Monoclonal Antibodies

### Methods and Results

### Antibody Discovery and Lead Selection Methods

Recombinant human nectin-4-His protein was used to immunize mice. 1500 clones were screened by ELISA to test reactivity to the antigen. Positive hits were confirmed through rescreening against human nectin-4-His, cynomolgus nectin-4-His, and human CD22-His proteins to identify clones with strong selective binding to human and cynomolgus nectin-4 protein. Lead clones were sequenced and produced via transient transfection as recombinant mouse-human chimeric antibodies bearing human kappa light chain and IgG1 constant regions carrying two aldehyde tag insertions. The antibodies were conjugated to an aldehyde-reactive linker-payloads prior to further analysis.

### Humanized Antibody Production Methods

The 5D9 clone was selected for humanization. Five heavy chain and five light chain variant sequences were designed (Tables 2-3) and antibody variants were constructed by pairing each of these heavy and light chains in all possible combinations. Human kappa light chain and IgG1 constant regions carrying two aldehyde tag insertions were used as the constant regions for variants production. The antibodies were produced via transient transfection and were conjugated to an aldehyde-reactive linker-payloads prior to further analysis.

### Antibody Discovery and Lead Selection Results

From 1500 initial clones screened, 27 passed the rescreening process. Of these 20 clone sequences were successfully recovered and were produced recombinantly as chimeric antibodies carrying two aldehyde tag insertions. Titers of the lead clones are shown in Table 6. ELISA results from the rescreening of the twenty clones (as hybridoma supernatants and recombinant proteins) are shown in Table 6.

TABLE 6
Titers of lead antibody clones

| | Titer |
|---|---|
| Antibody Clone | (mg/L) |
| 12E11 | 250.3 |
| 3C12 | 302.6 |
| 5D9 | 241.6 |
| 6C8 | 275.2 |
| 7E8 | 145.4 |
| 7H10 | 256 |

### ELISA Methods

TABLE 7
Nectin and NecL ELISA Reagents

| Consumables | Vendor | Item Num | Item Name | Use |
|---|---|---|---|---|
| Plates | Fisher | 12566121 | 96 DeepWell 2 mL plate, | For making |
| | | | polypropylene, non-sterile | dilutions |
| | VWR | 62409-024 | Nunc Maxisorp 96-well plates | For |
| | | | | performing |
| | | | | the assay |
| antigen | ACRO | PV1-h5223 | human Nectin-1-his (400 ug/ml in | coating the |
| | Biosystems | | H20) | wells |
| | ACRO | PV2-H52E2 | human Nectin-2 his (600 ug/ml H20) | |
| | Biosystems | | | |
| | ACRO | PV3-H52E4 | human Nectin-3-His (400 ul/ml in | |
| | Biosystems | | H20) | |
| | ACRO | NEA-H52H3 | human Nectin-4-His (stock 400 ug/ml | |
| | Biosystems | | in H20) | |
| | R&D systems | 3678-S4-050 | human Necl-1 (100 ug/ml in PBS) | |
| | ACRO | CA1-H5225 | human Necl-2 (100 ug/ml in H20) | |
| | Biosystems | | | |
| | R&D systems | 4290-S4-050 | human Necl-3 (100 ug/ml in PBS) | |
| | R&D systems | 4164-S4-050 | human Necl-4 (200 ug/ml in PBS) | |
| | R&D systems | 2530-CD-50 | human Necl-5 (100 ug/ml in PBS) | |
| Antibodies | Jackson | 109-035-098 | goat anti-human IgG Fc gamma | Detection for |
| | Immunoresearch | | specific HRP conjugate | total Ab |
| TMB | Thermo Fisher | 34028 | Ultra TMB One-Step ELISA substrate | |
| | (Pierce) | | | |
| Blocking | Thermo Fisher | 37528 | Blocker casein | |
| buffer | (Pierce) | | | |
| H2 SO4 | Sigma | 320501 | Sulfuric acid (dilute to make | |
| | | | quenching reagent) | |
| Hardware | | | | |
| Plate washer | BioTek | | ELx405 | |
| Plate reader | Molecular | | SpectraMax M5 with SoftMaxPro | |
| | Devices | | software | |

### Nectin and NecL ELISA Protocol

1. Plated 100 μL of antigen @1 ug/ml in PBS on a maxisorp 96 well plate.
2. Maxisorp plates. Incubated overnight at 4° C.
3. Washed 4× with 0.1% Tween PBS.
4. Blocked wells with 200 μL PBS casein 2 hours at RT with shaking.
5. Washed 4× with 0.1% Tween PBS.
6. dilute antibody samples to appropriate starting concentration.
7. Serially diluted mAbs 1:3 in PBS starting at 3 ug/ml (20 nM) in deep well plate. Mix 10-15 times. NOTE: mAbs at 300 ug/ml (2 μM) to test physiological concentrations can also be used.
8. Added 100 μL of mAb dilutions to 96 well plated.
9. Incubated at RT 1 hour with shaking.
10. Washed/Soak 6× with 0.1% Tween PBS.
11. Added 100 uL 1:15000 dilution goat anti human HRP to wells (dilute in PBS)×
12. Incubated at RT 0.5 hour with shaking.
13. Washed 4× with 0.1% Tween PBS.
14. Added 100 μL TMB, developed plates until top well dark blue color.
15. Added 100 μL 2N H₂SO₄.
16. Absorbance reading on plate reader.

### Nectin-4 Binding ELISA Clonal Selection Results

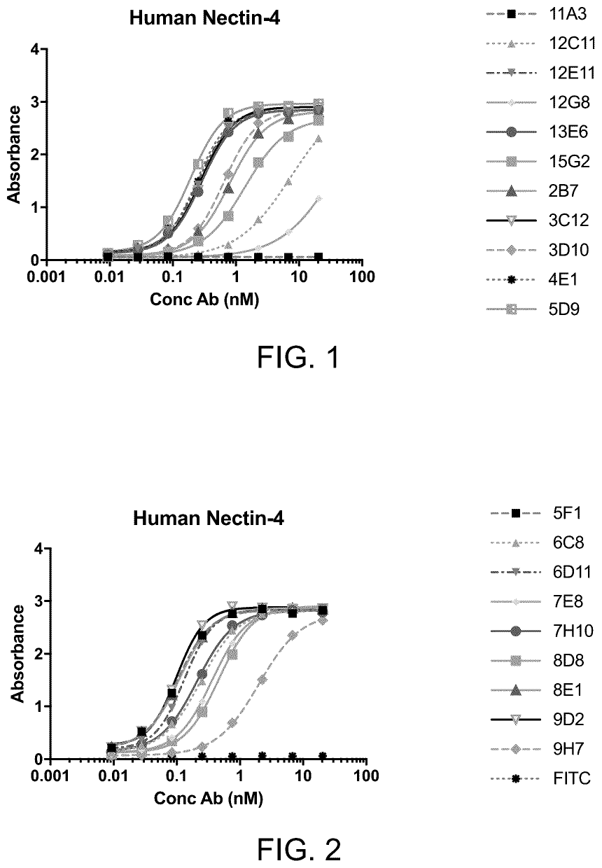

Twenty clones were tested by ELISA for binding to human nectin-4 (FIGS. 1 and 2 and Table 8). From these data, six clones were selected as lead binders: 12E11, 3C12, 5D9, 6C8, 7E8, and 7H10 (sequences shown in Tables 2 and 3).

TABLE 8
ELISA for clones of monoclonal antibodies against Nectin-4

| | Using Hybridoma Supernatants | | | Using |
|---|---|---|---|---|
| | ELISAs | | | Recombinantly |
| | human | cynomolgus- | | Antibody |
| | Nectin-4- | Nectin-4 His | human CD22- | human Nectin-4 |
| | His tagged | tagged | His tagged | His tagged |
| Clone | Ag1 | Ag2 | Ag3 | EC50 (nM) |
| Enfortumab | | | | 0.07-0.23 |
| Nectin-4 11A3H/11A3L | 1.707 | 1.908 | 0.638 | no call |
| Nectin-4 12C11H/12C11L | 3.251 | 3.297 | 0.599 | 7.408 |
| Nectin-4 12E11H/12E11L | 3.214 | 3.439 | 0.583 | 0.252 |
| Nectin-4 12G8H/12G8L | 2.277 | 3.381 | 0.575 | 36.600 |
| Nectin-4 13 E6H/13 E6L | 3.218 | 3.331 | 0.723 | 0.287 |
| Nectin-4 15G2H/15G2L | 3.087 | 3.497 | 0.532 | 1.387 |
| Nectin-4 2B7H/2B7L | 2.328 | 3.529 | 0.559 | 0.793 |
| Nectin-4 3C12H/3C12L | 3.204 | 3.454 | 0.448 | 0.282 |
| Nectin-4 3D10H/3D10L | 1.546 | 3.457 | 0.491 | 0.638 |
| Nectin-4 4 E1H/4 E1L | 3.042 | 3.431 | 0.511 | 0.239 |
| Nectin-4 5D9H/5D9L | 3.080 | 3.316 | 0.515 | 0.192 |
| Nectin-4 5F1H/5F1L | 3.268 | 3.359 | 0.460 | 0.104 |
| Nectin-4 Hybridoma 6C8H/ | 3.164 | 3.401 | 0.456 | 0.239 |
| 6C8L | | | | |
| Nectin-4 6D11H/6D112L | 0.607 | 0.447 | 2.961 | 0.132 |
| Nectin-4 7 E8H/7 E8L | 3.107 | 3.445 | 0.511 | 0.361 |
| Nectin-4 7H10H/7H10L | 3.260 | 3.488 | 0.568 | 0.210 |
| Nectin-4 8D8H/8D8L | 3.349 | 3.461 | 0.429 | 0.462 |
| Nectin-4 8 E1H/8 E1- | 3.179 | 3.533 | 0.514 | 0.112 |
| 9D2L | | | | |
| Nectin-4 9D2H/8 E1- | 3.261 | 3.069 | 0.450 | 0.100 |
| 9D2L | | | | |
| Nectin-4 9H7H/9H7L | 2.325 | 3.238 | 0.535 | 1.992 |

### Nectin-4 Binding ELISA Humanized Variant Results

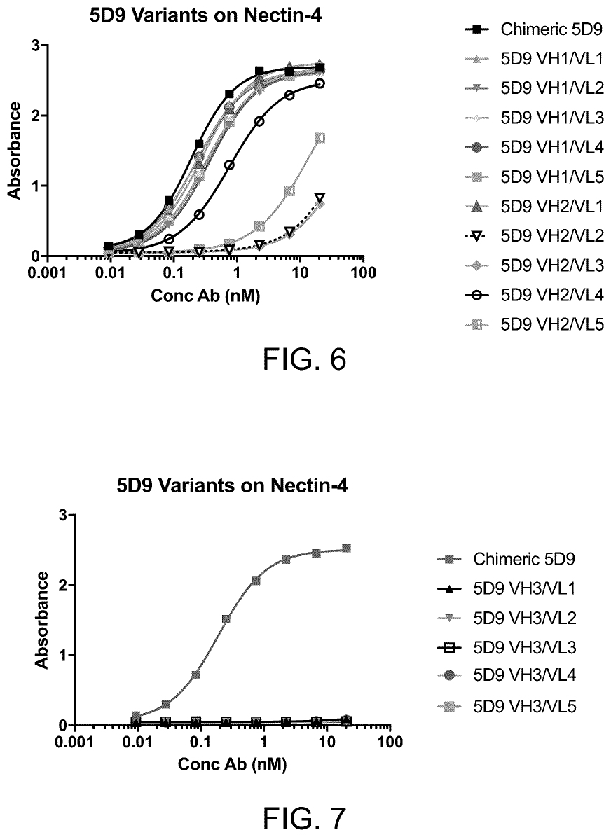

Humanized 5D9 variants (sequences shown in Tables 2 and 3) were produced (titers shown in Table 9) and tested by ELISA for binding to human nectin-4 (FIGS. 6-9). Binding affinity varied across the tested variants (Table 9).

TABLE 9
ELISA titers for 5D9 human variants

| | | ELISAS |
|---|---|---|
| | | EC50 (nM) |
| | Titers (mg/L) | human Nectin-4 |
| Nectin-4 5D9 chimeric | | 0.191 |
| mAb | | |
| Nectin-4 5D9 VH1/VL1 | 426.8 | 0.225 |
| Nectin-4 5D9 VH1/VL2 | 493.9 | 0.368 |
| Nectin-4 5D9 VH1/VL3 | 485.5 | 0.321 |
| Nectin-4 5D9 VH1/VL4 | 379.1 | 0.228 |
| Nectin-4 5D9 VH1/VL5 | 492.7 | 0.346 |
| Nectin-4 5D9 VH2/VL1 | 450.9 | 0.279 |
| Nectin-4 5D9 VH2/VL2 | 436.9 | 171.7 |
| Nectin-4 5D9 VH2/VL3 | 544.5 | 107.6 |
| Nectin-4 5D9 VH2/VL4 | 368.3 | 0.765 |
| Nectin-4 5D9 VH2/VL5 | 384.8 | 14.66 |
| Nectin-4 5D9 VH3/VL1 | 402 | 446629 |
| Nectin-4 5D9 VH3/VL2 | 384.1 | 8.618 |
| Nectin-4 5D9 VH3/VL3 | 464.9 | 23.92 |
| Nectin-4 5D9 VH3/VL4 | 277 | 43608 |
| Nectin-4 5D9 VH3/VL5 | 433.9 | 7.227 |
| Nectin-4 5D9 VH4/VL1 | 564.5 | 0.212 |
| Nectin-4 5D9 VH4/VL2 | 400.6 | 0.236 |
| Nectin-4 5D9 VH4/VL3 | 589.6 | 0.204 |
| Nectin-4 5D9 VH4/VL4 | 444.4 | 0.232 |
| Nectin-4 5D9 VH4/VL5 | 581.8 | 0.195 |
| Nectin-4 5D9 VH5/VL1 | 476.2 | 33.43 |
| Nectin-4 5D9 VH5/VL2 | 353.9 | 2.064 |
| Nectin-4 5D9 VH5/VL3 | 450.8 | 0.702 |
| Nectin-4 5D9 VH5/VL4 | 396.6 | 11.52 |
| Nectin-4 5D9 VH5/VL5 | 561.7 | 56.09 |
| MMAE | N/A | N/A |

*N/A, not applicable

### In Vitro Cytotoxicity Assay Methods

Cell lines were plated in 96-well plates (Costar 3610) at a density of 5×10⁴ cells/well in 100 ML of growth media. The next day cells were treated with 20 μL of test articles serially-diluted in media. After incubation at 37° C. with 5% CO2 for 5 days, viability was measured using the Promega CellTiter Glo® reagent according to the manufacturer's recommendations. GI50 curves were calculated in GraphPad Prism normalized to the payload concentration.

### In Vitro Cytotoxicity Assay Results-Lead Clones

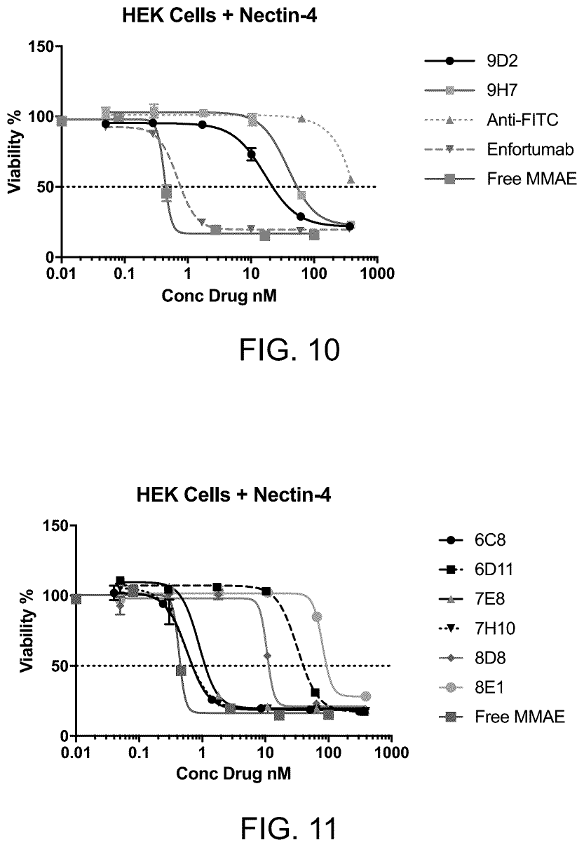

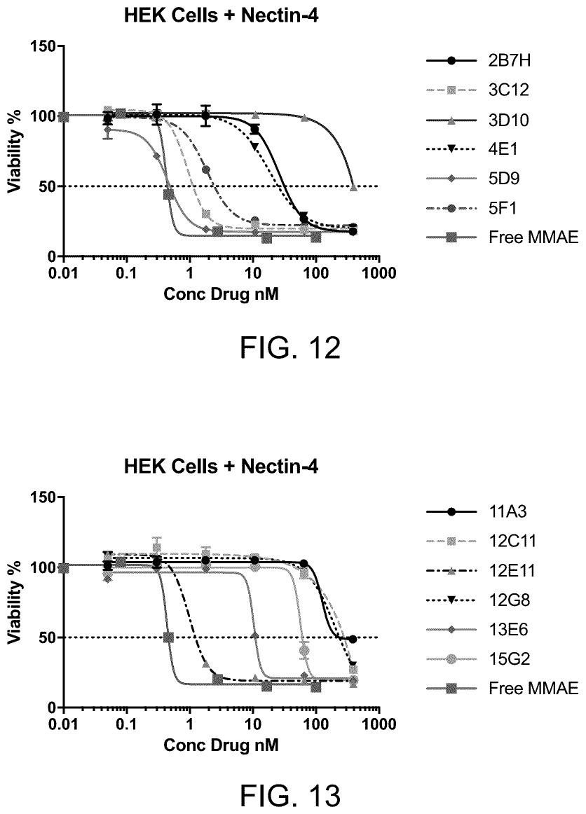

Twenty clones were tested for in vitro potency against HEK 293 cells overexpressing human nectin-4 (FIGS. 10-13, and Table 10). From these data, six clones were selected as lead ADC candidates: 12E11, 3C12, 5D9, 6C8, 7E8, and 7H10 (sequences shown in Tables 1 and 2).

TABLE 10
In vitro chimeric monoclonal antibody potency

| | HEK Cells + Nectin-4 |
|---|---|
| Clone | IC50 (nM) |
| Enfortumab | 0.685 |
| Nectin-4 11A3H/11A3L | 125.000 |
| Nectin-4 12C11H/12C11L | 7536.000 |
| Nectin-4 12E11H/12E11L | 0.987 |
| Nectin-4 12G8H/12G8L | 204.400 |
| Nectin-4 13 E6H/13 E6L | 10.370 |
| Nectin-4 15G2H/15G2L | 56.880 |
| Nectin-4 2B7H/2B7L | 26.180 |
| Nectin-4 3C12H/3C12L | 0.919 |
| Nectin-4 3D10H/3D10L | 5216.000 |
| Nectin-4 4 E1H/4 E1L | 18.320 |
| Nectin-4 5D9H/5D9L | 0.446 |
| Nectin-4 5F1H/5F1L | 1.875 |
| Nectin-4 Hybridoma 6C8H/6C8L | 0.573 |
| Nectin-4 6D11H/6D112L | 32.430 |
| Nectin-4 7 E8H/7 E8L | 0.890 |
| Nectin-4 7H10H/7H10L | 0.549 |
| Nectin-4 8D8H/8D8L | 10.610 |
| Nectin-4 8 E1H/8 E1-9D2L | 80.280 |
| Nectin-4 9D2H/8 E1-9D2L | 16.680 |
| Nectin-4 9H7H/9H7L | 39.030 |

### In Vitro Cytotoxicity Assay Results-Humanized Variants

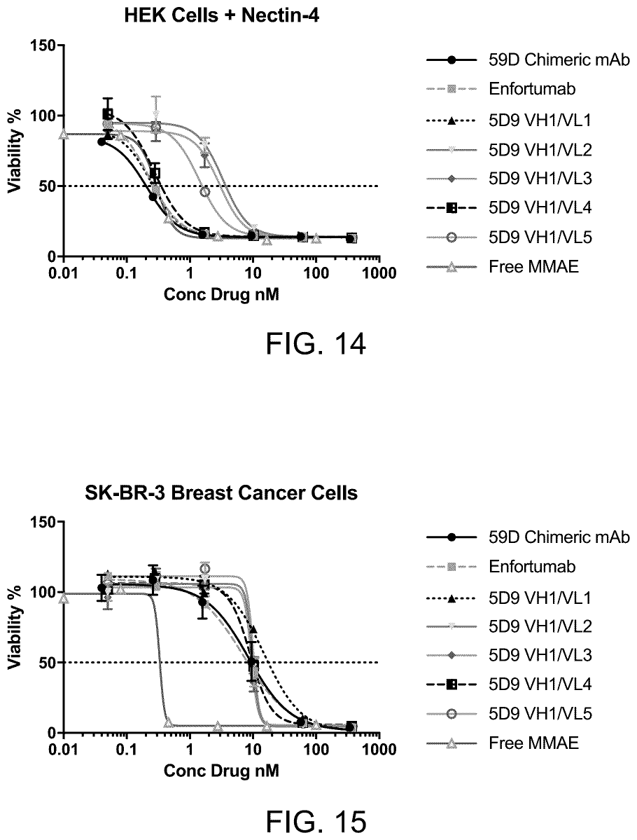

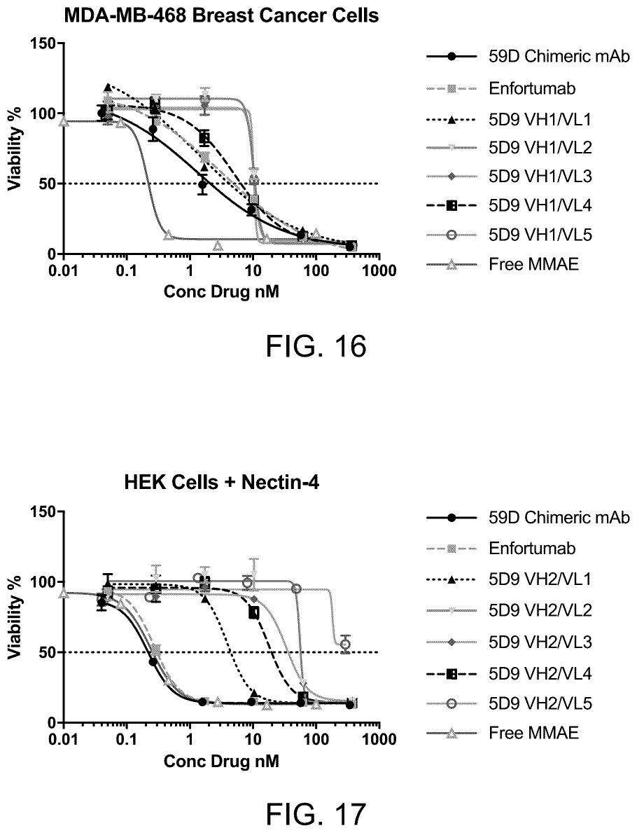

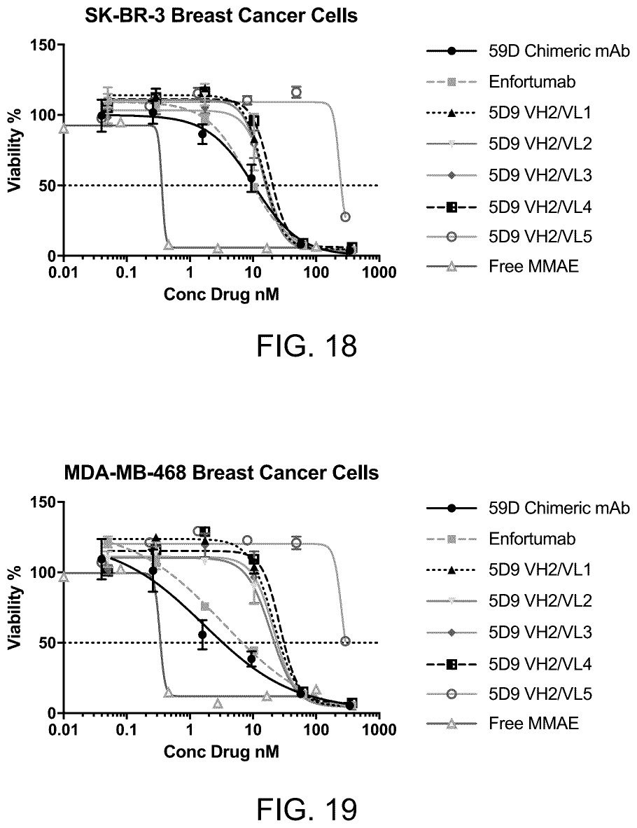

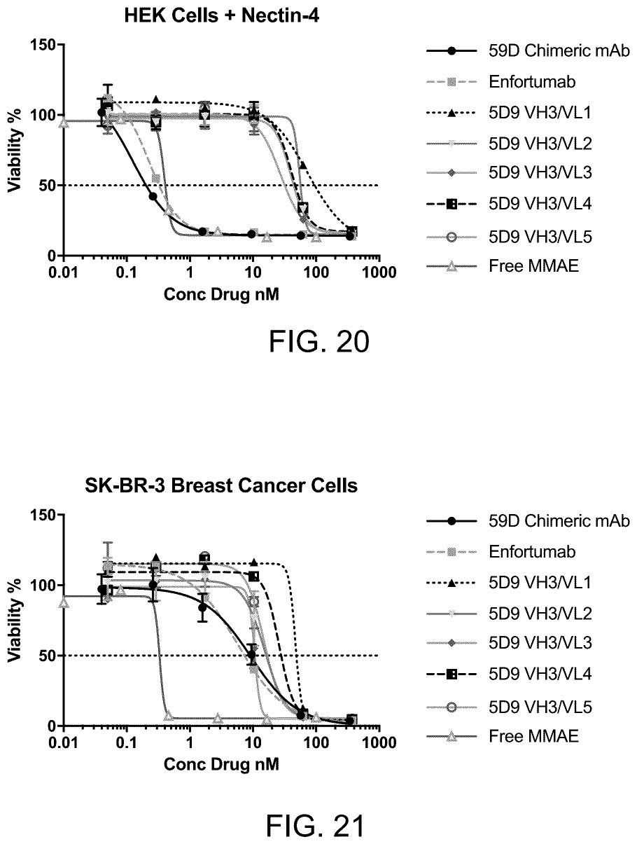

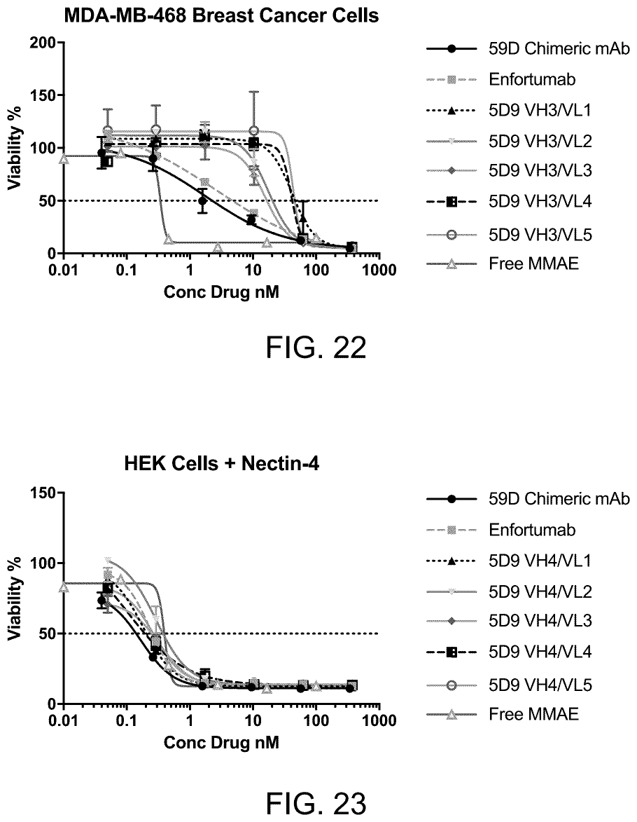

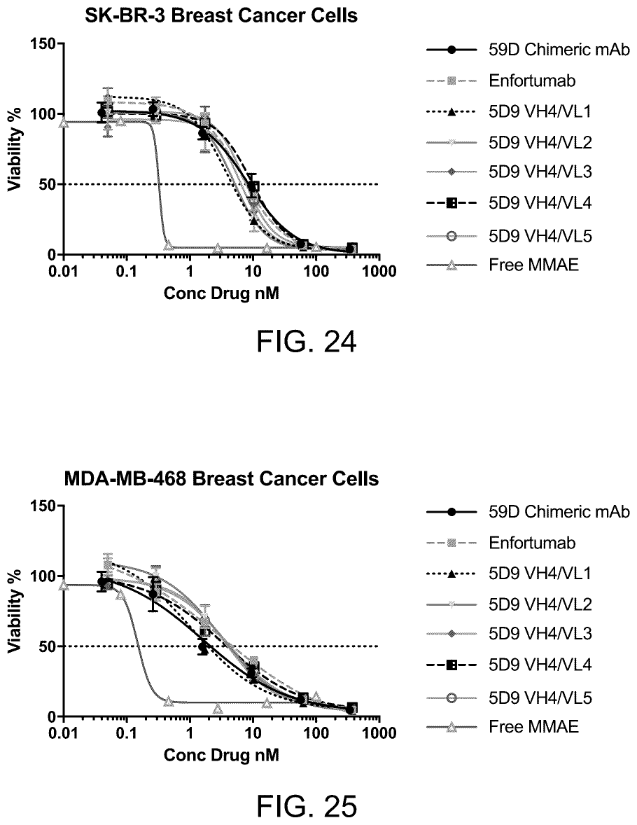

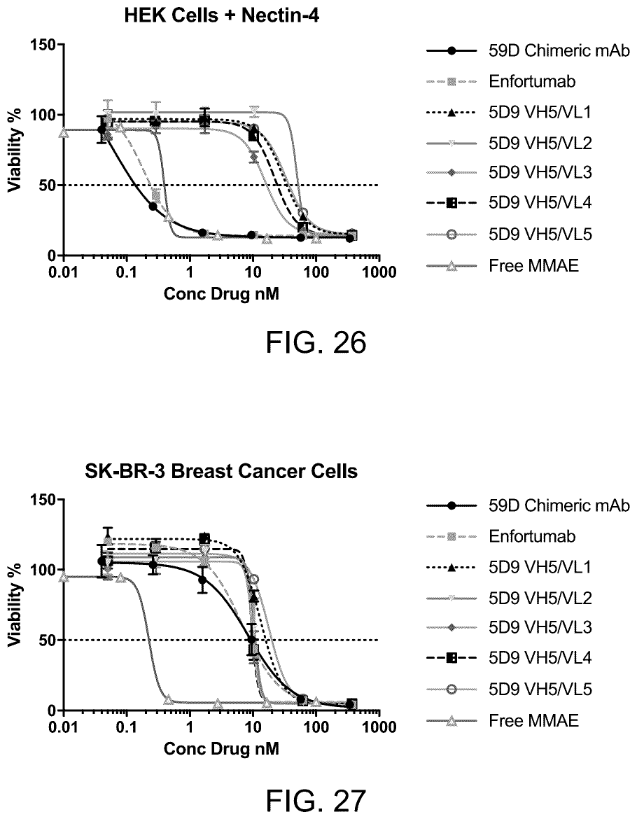

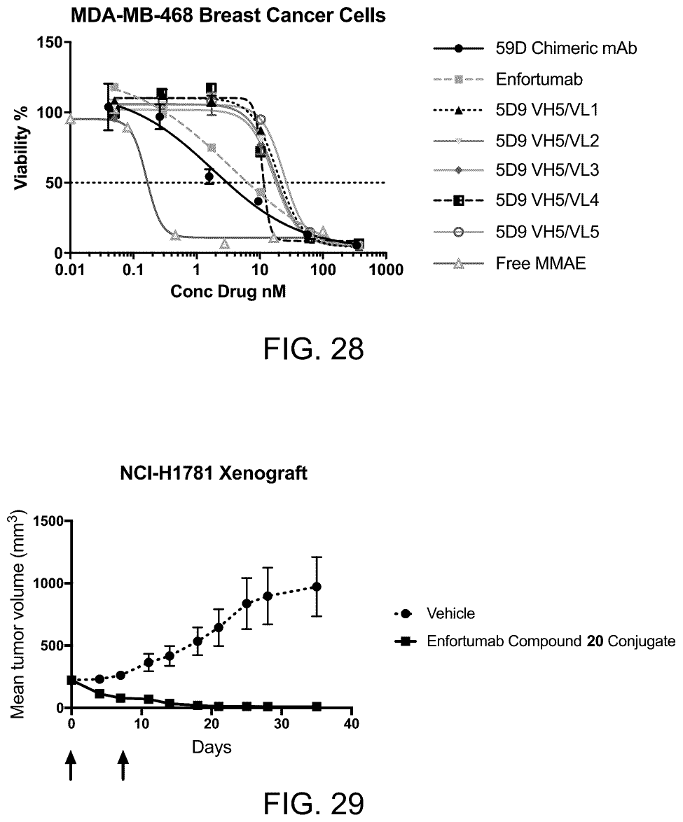

Humanized 5D9 variants (sequences shown in Tables 2 and 3) were produced (titers shown in Table 11) and tested for in vitro potency against HEK 293 cells overexpressing human nectin-4 (FIGS. 14, 17, 20, 23, and 26). Variants were also tested for in vitro potency against the nectin-4 expressing human breast cancer cell lines SK-BR-3 (FIGS. 15, 18, 21, 24, and 27) and MDA-MB-468 (FIGS. 16, 19, 22, 25, and 28). In vitro potency varied across the tested variants (Table 11).

TABLE 11
In vitro potency of tested antibody variants

| | Cell kill | | |
|---|---|---|---|
| | IC50 (nM) | | |
| | HekNectin4 | SKBR3 | MDA-MB468 |
| Nectin-4 5D9 chimeric mAb | 0.207 | 8.47 | 1.307 |
| Nectin-4 5D9 VH1/VL1 | 0.249 | 15.04 | 1.416 |
| Nectin-4 5D9 VH1/VL2 | 3.346 | 9.851 | 10.33 |
| Nectin-4 5D9 VH1/VL3 | 2.945 | 9.361 | 10.31 |
| Nectin-4 5D9 VH1/VL4 | 0.277 | 9.017 | 5.172 |
| Nectin-4 5D9 VH1/VL5 | 1.414 | 10.06 | 10.24 |
| Nectin-4 5D9 VH2/VL1 | 3.933 | 14.66 | 21.35 |
| Nectin-4 5D9 VH2/VL2 | 55.55 | 14.44 | 19.29 |
| Nectin-4 5D9 VH2/VL3 | 33.72 | 15.89 | 21.42 |
| Nectin-4 5D9 VH2/VL4 | 17.77 | 18.49 | 26.96 |
| Nectin-4 5D9 VH2/VL5 | 181.8 | 234.1 | 251.9 |
| Nectin-4 5D9 VH3/VL1 | 73.31 | 46.89 | 41.59 |
| Nectin-4 5D9 VH3/VL2 | 54.78 | 15.3 | 17.68 |
| Nectin-4 5D9 VH3/VL3 | 27.05 | 10.59 | 15.6 |
| Nectin-4 5D9 VH3/VL4 | 41.11 | 25.99 | 40.51 |
| Nectin-4 5D9 VH3/VL5 | 39.99 | 14.94 | 43.28 |
| Nectin-4 5D9 VH4/VL1 | 0.182 | 3.848 | 1.082 |
| Nectin-4 5D9 VH4/VL2 | 0.290 | 4.947 | 3.135 |
| Nectin-4 5D9 VH4/VL3 | 0.333 | 6.456 | 3.713 |
| Nectin-4 5D9 VH4/VL4 | 0.102 | 9.453 | 3.372 |
| Nectin-4 5D9 VH4/VL5 | 0.269 | 7.255 | 3.932 |
| Nectin-4 5D9 VH5/VL1 | 30.29 | 13.31 | 18.98 |
| Nectin-4 5D9 VH5/VL2 | 49.63 | 10.59 | 17.46 |
| Nectin-4 5D9 VH5/VL3 | 15.57 | 9.855 | 16.62 |
| Nectin-4 5D9 VH5/VL4 | 22.09 | 9.527 | 10.97 |
| Nectin-4 5D9 VH5/VL5 | 33.47 | 17.82 | 23.97 |
| MMAE | 0.396 | 0.325 | 0.150 |

### Nectin and Necl Protein Family Reactivity ELISA Results

Six lead clones were tested for reactivity to proteins with homology to nectin-4, specifically the other nectin and necl family members (nectin-1, nectin-2, nectin-3, necl-1, necl-2, necl-3, necl-4, and necl-5).

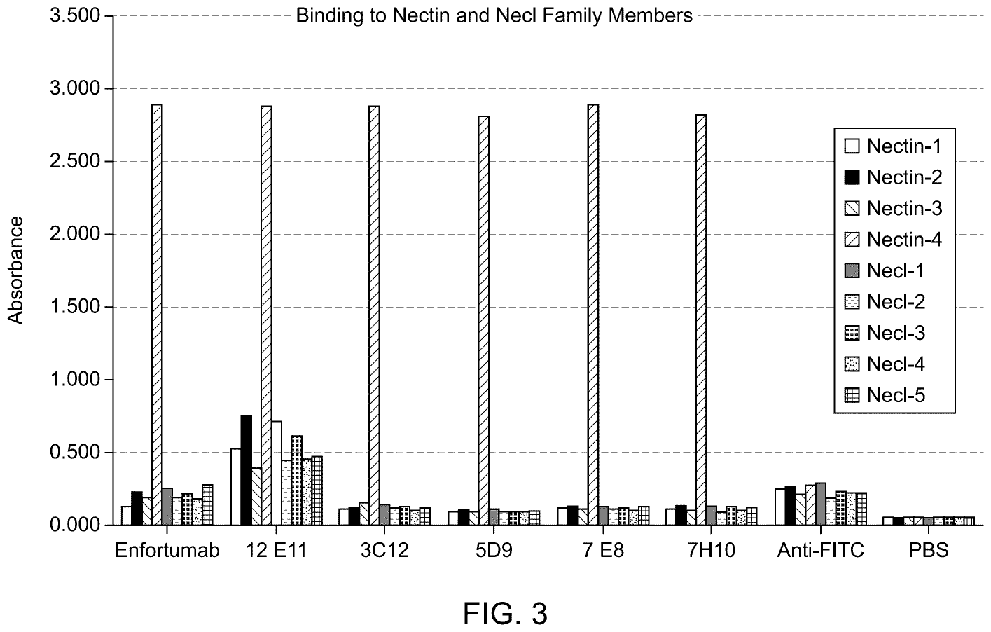

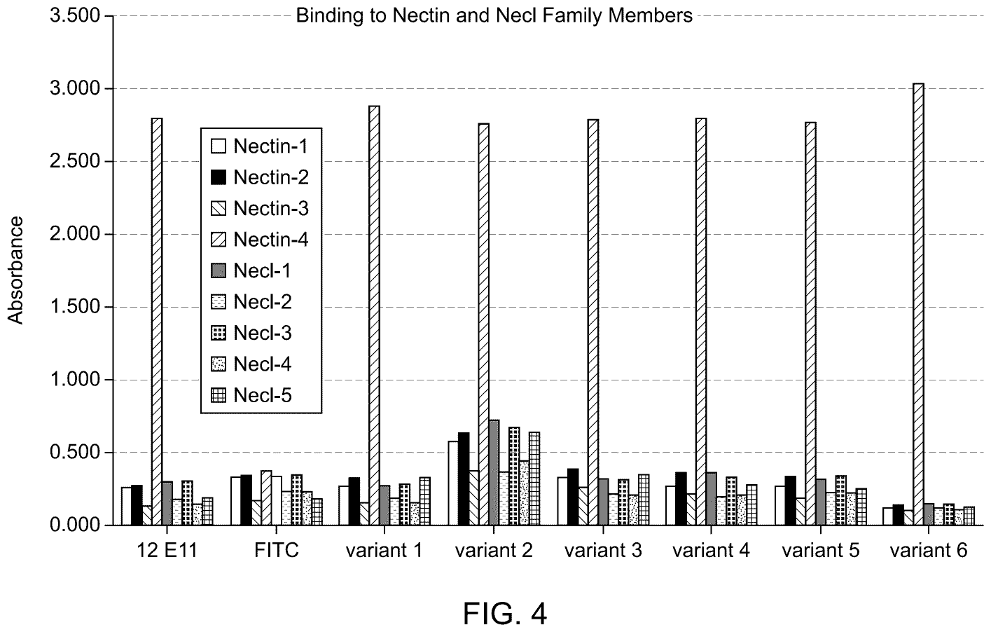

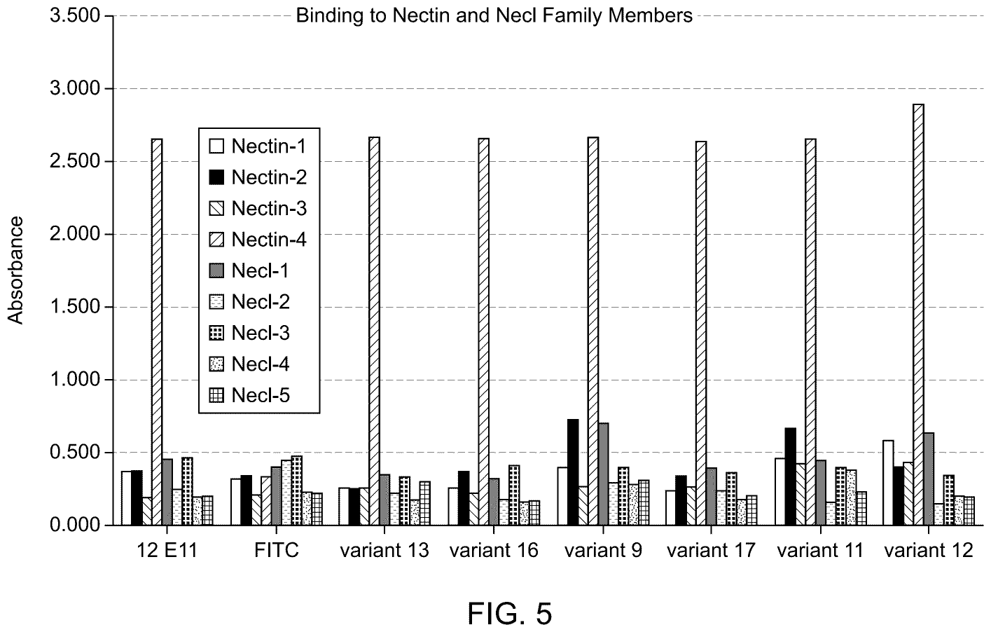

Most clones showed low binding (FIG. 3), with the exception of 12E11, which exhibited low-level cross-reactivity against most nectin-4-related proteins. Accordingly, variants of 12E11 were designed (sequences shown in Tables 2 and 3), produced and re-tested for binding to nectin-4-related proteins (FIGS. 4 and 5). Some variants showed lower reactivity to nectin-4-related proteins as compared to the parental 12E11 clone.

### Nectin-4 Species Reactivity Assessment by Flow Cytometry-Methods

Human embryonic kidney (HEK) 293 cells overexpressing human, cynomolgus monkey, rat, or mouse nectin-4 protein were produced. Cells were lifted with Versene to preserve cell surface proteins and were resuspended at 10e6/mL in PBS+2% FBS. 100 μl was added to a flow tube to test 10⁶ cells/test. Primary antibodies were diluted to 0.1 μg/mL and 10 μL was added for a total of 1 μg/test. Primary antibodies (or ADCs) were incubated with cells for 1 h on ice. Then, cells were washed 1× in 2 mL PBS+2% FBS and secondary antibody was added for detection. AF488-conjugated anti-human antibody from Jackson Immunoresearch, diluted according to the manufacturer's instructions+50% glycerol, was used at 1 μL/test-diluted to ⅕ the concentration in PBS+2% FBS and 5 μL were added per tube. The secondary reagent was incubated with the cells for 30 min, then cells were washed 2× in PBS+2% FBS and analyzed by flow cytometry on a BD FacsCanto instrument equipped with FACSDiva software.

### Nectin-4 Species Reactivity Assessment by Flow Cytometry-Results

Lead clones were tested for binding to human, cynomolgus monkey, rat, or mouse nectin-4 protein expressed on the surface of HEK 293 cells. Enfortumab was included as a positive control and an anti-FITC reactive antibody was included as a negative control. All lead clones bound to human and cynomolgus protein at comparable levels and to rat protein at lower levels (Table 12). Very little to no reactivity was observed to mouse nectin-4 protein, consistent with the fact that the antibodies were produced in mouse.

TABLE 12
Species cross-reactivity of antibody clones.

| | Species cross reactivity | | | | |
|---|---|---|---|---|---|
| | Flow data on Hek293 overexpressing cells | | | | |
| | Human | Cyno | Rat | Mouse | Parental |
| Enfortumab | 17142 | 19990 | 45476 | 3224 | 265 |
| 12E11 | 8622 | 10395 | 1898 | 230 | 232 |
| 3C12 | 8367 | 10617 | 5064 | 328 | 188 |
| 5D9 | 11235 | 12596 | 2143 | 189 | 121 |
| 6C8 | 10217 | 11443 | 8925 | 194 | 121 |
| 7E8 | 9203 | 10437 | 5966 | 279 | 202 |
| 7H10 | 12311 | 14091 | 1293 | 174 | 145 |
| Anti-FITC | 132 | 189 | 196 | 163 | 144 |
| Isotype Control | | | | | |

### Example 2: Synthesis of MMAE Construct 8

Compounds 1 and 4 were obtained commercially from Shanghai Medicilon and used as received. Monomethylauristatin A 5 (MMAE) was purchased from BroadPharm. All other reagents were obtained from commercial sources and used without purification.

### Preparation of (R)-2-(3-(2-((2-(((9H-fluoren-9-yl)methoxy)carbonyl)-1,2-dimethylhydrazineyl)methyl)-1H-pyrrolo[2,3-b]pyridin-1-yl)propanamido)-3-oxo-3-((2-(2-(3-oxo-3-(perfluorophenoxy)propoxy)ethoxy)ethyl)amino)propane-1-sulfonic acid (3)

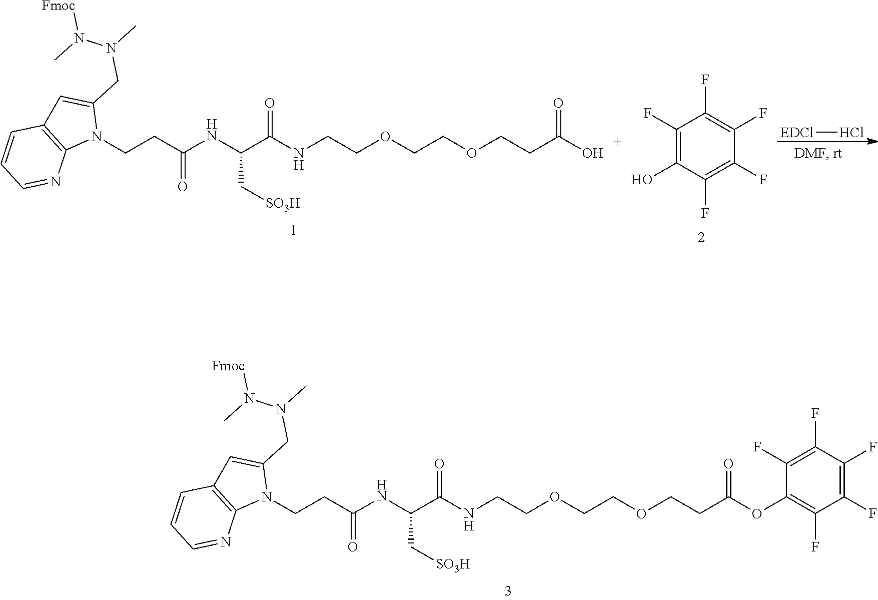

Carboxylic acid 1 (1.33 g, 1.67 mmol) was combined with pentafluorophenol 2 (1.23 g, 6.68 mmol) in 6.5 mL of anhydrous DMF. This mixture was treated with EDCI-HCl (0.64 g, 3.34 mmol) in one portion at room temperature and stirred for 20 h until 1 was fully consumed as judged by HPLC analysis. Reaction mixture was directly purified by reversed-phase chromatography (C18 column, 0-80% acetonitrile-water with 0.05% TFA). Pure fractions were combined, concentrated under vacuum until murky, and lyophilized to give PFP-ester product 3 (1.40 g, 1.46 mmol, 87% yield) as a tan powder. LRMS (ESI): m/z 961.2 [M+H]⁺, Calcd for C₄₄H₄₅F₅N₆O₁₁S m/z 961.3.

### Preparation of (2S,3R,4S,5S,6S)-2-(2-((S)-2-((S)-2-((((9H-fluoren-9-yl)methoxy)carbonyl)amino)-3-methylbutanamido)propanamido)-5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate (6)

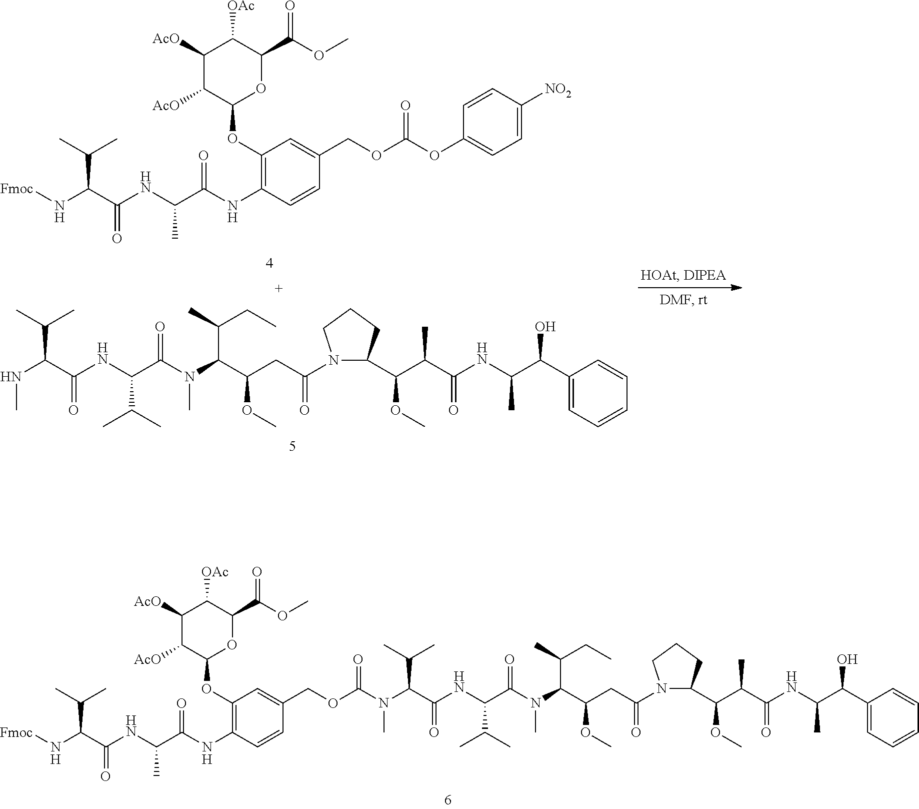

In a 20 mL glass vial were combined monomethyl auristatin A 5 (720 mg, 1.0 mmol), 5 mL of anhydrous DMF, and 0.35 mL of DIPEA (2.0 mmol) at room temperature. The resulting mixture was stirred and treated with PNP carbonate 4 (1014 mg, 1.0 mmol) as a solid in a few small portions, followed by the addition of HOAt (136 mg, 1.0 mmol) in one portion at room temperature. Reaction mixture was stirred for 6 h until reaction was judged complete (HPLC). Reaction mixture was poured into 30 mL of water, and the resulting precipitate was separated by spinning and collected, washed with 5 mL of water, and dried briefly under high vacuum to give 1.87 g of crude product 6 as a yellowish solid, which was taken to the next step without purification.

### Preparation of (2S,3S,4S,5R,6S)-6-(2-((S)-2-((S)-2-amino-3-methylbutanamido)propanamido)-5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (7)

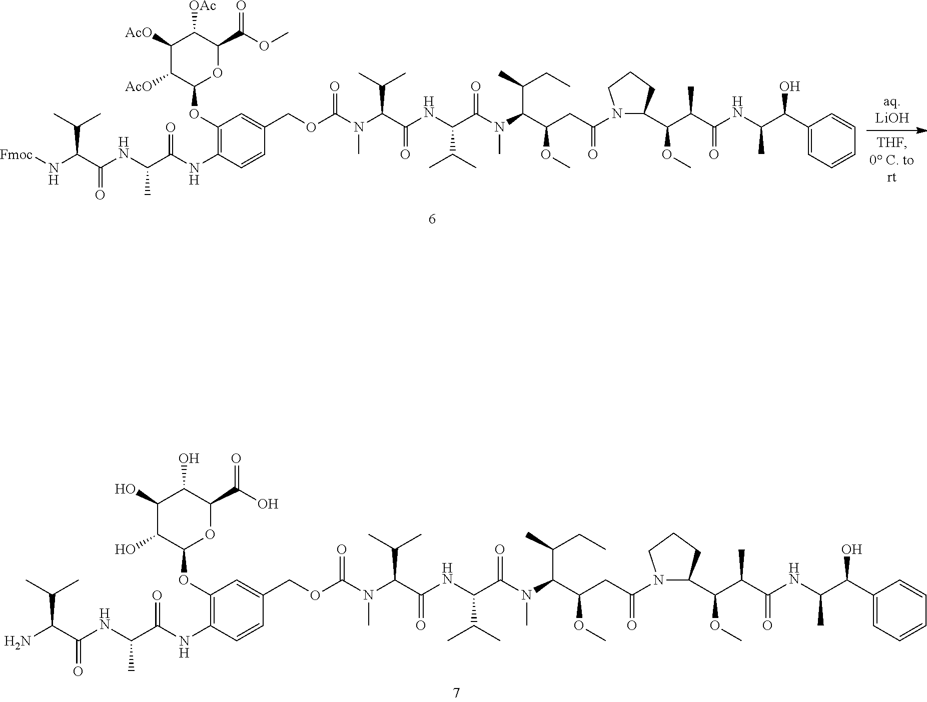

A solution of crude compound 6 (1.87 g) in 15 mL of THF was cooled down to 0° C. in an ice bath and treated slowly with 1 M aqueous lithium hydroxide solution (3 mL). Reaction mixture was stirred at 0° C. for 3 hours, then warmed up to ambient temperature, treated with 3 mL of 1 M aqueous lithium hydroxide and diluted with 3 mL of methanol. The resulting mixture was stirred at room temperature for 3 hours until hydrolysis was complete (HPLC), then quenched by adding 1 M aqueous HCl solution to pH 7. Reaction mixture was then concentrated under reduced pressure and washed with 10 mL of MTBE. Aqueous layer was purified by reversed-phase chromatography (C18 column, 0-40% acetonitrile-water with 0.05% TFA). Pure product fractions were combined, concentrated under reduced pressure, and lyophilized to give compound 7 as a white powder (735 mg, 0.60 mmol, 60% yield over 2 steps). LRMS (ESI): m/z 1229.7 [M+H]⁺, Calcd for C₆₁H₉₆N₈O₁₈ m/z 1229.7.

### Preparation of (2S,3S,4S,5R,6S)-6-(5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-((2S,5S,18R)-22-(2-((1,2-dimethylhydrazineyl)methyl)-1H-pyrrolo[2,3-b]pyridin-1-yl)-5-isopropyl-2-methyl-4,7,17,20-tetraoxo-18-(sulfomethyl)-10,13-dioxa-3,6,16,19-tetraazadocosanamido)phenoxy)-3,4,5-trihydroxytetrahydro-2H-pyran-2-carboxylic acid (8)

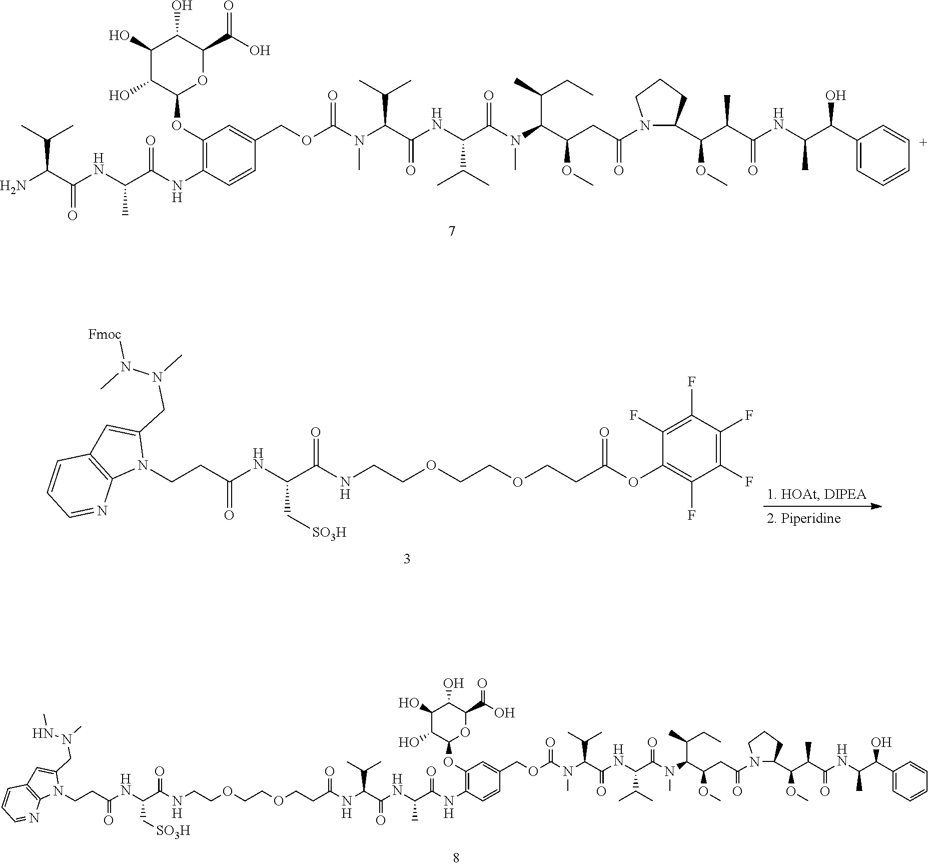

To a stirred solution of compound 7 (735 mg, 0.60 mmol) in 3 mL of anhydrous DMA were added DIPEA (0.21 mL, 1.2 mmol) and a solution PFP-ester 3 (575 mg, 0.60 mmol) in 2 mL of DMA at room temperature, followed by the addition of HOAt (84 mg, 0.60 mmol). The resulting mixture was stirred for 30 minutes until coupling was judged complete (HPLC analysis), then treated directly with 1.2 mL of piperidine at room temperature. After 15 minutes, reaction mixture was purified by reversed-phase chromatography (C18 column, 0-40% gradient of acetonitrile-water). Pure fractions were combined, concentrated under reduced pressure and room temperature, and lyophilized to give compound 8 (808 mg, 0.45 mmol, 75% yield) as a white fluffy powder. LRMS (ESI): m/z 1783.9 [M+H]⁺, Calcd for C₈₄H₁₃₀N₁₄O₂₆S m/z 1783.9.

### Example 3: Syntheses of Belotecan Constructs 20 and 25

Synthetic intermediates 4 and 9 were obtained commercially from Shanghai Medicilon and used as received. Belotecan 15 was purchased from AstaTech. All other reagents were obtained from commercial sources and used without purification.

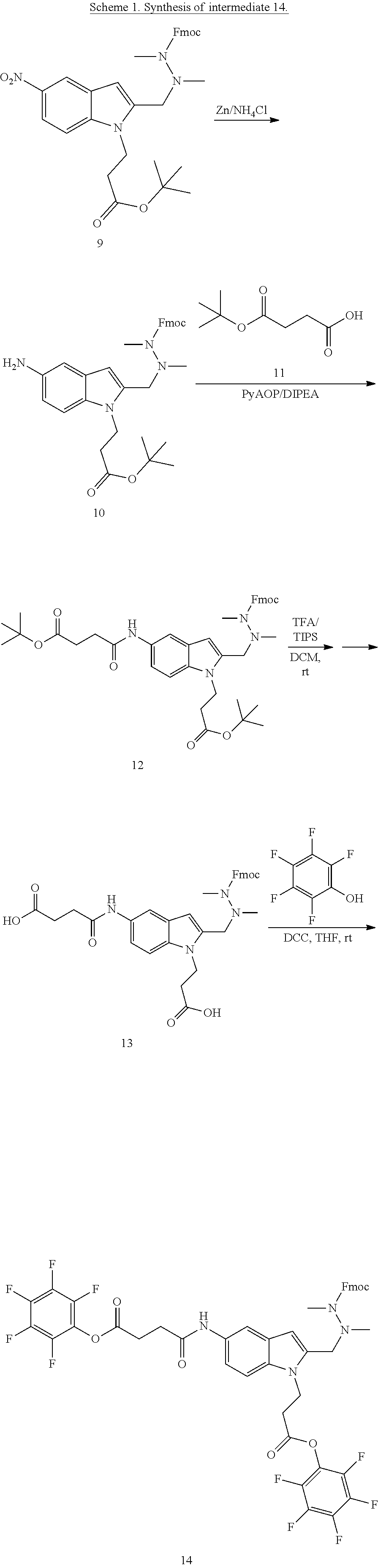

### Preparation of (9H-fluoren-9-yl)methyl 2-((5-amino-1-(3-(tert-butoxy)-3-oxopropyl)-1H-indol-2-yl)methyl)-1,2-dimethylhydrazine-1-carboxylate (10)

Nitro compound 9 (116 mg, 0.20 mmol) was dissolved in 1 mL of THF and combined with a solution of ammonium chloride (85 mg, 1.6 mmol) in 0.5 mL of water and 1 mL of methanol. The resulting mixture was vigorously stirred at room temperature and treated with zinc powder (104 mg, 1.6 mmol) in small portions over 5 minutes. Reaction mixture was stirred for 2 hours, solids were filtered off, filtrate was diluted with 20 mL of saturated aqueous ammonium chloride solution and extracted with ethyl acetate (2×25 mL). Organic extracts were dried over sodium sulfate, solvents removed under vacuum to give crude product 10 which was taken to the next step without purification. LRMS (ESI): m/z 555.3 [M+H]⁺, Calcd for C₃₃H₃₈N₄O₄ m/z 555.3.

### Preparation of (9H-fluoren-9-yl)methyl 2-((1-(3-(tert-butoxy)-3-oxopropyl)-5-(4-(tert-butoxy)-4-oxobutanamido)-1H-indol-2-yl)methyl)-1,2-dimethylhydrazine-1-carboxylate (12)

Crude compound 10 (˜0.20 mmol) was combined with 4-(tert-butoxy)-4-oxobutanoic acid 11 (40 mg, 0.23 mmol) in 2 mL of DMF. To this mixture were added DIPEA (0.12 mL, 0.6 mmol), followed by PyAOP (110 mg, 0.21 mmol) in one portion at room temperature. After 30 minutes, reaction was quenched by pouring into saturated aqueous ammonium chloride, extracted with ethyl acetate, washed with brine, dried over sodium sulfate. Solvent was removed under vacuum to give 120 mg (0.17 mmol, 85% yield over 2 steps) of product 12 as a dark oil which was used further without additional purification. LRMS (ESI): m/z 733.4 [M+Na]⁺, Calcd for C₄₁H₅₀N₄O₇ m/z 733.4.

### Preparation of 4-((2-((2-(((9H-fluoren-9-yl)methoxy)carbonyl)-1,2-dimethylhydrazinyl)methyl)-1-(2-carboxyethyl)-1H-indol-5-yl)amino)-4-oxobutanoic acid (13)

Bis-tert-butyl ester compound 12 (120 mg, 0.17 mmol) was dissolved in a mixture of 2 mL of anhydrous DCM, 2 mL of TFA, and 0.5 mL of trisopropylsilane. The resulting mixture was allowed to stand at room temperature for 4 hours. Solvents were removed under vacuum, and the residue was purified by reversed phase chromatography (C18 column, 0-70% v/v gradient of CH₃CN/H₂O with 0.05% TFA) to obtain 53 mg (0.09 mmol, 53% yield) of diacid product 13. LRMS (ESI): m/z 599.3 [M+H]⁺, Calcd for C₃₃H₃₄N₄O₇ m/z 599.2.

### Preparation of (9H-fluoren-9-yl)methyl 1,2-dimethyl-2-((1-(3-oxo-3-(perfluorophenoxy)propyl)-5-(4-oxo-4-(perfluorophenoxy)butanamido)-1H-indol-2-yl)methyl)hydrazine-1-carboxylate (14)

To a mixture of diacid 13 (50 mg, 0.084 mmol) and pentafluorophenol (46 mg, 0.25 mmol) in 2 mL of anhydrous THF were added DCC (51 mg, 0.25 mmol) in one portion at room temperature. The resulting mixture was stirred for 16 hours, solids were filtered off, filtrate concentrated, and purified by reversed phased chromatography (C18 column, 0-100% v/v gradient of CH₃CN/H₂O with 0.05% TFA). Fractions containing product were concentrated to about 20 mL, poured into 50 mL of 10% aqueous citric acid, and extracted with ethyl acetate (2×20 mL), dried over sodium sulfate. Solvents were removed under vacuum to give 67 mg (0.072 mmol, 86% yield) of bis-PFP ester product 14 as a dark viscous oil. LRMS (ESI): m/z 953.1 [M+Na]⁺, Calcd for C₄₅H₃₂F₁₀N₄O₇ m/z 953.2.

### Preparation of (2S,3S,4S,5R,6S)-6-(2-((S)-2-((S)-2-amino-3-methylbutanamido)propanamido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (16)

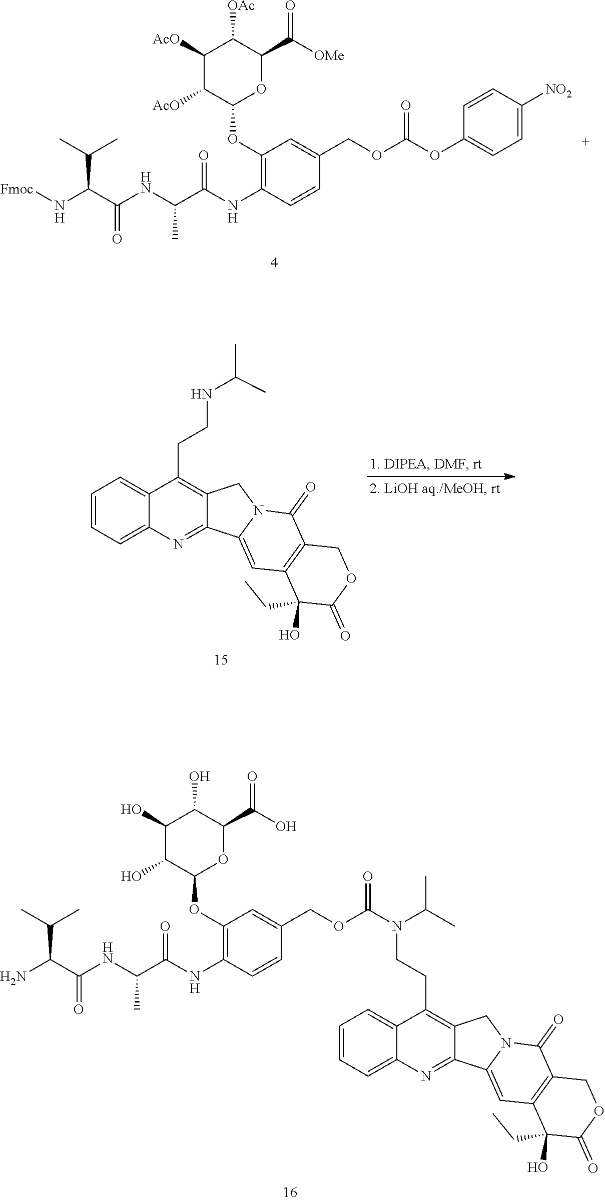

To a solution of belotecan 15 (HCl salt, 20 mg, 43 μmol) in 2 mL DMF were added 15 μL of DIPEA (86 μmol) and 6 mg of HOAt (43 μmol). The resulting mixture was treated with PNP carbonate 4 (43 mg, 43 μmol) at room temperature and stirred for one hour, then DMF was removed under vacuum. The residue was dissolved in 1 mL of MeOH and treated with 1 mL of 1M aqueous LiOH. After 10 minutes, 1 mL of 1M aqueous HCl was added to the mixture, followed by 1 mL of 0.5 M pH 4.7 acetate buffer. The resulting mixture was stirred for 30 minutes at room temperature and directly purified by reversed phase HPLC (C18 column, 0-50% v/v gradient of CH₃CN/H₂O with 0.05% TFA). Solvent was removed under vacuum to give 17 mg (18 μmol, 43% yield) of compound 16 as a glassy yellow solid. LRMS (ESI): m/z 945.4 [M+H]⁺, Calcd for C₄₇H₅₆N₆O₁₅ m/z 945.4.

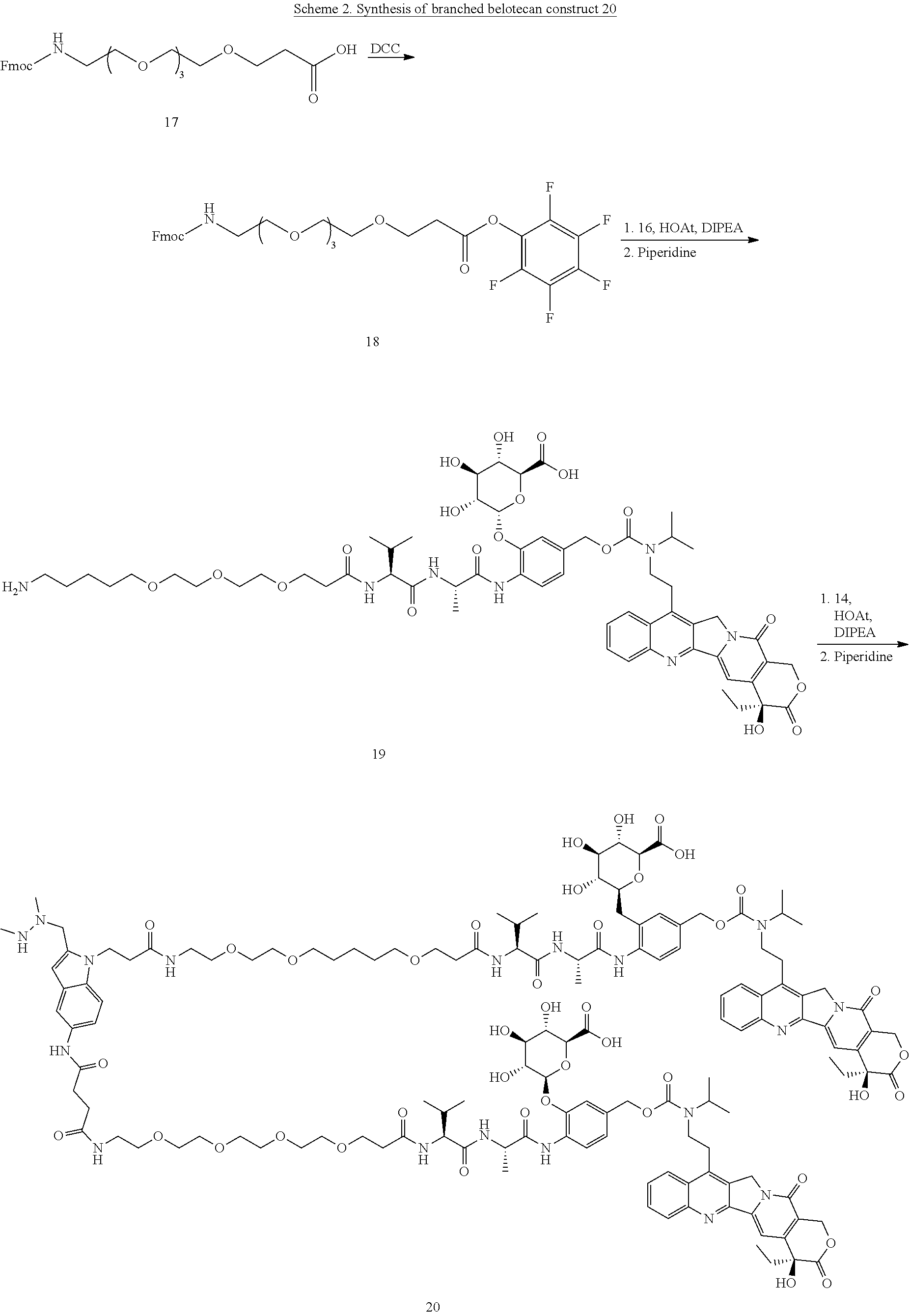

### Preparation of perfluorophenyl 1-(9H-fluoren-9-yl)-3-oxo-2,7,10,13,16-pentaoxa-4-azanonadecan-19-oate (18)

In an oven-dried scintillation vial were combined 1-(9H-fluoren-9-yl)-3-oxo-2,7,10,13,16-pentaoxa-4-azanonadecan-19-oic acid (17, 487 mg, 1 mmol) and pentafluorophenol (368 mg, 2 mmol) in 5 mL of anhydrous THF. The resulting mixture was treated with DCC (247 mg, 1.2 mmol) in one portion at room temperature, and reaction mixture was stirred overnight. Precipitated solids were filtered off, solvents removed under vacuum, and the residue was purified by reversed-phase chromatography (C18 column, 10-100% v/v gradient of CH₃CN/H₂O with 0.05% TFA) to give 670 mg of PFP ester 18 (570 mg, 0.87 mmol, 87% yield) as a colorless oil. LRMS (ESI): m/z 654.2 [M+H]⁺, Calcd for C₃₂H₃₂F₅NO₈ m/z 654.2.

### Preparation of (2S,3S,4S,5R,6S)-6-(2-((17S,20S)-1-amino-7-isopropyl-20-methyl-15,18-dioxo-3,6,9,12-tetraoxa-16,19-diazahenicosan-21-amido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (19)

Compound 16 (262 mg, 0.22 mmol) was dissolved in 4 mL of DMF. To this solution were added DIPEA (105 μL, 0.66 mmol) and PFP ester 18 (181 mg, 0.22 mmol) as a solution in 0.5 mL of DMF at room temperature, followed by the addition of HOAt (38 mg, 0.22 mmol). The resulting mixture was allowed to stand at room temperature for one hour, then treated directly with 4 mL of triethylamine. Reaction mixture was stirred for 5 hours, until Fmoc-deprotection was complete as judged by HPLC analysis. Reaction mixture was concentrated under vacuum and purified by reversed-phase chromatography (C18 column, 0-50% v/v gradient of CH₃CN/H₂O with 0.05% TFA) to give 185 mg (0.16 mmol, 73% yield) of compound 19 as a yellow solid. LRMS (ESI): m/z 1192.5 [M+H]⁺, Calcd for C₅₈H₇₇N₇O₂ m/z 1192.5.

### Preparation of (2S,3S,4S,5R,6S)-6-(2-((2S,5S)-25-(5-((2S,5S)-1-((2-(((2S,3R,4S,5S,6S)-6-carboxy-3,4,5-trihydroxytetrahydro-2H-pyran-2-yl)oxy)-4-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-5-isopropyl-2-methyl-1,4,7,23-tetraoxo-10,13,16,19-tetraoxa-3,6,22-triazahexacosan-26-amido)-2-((1,2-dimethylhydrazinyl)methyl)-1H-indol-1-yl)-5-isopropyl-2-methyl-4,7,23-trioxo-10,13,16,19-tetraoxa-3,6,22-triazapentacosanamido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (20)

Compound 19 (23 mg, 19 μmol) was dissolved in 2 mL of anhydrous DMA. To this solution were added DIPEA (10 μL, 57 μmol) and bis-PFP ester 14 (8 mg, 8.6 μmol) as solid in one portion at room temperature, followed by HOAt (2.6 mg, 19 μmol). The resulting mixture was allowed to stand at room temperature for one hour, then treated directly with 17 μL of piperidine (172 μmol). After 20 minutes, reaction mixture was purified by reversed-phase prep HPLC (C18 column, 0-50% v/v gradient of CH₃CN/H₂O with 0.05% TFA). Pure fractions were lyophilized to give 5.8 mg (2.1 μmol, 24% yield) of compound 20 as a yellow powder. LRMS (ESI): m/z 1363.1 [M+2H]⁺⁺, Calcd for C₁₃₄H₁₇₄N₁₈O₄₃ m/z 1362.6.

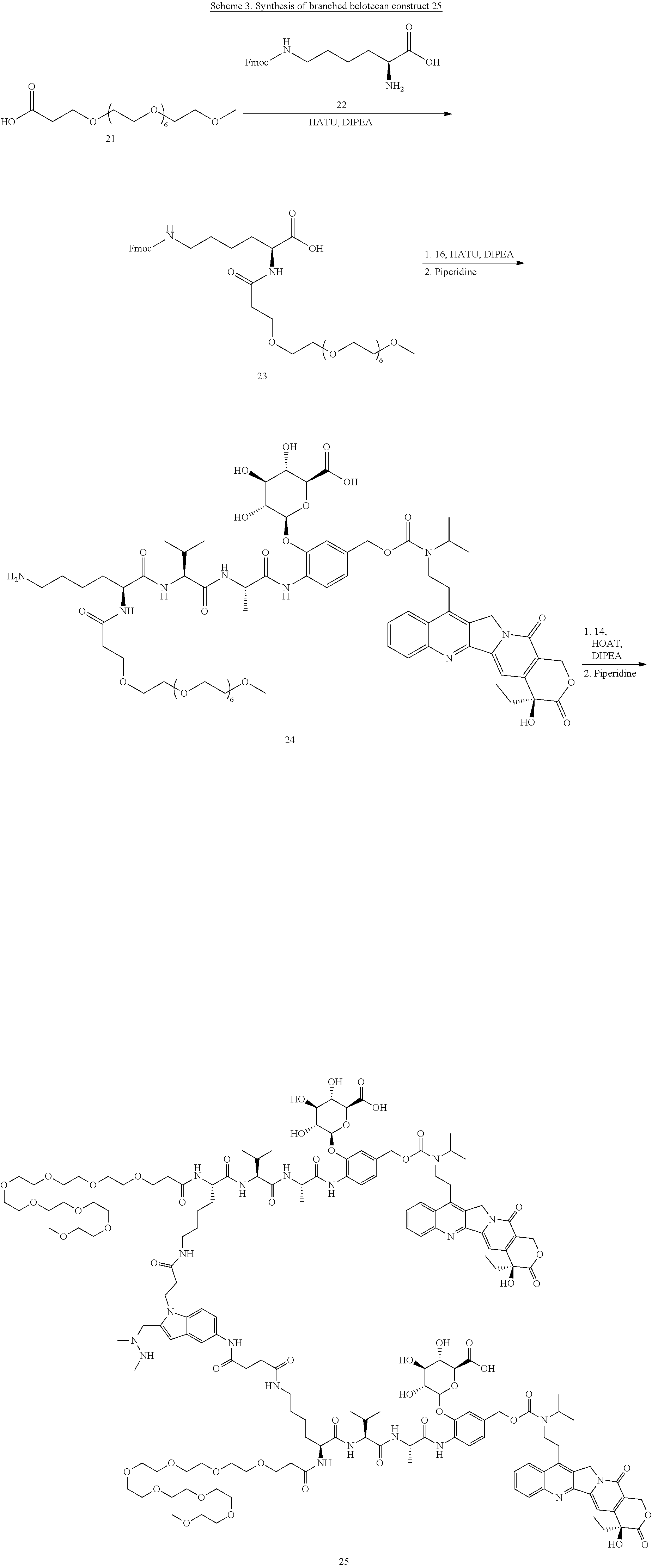

### Preparation of N⁶-(((9H-fluoren-9-yl)methoxy)carbonyl)-N²-(3-(2-(2-methoxyethoxy)ethoxy)propanoyl)-L-lysine (23)

To a solution of mPEG8-acid 21 (100 mg, 0.24 mmol) in 2 mL of anhydrous DMF were added DIPEA (0.13 mL, 0.72 mmol) and HATU (93 mg, 0.24 mmol) at room temperature. The resulting mixture was stirred for one hour, then Lys(Fmoc)-OH 22 (89 mg, 0.24 mmol) was added to the mixture, and stirring continued for one hour. Reaction mixture was directly purified by reversed-phase chromatography HPLC (C18, 0-70% v/v MeCN—H₂O with 0.05% TFA) to give 120 mg of compound 23 (0.16 mmol, 67% yield) as a colorless oil. LRMS (ESI): m/z 763.4 [M+H]⁺, Calcd for C₃₉H₅₈N₂O₁₃ m/z 763.4.

### Preparation of (2S,3S,4S,5R,6S)-6-(2-((28S,31S,34S)-28-(4-aminobutyl)-31-isopropyl-34-methyl-26,29,32-trioxo-2,5,8,11,14,17,20,23-octaoxa-27,30,33-triazapentatriacontan-35-amido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (24)

To a solution of carboxylic acid 23 (45 mg, 59 μmol) in 3 mL of anhydrous DMF were added DIPEA (21 μL, 120 μmol) and HATU (22 mg, 59 μmol) at room temperature. The resulting mixture was stirred for 20 minutes and combined with amine 16 (55 mg, 58 μmol) in 1 mL of DMF. Reaction mixture was stirred for 30 minutes, then piperidine (115 μL, 1.2 mmol) was added to the mixture at room temperature. After 20 minutes, reaction mixture was directly purified by reversed phase prep HPLC (C18, 0-50% v/v MeCN—H₂O with 0.05% TFA). Lyophilization of pure fractions afforded 34 mg (23 μmol, 40% yield) of compound 24 as a yellow powder. LRMS (ESI): m/z 1467.7 [M+H]⁺, Calcd for C₇₁H₁₀₂N₈O₂₅ m/z 1467.7.

### Preparation of (2S,3S,4S,5R,6S)-6-(2-((28S,31S,34S)-28-(4-(3-(5-((S)-28-(((S)-1-(((S)-1-((2-(((2S,3R,4S,5S,6S)-6-carboxy-3,4,5-trihydroxytetrahydro-2H-pyran-2-yl)oxy)-4-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1-oxopropan-2-yl)amino)-3-methyl-1-oxobutan-2-yl)carbamoyl)-26,34-dioxo-2,5,8,11,14,17,20,23-octaoxa-27,33-diazaheptatriacontan-37-amido)-2-((1,2-dimethylhydrazineyl)methyl)-1H-indol-1-yl)propanamido)butyl)-31-isopropyl-34-methyl-26,29,32-trioxo-2,5,8,11,14,17,20,23-octaoxa-27,30,33-triazapentatriacontan-35-amido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (25)

To a mixture of compound 24 (34 mg, 23 μmol) and DIPEA (8 μL, 46 μmol) in 2 mL of DMA were added bis-PFP ester 14 (9.4 mg, 10.5 μmol), followed by HOAt (3 mg, 23 μmol) at room temperature. The resulting mixture was allowed to stand for 30 minutes at room temperature, then piperidine (21 μL, 0.21 mmol) was added to the mixture at room temperature. After 20 minutes, reaction mixture was directly purified by reversed phase prep HPLC (C18, 0-50% v/v MeCN—H₂O with 0.05% TFA). Pure fractions were combined and lyophilized to afford compound 25 as a yellow solid (23 mg, 7 μmol, 67% yield). LRMS (ESI): m/z 1638.3 [M+H]²⁺, Calcd for C₁₆₀H₂₂₄N₂₀O₅₃ m/z 1638.8.

### Example 4

### Bioconjugation, Purification, and HPLC Analytics

Antibodies (15 mg/mL) bearing one aldehyde tag were conjugated to linker-payloads at 1.1 mM, respectively. Reactions proceeded for 72 h at 37° C. in 20 mM sodium citrate, 50 mM NaCl pH 5.5 (20/50 buffer) containing 0.85-2.5% DMA. After conjugation, free drug was removed using a 30 kD MWCO 0.5 mL Amicon spin concentrator. Samples were added to the spin concentrator, centrifuged at 15,000×g for 7 min, then diluted with 450 μL 20 mM sodium citrate, 50 mM NaCl pH 5.5 and centrifuged again. The process was repeated 10 times. To determine the DAR of the final product, ADCs were examined by analytical chromatography using HIC (Tosoh #14947) or PLRP-RP (Agilent PL1912-1802 1000 A, 8 μm, 50×2.1 mm) columns. HIC analysis used mobile phase A: 1.5 M ammonium sulfate, 25 mM sodium phosphate pH 7.0, and mobile phase B: 25% isopropanol, 18.75 mM sodium phosphate pH 7.0. PLRP analysis used mobile phase A: 0.1% trifluoroacetic acid in water, and mobile phase B: 0.1% trifluoroacetic acid in acetonitrile. Prior to PLRP analysis, sample was denatured with the addition of 50 mM DTT, 4 M guanidine HCl (final concentrations) and heating at 37° C. for 30 min. To determine aggregation, samples were analyzed using analytical size exclusion chromatography (SEC; Tosoh #08541) with a mobile phase of 300 mM NaCl, 25 mM sodium phosphate pH 6.8 with 5% isopropanol.

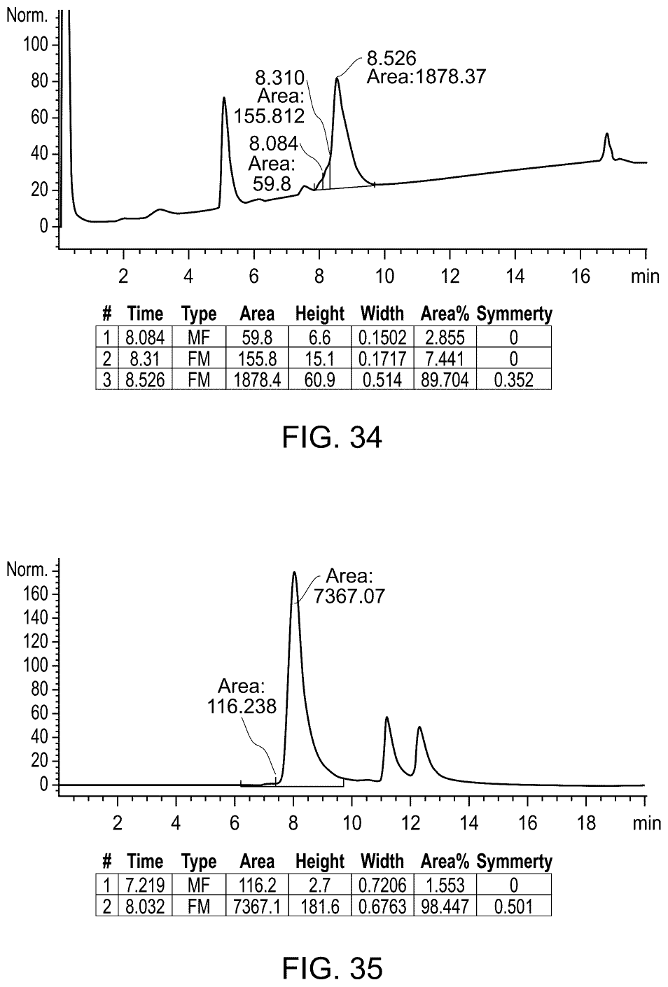

FIG. 34. Double-tagged Nectin-4 VH4/VL1 antibody conjugated to Compound 8 yields a DAR of 3.74 as determined by PLRP.

FIG. 35. Double-tagged Nectin-4 VH4/VL1 antibody conjugated to Compound 8 is 98.5% monomeric as determined by SEC.

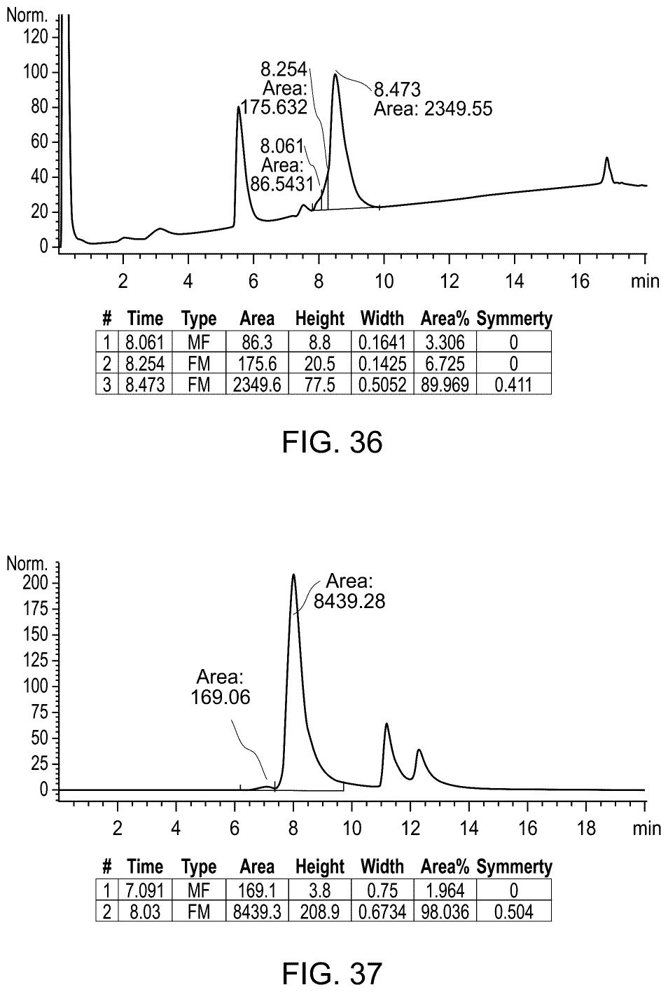

FIG. 36. Double-tagged Nectin-4 VH4/VL5 antibody conjugated to Compound 8 yields a DAR of 3.73 as determined by PLRP.

FIG. 37. Double-tagged Nectin-4 VH4/VL5 antibody conjugated to Compound 8 is 98.0% monomeric as determined by SEC.

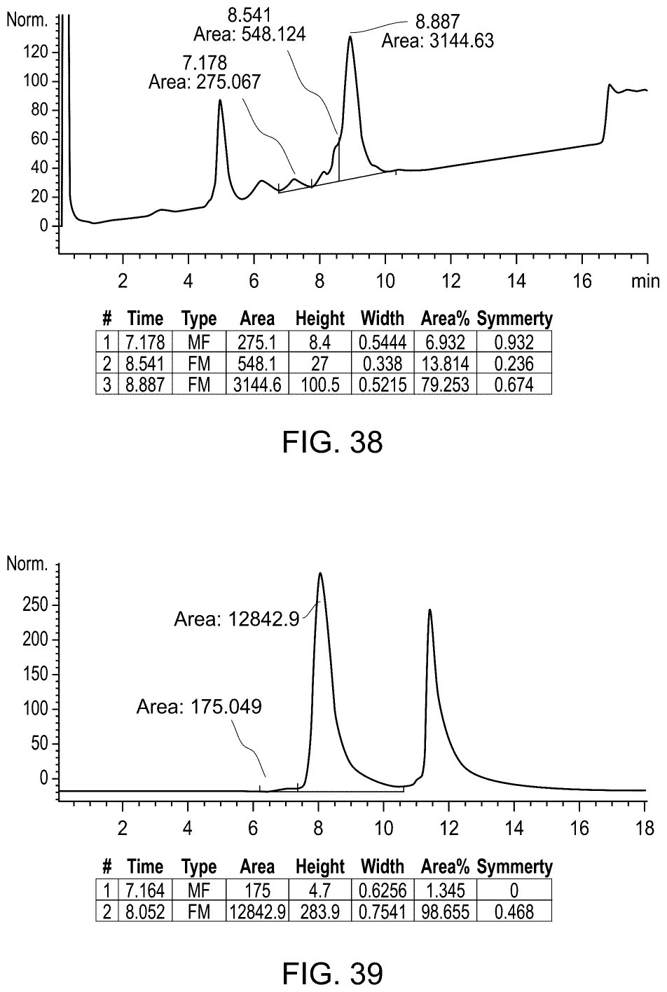

FIG. 38. Double-tagged Nectin-4 VH4/VL1 antibody conjugated to Compound 25 yields a DAR of 6.89 as determined by PLRP.

FIG. 39. Double-tagged Nectin-4 VH4/VL1 antibody conjugated to Compound 25 is 98.7% monomeric as determined by SEC.

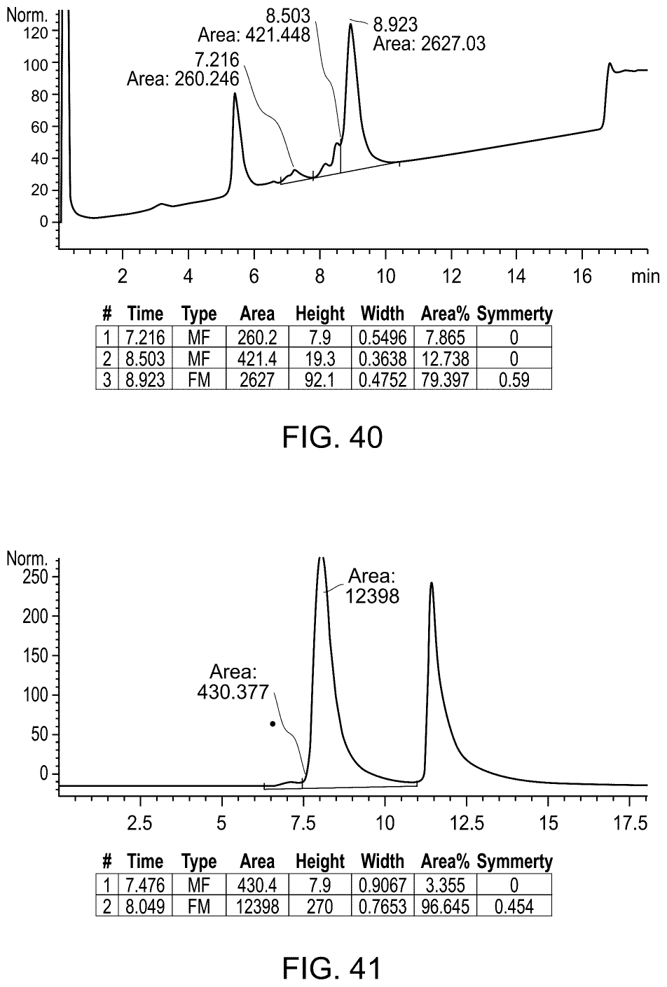

FIG. 40. Double-tagged Nectin-4 VH4/VL5 antibody conjugated to Compound 25 yields a DAR of 6.86 as determined by PLRP.

FIG. 41. Double-tagged Nectin-4 VH4/VL5 antibody conjugated to Compound 25 is 96.6% monomeric as determined by SEC.

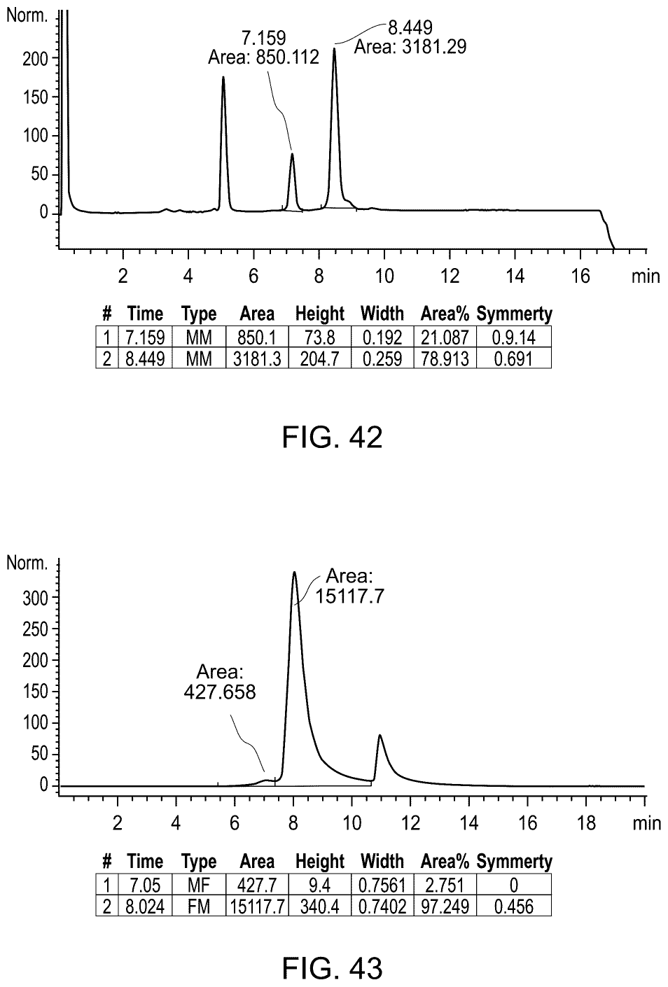

FIG. 42. Single-tagged Nectin-4 VH4/VL1 antibody conjugated to Compound 25 yields a DAR of 3.16 as determined by PLRP.

FIG. 43. Single-tagged Nectin-4 VH4/VL1 antibody conjugated to Compound 25 is 97.2% monomeric as determined by SEC.

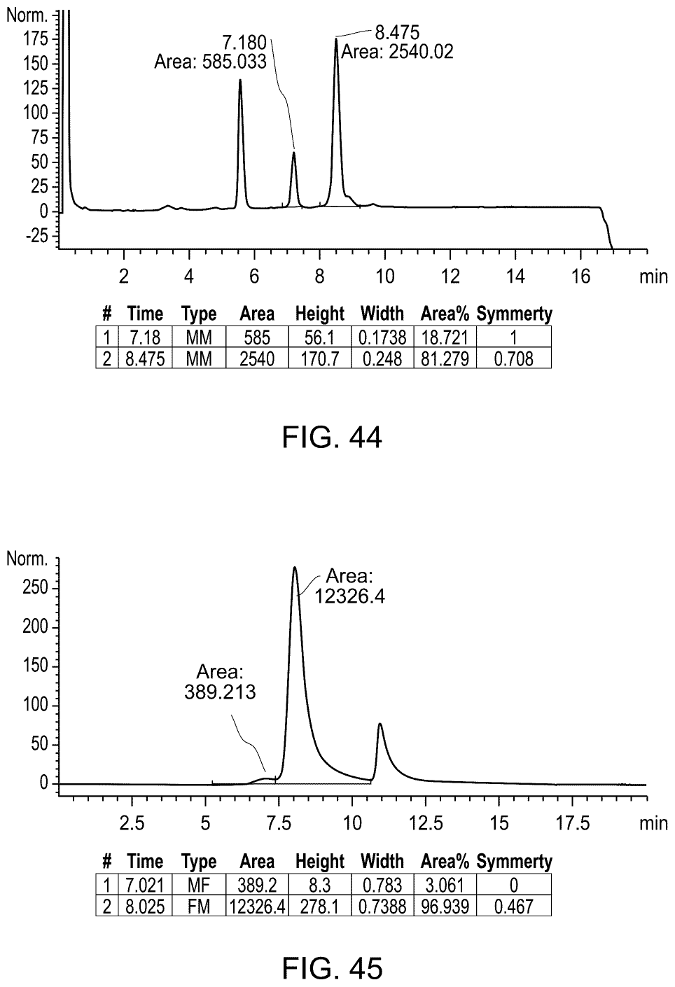

FIG. 44. Single-tagged Nectin-4 VH4/VL5 antibody conjugated to Compound 25 yields a DAR of 3.25 as determined by PLRP.

FIG. 45. Single-tagged Nectin-4 VH4/VL5 antibody conjugated to Compound 25 yields a DAR of 3.25 as determined by PLRP.

### Example 5: Xenograft Studies

### NCI-H1781 Xenograft Methods:

Female BALB/c nude mice (5 per group) were inoculated subcutaneously with 20 million NCI-H1781 cells in PBS. Treatment began when the tumors reached an average of 222 mm³ (Day 1). Animals were dosed intravenously with vehicle alone or with an enfortumab antibody carrying two aldehyde tag insertions conjugated to RED-674 bearing a DAR of 6.8. ADCs were dosed intravenously at 5 mg/kg on Days 0 and 7. The animals were monitored twice weekly for body weight and tumor size. Animals were euthanized when tumors reached 2000 mm3 or body weight loss exceeded 15%.

### NCI-H1781 Xenograft Results:

The aldehyde conjugated nectin-4 targeted ADC bearing a topoisomerase I inhibitor payload showed strong tumor regression, including complete responses in two out of five animals carrying NCI-H1781 xenografts (FIG. 29). By the end of the study (Day 35), the average tumor size (±SD) in the vehicle control and ADC-treated groups was 971±237 mm³ and 10±10 mm³, respectively.

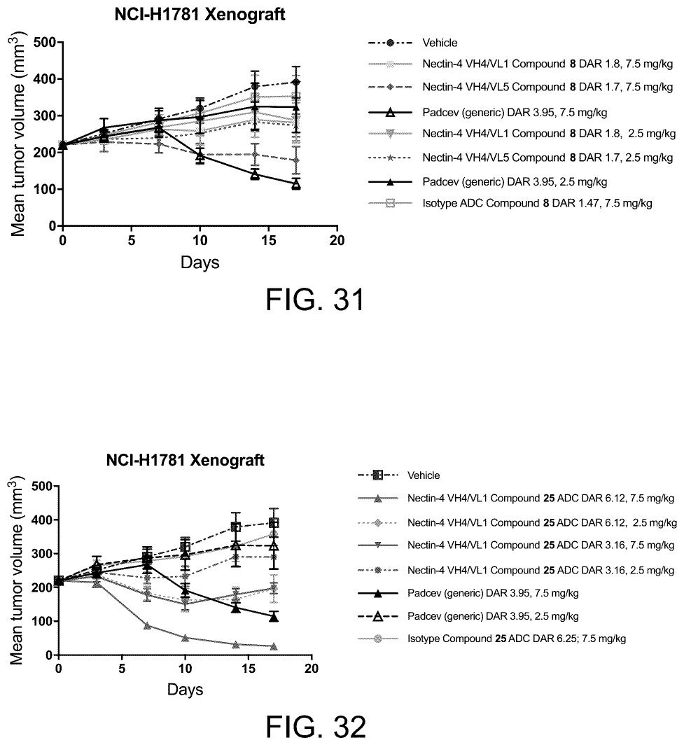

FIG. 31 shows a graph of an NCI-H1781 xenograft study with a single 2.5 or 7.5 mg/kg intravenous dose of the listed anti-nectin-4 ADC on Day 0. VH4/VL1 Compound 8 (RED-601) and VH4/VL5 Compound 8 both use the internal 91N tag and deliver half the payload dose as compared to Padcev. The isotype control ADC had minimal activity.

FIG. 32 shows a graph of an NCI-H1781 xenograft study with a single 2.5 or 7.5 mg/kg intravenous dose of the listed anti-nectin-4 or isotype control ADC on Day 0. VH4/VL1 Compound 25 (RED-694) was made in a DAR4 format using the 91N tag and in a DAR8 format using the 91N/116E double tag combination. Padcev (generic) was included as a comparator. The isotype control Compound 25 ADC had minimal activity.

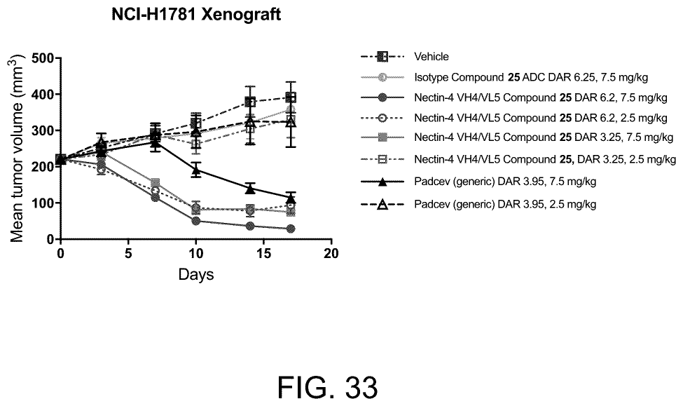

FIG. 33 shows a graph of an NCI-H1781 xenograft study with a single 2.5 or 7.5 mg/kg intravenous dose of the listed anti-nectin-4 or isotype control ADC on Day 0. VH4/VL5 Compound 25 (RED-694) was made in a DAR4 format using the 91N tag and in a DAR8 format using the 91N/116E double tag combination. Padcev (generic) was included as a comparator. The isotype control Compound 25 ADC had minimal activity.

### Example 6: Toxicity Studies

Toxicity studies were performed which showed improved tolerability of a nectin-4 CH1/CT aldehyde-tagged enfortumab antibody conjugated to a topo I linker-payload (Compound 25) as compared to a vedotin-conjugated enfortumab antibody.

### ADCs Used in Multi-Dose Rat Toxicity Study

| Linker- | | | |
|---|---|---|---|
| Payload | Antibody | DAR | % Monomer |
| Compound 25 | Heavy chain CH1/CT-tagged | 6.47 | 96.6 |
| | enfortumab | | |
| Vedotin | Wild-type enfortumab | 4.17 | 96.3 |

### Methods

Multi-dose non-GLP rat toxicology study. Male Sprague-Dawley rats (8-9 wk old at study start, 5 animals/group) were dosed intravenously with either vehicle alone or with nectin-4 conjugates made using antibodies carrying the variable regions of the rat cross-reactive antibody, enfortumab. The tested ADCs were nectin-4 vedotin ADC (Padcev, generic) and enfortumab Compound 25 ADC. Dosing at 10 mg/kg occurred weekly for a total of 4 doses (days 1, 8, 15, and 22). Animals were observed for 7 days post last dose. Body weights were recorded four times/week. Blood was collected from all animals for clinical pathology on days 5, 12, 19, and 26, and for toxicokinetic analysis at 8 h and days 4 and 7 post-dose (for all doses). Clinical observations were conducted daily. The clinical observation scoring system scale ranged from 0 (normal) to 3 (severe) is shown in Table 13.

TABLE 13
Clinical observation scoring system scale

| Parameter | 0 | 1 | 2 | 3 |
|---|---|---|---|---|
| Activity | Bright and | Minor changes, | Reduced mobility, | Comatose |
| Level/ | alert | Stereotypic | Inactive, | |
| Unprovoked | | behavior, | Huddled in cage, | |
| Behavior | | chirping | Lethargic | |
| Provoked | Inquisitive | Minor | Moderately reduced | Violent reactions, |
| Behavior | about | depression or | response, | Loud and |
| | environment | exaggeration of | Moderate | continuous |
| | | response; | vocalization, | vocalizations |
| | | Burrowing or | No exploration | |
| | | hiding, but | when lid removed | |
| | | rouses when | | |
| | | touched. | | |
| Locomotion/ | Normal | Tail | Teetering or | Inability to move, |
| Neurological | | stiff/upright, | stumbling, | Paralysis, |
| | | Tail drags, Head | Back | Dragging limbs, |
| | | tilt, | hunched/abdomen | Severe/Prolonged |
| | | Circling | tucked while | convulsions |
| | | | walking, Tremor | |
| Sneezing | none | once a minute | 2-3 times/minute | >3 times/minute |
| Respiration | Normal | Mildly | Open mouth | Severely |
| | | pronounced or | breathing, | pronounced or |
| | | reduced chest | Moderately | reduced chest |
| | | movement, | pronounced or | movement |
| | | minimal/mild, | reduced chest | |
| | | audible rales | movement, | |
| | | | moderate to severe | |
| | | | rales | |
| Posture | Normal | Head tucked | Hunched | Prostrate |
| | | down | back/tucked | |
| | | | abdomen | |
| Body | Normal | Spinal column | Noticeable | Missing anatomy, |
| Condition | | evident, | distended abdomen, | Skeletal structure |
| | | Mild edema | Moderate edema | extremely |
| | | | | prominent, |
| | | | | Distended abdomen, |
| | | | | Severe edema |
| Fur & Skin | Shiny, well | Signs of | Rough, starry coat, | Deep wounds |
| | groomed | minimal lack of | Severe piloerection, | (severe fighting |
| | coat. | grooming, | Moderate skin | lesions, |
| | | Signs of mild | lesions, | Skin ulceration, |
| | | hair loss, | Soiled anogenital | Freund's complete |
| | | Inflamed skin, | area, | adjuvant ulcer) |
| | | Mild | Anal prolapse | |
| | | piloerection | | |
| Eyes | Normal | Mild porphyrin | Obvious porphyrin | N/A |
| | | staining around | staining around eyes | |
| | | eyes | or on paws | |
| Tumors or | Normal | Small (abscess | Moderate abscess or | Large abscess or |
| Infections* | | or tumor (non- | tumor (non-cancer | tumor (non-cancer |
| *unrelated to | | cancer studies) | studies) | studies) |
| disease | | | | |
| models | | | | |
| Body Weight | <10% loss | 10-15% loss | 15-20% loss | >20% loss |
| Loss in | | | | |
| recent 7 days | | | | |

### Multi-Dose Non-GLP Rat Toxicology Study Results:

Enfortumab ADCs conjugated to either vedotin or to Compound 25 at CH1/CT-tag sites were compared for tolerability at equal payload/equal antibody dosing levels in a multi-dose rat study. The Padcev (generic) ADC was toxic to rats at the administered dose, with one animal death. Other animals in the Padcev (generic) dosing group exhibited multiple clinical observations—most fur and skin related as well as clinical pathology readouts indicating effects of the ADC on the liver and hematopoietic system. By contrast, animals receiving the Compound 25 ADC tolerated the treatment very well, with no mortalities, no clinical observations, and clinical pathology readouts that more closely mirrored the vehicle control group compared to the Padcev (generic) groups.

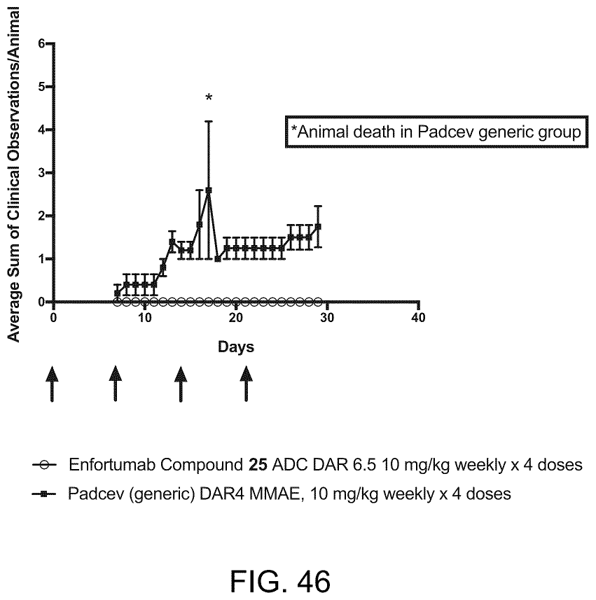

FIG. 46. Clinical observations in rats repeatedly dosed with rat cross-reactive nectin-4 ADCs. Arrows indicate dosing days. There were no observations in animals dosed with the Compound 25 conjugate, whereas the clinical observations in the vedotin dosing group averaged 2.5 on Day 17 and culminated in the death of an animal.

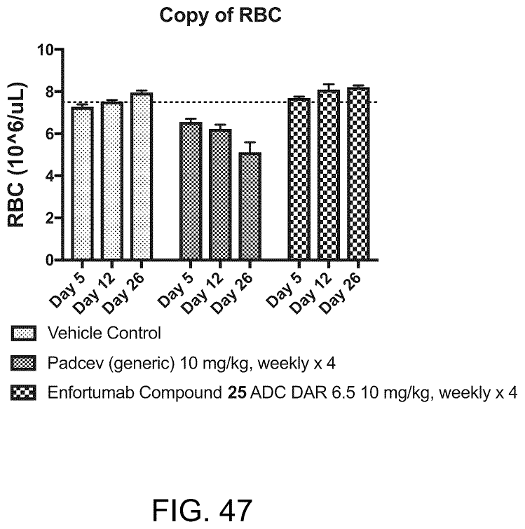

FIG. 47. Red blood cell counts in rats repeatedly dosed with vehicle or ADCs.

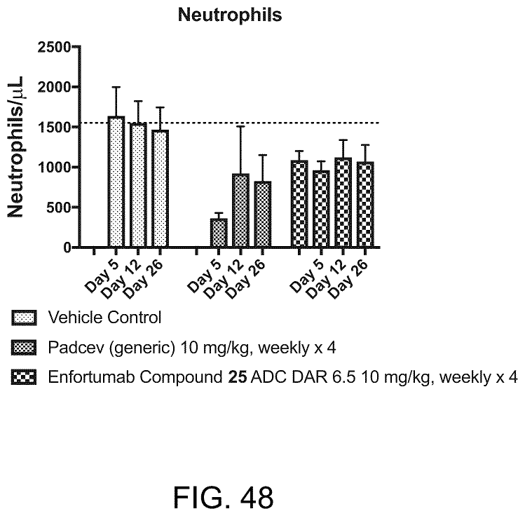

FIG. 48. Neutrophil counts in rats repeatedly dosed with vehicle or ADCs.

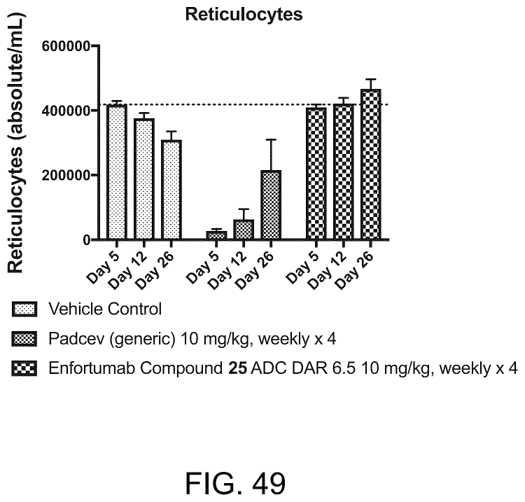

FIG. 49. Reticulocyte counts in rats repeatedly dosed with vehicle or ADCs.

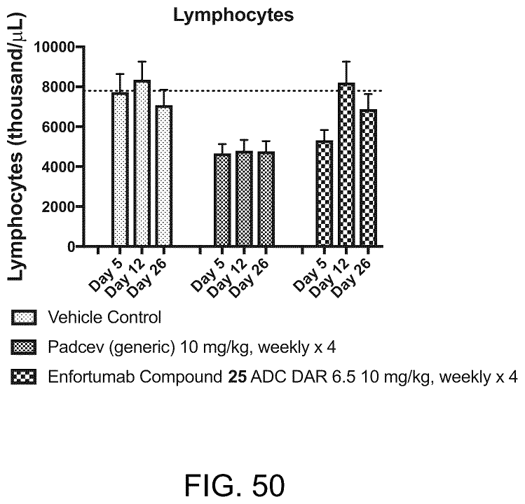

FIG. 50. Lymphocyte counts in rats repeatedly dosed with vehicle or ADCs.

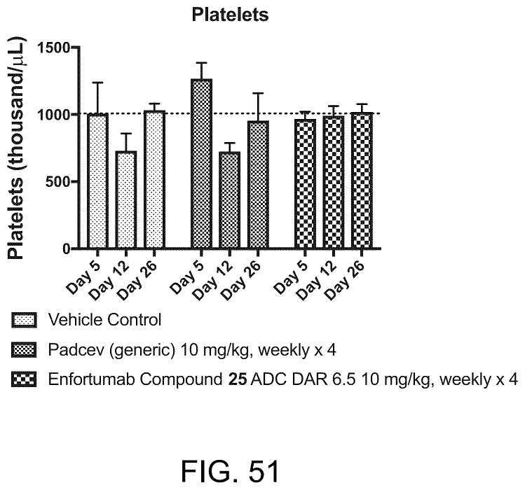

FIG. 51. Platelet counts in rats repeatedly dosed with vehicle or ADCs.

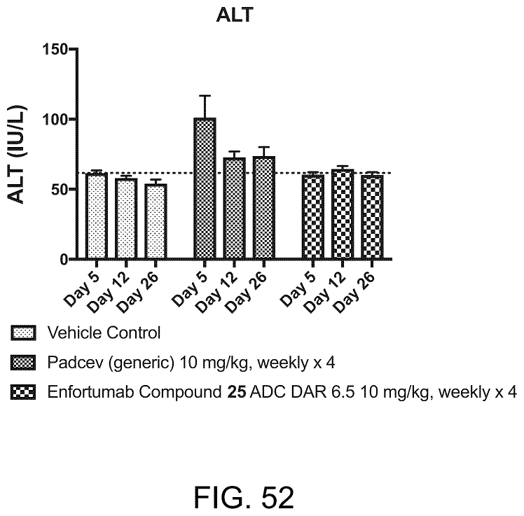

FIG. 52. Alanine amino transferase counts in rats repeatedly dosed with vehicle or ADCs.

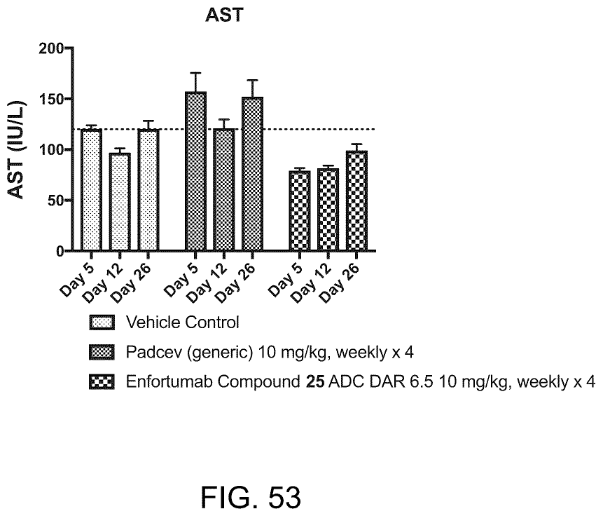

FIG. 53. Aspartate amino transferase counts in rats repeatedly dosed with vehicle or ADCs.

### Example 7: Toxicokinetic Sample Analysis

### Methods

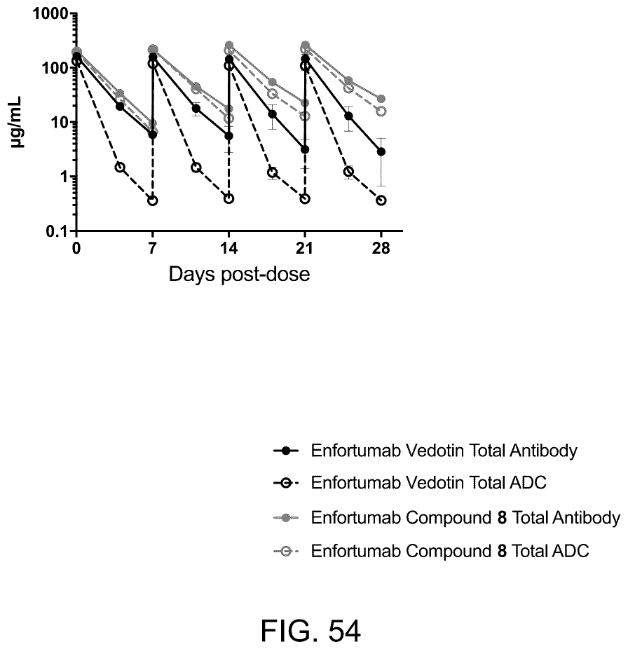

Total antibody and total ADC concentrations were quantified by ELISA as previously described and diagrammed in FIG. 54. For total antibody, conjugates were captured with an anti-human IgG-specific antibody and detected with an HRP-conjugated anti-human Fc-specific antibody. For total ADC, conjugates were captured with an anti-human Fab-specific antibody and detected with a mouse anti-maytansine primary antibody, followed by an HRP-conjugated anti-mouse IgG-subclass 1-specific secondary antibody. Bound secondary antibody was detected using Ultra TMB One-Step ELISA substrate (Thermo Fisher). After quenching the reaction with sulfuric acid, signals were read by taking the absorbance at 450 nm on a Molecular Devices Spectra Max M5 plate reader equipped with SoftMax Pro software. Data were analyzed using GraphPad Prism and Microsoft Excel software.

Results: Toxicokinetic analysis of plasma samples from animals in the Multi-dose non-GLP rat toxicology study #2 confirmed dosing levels and exposure, and demonstrated improved stability of the Compound 8 conjugate as compared to the vedotin ADC (FIG. 54).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A conjugate of formula (I): 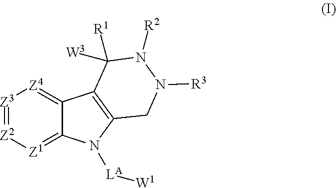
wherein:
Z¹, Z², Z³ and Z⁴ are each independently selected from CR⁴, N and C-L^{B}-W²;
R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L^{A} is a first linker;
L^{B} is a second linker;
W¹ is a first drug;
W² is a second drug; and
W³ is an anti-Nectin-4 antibody.

2. The conjugate of claim 1, wherein Z¹ is CR⁴.

3. The conjugate of claim 1, wherein Z¹ is N.

4. The conjugate of claim 1, wherein Z³ is CR⁴.

5. The conjugate of claim 1, wherein Z³ is C-L^{B}-W².

6. The conjugate of any of claims 1-5, wherein L^{A} comprises:
-(T¹-V¹)ₐ-(T²-V²)_{b}-(T³-V³)_{c}-(T⁴-V⁴)_{d}-(T⁵-V⁵)ₑ-(T⁶-V⁶)_{f}-,
wherein
a, b, c, d, e and f are each independently 0 or 1;
T¹, T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
V¹, V², V³, V⁴, V⁵ and V⁶ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, an alkyl, a substituted alkyl, an aryl, and a substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

7. The conjugate of claim 6, wherein T¹, T², T³, T⁴, T⁵ and T⁶ are each optionally substituted with a glycoside.

8. The conjugate of claim 6, wherein MABO, MABC, PABO, PABC, PAB, PABA, PAP and PHP are each optionally substituted with a glycoside.

9. The conjugate of any one of claims 7-8, wherein the glycoside is selected from a glucuronide, a galactoside, a glucoside, a mannoside, a fucoside, O-GlcNAc, and O-GalNAc.

10. The conjugate of any one of claims 6-9,
wherein:
T¹ is (C₁-C₁₂)alkyl and V¹ is —CO—;
T² is an amino acid analog and V² is —NH—;
T³ is (PEG)ₙ and V³ is —CO—;
T⁴ is AA and V⁴ is absent;
T⁵ is PABC and V⁵ is absent; and
f is 0; or
wherein:
T¹ is (C₁-C₁₂)alkyl and V¹ is —CONH—;
T² is (PEG)ₙ and V² is —CO—;
T³ is AA and V³ is absent;
T⁴ is PABC and V⁴ is absent; and
e and f are each 0; or
wherein:
T¹ is (C₁-C₁₂)alkyl and V¹ is —CONH—;
T² is substituted (C₁-C₁₂)alkyl and V² is —CO—;
T³ is AA and V³ is absent;
T⁴ is PABC and V⁴ is absent; and
e and f are each 0.

11. The conjugate of any one of claims 1-10, wherein L^{B} comprises:
-(T⁷-V⁷)_{g}-(T⁸-V⁸)ₕ-(T⁹-V⁹)ᵢ-(T¹⁰-V¹⁰)ⱼ-(T¹¹-V¹¹)ₖ-(T¹²-V¹²)ₗ-(T¹³-V¹³)ₘ-,
wherein
g, h, i, j, k, l and m are each independently 0 or 1;
T⁷, T⁸, T⁹, T¹⁰, T¹¹, T¹² and T¹³ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
V⁷, V⁸, V⁹, V¹⁰, V¹¹, V¹² and V¹³ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, an alkyl, a substituted alkyl, an aryl, and a substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

12. The conjugate of claim 11, wherein T⁷, T⁸, T⁹, T¹⁰, T¹¹, T¹² and T¹³ are each optionally substituted with a glycoside.

13. The conjugate of claim 11, wherein MABO, MABC, PABO, PABC, PAB, PABA, PAP and PHP are each optionally substituted with a glycoside.

14. The conjugate of any one of claims 12-13, wherein the glycoside is selected from a glucuronide, a galactoside, a glucoside, a mannoside, a fucoside, O-GlcNAc, and O-GalNAc.

15. The conjugate of any one of claims 11-14,
wherein:
T⁷ is absent and V⁷ is —NHCO—;
T⁸ is (C₁-C₁₂)alkyl and V⁸ is —CONH—;
T⁹ is (PEG)ₙ and V⁹ is —CO—;
T¹⁰ is AA and V¹⁰ is absent; and
T¹¹ is PABC and V¹¹ is absent; and
l and m are each 0; or
wherein:
T⁷ is absent and V⁷ is —NHCO—;
T⁸ is (C₁-C₁₂)alkyl and V⁸ is —CONH—;
T⁹ is substituted (C₁-C₁₂)alkyl and V⁹ is —CO—;
T¹⁰ is AA and V¹⁰ is absent;
T¹¹ is PABC and V¹¹ is absent; and
l and m are each 0.

16. The conjugate of any one of claims 1-15, wherein the conjugate is selected from:

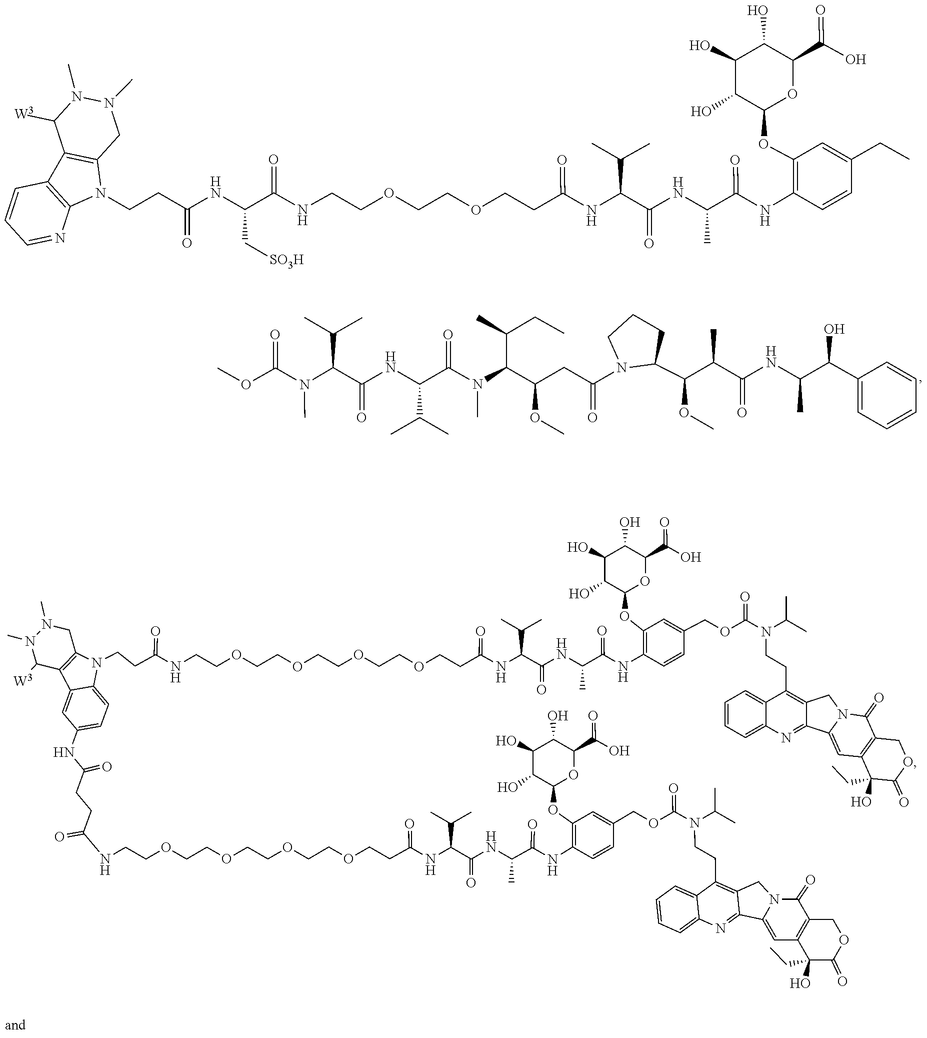

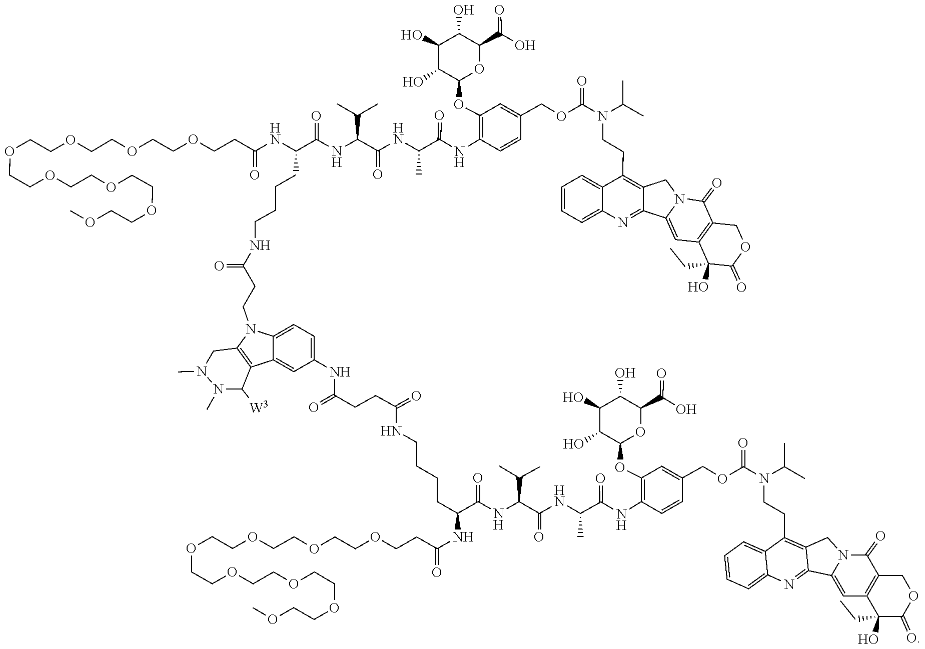

17. The conjugate of any one of claims 1 to 16, wherein the anti-Nectin-4 antibody is an IgG1 antibody.

18. The conjugate of claim 17, wherein the anti-Nectin-4 antibody is an IgG1 kappa antibody.

19. The conjugate of any one of claims 1 to 18, wherein the anti-Nectin-4 antibody comprises a sequence of the formula (II):
(II)
(SEQ ID NO: 128)
X¹(fGly′)X²Z²X³Z³,
wherein
X¹ is present or absent and, when present, can be any amino acid, with the proviso that when the sequence is at the N-terminus of the conjugate, X¹ is present;
fGly′ is an amino acid residue coupled to the first drug or the second drug through the first linker or the second linker, respectively;
X² and X³ are each independently any amino acid;
Z² is either a proline or alanine residue; and
Z³ is a basic amino acid or an aliphatic amino acid.

20. The conjugate of claim 19, wherein the sequence is L(fGly′)TPSR (SEQ ID NO: 246).

21. The conjugate of claim 19, wherein
Z³ is selected from R, K, H, A, G, L, V, I, and P;
X¹ is selected from L, M, S, and V; and
X² and X³ are each independently selected from S, T, A, V, G, and C.

22. The conjugate of any one of claims 19 to 21, wherein the sequence is positioned at a C-terminus of a heavy chain constant region of the anti-Nectin-4 antibody.

23. The conjugate of claim 22, wherein the heavy chain constant region comprises a sequence of the formula (II):
(II)
(SEQ ID NO: 128)
X¹(fGly′)X²Z²X³Z³,
wherein
X¹ is present or absent and, when present, can be any amino acid, with the proviso that when the sequence is at the N-terminus of the conjugate, X¹ is present;
fGly′ is an amino acid residue coupled to the first drug or the second drug through the first linker or the second linker, respectively
X² and X³ are each independently any amino acid;
Z² is either a proline or alanine residue;
Z³ is a basic amino acid or an aliphatic amino acid, and
wherein the sequence is C-terminal to the amino acid sequence SLSLSPG (SEQ ID NO: 247).

24. The conjugate of claim 23, wherein the heavy chain constant region comprises the sequence SPGSL(fGly′)TPSRGS (SEQ ID NO: 130).

25. The conjugate of claim 23, wherein
Z³ is selected from R, K, H, A, G, L, V, I, and P;
X¹ is selected from L, M, S, and V; and
X² and X³ are each independently selected from S, T, A, V, G, and C.

26. The conjugate of any one of claims 22 to 25, wherein the heavy chain constant region of the anti-Nectin-4 antibody comprises an amino acid sequence at least 85%, at least 90%, at least 95%, at least 96%, at least 97%, at least 98%, or at least 99% identical to the amino acid sequence set forth in a sequence selected from SEQ ID NOs: 70 and comprises the fGly′ residue instead of C in the sequence LCTPSR (SEQ ID NO: 104).

27. The conjugate of any one of claims 19 to 21, wherein the heavy chain constant region of the anti-Nectin-4 antibody comprises an amino acid sequence at least 85%, at least 90%, at least 95%, at least 96%, at least 97%, at least 98%, or at least 99% identical to the amino acid sequence set forth in any one of SEQ ID NOs: 71, 75, 79, and 83 and comprises the fGly′ residue instead of C in the sequence LCTPSR (SEQ ID NO: 104).

28. The conjugate of any one of claims 19 to 21, wherein the heavy chain constant region of the anti-Nectin-4 antibody comprises an amino acid sequence at least 85%, at least 90%, at least 95%, at least 96%, at least 97%, at least 98%, or at least 99% identical to the amino acid sequence set forth in any one of SEQ ID NOs: 72, 76, 80, and 84 and comprises the fGly′ residue instead of C in the sequence LCTPSR (SEQ ID NO: 104).

29. The conjugate of any one of claims 19 to 21, wherein the heavy chain constant region of the anti-Nectin-4 antibody comprises an amino acid sequence at least 85%, at least 90%, at least 95%, at least 96%, at least 97%, at least 98%, or at least 99% identical to the amino acid sequence set forth in any one of SEQ ID NOs: 73, 77, 81, and 85 and comprises the fGly′ residue instead of C in the sequence LCTPSR (SEQ ID NO: 104).

30. The conjugate of any one of claims 19 to 21, wherein the heavy chain constant region of the anti-Nectin-4 antibody comprises an amino acid sequence at least 85%, at least 90%, at least 95%, at least 96%, at least 97%, at least 98%, or at least 99% identical to the amino acid sequence set forth in any one of SEQ ID NOs: 74, 78, 82, and 86 and comprises the fGly′ residue instead of C in the sequence LCTPSR (SEQ ID NO: 104).

31. The conjugate of any one of claims 19 to 21, wherein the fGly′ residue is positioned in a light chain constant region of the anti-Nectin-4 antibody.

32. The conjugate of claim 31, wherein the light chain constant region comprises a sequence of the formula (II):
(II)
(SEQ ID NO: 128)
X¹(fGly′)X²Z²X³Z³,
wherein
X¹ is present or absent and, when present, can be any amino acid, with the proviso that when the sequence is at the N-terminus of the conjugate, X¹ is present;
fGly′ is the amino acid residue coupled to the drug through the linker;
X² and X³ are each independently any amino acid;
Z² is either a proline or alanine residue;
Z³ is a basic amino acid or an aliphatic amino acid, and
wherein the sequence is C-terminal to the amino acid sequence KVDNAL (SEQ ID NO: 132), and/or is N-terminal to the sequence QSGNSQ (SE ID NO: 133).

33. The conjugate of claim 32, wherein the light chain constant region comprises the sequence KVDNAL(fGly′)TPSRQSGNSQ (SEQ ID NO: 134).

34. The conjugate of claim 33, wherein
Z³ is selected from R, K, H, A, G, L, V, I, and P;
X¹ is selected from L, M, S, and V; and
X² and X³ are each independently selected from S, T, A, V, G, and C.

35. The conjugate of any one of claims 19 to 21, wherein the fGly′ residue is positioned in a heavy chain CH1 region of the anti-Nectin-4 antibody.

36. The conjugate of claim 35, wherein the light chain constant region comprises a sequence of the formula (II):
(II)
(SEQ ID NO: 128)
X¹(fGly′)X²Z²X³Z³,
wherein
X¹ is present or absent and, when present, can be any amino acid, with the proviso that when the sequence is at the N-terminus of the conjugate, X¹ is present;
fGly′ is the amino acid residue coupled to the drug through the linker;
X² and X³ are each independently any amino acid;
Z² is either a proline or alanine residue;
Z³ is a basic amino acid or an aliphatic amino acid, and
wherein the sequence is C-terminal to the amino acid sequence SWNSGA (SEQ ID NO: 135) and/or is N-terminal to the amino acid sequence GVHTFP (SEQ ID NO: 136).

37. The conjugate of claim 36, wherein the heavy chain CH1 region comprises the sequence SWNSGAL(fGly′)TPSRGVHTFP (SEQ ID NO: 137).

38. The conjugate of claim 29, wherein
Z³ is selected from R, K, H, A, G, L, V, I, and P;
X¹ is selected from L, M, S, and V; and
X² and X³ are each independently selected from S, T, A, V, G, and C.

39. The conjugate of any one of claims 19 to 21, wherein the fGly′ residue is positioned in a heavy chain CH2 region of the anti-Nectin-4 antibody.

40. The conjugate of any one of claims 19 to 21, wherein the fGly′ residue is positioned in a heavy chain CH3 region of the anti-Nectin-4 antibody.

41. The conjugate of any one of claims 1 to 40, wherein the anti-Nectin-4 antibody competes for binding to Nectin-4 with an anti-Nectin-4 antibody comprising:
a variable heavy chain (VH) chain comprising heavy chain CDRs 1-3 (HCDRs 1-3) of a VH chain having a sequence selected from SEQ ID NOs: 1 to 17; and
a variable light chain (VL) chain comprising light chain CDRs 1-3 (LCDRs 1-3) of a VL chain having a sequence selected from SEQ ID NOs: 18 to 31.

42. The conjugate of any one of claims 1 to 40, wherein the anti-Nectin-4 antibody comprises:
a VH chain comprising heavy chain CDRs 1-3 (HCDRs 1-3) of a VH chain having a sequence selected from SEQ ID NOs: 1 to 17; and
a VL chain comprising light chain CDRs 1-3 (LCDRs 1-3) of a VL chain having a sequence selected from SEQ ID NOs: 18 to 31.

43. The conjugate of claim 42, wherein the antibody that specifically binds to Nectin-4 comprises:
a VH chain comprising a sequence selected from SEQ ID NOs: 1 to 17; and
a VL chain comprising a sequence selected from SEQ ID NOs: 18 to 31.

44. The conjugate of claim 42, wherein the antibody that specifically binds to Nectin-4 comprises:
a VH chain comprising HCDRs 1-3 of a VH chain having a sequence selected from SEQ ID NOs: 1 to 6; and
a VL chain comprising LCDRs 1-3 of a VL chain having a sequence selected from SEQ ID NOs: 18 to 23.

45. The conjugate of claim 42, wherein the antibody that specifically binds to Nectin-4 comprises:
a VH chain comprising a sequence selected from SEQ ID NOs: 1 to 6; and
a VL chain comprising a sequence selected from SEQ ID NOs: 18 to 23.

46. The conjugate of claim 42, wherein the antibody that specifically binds to Nectin-4 comprises:
a VH chain comprising HCDRs 1-3 of a VH chain having a sequence selected from SEQ ID NOs: 7 to 13; and
a VL chain comprising LCDRs 1-3 of a VL chain having a sequence selected from SEQ ID NOs: 24 to 27.

47. The conjugate of claim 42, wherein the antibody that specifically binds to Nectin-4 comprises:
a VH chain comprising a sequence selected from SEQ ID NOs: 7 to 13; and
a VL chain comprising a sequence selected from SEQ ID NOs: 24 to 27.

48. The conjugate of claim 42, wherein the antibody that specifically binds to Nectin-4 comprises:
a VH chain comprising HCDRs 1-3 of a VH chain having a sequence selected from SEQ ID NOs: 14 to 17; and
a VL chain comprising LCDRs 1-3 of a VL chain having a sequence selected from SEQ ID NOs: 28 to 31.

49. The conjugate of claim 42, wherein the antibody that specifically binds to Nectin-4 comprises:
a VH chain comprising a sequence selected from SEQ ID NOs: 14 to 17; and
a VL chain comprising a sequence selected from SEQ ID NOs: 28 to 31.

50. The conjugate of claim 42, wherein the antibody that specifically binds to Nectin-4 comprises:
the VH chain of an anti-Nectin-4 antibody comprising the HCDRs 1-3 of a VH chain having a sequence selected from SEQ ID NOs: 1 to 17 and an amino acid sequence having 80% or greater, 85% or greater, 90% or greater, 95% or greater, 99% or greater, or 100% sequence identity to the amino acid sequence set forth in a sequence selected from SEQ ID NOs:1 to 17, wherein any amino acid differences between the VH chain of an anti-Nectin-4 antibody and a sequence selected from SEQ ID NOs: 1 to 17 is in the regions outside of the CDRs; and
the VL chain of an anti-Nectin-4 antibody comprises the LCDRs 1-3 of a VL chain having a sequence selected from SEQ ID NOs: 18 to 31 and comprises an amino acid sequence having 80% or greater, 85% or greater, 90% or greater, 95% or greater, 99% or greater, or 100% sequence identity to the amino acid sequence set forth in a sequence selected from SEQ ID NOs: 18 to 31, wherein any amino acid differences between the VL chain of an anti-Nectin-4 antibody and a sequence selected from SEQ ID NOs: 18 to 31 is within the regions outside of the CDRs.

51. The conjugate of any one of claims 42-50, wherein the anti-Nectin-4 antibody comprises: a heavy chain constant region having the amino acid sequence set forth in any one of SEQ ID NOs: 70 to 86, wherein the C present in the sequence LCTPSR in the constant region is replaced by fGly.

52. A pharmaceutical composition comprising:
a conjugate of any one of claims 1 to 51; and
a pharmaceutically-acceptable excipient.

53. A method comprising:
administering to a subject an effective amount of the conjugate of any one of claims 1 to 51 or the pharmaceutical composition of claim 52.

54. A method of treating cancer in a subject, the method comprising:
administering to the subject a therapeutically effective amount of the conjugate of any one of claims 1 to 51 or the pharmaceutical composition of claim 52, wherein the administering is effective to treat cancer in the subject.

55. The method according to claim 54, wherein the cancer is ovarian cancer, ductal breast carcinoma, lung adenocarcinoma, and pancreatic cancer.

56. The method according to claim 55, wherein the cancer is characterized by cancer cells expressing Nectin-4.

57. The method according to claim 56, wherein the conjugate binds to Nectin-4.

58. A method of delivering a drug to a target site in a subject, the method comprising:
administering to the subject the conjugate of any one of claims 1 to 51 or the pharmaceutical composition of claim 52, wherein the administering is effective to release a therapeutically effective amount of the drug from the conjugate at the target site in the subject.

59. An anti-Nectin-4 antibody, comprising:
a variable heavy chain (VH) chain comprising heavy chain CDRs 1-3 (HCDRs 1-3) of a VH chain having a sequence selected from SEQ ID NOs: 1 to 17; and
a variable light chain (VL) chain comprising light chain CDRs 1-3 (LCDRs 1-3) of a VL chain having a sequence selected from SEQ ID NOs: 18 to 31.

60. The anti-Nectin-4 antibody of claim 59, comprising:
a VH chain comprising a sequence selected from SEQ ID NOs: 1 to 17; and
a VL chain comprising a sequence selected from SEQ ID NOs: 18 to 31.

61. The anti-Nectin-4 antibody of claim 59, comprising:
a VH chain comprising HCDRs 1-3 of a VH chain having a sequence selected from SEQ ID NOs: 1 to 6; and
a VL chain comprising LCDRs 1-3 of a VL chain having a sequence selected from SEQ ID NOs: 18 to 23.

62. The anti-Nectin-4 antibody of claim 59, comprising:
a VH chain comprising a sequence selected from SEQ ID NOs: 1 to 6; and
a VL chain comprising a sequence selected from SEQ ID NOs: 18 to 23.

63. The anti-Nectin-4 antibody of claim 59, comprising:
a VH chain comprising HCDRs 1-3 of a VH chain having a sequence selected from SEQ ID NOs: 7 to 13; and
a VL chain comprising LCDRs 1-3 of a VL chain having a sequence selected from SEQ ID NOs: 24 to 27.

64. The anti-Nectin-4 antibody of claim 59, comprising:
a VH chain comprising a sequence selected from SEQ ID NOs: 7 to 13; and
a VL chain comprising a sequence selected from SEQ ID NOs: 24 to 27.

65. The anti-Nectin-4 antibody of claim 59, comprising:
a VH chain comprising HCDRs 1-3 of a VH chain having a sequence selected from SEQ ID NOs: 14 to 17; and
a VL chain comprising LCDRs 1-3 of a VL chain having a sequence selected from SEQ ID NOs: 28 to 31.

66. The anti-Nectin-4 antibody of claim 59, comprising:
a VH chain comprising a sequence selected from SEQ ID NOs: 14 to 17; and
a VL chain comprising a sequence selected from SEQ ID NOs: 28 to 31.

67. The anti-Nectin-4 antibody of claim 59, comprising:
the VH chain of an anti-Nectin-4 antibody comprising the HCDRs 1-3 of a VH chain having a sequence selected from SEQ ID NOs: 1 to 17 and an amino acid sequence having 80% or greater, 85% or greater, 90% or greater, 95% or greater, 99% or greater, or 100% sequence identity to the amino acid sequence set forth in a sequence selected from SEQ ID NOs: 1 to 17, wherein any amino acid differences between the VH chain of an anti-Nectin-4 antibody and a sequence selected from SEQ ID NOs: 1 to 17 is in the regions outside of the CDRs; and
the VL chain of an anti-Nectin-4 antibody comprises the LCDRs 1-3 of a VL chain having a sequence selected from SEQ ID NOs: 18 to 31 and comprises an amino acid sequence having 80% or greater, 85% or greater, 90% or greater, 95% or greater, 99% or greater, or 100% sequence identity to the amino acid sequence set forth in a sequence selected from SEQ ID NOs: 18 to 31, wherein any amino acid differences between the VL chain of an anti-Nectin-4 antibody and a sequence selected from SEQ ID NOs: 18 to 31 is within the regions outside of the CDRs.

68. The anti-Nectin-4 antibody of any one of claims 59-67, comprising: a heavy chain constant region having the amino acid sequence set forth in any one of SEQ ID NOs: 70 to 86, wherein the C present in the sequence LCTPSR in the constant region is replaced by fGly.

69. A pharmaceutical composition comprising:
an antibody of any one of claims 59 to 68; and
a pharmaceutically-acceptable excipient.

70. A method comprising:
administering to a subject an effective amount of the antibody of any one of claims 59 to 68 or the pharmaceutical composition of claim 69.

71. A method of treating cancer in a subject, the method comprising:
administering to the subject a therapeutically effective amount of the antibody of any one of claims 59 to 68 or the pharmaceutical composition of claim 69, wherein the administering is effective to treat cancer in the subject.

72. The method according to claim 71, wherein the cancer is ovarian cancer, ductal breast carcinoma, lung adenocarcinoma, and pancreatic cancer.

73. The method according to claim 72, wherein the cancer is characterized by cancer cells expressing Nectin-4.

74. The method according to claim 73, wherein the conjugate binds to Nectin-4.
